(19) United States
(12) Patent Application Publication
(10) Pub. No.: US 2018/0282323 A1
(43) Pub. Date: Oct. 4, 2018

(54) Mesoionic imidazole derivatives as insecticides

(71) Applicant: BAYER CROPSCIENCE AKTIENGESELLSCHAFT, Monheim am Rhein (DE)

(72) Inventors: Markus HEIL, Leichlingen (DE); Daniela PORTZ, Vettweiß (DE); Kerstin ILG, Köln (DE); Ulrich GÖRGENS, Ratingen (DE); Silvia CEREZO-GALVEZ, Langenfeld (DE); Marc MOSRIN, Köln (DE); Sascha EILMUS, Leichlingen (DE); Andreas TURBERG, Haan (DE)

(21) Appl. No.: 15/780,883

(22) PCT Filed: Nov. 29, 2016
(86) PCT No.: PCT/EP2016/079071

§ 371 Date: Jun. 1, 2018

(30) Foreign Application Priority Data
Dec. 3, 2015 (EP) 15197803.8
Dec. 11, 2015 (EP) 15199457.1

(51) Int. Cl. C07D 471/04; C07D 487/04; C07D 513/04; A01N 43/90

(52) U.S. Cl. CPC C07D 471/04; C07D 487/04; A01N 25/30; A01N 43/90; C07D 513/04

(57) ABSTRACT

What are demonstrated are compounds of the formula (I)

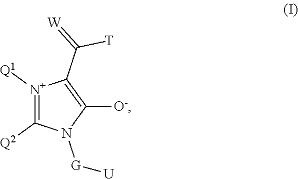

which are suitable for controlling animal pests, including arthropods and especially insects, arachnids and nematodes, and in which the structural elements Q¹, Q², W, T, Y, G and U have the definitions given in the description, as are processes for preparation thereof and the use thereof as insecticides.

The present invention relates to novel mesoionic imidazole derivatives, to processes for preparation thereof and to the use thereof for controlling animal pests, particularly arthropods and especially insects, arachnids and nematodes.

Particular mesoionic imidazole derivatives are already known; see, for example, J. Chem. Soc. Perkin Trans I, 1984, 69-73; Tetrahedron 1990, 46, 6033-6046; Can. J. Chem. 1971, 49, 668-671; Tetrahedron 1986, 42, 1169-1177; Tetrahedron 2009, 65, 7591-7596; Tetrahedron 1998, 54, 9689-9700; Acta Crystallographica, Section E: Structure Reports Online (2011), 67(10), 2814. No biological action has been described in the publications cited. Some examples are cited in the following table:

| Structure | CAS-No./Lit. |
|---|---|
| 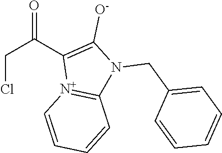 [SMILES: O=C(CCl)c1c([O-])n(Cc2ccccc2)c2cccc[n+]12] | 31757-80-9 Canadian Journal of Chemistry (1971), 49(4), 668-671 |
| 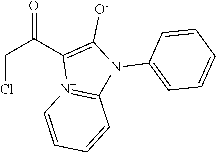 [SMILES: O=C(CCl)c1c([O-])n(-c2ccccc2)c2cccc[n+]12] | 31757-81-0 Canadian Journal of Chemistry (1971), 49(4), 668-671 |
| 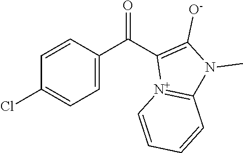 [SMILES: Cn1c([O-])c(C(=O)c2ccc(Cl)cc2)[n+]2ccccc12] | 1352167-00-0 Acta Crystallographica, Section E: Structure Reports Online (2011), 67(10), 2814 |
| 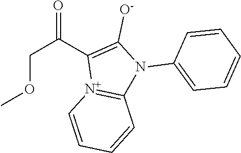 [SMILES: COCC(=O)c1c([O-])n(-c2ccccc2)c2cccc[n+]12] | 1190334-13-4 Tetrahedron (2009), 65(36), 7591-7596 |
| 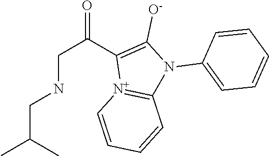 [SMILES: CC(C)CNCC(=O)c1c([O-])n(-c2ccccc2)c2cccc[n+]12] | 1190334-08-7 Tetrahedron (2009), 65(36), 7591-7596 |

Modern crop protection compositions have to meet many demands, for example in relation to the level, duration and spectrum of their action and possible use. Questions of toxicity and of combinability with other active ingredients or formulation auxiliaries play a role, as does the question of the expense that the synthesis of an active ingredient requires. In addition, resistances can occur. For all these reasons, the search for novel crop protection agents can never be considered to be complete, and there is a constant need for novel compounds having properties improved over the known compounds at least in relation to individual aspects.

It was an object of the present invention to provide compounds which broaden the spectrum of the pesticides in various aspects and/or improve their activity.

It has now been found that, surprisingly, particular novel mesoionic imidazole derivatives have significant insecticidal properties and are at the same time well tolerated by plants and have favourable homeotherm toxicity and good environmental compatibility. The novel compounds according to the invention have not been disclosed to date.

The present invention therefore provides compounds of the general formula (I)

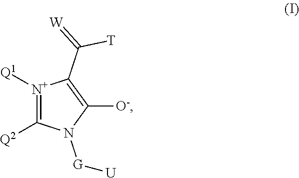

in which (Embodiment 1-2):
Q¹ and Q² together are one of the A-1 to A-5 radicals which, together with the carbon and nitrogen atom to which they are bonded, form a 5- or 6-membered aromatic ring,

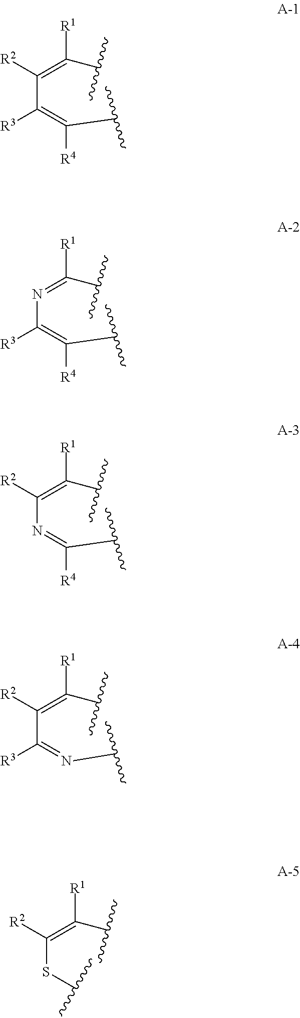

T is R⁵, —OR⁶, —N(R⁷)(R⁸) or —N(R^{7a})—N(R⁷)(R⁸)
   and
W is O,
   or
T is —N(R⁷)(R⁸) or —N(R^{7a})—N(R⁷)(R⁸)
   and
W is S,
G is a bond, is —C(R⁹)(R¹⁰)—, or is C(R⁹)(R¹⁰)C(R^{9a})(R^{10a}),
U is a cycle from the group of U-1 to U-28

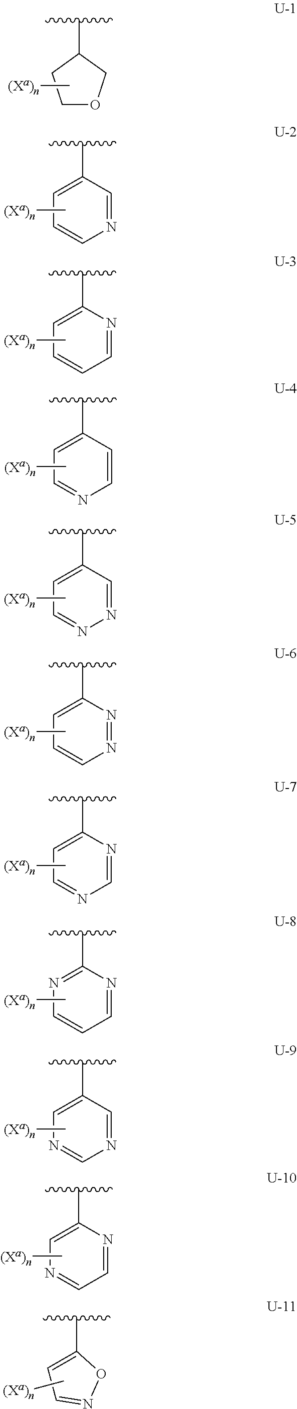

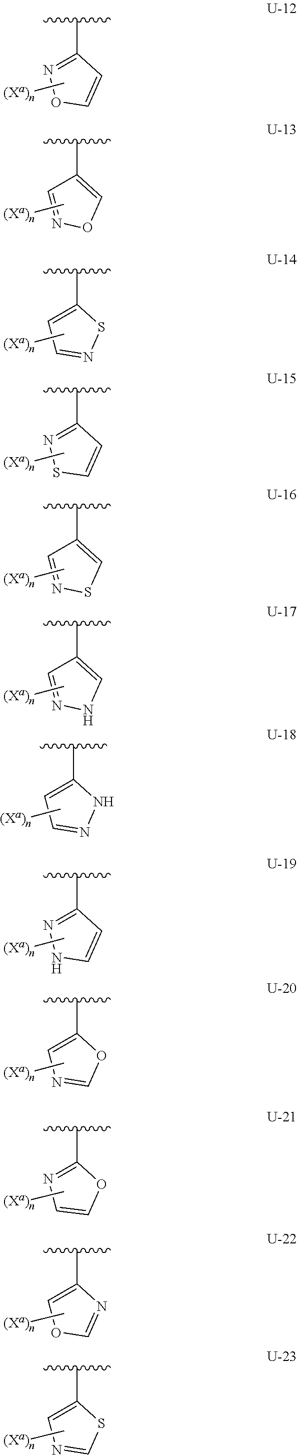

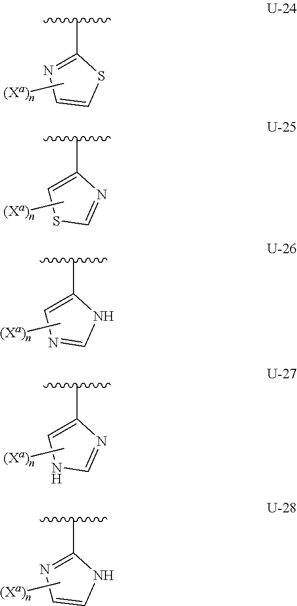

X^{a} is halogen, nitro, OH, cyano, SCN, SF₅, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkoxyimino-C₁-C₆-alkyl, C₁-C₆-alkylsulphinyl, C₁-C₆-alkylsulphonyl, C₁-C₆-haloalkylsulphonyl, C₁-C₆-alkylaminocarbonyl or di-(C₁-C₆)-alkylaminocarbonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy and where the ring nitrogen atoms in U-17, U-18, U-19, U-26, U-27 and U-28 are not substituted by halogen, nitro, OH, cyano, SCN, SF₅, C₁-C₆-alkoxy, C₁-C₆-haloalkoxy, C₁-C₆-alkylthio, C₁-C₆-alkylsulphinyl or C₁-C₄-alkoxy-C₁-C₄-alkyloxy,
n is 0, 1, 2 or 3,
R¹, R², R³, R⁴ are each independently hydrogen, halogen, cyano, nitro, SF₅, SCN, amino, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₂-C₆-alkenyloxy, C₃-C₆-alkynyl, C₃-C₆-alkynyloxy, SH, C₁-C₆-alkylthio, C₁-C₆-alkylsulphinyl, C₁-C₆-alkylsulphonyl, C₁-C₆-haloalkylsulphonyl, C₁-C₆-alkylcarbonyl, C₃-C₆-cycloalkylcarbonyl, C₁-C₆-haloalkylcarbonyl, C₁-C₆-alkoxyimino-C₁-C₆-alkyl, C₁-C₆-alkoxycarbonyl, C₁-C₆-alkylaminocarbonyl, di-(C₁-C₆)-alkylaminocarbonyl, aryl, hetaryl, aryl-C₁-C₆-alkyl or hetaryl-C₁-C₆-alkyl,
   where aryl, hetaryl, aryl-C₁-C₆-alkyl and hetaryl-C₁-C₆-alkyl may optionally be mono- or polysubstituted identically or differently by halogen, cyano, nitro, hydroxyl, C₁-C₆-alkyl, C₂-C₆-alkenyl, C₂-C₆-alkynyl, C₃-C₆-cycloalkyl, C₁-C₆-alkoxy, C₁-C₆-haloalkyl, C₁-C₆-haloalkoxy or C₁-C₆-alkylthio,
   or
R¹ and R² or
R² and R³ or
R³ and R⁴ in each case together form a 5- or 6-membered aliphatic, aromatic, heteroaromatic or heterocyclic ring which may optionally contain 1 to 2 atoms from the group of O, S and N and which may optionally be mono- or polysubstituted by halogen or by C₁-C₄-alkyl,
R⁵ is hydrogen or
   is C₁-C₆-alkyl, C₂-C₆-alkenyl or C₂-C₆-alkynyl, where the aforementioned radicals may optionally independently be mono- to pentasubstituted by halogen or monosubstituted by nitro, cyano, C₃-C₆-cycloalkyl, —O—R⁵¹, —S(O)ₚ—R⁵², —N(R⁵³)(R⁵⁴), —C(═O)N(R⁵³)(R⁵⁴), —O—C(═O)—R⁵⁵, —C(═O)—R⁵⁵, O—SO₂—R⁵⁶, aryl, hetaryl, heterocyclyl or oxoheterocyclyl, where aryl, hetaryl, heterocyclyl or oxoheterocyclyl may in turn each be mono- to trisubstituted by halogen, nitro, cyano, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, C₁-C₆-alkylcarbonylamino, aryl or hetaryl, where aryl and hetaryl may in turn optionally be mono- or polysubstituted identically or differently by halogen, cyano, nitro, hydroxyl, C₁-C₆-alkyl, C₂-C₆-alkenyl, C₂-C₆-alkynyl, C₁-C₆-alkoxy, C₁-C₆-haloalkyl, C₁-C₆-haloalkoxy or C₁-C₆-alkylthio,
   or
R⁵ is C₃-C₆-cycloalkyl or C₃-C₆-heterocyclyl, where the aforementioned radicals may optionally independently be mono- to trisubstituted by halogen, or monosubstituted by cyano, C₁-C₆-alkyl, C₁-C₆-haloalkyl, C₁-C₆-alkoxy, C₁-C₆-haloalkoxy, C₃-C₆-cycloalkyl, C₁-C₆-alkylthio, C₁-C₆-alkylsulphinyl, C₁-C₆-alkylsulphonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy,
   or
R⁵ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
p is 0, 1 or 2,
R⁵¹ is hydrogen or
   is C₁-C₆-alkyl, C₂-C₆-alkenyl, C₂-C₆-alkynyl, C₃-C₆-cycloalkyl or C₃-C₆-heterocyclyl, where the aforementioned radicals may optionally independently be mono- to trisubstituted by halogen or monosubstituted by nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
   or
R⁵¹ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵² is C₁-C₆-alkyl, C₂-C₆-alkenyl, C₂-C₆-alkynyl, C₃-C₆-cycloalkyl or C₃-C₆-heterocyclyl, where the aforementioned radicals may optionally be mono- to trisubstituted and the substituents are independently selected from halogen, nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbony, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
   or
R⁵² is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵³ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl, C₁-C₆-alkylcarbonyl, aryl, hetaryl, arylcarbonyl or hetarylcarbonyl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
R⁵⁴ is hydrogen or
   is C₁-C₆-alkyl, C₂-C₆-alkenyl, C₂-C₆-alkynyl, C₃-C₆-cycloalkyl or C₃-C₆-cycloalkyl-C₁-C₆-alkyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₆-alkoxycarbonyl, C₁-C₆-alkylcarbonyl or C₃-C₆-trialkylsilyl,
   or
R⁵⁴ is aryl, hetaryl, aryl-C₁-C₆-alkyl or heteroaryl-C₁-C₆-alkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
   or
R⁵³ and R⁵⁴ may be joined to one another via two to six carbon atoms and form a ring which optionally additionally contains a further atom from the group of O, S and N and may optionally be mono- to tetrasubstituted by C₁-C₂-alkyl, halogen, cyano, amino or C₁-C₂-alkoxy,
R⁵⁵ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁵⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl or aryl-C₁-C₆-alkyl, where aryl, hetaryl and aryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁷ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl or C₁-C₆-alkylcarbonyl,
R^{7a} is hydrogen, C₁-C₆-alkyl, C₂-C₆-alkenyl, C₂-C₆-alkynyl, C₃-C₆-cycloalkyl, C₁-C₄-alkoxy-C₁-C₆-alkyl, C₁-C₆-alkoxycarbonyl or C₁-C₆-alkylcarbonyl,
R⁸ is hydrogen or
   is C₁-C₆-alkyl, C₂-C₆-alkenyl or C₂-C₆-alkynyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₁-C₄-alkylsulphimino-C₁-C₄-alkyl, C₁-C₄-alkylsulphimino-C₂-C₅-alkylcarbonyl, C₁-C₄-alkylsulphoximino, C₁-C₄-alkylsulphoximino-C₁-C₄-alkyl, C₁-C₄-alkylsulphoximino-C₂-C₅-alkylcarbonyl, C₁-C₆-alkoxycarbonyl, C₁-C₆-alkylcarbonyl or C₃-C₆-trialkylsilyl,
   or
R⁸ is aryl-C₁-C₆-alkyl, heteroaryl-C₁-C₆-alkyl, C₃-C₁₂-cycloalkyl, C₃-C₁₂-cycloalkyl-C₁-C₆-alkyl or C₄-C₁₂-bicycloalkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
   or
R⁸ is an optionally singly or multiply, identically or differently substituted 5- or 6-membered aromatic or heteroaromatic ring, a 4- to 6-membered, partly saturated ring or saturated heterocyclic ring, or a saturated or aromatic heterobicyclic ring which may optionally contain 1 to 3 heteroatoms from the group of O, S or N and which may optionally be mono- or polysubstituted, where the substituents are independently selected from halogen, cyano, nitro, hydroxyl, amino, C₁-C₆-alkyl, C₂-C₆-alkynyl, C₁-C₆-haloalkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
   or
R⁷ and R⁸ may be joined to one another via two to six carbon atoms and form a ring which optionally additionally contains a further nitrogen, sulphur or oxygen atom and may optionally be mono- to tetrasubstituted by C₁-C₂-alkyl, halogen, cyano, amino or C₁-C₂-alkoxy,
R⁹ is hydrogen, fluorine, chlorine, C₁-C₄-alkyl, C₃-C₆-cycloalkyl or C₁-C₄-haloalkyl,
R^{9a} is hydrogen, fluorine, chlorine, C₁-C₄-alkyl, C₃-C₆-cycloalkyl or C₁-C₄-haloalkyl,
R¹⁰ is hydrogen, fluorine, chlorine or C₁-C₄-alkyl,
   and
R^{1a} is hydrogen, fluorine, chlorine or C₁-C₄-alkyl.

The compounds of the formula (I) likewise encompass any diastereomers or enantiomers and E/Z isomers which exist, and also salts and N-oxides of compounds of the formula (I), and the use thereof for control of animal pests.

The substituted mesoionic imidazole derivatives are defined in general terms by the formula (I). Preferred radical definitions for the formulae specified above and hereinafter are given below.

A further configuration (Configuration 1-1) is that of compounds of the formula (I) in which
Q¹ and Q² together are one of the A-1 to A-5 radicals which, together with the carbon and nitrogen atom to which they are bonded, form a 5- or 6-membered aromatic ring,
T is R⁵, —OR⁶, —N(R⁷)(R⁸) or —N(R^{7a})—N(R⁷)(R⁸)
   and
W is O,
   or
T is —N(R^{T})(R⁸) or —N(R^{7a})—N(R⁷)(R⁸)
   and
W is S,
G is a bond, is —C(R⁹)(R¹⁰)—, or is C(R⁹)(R¹⁰)C(R^{9a})(R^{10a}),
U is a cycle from the group of U-1 to U-28,
X^{a} is halogen, nitro, OH, cyano, SCN, SF₅, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkoxyimino-C₁-C₆-alkyl, C₁-C₆-alkylsulphinyl, C₁-C₆-alkylsulphonyl, C₁-C₆-haloalkylsulphonyl, C₁-C₆-alkylaminocarbonyl or di-(C₁-C₆)-alkylaminocarbonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy and where the ring nitrogen atoms in U-17, U-18, U-19, U-26, U-27 and U-28 are not substituted by halogen, nitro, OH, cyano, SCN, SF₅, C₁-C₆-alkoxy, C₁-C₆-haloalkoxy, C₁-C₆-alkylthio, C₁-C₆-alkylsulphinyl or C₁-C₄-alkoxy-C₁-C₄-alkyloxy,
n is 0, 1, 2 or 3,
R¹, R², R³, R⁴ are each independently hydrogen, halogen, cyano, nitro, SF₅, SCN, amino, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₂-C₆-alkenyloxy, C₃-C₆-alkynyl, C₃-C₆-alkynyloxy, SH, C₁-C₆-alkylthio, C₁-C₆-alkylsulphinyl, C₁-C₆-alkylsulphonyl, C₁-C₆-haloalkylsulphonyl, C₁-C₆-alkylcarbonyl, C₃-C₆-cycloalkylcarbonyl, C₁-C₆-haloalkylcarbonyl, C₁-C₆-alkoxyimino-C₁-C₆-alkyl, C₁-C₆-alkoxycarbonyl, C₁-C₆-alkylaminocarbonyl, di-(C₁-C₆)-alkylaminocarbonyl, aryl, hetaryl, aryl-C₁-C₆-alkyl or hetaryl-C₁-C₆-alkyl,
   where aryl, hetaryl, aryl-C₁-C₆-alkyl and hetaryl-C₁-C₆-alkyl may optionally be mono- or polysubstituted identically or differently by halogen, cyano, nitro, hydroxyl, C₁-C₆-alkyl, C₂-C₆-alkenyl, C₂-C₆-alkynyl, C₃-C₆-cycloalkyl, C₁-C₆-alkoxy, C₁-C₆-haloalkyl, C₁-C₆-haloalkoxy or C₁-C₆-alkylthio,
   or
R¹ and R² or
R² and R³ or
R³ and R⁴ in each case together form a 5- or 6-membered aliphatic, aromatic, heteroaromatic or heterocyclic ring which may optionally contain 1 to 2 atoms from the group of O, S and N and which may optionally be mono- or polysubstituted by halogen or by C₁-C₄-alkyl,
R⁵ is hydrogen or
   is C₁-C₆-alkyl, C₂-C₆-alkenyl or C₂-C₆-alkynyl, where the aforementioned radicals may optionally independently be mono- to pentasubstituted by halogen or monosubstituted by nitro, cyano, C₃-C₆-cycloalkyl, —O—R⁵¹, —S(O)ₚ—R⁵², —N(R⁵³)(R⁵⁴), —C(═O)N(R⁵³)(R⁵⁴), —O—C(═O)—R⁵⁵, —C(═O)—R⁵⁵, O—SO₂—R⁵⁶, aryl, hetaryl, heterocyclyl or oxoheterocyclyl, where aryl, hetaryl, heterocyclyl or oxoheterocyclyl may in turn each be mono- to trisubstituted by halogen, nitro, cyano, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, C₁-C₆-alkylcarbonylamino, aryl or hetaryl, where aryl and hetaryl may in turn optionally be mono- or polysubstituted identically or differently by halogen, cyano, nitro, hydroxyl, C₁-C₆-alkyl, C₂-C₆-alkenyl, C₂-C₆-alkynyl, C₁-C₆-alkoxy, C₁-C₆-haloalkyl, C₁-C₆-haloalkoxy or C₁-C₆-alkylthio,
   or
R⁵ is C₃-C₆-cycloalkyl or C₃-C₆-heterocyclyl, where the aforementioned radicals may optionally independently be mono- to trisubstituted by halogen, or monosubstituted by cyano, C₁-C₆-alkyl, C₁-C₆-haloalkyl, C₁-C₆-alkoxy, C₁-C₆-haloalkoxy, C₃-C₆-cycloalkyl, C₁-C₆-alkylthio, C₁-C₆-alkylsulphinyl, C₁-C₆-alkylsulphonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy,
   or
R⁵ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
p is 0, 1 or 2,
R⁵¹ is hydrogen or
   is C₁-C₆-alkyl, C₂-C₆-alkenyl, C₂-C₆-alkynyl, C₃-C₆-cycloalkyl or C₃-C₆-heterocyclyl, where the aforementioned radicals may optionally independently be mono- to trisubstituted by halogen or monosubstituted by nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
   or
R⁵¹ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵² is C₁-C₆-alkyl, C₂-C₆-alkenyl, C₂-C₆-alkynyl, C₃-C₆-cycloalkyl or C₃-C₆-heterocyclyl, where the aforementioned radicals may optionally be mono- to trisubstituted and the substituents are independently selected from halogen, nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbony, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
   or
R⁵² is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵³ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl, C₁-C₆-alkylcarbonyl, aryl, hetaryl, arylcarbonyl or hetarylcarbonyl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
R⁵⁴ is hydrogen or
   is C₁-C₆-alkyl, C₂-C₆-alkenyl, C₂-C₆-alkynyl, C₃-C₆-cycloalkyl or C₃-C₆-cycloalkyl-C₁-C₆-alkyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₆-alkoxycarbonyl, C₁-C₆-alkylcarbonyl or C₃-C₆-trialkylsilyl,
   or
R⁵⁴ is aryl, hetaryl, aryl-C₁-C₆-alkyl or heteroaryl-C₁-C₆-alkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
   or
R⁵³ and R⁵⁴ may be joined to one another via two to six carbon atoms and form a ring which optionally additionally contains a further atom from the group of O, S and N and may optionally be mono- to tetrasubstituted by C₁-C₂-alkyl, halogen, cyano, amino or C₁-C₂-alkoxy,
R⁵⁵ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁵⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl or aryl-C₁-C₆-alkyl, where aryl, hetaryl and aryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁷ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl or C₁-C₆-alkylcarbonyl,
R^{7a} is hydrogen, C₁-C₆-alkyl, C₂-C₆-alkenyl, C₂-C₆-alkynyl, C₃-C₆-cycloalkyl, C₁-C₄-alkoxy-C₁-C₆-alkyl, C₁-C₆-alkoxycarbonyl or C₁-C₆-alkylcarbonyl,
R⁸ is hydrogen or
   is C₁-C₆-alkyl, C₂-C₆-alkenyl or C₂-C₆-alkynyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₁-C₄-alkylsulphimino-C₁-C₄-alkyl, C₁-C₄-alkylsulphimino-C₂-C₅-alkylcarbonyl, C₁-C₄-alkylsulphoximino, C₁-C₄-alkylsulphoximino-C₁-C₄-alkyl, C₁-C₄-alkylsulphoximino-C₂-C₅-alkylcarbonyl, C₁-C₆-alkoxycarbonyl, C₁-C₆-alkylcarbonyl or C₃-C₆-trialkylsilyl,
   or
R⁸ is aryl-C₁-C₆-alkyl, heteroaryl-C₁-C₆-alkyl, C₃-C₁₂-cycloalkyl, C₃-C₁₂-cycloalkyl-C₁-C₆-alkyl or C₄-C₁₂-bicycloalkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
   or
R⁸ is a singly or multiply, identically or differently substituted 5- or 6-membered aromatic or heteroaromatic ring, a 4- to 6-membered, partly saturated ring or saturated heterocyclic ring, or a saturated or aromatic heterobicyclic ring which may optionally contain 1 to 3 heteroatoms from the group of O, S or N and which may optionally be mono- or polysubstituted, where the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
   or
R and R⁸ may be joined to one another via two to six carbon atoms and form a ring which optionally additionally contains a further nitrogen, sulphur or oxygen atom and may optionally be mono- to tetrasubstituted by C₁-C₂-alkyl, halogen, cyano, amino or C₁-C₂-alkoxy,
R⁹ is hydrogen, fluorine, chlorine, C₁-C₄-alkyl, C₃-C₆-cycloalkyl or C₁-C₄-haloalkyl,
R^{9a} is hydrogen, fluorine, chlorine, C₁-C₄-alkyl, C₃-C₆-cycloalkyl or C₁-C₄-haloalkyl,
R¹⁰ is hydrogen, fluorine, chlorine or C₁-C₄-alkyl,
and
R^{10a} is hydrogen, fluorine, chlorine or C₁-C₄-alkyl.

Preference (Configuration 2-2) is given to the compounds of the formula (I) in which
Q¹ and Q² together are one of the A-1, A-4 or A-5 radicals which, together with the carbon and nitrogen atom to which they are bonded, form a 5- or 6-membered aromatic ring,
T is R⁵, —OR⁶, —N(R⁷)(R⁸) or —N(R^{7a})—N(R⁷)(R⁸),
W is O,
G is a bond, is —C(R⁹)(R¹⁰)—, or is C(R⁹)(R¹⁰)C(R^{9a})(R^{10a}),
U is a cycle from the group of U-1, U-2, U-5, U-6, U-9, U-10, U-20 or U-23,
X^{a} is halogen, nitro, cyano, SF₅, C₁-C₄-alkyl, C₁-C₄-haloalkyl, C₁-C₄-alkoxy, C₁-C₄-haloalkoxy, C₁-C₄-alkoxy-C₁-C₄-alkyl, cyano-C₁-C₄-alkyl, C₂-C₄-alkenyl, C₃-C₄-alkynyl, C₁-C₄-alkylthio, C₁-C₄-alkylcarbonyl, C₁-C₄-alkoxycarbonyl, C₁-C₄-alkoxyimino-C₁-C₄-alkyl, C₁-C₄-alkylsulphinyl, C₁-C₄-alkylsulphonyl, C₁-C₄-haloalkylsulphonyl, phenyl, pyridyl, phenyl-C₁-C₄-alkyl or pyridyl-C₁-C₄-alkyl, where phenyl, pyridyl, phenyl-C₁-C₄-alkyl and pyridyl-C₁-C₄-alkyl may each be mono- to trisubstituted by halogen, cyano, nitro, C₁-C₄-alkyl, C₁-C₄-haloalkyl, C₁-C₄-alkoxy or C₁-C₄-haloalkoxy,
n is 0, 1, 2 or 3,
R¹, R², R³, R⁴ are each independently hydrogen, halogen, cyano, C₁-C₄-alkyl, C₃-C₆-cycloalkyl, C₁-C₄-haloalkyl, C₁-C₄-alkoxy, C₁-C₄-haloalkoxy, C₁-C₄-alkoxy-C₁-C₄-alkyl, C₂-C₄-alkenyl, C₂-C₄-alkenyloxy, C₃-C₄-alkynyl, C₃-C₄-alkynyloxy, C₁-C₄-alkylthio, C₁-C₄-alkylsulphinyl, C₁-C₄-alkylsulphonyl, C₁-C₄-alkoxyimino-C₁-C₄-alkyl, C₁-C₄-alkoxycarbonyl, aryl or hetaryl,
   where aryl and hetaryl may optionally be mono- to trisubstituted identically or differently by halogen, cyano, C₁-C₄-alkyl, C₁-C₄-alkoxy, C₁-C₄-haloalkyl, C₁-C₄-haloalkoxy or C₁-C₄-alkylthio,
R⁵ is CH₂Y,
Y is hydrogen, halogen, C₁-C₆-alkyl, C₁-C₆-haloalkyl, —O—R⁵¹, —S(O)ₚ—R⁵², —N(R⁵³)(R⁵⁴), —O—C(═O)—R⁵⁵, O—SO₂—R⁵⁶ or one of the Y-1 to Y-23 radicals:

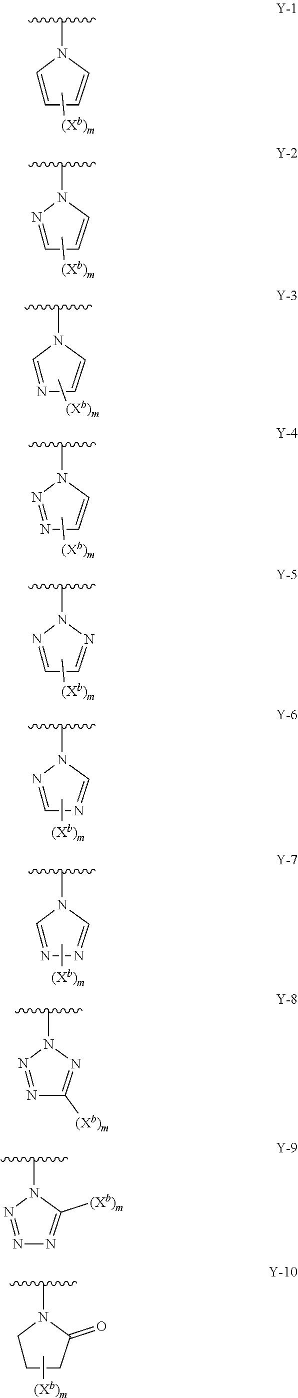

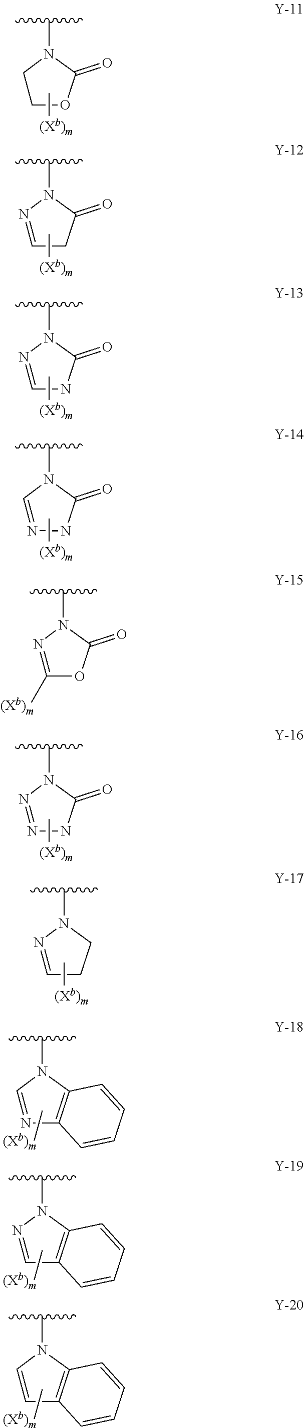

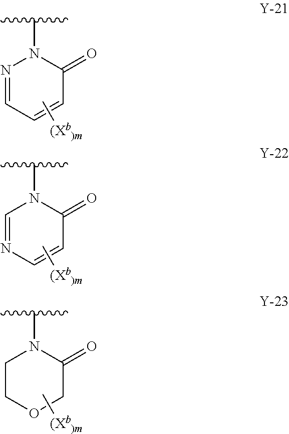

X^{b} is halogen, nitro, cyano, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, C₁-C₆-alkylcarbonylamino, aryl or hetaryl, where aryl and hetaryl may optionally be mono- or polysubstituted identically or differently by halogen, cyano, nitro, hydroxyl, C₁-C₆-alkyl, C₂-C₆-alkenyl, C₂-C₆-alkynyl, C₁-C₆-alkoxy, C₁-C₆-haloalkyl, C₁-C₆-haloalkoxy or C₁-C₆-alkylthio and where the ring nitrogen atoms in Y-13, Y-14 and Y-16 are not substituted by halogen, nitro, cyano, C₁-C₄-alkoxy, C₁-C₄-haloalkoxy or C₁-C₄-alkoxy-C₁-C₄-alkyloxy,
m is 0, 1 or 2,
p is 0, 1 or 2,
R⁵¹ is hydrogen or
   is C₁-C₆-alkyl, C₂-C₄-alkenyl, C₂-C₄-alkynyl, C₃-C₆-cycloalkyl or C₃-C₆-heterocyclyl, where the aforementioned radicals may optionally independently be mono- to trisubstituted by halogen or monosubstituted by cyano, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxyimino-C₁-C₄-alkyl, C₁-C₄-alkoxycarbonyl, C₁-C₄-alkylsulphonyl, C₁-C₄-haloalkylsulphonyl, C₁-C₄-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
   or
R⁵¹ is aryl or hetaryl, where the aforementioned radicals may optionally be mono- to trisubstituted and the substituents are independently selected from halogen, nitro, cyano, C₁-C₄-alkyl, C₁-C₄-haloalkyl, C₁-C₄-alkoxy, C₁-C₄-haloalkoxy, C₁-C₄-alkylthio or C₁-C₄-alkylsulphonyl,
R⁵² is C₁-C₄-alkyl, C₂-C₄-alkenyl, C₂-C₄-alkynyl, C₃-C₆-cycloalkyl or C₃-C₆-heterocyclyl, where the aforementioned radicals may optionally independently be mono- to trisubstituted by halogen or monosubstituted by cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxyimino-C₁-C₄-alkyl, C₁-C₄-alkoxycarbonyl, C₁-C₄-alkylsulphonyl, C₁-C₄-haloalkylsulphonyl, C₁-C₄-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
   or
R⁵² is aryl or hetaryl, where the aforementioned radicals may optionally be mono- to trisubstituted and the substituents are independently selected from halogen, nitro, cyano, C₁-C₄-alkyl, C₁-C₄-haloalkyl, C₁-C₄-alkoxy, C₁-C₄-haloalkoxy, C₁-C₄-alkylthio or C₁-C₄-alkylsulphonyl,
R⁵³ is hydrogen, C₁-C₄-alkyl, C₁-C₄-alkoxycarbonyl, C₁-C₆-alkylcarbonyl, aryl, hetaryl, arylcarbonyl or hetarylcarbonyl, where aryl, hetaryl, arylcarbonyl or hetarylcarbonyl may each be mono- to trisubstituted by halogen, cyano, nitro, C₁-C₄-alkyl, C₁-C₄-haloalkyl, C₁-C₄-alkoxy or C₁-C₄-haloalkoxy,
R⁵⁴ is hydrogen or
   is C₁-C₄-alkyl, C₂-C₄-alkenyl, C₂-C₄-alkynyl or C₃-C₄-cycloalkyl, where the aforementioned radicals may optionally independently be mono- to pentasubstituted by halogen or monosubstituted by cyano, C₁-C₄-alkyl, C₃-C₄-cycloalkyl, C₁-C₄-alkoxy, C₁-C₄-haloalkoxy, C₁-C₄-alkylthio, C₁-C₄-alkylsulphinyl, C₁-C₄-alkylsulphonyl or phenyl,
R⁶ is C₁-C₄-alkyl, C₁-C₄-haloalkyl, C₃-C₆-cycloalkyl-C₁-C₄-alkyl, C₁-C₄-alkoxy-C₁-C₄-alkyl, aryl-C₁-C₄-alkyl or hetaryl-C₁-C₄-alkyl, where aryl-C₁-C₄-alkyl and hetaryl-C₁-C₄-alkyl may optionally be mono- to trisubstituted identically or differently by halogen, cyano, nitro, C₁-C₄-alkyl, C₁-C₄-haloalkyl, C₁-C₄-alkoxy or C₁-C₄-haloalkoxy,
R⁷ is hydrogen, C₁-C₄-alkyl, C₂-C₄-alkenyl, C₂-C₄-alkynyl, C₃-C₆-cycloalkyl, C₁-C₄-alkoxy, C₁-C₄-alkoxy-C₁-C₄-alkyl, C₁-C₄-alkoxycarbonyl or C₁-C₄-alkylcarbonyl,
R^{7a} is hydrogen, C₁-C₄-alkyl, C₁-C₄-alkoxycarbonyl or C₁-C₄-alkylcarbonyl,
R⁸ is hydrogen or
   is C₁-C₄-alkyl, C₂-C₄-alkenyl or C₂-C₄-alkynyl, where the aforementioned radicals may optionally independently be mono- to trisubstituted by halogen, or monosubstituted by cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkoxycarbonyl, or C₁-C₄-alkylcarbonyl,
   or
R⁸ is phenylmethyl, phenylethyl, pyridylmethyl, C₃-C₆-cycloalkyl, C₃-C₆-cycloalkyl-C₁-C₄-alkyl or C₄-C₈-bicycloalkyl, where the aforementioned radicals may optionally be mono- to trisubstituted identically or differently and the substituents are independently selected from halogen, cyano, nitro, hydroxyl, amino, C₁-C₄-alkyl, C₁-C₄-alkoxy, C₁-C₄-haloalkoxy, C₁-C₄-alkylthio, C₁-C₄-alkylsulphinyl, C₁-C₄-alkylsulphonyl, C₂-C₆-alkoxycarbonyl or C₂-C₆-alkylcarbonyl,
   or
R⁸ is phenyl, pyridyl, pyrimidinyl, thiazolyl, thiadiazolyl, oxazolyl, pyrazolyl, thienyl or furanyl, where the aforementioned radicals may optionally be mono- to trisubstituted identically or differently and the substituents are independently selected from halogen, cyano, nitro, hydroxyl, amino, C₁-C₄-alkyl, C₂-C₄-alkynyl, C₁-C₄-haloalkyl, C₁-C₄-alkoxy, C₁-C₄-haloalkoxy, C₁-C₄-alkylthio, C₁-C₄-alkylsulphinyl or C₁-C₄-alkylsulphonyl, C₂-C₆-alkoxycarbonyl or C₂-C₆-alkylcarbonyl,
   or
R⁷ and R⁸ together are a C₃-C₆-alkyl chain,
R⁹ is hydrogen, fluorine, chlorine or C₁-C₄-alkyl,
R^{9a} is hydrogen, fluorine, chlorine or C₁-C₄-alkyl,
R¹⁰ is hydrogen, fluorine, chlorine or C₁-C₄-alkyl,
and
R^{10a} is hydrogen, fluorine, chlorine or C₁-C₄-alkyl.

Likewise preferred (Configuration 2-1) are the compounds of the formula (I) in which
Q¹ and Q² together are the A-1 radical which, together with the carbon and nitrogen atom to which it is bonded, forms a 6-membered aromatic ring,
T is R⁵, —OR⁶, —N(R⁷)(R⁸) or —N(R^{7a})—N(R⁷)(R⁸),
W is O,
G is a bond, is —C(R⁹)(R¹⁰)—, or is C(R⁹)(R¹⁰)C(R^{9a})(R^{10a}),
U is a cycle from the group of U-1, U-2, U-5, U-6, U-9, U-20 or U-23,
X^{a} is halogen, nitro, cyano, SF₅, C₁-C₄-alkyl, C₁-C₄-haloalkyl, C₁-C₄-alkoxy, C₁-C₄-haloalkoxy, C₁-C₄-alkoxy-C₁-C₄-alkyl, cyano-C₁-C₄-alkyl, C₂-C₄-alkenyl, C₃-C₄-alkynyl, C₁-C₄-alkylthio, C₁-C₄-alkylcarbonyl, C₁-C₄-alkoxycarbonyl, C₁-C₄-alkoxyimino-C₁-C₄-alkyl, C₁-C₄-alkylsulphinyl, C₁-C₄-alkylsulphonyl, C₁-C₄-haloalkylsulphonyl, phenyl, pyridyl, phenyl-C₁-C₄-alkyl or pyridyl-C₁-C₄-alkyl, where phenyl, pyridyl, phenyl-C₁-C₄-alkyl and pyridyl-C₁-C₄-alkyl may each be mono- to trisubstituted by halogen, cyano, nitro, C₁-C₄-alkyl, C₁-C₄-haloalkyl, C₁-C₄-alkoxy or C₁-C₄-haloalkoxy,
n is 0, 1, 2 or 3,
R¹, R², R³, R⁴ are each independently hydrogen, halogen, cyano, C₁-C₄-alkyl, C₃-C₆-cycloalkyl, C₁-C₄-haloalkyl, C₁-C₄-alkoxy, C₁-C₄-haloalkoxy, C₁-C₄-alkoxy-C₁-C₄-alkyl, C₂-C₄-alkenyl, C₂-C₄-alkenyloxy, C₃-C₄-alkynyl, C₃-C₄-alkynyloxy, C₁-C₄-alkylthio, C₁-C₄-alkylsulphinyl, C₁-C₄-alkylsulphonyl, C₁-C₄-alkoxyimino-C₁-C₄-alkyl, C₁-C₄-alkoxycarbonyl, aryl or hetaryl,
   where aryl and hetaryl may optionally be mono- to trisubstituted identically or differently by halogen, cyano, C₁-C₄-alkyl, C₁-C₄-alkoxy, C₁-C₄-haloalkyl, C₁-C₄-haloalkoxy or C₁-C₄-alkylthio,
R⁵ is CH₂Y,
Y is hydrogen, halogen, C₁-C₆-alkyl, C₁-C₆-haloalkyl, —O—R⁵¹, —S(O)ₚ—R⁵², —N(R⁵³)(R⁵⁴), —O—C(═O)—R⁵⁵, O—SO₂—R⁵⁶ or one of the Y-1 to Y-23 radicals,
X^{b} is halogen, nitro, cyano, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, C₁-C₆-alkylcarbonylamino, aryl or hetaryl, where aryl and hetaryl may optionally be mono- or polysubstituted identically or differently by halogen, cyano, nitro, hydroxyl, C₁-C₆-alkyl, C₂-C₆-alkenyl, C₂-C₆-alkynyl, C₁-C₆-alkoxy, C₁-C₆-haloalkyl, C₁-C₆-haloalkoxy or C₁-C₆-alkylthio and where the ring nitrogen atoms in Y-13, Y-14 and Y-16 are not substituted by halogen, nitro, cyano, C₁-C₄-alkoxy, C₁-C₄-haloalkoxy or C₁-C₄-alkoxy-C₁-C₄-alkyloxy,
m is 0, 1 or 2,
p is 0, 1 or 2,
R⁵¹ is hydrogen or
   is C₁-C₄-alkyl, C₂-C₄-alkenyl, C₂-C₄-alkynyl, C₃-C₆-cycloalkyl or C₃-C₆-heterocyclyl, where the aforementioned radicals may optionally independently be mono- to trisubstituted by halogen or monosubstituted by cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxyimino-C₁-C₄-alkyl, C₁-C₄-alkoxycarbonyl, C₁-C₄-alkylsulphonyl, C₁-C₄-haloalkylsulphonyl, C₁-C₄-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
   or
R⁵¹ is aryl or hetaryl, where the aforementioned radicals may optionally be mono- to trisubstituted and the substituents are independently selected from halogen, nitro, cyano, C₁-C₄-alkyl, C₁-C₄-haloalkyl, C₁-C₄-alkoxy, C₁-C₄-haloalkoxy, C₁-C₄-alkylthio or C₁-C₄-alkylsulphonyl,
R⁵² is C₁-C₄-alkyl, C₂-C₄-alkenyl, C₂-C₄-alkynyl, C₃-C₆-cycloalkyl or C₃-C₆-heterocyclyl, where the aforementioned radicals may optionally independently be mono- to trisubstituted by halogen or monosubstituted by cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxyimino-C₁-C₄-alkyl, C₁-C₄-alkoxycarbonyl, C₁-C₄-alkylsulphonyl, C₁-C₄-haloalkylsulphonyl, C₁-C₄-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
   or
R⁵² is aryl or hetaryl, where the aforementioned radicals may optionally be mono- to trisubstituted and the substituents are independently selected from halogen, nitro, cyano, C₁-C₄-alkyl, C₁-C₄-haloalkyl, C₁-C₄-alkoxy, C₁-C₄-haloalkoxy, C₁-C₄-alkylthio or C₁-C₄-alkylsulphonyl,
R⁵³ is hydrogen, C₁-C₄-alkyl, C₁-C₄-alkoxycarbonyl, C₁-C₆-alkylcarbonyl, aryl, hetaryl, arylcarbonyl or hetarylcarbonyl, where aryl, hetaryl, arylcarbonyl or hetarylcarbonyl may each be mono- to trisubstituted by halogen, cyano, nitro, C₁-C₄-alkyl, C₁-C₄-haloalkyl, C₁-C₄-alkoxy or C₁-C₄-haloalkoxy,
R⁵⁴ is hydrogen or
   is C₁-C₄-alkyl, C₂-C₄-alkenyl, C₂-C₄-alkynyl or C₃-C₄-cycloalkyl, where the aforementioned radicals may optionally independently be mono- to pentasubstituted by halogen or monosubstituted by cyano, C₁-C₄-alkyl, C₃-C₄-cycloalkyl, C₁-C₄-alkoxy, C₁-C₄-haloalkoxy, C₁-C₄-alkylthio, C₁-C₄-alkylsulphinyl, C₁-C₄-alkylsulphonyl or phenyl,
R⁶ is C₁-C₄-alkyl, C₁-C₄-haloalkyl, C₃-C₆-cycloalkyl-C₁-C₄-alkyl, C₁-C₄-alkoxy-C₁-C₄-alkyl, aryl-C₁-C₄-alkyl or hetaryl-C₁-C₄-alkyl, where aryl-C₁-C₄-alkyl and hetaryl-C₁-C₄-alkyl may optionally be mono- to trisubstituted identically or differently by halogen, cyano, nitro, C₁-C₄-alkyl, C₁-C₄-haloalkyl, C₁-C₄-alkoxy or C₁-C₄-haloalkoxy,
R⁷ is hydrogen, C₁-C₄-alkyl, C₂-C₄-alkenyl, C₂-C₄-alkynyl, C₃-C₆-cycloalkyl, C₁-C₄-alkoxy, C₁-C₄-alkoxy-C₁-C₄-alkyl, C₁-C₄-alkoxycarbonyl or C₁-C₄-alkylcarbonyl,
R^{7a} is hydrogen, C₁-C₄-alkyl, C₁-C₄-alkoxycarbonyl or C₁-C₄-alkylcarbonyl,
R⁸ is hydrogen or
   is C₁-C₄-alkyl, C₂-C₄-alkenyl or C₂-C₄-alkynyl, where the aforementioned radicals may optionally independently be mono- to trisubstituted by halogen, or monosubstituted by cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkoxycarbonyl, or C₁-C₄-alkylcarbonyl,
   or
R⁸ is phenylmethyl, phenylethyl, pyridylmethyl, C₃-C₆-cycloalkyl, C₃-C₆-cycloalkyl-C₁-C₄-alkyl or C₄-C₈-bicycloalkyl, where the aforementioned radicals may optionally be mono- to trisubstituted identically or differently and the substituents are independently selected from halogen, cyano, nitro, hydroxyl, amino, C₁-C₄-alkyl, C₁-C₄-alkoxy, C₁-C₄-haloalkoxy, C₁-C₄-alkylthio, C₁-C₄-alkylsulphinyl, C₁-C₄-alkylsulphonyl, C₂-C₆-alkoxycarbonyl or C₂-C₆-alkylcarbonyl,
   or
R⁸ is phenyl, pyridyl, pyrimidinyl, thiazolyl, oxazolyl, pyrazolyl, thienyl or furanyl, where the aforementioned radicals may optionally be mono- to trisubstituted identically or differently and the substituents are independently selected from halogen, cyano, nitro, hydroxyl, amino, C₁-C₄-alkyl, C₁-C₄-alkoxy, C₁-C₄-haloalkoxy, C₁-C₄-alkylthio, C₁-C₄-alkylsulphinyl or C₁-C₄-alkylsulphonyl, C₂-C₆-alkoxycarbonyl or C₂-C₆-alkylcarbonyl,
R⁹ is hydrogen, fluorine, chlorine or C₁-C₄-alkyl,
R^{9a} is hydrogen, fluorine, chlorine or C₁-C₄-alkyl,
R¹⁰ is hydrogen, fluorine, chlorine or C₁-C₄-alkyl,
and
R^{10a} is hydrogen, fluorine, chlorine or C₁-C₄-alkyl.

Further preferred (Configuration 3-2) are the compounds of the formula (I) in which
Q¹ and Q² together are one of the A-1, A-4 or A-5 radicals which, together with the carbon and nitrogen atom to which they are bonded, form a 5- or 6-membered aromatic ring,
T is R⁵, —OR⁶, —N(R⁷)(R⁸) or —N(R^{7a})—N(R⁷)(R⁸),
W is O,
G is a bond or is —C(R⁹)(R¹⁰)—,
U is a cycle from the group of U-2, U-9, U-10 or U-23,
X^{a} is halogen, cyano, methyl, ethyl, trifluoromethyl, methoxy, ethoxy, trifluoromethoxy, methylthio, methylsulphinyl, methylsulphonyl or methoxycarbonyl,
n is 0, 1 or 2,
R¹, R², R³, R⁴ are each independently a radical from the group of hydrogen, halogen, cyano, C₁-C₄-alkyl, C₁-C₄-haloalkyl, C₁-C₄-alkoxy, C₁-C₄-haloalkoxy, C₁-C₄-alkoxy-C₁-C₄-alkyl, C₂-C₄-alkenyl, C₂-C₄-alkenyloxy, C₃-C₄-alkynyl, C₃-C₄-alkynyloxy, C₁-C₄-alkylthio, C₁-C₄-alkylsulphinyl or C₁-C₄-alkylsulphonyl,
R⁵ is CH₂Y,
Y is hydrogen, Cl, Br, —O—R⁵¹, —S(O)ₚ—R⁵², —N(R⁵³)(R⁵⁴) or is one of the Y-2, Y-3, Y-4, Y-5, Y-6 or Y-7 radicals,
X^{b} is halogen, cyano, methyl, ethyl, trifluoromethyl, difluoromethyl, methylaminocarbonyl, methylcarbonylamino, methoxy, ethoxy, trifluoromethoxy, methylthio, ethylthio, methylsulphinyl, methylsulphonyl or methoxycarbonyl,
m is 0, 1 or 2,
p is 0, 1 or 2,
R⁵¹ is hydrogen or
   is C₁-C₆-alkyl, C₂-C₄-alkenyl, C₂-C₄-alkynyl or C₃-C₆-cycloalkyl, where the aforementioned radicals may optionally be mono- to trisubstituted by halogen or monosubstituted by cyano, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxycarbonyl or C₁-C₄-alkylsulphonyl, or may be monosubstituted by phenyl, thienyl, pyridyl, pyrimidinyl or pyrazolyl, which may in turn each independently be mono- to trisubstituted by halogen, cyano, methyl, ethyl, trifluoromethyl, methoxy, ethoxy or trifluoromethoxy,
   or
R⁵¹ is phenyl, thienyl, pyridyl, pyrimidinyl or pyrazolyl, where the aforementioned radicals may optionally be mono- to trisubstituted and the substituents are independently selected from halogen, cyano, methyl, ethyl, trifluoromethyl, methoxy, ethoxy or trifluoromethoxy,
R⁵² is C₁-C₄-alkyl or C₃-C₆-cycloalkyl,
   where the aforementioned radicals may optionally be mono- to trisubstituted and the substituents are independently selected from halogen, cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxycarbonyl or C₁-C₄-alkylsulphonyl, and from phenyl, thienyl, pyridyl, pyrimidinyl, pyrazolyl, each of which may independently be mono- to trisubstituted by halogen, cyano, methyl, ethyl, trifluoromethyl, methoxy, ethoxy or trifluoromethoxy,
   or
R⁵² is phenyl, thienyl, pyridyl, pyrimidinyl or pyrazolyl, where the aforementioned radicals may optionally be mono- to trisubstituted and the substituents are independently selected from halogen, cyano, methyl, ethyl, trifluoromethyl, methoxy, ethoxy or trifluoromethoxy,
R⁵³ is hydrogen, C₁-C₄-alkyl, C₁-C₄-alkoxycarbonyl or C₁-C₆-alkylcarbonyl,
R⁵⁴ is hydrogen or
   is C₁-C₄-alkyl, C₂-C₄-alkenyl, C₂-C₄-alkynyl or C₃-C₄-cycloalkyl, where the aforementioned radicals may optionally be mono- to trisubstituted by halogen, or monosubstituted by cyano, methyl, ethyl, cyclopropyl, methoxy, ethoxy, trifluoromethoxy, methylthio, methylsulphinyl, methylsulphonyl or phenyl,
R⁶ is C₁-C₄-alkyl, C₁-C₄-haloalkyl, C₃-C₆-cycloalkyl-C₁-C₄-alkyl, C₁-C₄-alkoxy-C₁-C₄-alkyl,
R⁷ is hydrogen, C₁-C₄-alkyl, C₁-C₄-alkoxy, C₁-C₄-alkoxy-C₁-C₄-alkyl, C₁-C₄-alkoxycarbonyl or C₁-C₄-alkylcarbonyl,
R^{7a} is hydrogen, C₁-C₄-alkyl, C₁-C₄-alkoxycarbonyl or C₁-C₄-alkylcarbonyl,
R⁸ is hydrogen or
   is C₁-C₄-alkyl, C₂-C₄-alkenyl or C₂-C₄-alkynyl, where the aforementioned radicals may optionally independently be mono- to trisubstituted by halogen or monosubstituted by cyano, methoxy, ethoxy, difluoromethoxy, trifluoromethoxy, trifluoroethoxy, methylthio, ethylthio, methylsulphinyl, ethylsulphinyl, methylsulphonyl or ethylsulphonyl,
   or
R⁸ is phenylmethyl, phenylethyl, pyridylmethyl, pyridylethyl, C₃-C₆-cycloalkyl or C₃-C₆-cycloalkyl-C₁-C₄-alkyl, where the aforementioned radicals may optionally be mono- to trisubstituted and the substituents are independently selected from halogen, cyano, methyl, ethyl, trifluoromethyl, methoxy, ethoxy, trifluoromethoxy, methylthio, methylsulphinyl, methylsulphonyl or methoxycarbonyl,
   or
R⁸ is phenyl, pyridyl, pyrimidinyl, thiazolyl, thiadiazolyl, oxazolyl, pyrazolyl, thienyl or furanyl, where the aforementioned radicals may optionally be mono- to trisubstituted identically or differently and the substituents are independently selected from halogen, cyano, nitro, methyl, ethyl, ethynyl, trifluoromethyl, pentafluoroethyl, methoxy, ethoxy, trifluoromethoxy, methylthio, methylsulphinyl, methylsulphonyl or methoxycarbonyl,
   or
R⁷ and R⁸ together are a C₃-C₆-alkyl chain,
R⁹ is hydrogen, fluorine, chlorine, methyl or ethyl,
and
R¹⁰ is hydrogen, fluorine, chlorine, methyl or ethyl.

Likewise further preferred (Configuration 3-1) are the compounds of the formula (I) in which
Q¹ and Q² together are the A-1 radical which, together with the carbon and nitrogen atom to which it is bonded, forms a 6-membered aromatic ring,
T is R⁵, —OR⁶, —N(R⁷)(R⁸) or —N(R^{7a})—N(R⁷)(R⁸),
W is O,
G is a bond or is —C(R⁹)(R¹⁰)—,
U is a cycle from the group of U-2, U-9 or U-23,
X^{a} is halogen, cyano, methyl, ethyl, trifluoromethyl, methoxy, ethoxy, trifluoromethoxy, methylthio, methylsulphinyl, methylsulphonyl or methoxycarbonyl,
n is 0, 1 or 2,
R¹, R², R³, R⁴ are each independently a radical from the group of hydrogen, halogen, cyano, C₁-C₄-alkyl, C₁-C₄-haloalkyl, C₁-C₄-alkoxy, C₁-C₄-haloalkoxy, C₁-C₄-alkoxy-C₁-C₄-alkyl, C₂-C₄-alkenyl, C₂-C₄-alkenyloxy, C₃-C₄-alkynyl, C₃-C₄-alkynyloxy, C₁-C₄-alkylthio, C₁-C₄-alkylsulphinyl or C₁-C₄-alkylsulphonyl,
R⁵ is CH₂Y,
Y is hydrogen, Cl, Br, —O—R⁵¹, —S(O)ₚ—R⁵², —N(R⁵³)(R⁵⁴) or is one of the Y-2, Y-3, Y-4, Y-5, Y-6 or Y-7 radicals,
X^{b} is halogen, cyano, methyl, ethyl, trifluoromethyl, methylaminocarbonyl, methylcarbonylamino, methoxy, ethoxy, trifluoromethoxy, methylthio, methylsulphinyl, methylsulphonyl or methoxycarbonyl,
m is 0, 1 or 2,
p is 0, 1 or 2,
R⁵¹ is hydrogen or
   is C₁-C₄-alkyl, C₂-C₄-alkenyl or C₂-C₄-alkynyl, where the aforementioned radicals may optionally be mono- to trisubstituted by halogen or monosubstituted by cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxycarbonyl or C₁-C₄-alkylsulphonyl, or may be monosubstituted by phenyl, thienyl, pyridyl, pyrimidinyl or pyrazolyl, each of which may in turn independently be mono- to trisubstituted by halogen, cyano, methyl, ethyl, trifluoromethyl, methoxy, ethoxy or trifluoromethoxy,
   or
R⁵¹ is phenyl, thienyl, pyridyl, pyrimidinyl or pyrazolyl, where the aforementioned radicals may optionally be mono- to trisubstituted and the substituents are independently selected from halogen, cyano, methyl, ethyl, trifluoromethyl, methoxy, ethoxy or trifluoromethoxy,
R⁵² is C₁-C₄-alkyl or C₃-C₆-cycloalkyl,
   where the aforementioned radicals may optionally be mono- to trisubstituted and the substituents are independently selected from halogen, cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxycarbonyl or C₁-C₄-alkylsulphonyl, and from phenyl, thienyl, pyridyl, pyrimidinyl, pyrazolyl, each of which may independently be mono- to trisubstituted by halogen, cyano, methyl, ethyl, trifluoromethyl, methoxy, ethoxy or trifluoromethoxy,
   or
R⁵² is phenyl, thienyl, pyridyl, pyrimidinyl or pyrazolyl, where the aforementioned radicals may optionally be mono- to trisubstituted and the substituents are independently selected from halogen, cyano, methyl, ethyl, trifluoromethyl, methoxy, ethoxy or trifluoromethoxy,
R⁵³ is hydrogen, C₁-C₄-alkyl, C₁-C₄-alkoxycarbonyl or C₁-C₆-alkylcarbonyl, R⁵⁴ is hydrogen or
   is C₁-C₄-alkyl, C₂-C₄-alkenyl, C₂-C₄-alkynyl or C₃-C₄-cycloalkyl, where the aforementioned radicals may optionally be mono- to trisubstituted by halogen, or monosubstituted by cyano, methyl, ethyl, cyclopropyl, methoxy, ethoxy, trifluoromethoxy, methylthio, methylsulphinyl, methylsulphonyl or phenyl,
R⁶ is C₁-C₄-alkyl, C₁-C₄-haloalkyl, C₃-C₆-cycloalkyl-C₁-C₄-alkyl, C₁-C₄-alkoxy-C₁-C₄-alkyl,
R⁷ is hydrogen, C₁-C₄-alkyl, C₁-C₄-alkoxy, C₁-C₄-alkoxy-C₁-C₄-alkyl, C₁-C₄-alkoxycarbonyl or C₁-C₄-alkylcarbonyl,
R^{7a} is hydrogen, C₁-C₄-alkyl, C₁-C₄-alkoxycarbonyl or C₁-C₄-alkylcarbonyl,
R⁸ is hydrogen or
   is C₁-C₄-alkyl, C₂-C₄-alkenyl or C₂-C₄-alkynyl, where the aforementioned radicals may optionally independently be mono- to trisubstituted by halogen or monosubstituted by cyano, methoxy, ethoxy, difluoromethoxy, trifluoromethoxy, trifluoroethoxy, methylthio, ethylthio, methylsulphinyl, ethylsulphinyl, methylsulphonyl or ethylsulphonyl,
   or
R⁸ is phenylmethyl, phenylethyl, pyridylmethyl, pyridylethyl, C₃-C₆-cycloalkyl or C₃-C₆-cycloalkyl-C₁-C₄-alkyl, where the aforementioned radicals may optionally be mono- to trisubstituted and the substituents are independently selected from halogen, cyano, methyl, ethyl, trifluoromethyl, methoxy, ethoxy, trifluoromethoxy, methylthio, methylsulphinyl, methylsulphonyl or methoxycarbonyl,
   or
R⁸ is phenyl, pyridyl, pyrimidinyl, thiazolyl, oxazolyl, pyrazolyl, thienyl or furanyl, where the aforementioned radicals may optionally be mono- to trisubstituted identically or differently and the substituents are independently selected from halogen, cyano, methyl, ethyl, trifluoromethyl, methoxy, ethoxy, trifluoromethoxy, methylthio, methylsulphinyl, methylsulphonyl or methoxycarbonyl,
R⁹ is hydrogen, fluorine, chlorine, methyl or ethyl,
and
R¹⁰ is hydrogen, fluorine, chlorine, methyl or ethyl.

Particularly preferred (Configuration 4-2) are the compounds of the formula (I) in which
Q¹ and Q² together are one of the A-1, A-4 or A-5 radicals which, together with the carbon and nitrogen atom to which they are bonded, form a 5- or 6-membered aromatic ring,
T is R⁵, —OR⁶ or —N(R⁷)(R⁸),
W is O,
G is a bond or is —C(R⁹)(R¹⁰)—,
U is U-2, U-9, U-10 or U-23,
X^{a} is fluorine, chlorine, bromine, methyl, ethyl, trifluoromethyl, methoxy, trifluoromethoxy or cyano,
n is 0, 1 or 2,
R¹, R², R³, R⁴ are each independently a radical from the group of hydrogen, fluorine, chlorine, bromine, iodine, cyano, methyl, ethyl, n- or isopropyl, methoxy, ethoxy, n- or isopropoxy, methylthio, ethylthio, difluoromethyl, trifluoromethyl, trifluoromethoxy or difluoromethoxy,
R⁵ is CH₂Y,
Y is hydrogen, chlorine, —O—R⁵¹, —S(O)ₚ—R⁵², —N(R⁵³)(R⁵⁴) or is one of the Y-2, Y-3, Y-4, Y-5, Y-6 or Y-7 radicals,
X^{b} is fluorine, chlorine, bromine, cyano, methyl, ethyl, n- and isopropyl, methoxy, ethoxy, methylthio, ethylthio, difluoromethyl, trifluoromethyl, methylaminocarbonyl or methylcarbonylamino,
m is 0 or 1,
p is 0, 1 or 2,
R⁵¹ is hydrogen, methyl, ethyl, n- or isopropyl, n-, iso-, sec- or tert-butyl, pent-3-yl, cyclopropyl, cyclobutyl, cyclopentyl, cyclohexyl, cyclopropylmethyl, cyclobutylmethyl, cyclopentylmethyl, cyclohexylmethyl, 2,2,2-trifluoroethyl, 2-methoxyethyl, methoxycarbonylmethyl, phenyl, 2-fluorophenyl, 3-fluorophenyl, 4-fluorophenyl, 2-chlorophenyl, 3-chlorophenyl, 4-chlorophenyl, phenylmethyl, 4-fluorophenylmethyl, 3-fluorophenylmethyl, 2-fluorophenylmethyl, pyrid-3-yl, 6-chloropyrid-3-yl, pyridyl-2-yl or 1-methylpyrazol-5-yl,
R⁵² is methyl, ethyl, n- or isopropyl, phenyl or phenylmethyl,
R⁵³ is hydrogen, methyl, ethyl, methylcarbonyl or ethylcarbonyl,
R⁵⁴ is methyl, ethyl, n- or isopropyl, cyclopropyl or phenylmethyl,
R⁶ is methyl, ethyl or n- or isopropyl,
R⁷ is hydrogen, methyl or ethyl,
R⁸ is methyl, ethyl, n- or isopropyl, n-, iso-, sec- or tert-butyl, trifluoroethyl, cyclopropyl, fluorocyclopropyl, chlorocyclopropyl, cyanocyclopropyl, cyclobutyl, cyclopentyl, cyclohexyl, cyanocyclohexyl, cyclopropylmethyl, cyclopentylmethyl, cyclohexylmethyl, phenyl optionally mono- to disubstituted independently by fluorine, chlorine, methyl, ethyl, methoxy, ethoxy, trifluoromethyl, pentafluoroethyl, trifluoromethoxy, cyano or nitro, phenylmethyl optionally mono- to disubstituted independently by fluorine or chlorine, 1-phenylethyl optionally mono- to disubstituted independently by fluorine or chlorine, 2-phenylethyl optionally mono- to disubstituted independently by fluorine or chlorine, pyrid-3-yl optionally mono- to disubstituted independently by fluorine, chlorine or methyl, pyridyl-2-yl optionally mono- to disubstituted independently by fluorine, chlorine, bromine, methyl, trifluoromethyl, cyano, nitro or ethynyl, pyrid-4-yl optionally mono- to disubstituted independently by fluorine, chlorine or methoxy, 1,3-pyrimidin-2-yl optionally mono- to disubstituted independently by fluorine or chlorine, 1,3-pyrimidin-5-yl optionally mono- to disubstituted independently by fluorine or chlorine, thiazol-2-yl optionally mono- to disubstituted independently by fluorine, chlorine or methyl, 1,2,4-thiadiazol-5-yl, or 1-methylpyrazol-5-yl optionally substituted by chlorine, bromine or methyl,
R⁹ is hydrogen or methyl,
and
R¹⁰ is hydrogen.

Likewise particularly preferred (Configuration 4-1) are the compounds of the formula (I) in which
Q¹ and Q² together are the A-1 radical which, together with the carbon and nitrogen atom to which it is bonded, forms a 6-membered aromatic ring,
T is R⁵, —OR⁶ or —N(R⁷)(R⁸),
W is O,
G is a bond or is —C(R⁹)(R¹⁰)—,
U is U-2, U-9 or U-23,
X^{a} is fluorine, chlorine, bromine, methyl, ethyl, trifluoromethyl, methoxy, trifluoromethoxy or cyano,
n is 0, 1 or 2,
R¹, R², R³, R⁴ are each independently a radical from the group of hydrogen, fluorine, chlorine, bromine, iodine, cyano, methyl, ethyl, n- or isopropyl, methoxy, ethoxy, n- or isopropoxy, methylthio, ethylthio, difluoromethyl, trifluoromethyl, trifluoromethoxy or difluoromethoxy,
R⁵ is CH₂Y,
Y is hydrogen, chlorine, —O—R⁵¹, —S(O)ₚ—R⁵², —N(R⁵³)(R⁵⁴) or is one of the Y-2, Y-3, Y-4, Y-5, Y-6 or Y-7 radicals,
X^{b} is fluorine, chlorine, bromine, cyano, methyl, ethyl, n- and isopropyl, methoxy, ethoxy, methylthio, ethylthio, difluoromethyl, trifluoromethyl, methylaminocarbonyl or methylcarbonylamino,
m is 0 or 1,
p is 0, 1 or 2,
R⁵¹ is hydrogen, methyl, ethyl, n- or isopropyl, n-, iso-, sec- or tert-butyl, 2-methoxyethyl, methoxycarbonylmethyl, phenyl, 2-F-phenyl, 3-F-phenyl, 4-F-phenyl, 2-Cl-phenyl, 3-Cl-phenyl, 4-Cl-phenyl, phenylmethyl, 4-F-phenylmethyl, 3-F-phenylmethyl, 2-F-phenylmethyl, pyrid-3-yl, 6-chloropyrid-3-yl, pyrid-2-yl or 1-methylpyrazol-5-yl,
R⁵² is methyl, ethyl, n- or isopropyl, phenyl or phenylmethyl,
R⁵³ is hydrogen, methyl, ethyl, methylcarbonyl or ethylcarbonyl,
R⁵⁴ is methyl, ethyl, n- or isopropyl, cyclopropyl or phenylmethyl,
R⁶ is methyl, ethyl or n- or isopropyl,
R⁷ is hydrogen, methyl or ethyl,
R⁸ is methyl, ethyl, n- or isopropyl, n-, iso-, sec- or tert-butyl, trifluoroethyl, cyclopropyl, cyclobutyl, cyclopentyl, cyclopropylmethyl, optionally fluorine- or chlorine-substituted phenyl, optionally fluorine- or chlorine-substituted phenylmethyl, optionally fluorine- or chlorine-substituted 1-phenylethyl, optionally fluorine- or chlorine-substituted 2-phenylethyl, optionally fluorine- or chlorine-substituted pyrid-3-yl, optionally fluorine- or chlorine-substituted pyridyl-2-yl or optionally fluorine- or chlorine-substituted pyrid-4-yl,
R⁹ is hydrogen or methyl,
and
R¹⁰ is hydrogen.

Very particularly preferred (Configuration 5-2) are the compounds of the formula (I) in which
Q¹ and Q² together are one of the A-1, A-4 or A-5 radicals which, together with the carbon and nitrogen atom to which they are bonded, form a 5- or 6-membered aromatic ring,
T is R⁵, —OR⁶ or —N(R⁷)(R⁸),
W is O,
G is —C(R⁹)(R¹⁰)—,
U is U-2, U-9, U-10 or U-23,
X^{a} is fluorine, chlorine, methyl, trifluoromethyl or methoxy,
n is 0, 1 or 2,
R¹, R², R³, R⁴ are each independently a radical from the group of hydrogen, fluorine, chlorine, cyano, methyl, methoxy, ethoxy, methylthio, trifluoromethyl or trifluoromethoxy,
R⁵ is CH₂Y,
Y is hydrogen, chlorine, —O—R⁵¹, —S(O)ₚ—R⁵², —N(R⁵³)(R⁵⁴) or is one of the Y-2, Y-3, Y-4, Y-5 or Y-6 radicals,
X^{b} is chlorine, cyano, methyl, methoxy, methylthio, trifluoromethyl, methylaminocarbonyl or methylcarbonylamino,
m is 0 or 1,
p is 0, 1 or 2,
R⁵¹ is hydrogen, methyl, ethyl, n- or isopropyl, t-butyl, pent-3-yl, cyclopentyl, cyclohexyl, 2,2,2-trifluoroethyl, 2-methoxyethyl, methoxycarbonylmethyl, phenyl, 4-fluorophenyl, 2-chlorophenyl, 3-chlorophenyl, 4-chlorophenyl, phenylmethyl, 4-fluorophenylmethyl, pyrid-3-yl, 6-chloropyrid-3-yl, pyrid-2-yl or 1-methylpyrazol-5-yl,
R⁵² is methyl, ethyl, isopropyl, phenyl or phenylmethyl,
R⁵³ is hydrogen, methylcarbonyl or ethylcarbonyl,
R⁵⁴ is methyl, ethyl, cyclopropyl or phenylmethyl;
R⁶ is methyl,
R⁷ is hydrogen,
R⁸ is methyl, ethyl, n- or isopropyl, iso- or tert-butyl, trifluoroethyl, cyclopropyl, 1-cyanocyclopropyl, cyclobutyl, cyclopentyl, cyclohexyl, 1-cyanocyclohexyl, cyclopropylmethyl, cyclopentylmethyl, cyclohexylmethyl, phenyl optionally mono- to disubstituted independently by fluorine, chlorine, methyl, methoxy, trifluoromethyl, trifluoromethoxy or cyano, optionally chlorine-substituted phenylmethyl, optionally chlorine-substituted 1-phenylethyl, optionally chlorine-substituted 2-phenylethyl, pyrid-3-yl optionally mono- to disubstituted independently by chlorine or methyl, pyrid-2-yl optionally mono- to disubstituted independently by fluorine or chlorine, optionally mono-bromine-, -methyl-, -trifluoromethyl-, -cyano-, -nitro- or -ethynyl-substituted pyrid-2-yl, mono-methoxy-substituted pyrid-4-yl, 1,3-pyrimidin-2-yl, optionally mono- to di-chlorine-substituted 1,3-pyrimidin-5-yl, optionally mono-chlorine- or -methyl-substituted thiazol-2-yl, 1,2,4-thiadiazol-5-yl, or optionally bromine-substituted 1-methylpyrazol-5-yl,
   or
R⁷ and R⁸ together are —(CH₂)₄—,
R⁹ is hydrogen or methyl,
and
R¹⁰ is hydrogen.

Likewise very particularly preferred (Configuration 5-1) are the compounds of the formula (I) in which
Q¹ and Q² together are the A-1 radical which, together with the carbon and nitrogen atom to which it is bonded, forms a 6-membered aromatic ring,
T is R⁵, —OR⁶ or —N(R⁷)(R⁸),
W is O,
G is —C(R⁹)(R¹⁰)—,
U is U-2, U-9 or U-23,
X^{a} is chlorine, trifluoromethyl or methoxy,
n is 0, 1 or 2,
R¹, R², R³, R⁴ are each independently a radical from the group of hydrogen, fluorine, chlorine, cyano, methyl, ethoxy, methylthio, trifluoromethyl or trifluoromethoxy,
R⁵ is CH₂Y,
Y is hydrogen, chlorine, —O—R⁵¹, —S(O)ₚ—R⁵², —N(R⁵³)(R⁵⁴) or is one of the Y-2, Y-3, Y-4, Y-5, Y-6 radicals,
X^{b} is chlorine, cyano, methyl, methoxy, methylthio, trifluoromethyl, methylaminocarbonyl or methylcarbonylamino,
m is 0 or 1,
p is 0, 1 or 2,
R⁵¹ is hydrogen, methyl, ethyl, n- or isopropyl, 2-methoxyethyl, methoxycarbonylmethyl, phenyl, 4-F-phenyl, 2-Cl-phenyl, 3-Cl-phenyl, 4-Cl-phenyl, phenylmethyl, 4-F-phenylmethyl, pyrid-3-yl, 6-chloropyrid-3-yl, pyrid-2-yl or 1-methylpyrazol-5-yl,
R⁵² is methyl, ethyl, isopropyl, phenyl or phenylmethyl,
R⁵³ is hydrogen, methylcarbonyl or ethylcarbonyl,
R⁵⁴ is methyl, ethyl, cyclopropyl or phenylmethyl,
R⁶ is methyl,
R⁷ is hydrogen,
R⁸ is methyl, ethyl, n- or isopropyl, iso- or tert-butyl, trifluoroethyl, cyclopropyl, cyclobutyl, cyclopentyl, cyclopropylmethyl, optionally chlorine-substituted phenyl, optionally chlorine-substituted phenylmethyl, optionally chlorine-substituted 1-phenylethyl, optionally chlorine-substituted 2-phenylethyl or optionally chlorine-substituted pyrid-3-yl,
R⁹ is hydrogen or methyl,
and
R¹⁰ is hydrogen.

In a preferred embodiment, the invention relates to the compounds of the formula (I) in which W is O.

In a further preferred embodiment, the invention relates to the compounds of the formula (I) in which W is S.

In the preferred embodiments I-1 to I-8 which follow, U is U-2, U-9, U-10 or U-23, where all other groups, radicals and substituents have the definition described above in Configuration (1-1) or in Configuration (1-2) or in Configuration (2-1) or in Configuration (2-2) or in Configuration (3-1) or in Configuration (3-2) or in Configuration (4-1) or in Configuration (4-2) or in Configuration (5-1) or in Configuration (5-2).

In a further preferred embodiment, the invention relates to the compounds of the formula (I-1).

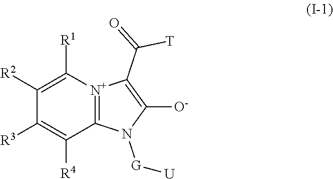

Among these, particular preference is given to the configurations shown below:

| compounds of | with U | all other structural |
|---|---|---|
| the formula | as per | elements as per |
| I-1 | U-2 | Configuration (1-1) |
| I-1 | U-2 | Configuration (2-1) |
| I-1 | U-2 | Configuration (3-1) |
| I-1 | U-2 | Configuration (4-1) |
| I-1 | U-2 | Configuration (5-1) |
| I-1 | U-2 | Configuration (1-2) |
| I-1 | U-2 | Configuration (2-2) |
| I-1 | U-2 | Configuration (3-2) |
| I-1 | U-2 | Configuration (4-2) |
| I-1 | U-2 | Configuration (5-2) |
| I-1 | U-9 | Configuration (1-1) |
| I-1 | U-9 | Configuration (2-1) |
| I-1 | U-9 | Configuration (3-1) |
| I-1 | U-9 | Configuration (4-1) |
| I-1 | U-9 | Configuration (5-1) |
| I-1 | U-9 | Configuration (1-2) |
| I-1 | U-9 | Configuration (2-2) |
| I-1 | U-9 | Configuration (3-2) |
| I-1 | U-9 | Configuration (4-2) |
| I-1 | U-9 | Configuration (5-2) |
| I-1 | U-10 | Configuration (1-1) |
| I-1 | U-10 | Configuration (2-1) |
| I-1 | U-10 | Configuration (3-1) |
| I-1 | U-10 | Configuration (4-1) |
| I-1 | U-10 | Configuration (5-1) |
| I-1 | U-10 | Configuration (1-2) |
| I-1 | U-10 | Configuration (2-2) |
| I-1 | U-10 | Configuration (3-2) |
| I-1 | U-10 | Configuration (4-2) |
| I-1 | U-10 | Configuration (5-2) |
| I-1 | U-23 | Configuration (1-1) |
| I-1 | U-23 | Configuration (2-1) |
| I-1 | U-23 | Configuration (3-1) |
| I-1 | U-23 | Configuration (4-1) |
| I-1 | U-23 | Configuration (5-1) |
| I-1 | U-23 | Configuration (1-2) |
| I-1 | U-23 | Configuration (2-2) |
| I-1 | U-23 | Configuration (3-2) |
| I-1 | U-23 | Configuration (4-2) |
| I-1 | U-23 | Configuration (5-2) |

In a further preferred embodiment, the invention relates to the compounds of the formula (I-2).

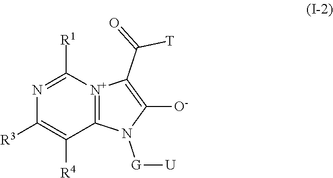

Among these, particular preference is given to the configurations shown below:

| compounds of | with U | all other structural |
|---|---|---|
| the formula | as per | elements as per |
| I-2 | U-2 | Configuration (1-1) |
| I-2 | U-2 | Configuration (2-1) |
| I-2 | U-2 | Configuration (3-1) |
| I-2 | U-2 | Configuration (4-1) |
| I-2 | U-2 | Configuration (5-1) |
| I-2 | U-2 | Configuration (1-2) |
| I-2 | U-2 | Configuration (2-2) |
| I-2 | U-2 | Configuration (3-2) |
| I-2 | U-2 | Configuration (4-2) |
| I-2 | U-2 | Configuration (5-2) |
| I-2 | U-9 | Configuration (1-1) |
| I-2 | U-9 | Configuration (2-1) |
| I-2 | U-9 | Configuration (3-1) |
| I-2 | U-9 | Configuration (4-1) |
| I-2 | U-9 | Configuration (5-1) |
| I-2 | U-9 | Configuration (1-2) |
| I-2 | U-9 | Configuration (2-2) |
| I-2 | U-9 | Configuration (3-2) |
| I-2 | U-9 | Configuration (4-2) |
| I-2 | U-9 | Configuration (5-2) |
| I-2 | U-10 | Configuration (1-1) |
| I-2 | U-10 | Configuration (2-1) |
| I-2 | U-10 | Configuration (3-1) |
| I-2 | U-10 | Configuration (4-1) |
| I-2 | U-10 | Configuration (5-1) |
| I-2 | U-10 | Configuration (1-2) |
| I-2 | U-10 | Configuration (2-2) |
| I-2 | U-10 | Configuration (3-2) |
| I-2 | U-10 | Configuration (4-2) |
| I-2 | U-10 | Configuration (5-2) |
| I-2 | U-23 | Configuration (1-1) |
| I-2 | U-23 | Configuration (2-1) |
| I-2 | U-23 | Configuration (3-1) |
| I-2 | U-23 | Configuration (4-1) |
| I-2 | U-23 | Configuration (5-1) |
| I-2 | U-23 | Configuration (1-2) |
| I-2 | U-23 | Configuration (2-2) |
| I-2 | U-23 | Configuration (3-2) |
| I-2 | U-23 | Configuration (4-2) |
| I-2 | U-23 | Configuration (5-2) |

In a further preferred embodiment, the invention relates to the compounds of the formula (I-3).

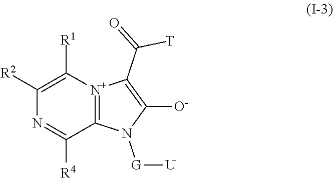

Among these, particular preference is given to the configurations shown below:

| compounds of | with U | all other structural |
|---|---|---|
| the formula | as per | elements as per |
| I-3 | U-2 | Configuration (1-1) |
| I-3 | U-2 | Configuration (2-1) |
| I-3 | U-2 | Configuration (3-1) |
| I-3 | U-2 | Configuration (4-1) |
| I-3 | U-2 | Configuration (5-1) |
| I-3 | U-2 | Configuration (1-2) |
| I-3 | U-2 | Configuration (2-2) |
| I-3 | U-2 | Configuration (3-2) |
| I-3 | U-2 | Configuration (4-2) |
| I-3 | U-2 | Configuration (5-2) |
| I-3 | U-9 | Configuration (1-1) |
| I-3 | U-9 | Configuration (2-1) |
| I-3 | U-9 | Configuration (3-1) |
| I-3 | U-9 | Configuration (4-1) |
| I-3 | U-9 | Configuration (5-1) |
| I-3 | U-9 | Configuration (1-2) |
| I-3 | U-9 | Configuration (2-2) |
| I-3 | U-9 | Configuration (3-2) |
| I-3 | U-9 | Configuration (4-2) |
| I-3 | U-9 | Configuration (5-2) |
| I-3 | U-10 | Configuration (1-1) |
| I-3 | U-10 | Configuration (2-1) |
| I-3 | U-10 | Configuration (3-1) |
| I-3 | U-10 | Configuration (4-1) |
| I-3 | U-10 | Configuration (5-1) |
| I-3 | U-10 | Configuration (1-2) |
| I-3 | U-10 | Configuration (2-2) |
| I-3 | U-10 | Configuration (3-2) |
| I-3 | U-10 | Configuration (4-2) |
| I-3 | U-10 | Configuration (5-2) |
| I-3 | U-23 | Configuration (1-1) |
| I-3 | U-23 | Configuration (2-1) |
| I-3 | U-23 | Configuration (3-1) |
| I-3 | U-23 | Configuration (4-1) |
| I-3 | U-23 | Configuration (5-1) |
| I-3 | U-23 | Configuration (1-2) |
| I-3 | U-23 | Configuration (2-2) |
| I-3 | U-23 | Configuration (3-2) |
| I-3 | U-23 | Configuration (4-2) |
| I-3 | U-23 | Configuration (5-2) |

In a further preferred embodiment, the invention relates to the compounds of the formula (I-4)

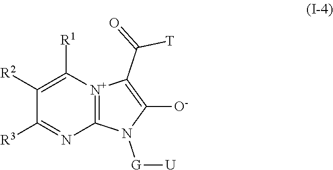

Among these, particular preference is given to the configurations shown below:

| compounds of | with U | all other structural |
|---|---|---|
| the formula | as per | elements as per |
| I-4 | U-2 | Configuration (1-1) |
| I-4 | U-2 | Configuration (2-1) |
| I-4 | U-2 | Configuration (3-1) |
| I-4 | U-2 | Configuration (4-1) |
| I-4 | U-2 | Configuration (5-1) |
| I-4 | U-2 | Configuration (1-2) |
| I-4 | U-2 | Configuration (2-2) |
| I-4 | U-2 | Configuration (3-2) |
| I-4 | U-2 | Configuration (4-2) |
| I-4 | U-2 | Configuration (5-2) |
| I-4 | U-9 | Configuration (1-1) |
| I-4 | U-9 | Configuration (2-1) |
| I-4 | U-9 | Configuration (3-1) |
| I-4 | U-9 | Configuration (4-1) |
| I-4 | U-9 | Configuration (5-1) |
| I-4 | U-9 | Configuration (1-2) |
| I-4 | U-9 | Configuration (2-2) |
| I-4 | U-9 | Configuration (3-2) |
| I-4 | U-9 | Configuration (4-2) |
| I-4 | U-9 | Configuration (5-2) |
| I-4 | U-10 | Configuration (1-1) |
| I-4 | U-10 | Configuration (2-1) |
| I-4 | U-10 | Configuration (3-1) |
| I-4 | U-10 | Configuration (4-1) |
| I-4 | U-10 | Configuration (5-1) |
| I-4 | U-10 | Configuration (1-2) |
| I-4 | U-10 | Configuration (2-2) |
| I-4 | U-10 | Configuration (3-2) |
| I-4 | U-10 | Configuration (4-2) |
| I-4 | U-10 | Configuration (5-2) |
| I-4 | U-23 | Configuration (1-1) |
| I-4 | U-23 | Configuration (2-1) |
| I-4 | U-23 | Configuration (3-1) |
| I-4 | U-23 | Configuration (4-1) |
| I-4 | U-23 | Configuration (5-1) |
| I-4 | U-23 | Configuration (1-2) |
| I-4 | U-23 | Configuration (2-2) |
| I-4 | U-23 | Configuration (3-2) |
| I-4 | U-23 | Configuration (4-2) |
| I-4 | U-23 | Configuration (5-2) |

In a further preferred embodiment, the invention relates to the compounds of the formula (I-5)

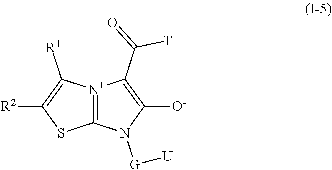

Among these, particular preference is given to the configurations shown below:

| compounds of | with U | all other structural |
|---|---|---|
| the formula | as per | elements as per |
| I-5 | U-2 | Configuration (1-1) |
| I-5 | U-2 | Configuration (2-1) |
| I-5 | U-2 | Configuration (3-1) |
| I-5 | U-2 | Configuration (4-1) |
| I-5 | U-2 | Configuration (5-1) |
| I-5 | U-2 | Configuration (1-2) |
| I-5 | U-2 | Configuration (2-2) |
| I-5 | U-2 | Configuration (3-2) |
| I-5 | U-2 | Configuration (4-2) |
| I-5 | U-2 | Configuration (5-2) |
| I-5 | U-9 | Configuration (1-1) |
| I-5 | U-9 | Configuration (2-1) |
| I-5 | U-9 | Configuration (3-1) |
| I-5 | U-9 | Configuration (4-1) |
| I-5 | U-9 | Configuration (5-1) |
| I-5 | U-9 | Configuration (1-2) |
| I-5 | U-9 | Configuration (2-2) |
| I-5 | U-9 | Configuration (3-2) |
| I-5 | U-9 | Configuration (4-2) |
| I-5 | U-9 | Configuration (5-2) |
| I-5 | U-10 | Configuration (1-1) |
| I-5 | U-10 | Configuration (2-1) |
| I-5 | U-10 | Configuration (3-1) |
| I-5 | U-10 | Configuration (4-1) |
| I-5 | U-10 | Configuration (5-1) |
| I-5 | U-10 | Configuration (1-2) |
| I-5 | U-10 | Configuration (2-2) |
| I-5 | U-10 | Configuration (3-2) |
| I-5 | U-10 | Configuration (4-2) |
| I-5 | U-10 | Configuration (5-2) |
| I-5 | U-23 | Configuration (1-1) |
| I-5 | U-23 | Configuration (2-1) |
| I-5 | U-23 | Configuration (3-1) |
| I-5 | U-23 | Configuration (4-1) |
| I-5 | U-23 | Configuration (5-1) |
| I-5 | U-23 | Configuration (1-2) |
| I-5 | U-23 | Configuration (2-2) |
| I-5 | U-23 | Configuration (3-2) |
| I-5 | U-23 | Configuration (4-2) |
| I-5 | U-23 | Configuration (5-2) |

In a further preferred embodiment, the invention relates to the compounds of the formula (I-6)

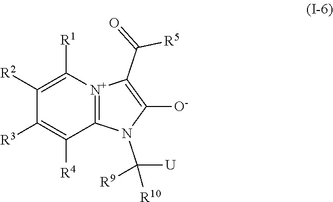

Among these, particular preference is given to the configurations shown below:

| compounds of | with U | all other structural |
|---|---|---|
| the formula | as per | elements as per |
| I-6 | U-2 | Configuration (1-1) |
| I-6 | U-2 | Configuration (2-1) |
| I-6 | U-2 | Configuration (3-1) |
| I-6 | U-2 | Configuration (4-1) |
| I-6 | U-2 | Configuration (5-1) |
| I-6 | U-2 | Configuration (1-2) |
| I-6 | U-2 | Configuration (2-2) |
| I-6 | U-2 | Configuration (3-2) |
| I-6 | U-2 | Configuration (4-2) |
| I-6 | U-2 | Configuration (5-2) |
| I-6 | U-9 | Configuration (1-1) |
| I-6 | U-9 | Configuration (2-1) |
| I-6 | U-9 | Configuration (3-1) |
| I-6 | U-9 | Configuration (4-1) |
| I-6 | U-9 | Configuration (5-1) |
| I-6 | U-9 | Configuration (1-2) |
| I-6 | U-9 | Configuration (2-2) |
| I-6 | U-9 | Configuration (3-2) |
| I-6 | U-9 | Configuration (4-2) |
| I-6 | U-9 | Configuration (5-2) |
| I-6 | U-10 | Configuration (1-1) |
| I-6 | U-10 | Configuration (2-1) |
| I-6 | U-10 | Configuration (3-1) |
| I-6 | U-10 | Configuration (4-1) |
| I-6 | U-10 | Configuration (5-1) |
| I-6 | U-10 | Configuration (1-2) |
| I-6 | U-10 | Configuration (2-2) |
| I-6 | U-10 | Configuration (3-2) |
| I-6 | U-10 | Configuration (4-2) |
| I-6 | U-10 | Configuration (5-2) |
| I-6 | U-23 | Configuration (1-1) |
| I-6 | U-23 | Configuration (2-1) |
| I-6 | U-23 | Configuration (3-1) |
| I-6 | U-23 | Configuration (4-1) |
| I-6 | U-23 | Configuration (5-1) |
| I-6 | U-23 | Configuration (1-2) |
| I-6 | U-23 | Configuration (2-2) |
| I-6 | U-23 | Configuration (3-2) |
| I-6 | U-23 | Configuration (4-2) |
| I-6 | U-23 | Configuration (5-2) |

In a further preferred embodiment, the invention relates to the compounds of the formula (I-7)

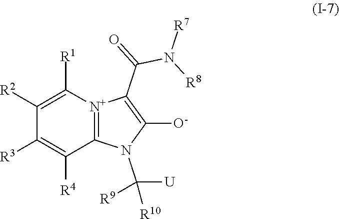

Among these, particular preference is given to the configurations shown below:

| compounds of | with U | all other structural |
|---|---|---|
| the formula | as per | elements as per |
| I-7 | U-2 | Configuration (1-1) |
| I-7 | U-2 | Configuration (2-1) |
| I-7 | U-2 | Configuration (3-1) |
| I-7 | U-2 | Configuration (4-1) |
| I-7 | U-2 | Configuration (5-1) |
| I-7 | U-2 | Configuration (1-2) |
| I-7 | U-2 | Configuration (2-2) |
| I-7 | U-2 | Configuration (3-2) |
| I-7 | U-2 | Configuration (4-2) |
| I-7 | U-2 | Configuration (5-2) |
| I-7 | U-9 | Configuration (1-1) |
| I-7 | U-9 | Configuration (2-1) |
| I-7 | U-9 | Configuration (3-1) |
| I-7 | U-9 | Configuration (4-1) |
| I-7 | U-9 | Configuration (5-1) |
| I-7 | U-9 | Configuration (1-2) |
| I-7 | U-9 | Configuration (2-2) |
| I-7 | U-9 | Configuration (3-2) |
| I-7 | U-9 | Configuration (4-2) |
| I-7 | U-9 | Configuration (5-2) |
| I-7 | U-10 | Configuration (1-1) |
| I-7 | U-10 | Configuration (2-1) |
| I-7 | U-10 | Configuration (3-1) |
| I-7 | U-10 | Configuration (4-1) |
| I-7 | U-10 | Configuration (5-1) |
| I-7 | U-10 | Configuration (1-2) |
| I-7 | U-10 | Configuration (2-2) |
| I-7 | U-10 | Configuration (3-2) |
| I-7 | U-10 | Configuration (4-2) |
| I-7 | U-10 | Configuration (5-2) |
| I-7 | U-23 | Configuration (1-1) |
| I-7 | U-23 | Configuration (2-1) |
| I-7 | U-23 | Configuration (3-1) |
| I-7 | U-23 | Configuration (4-1) |
| I-7 | U-23 | Configuration (5-1) |
| I-7 | U-23 | Configuration (1-2) |
| I-7 | U-23 | Configuration (2-2) |
| I-7 | U-23 | Configuration (3-2) |
| I-7 | U-23 | Configuration (4-2) |
| I-7 | U-23 | Configuration (5-2) |

In a further preferred embodiment, the invention relates to the compounds of the formula (I-8)

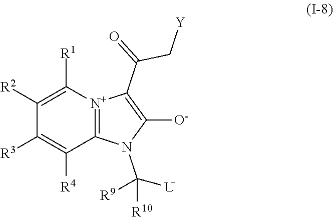

Among these, particular preference is given to the configurations shown below:

| compounds of | with U | all other structural |
|---|---|---|
| the formula | as per | elements as per |
| I-8 | U-2 | Configuration (1-1) |
| I-8 | U-2 | Configuration (2-1) |
| I-8 | U-2 | Configuration (3-1) |
| I-8 | U-2 | Configuration (4-1) |
| I-8 | U-2 | Configuration (5-1) |
| I-8 | U-2 | Configuration (1-2) |
| I-8 | U-2 | Configuration (2-2) |
| I-8 | U-2 | Configuration (3-2) |
| I-8 | U-2 | Configuration (4-2) |
| I-8 | U-2 | Configuration (5-2) |
| I-8 | U-9 | Configuration (1-1) |
| I-8 | U-9 | Configuration (2-1) |
| I-8 | U-9 | Configuration (3-1) |
| I-8 | U-9 | Configuration (4-1) |
| I-8 | U-9 | Configuration (5-1) |
| I-8 | U-9 | Configuration (1-2) |
| I-8 | U-9 | Configuration (2-2) |
| I-8 | U-9 | Configuration (3-2) |
| I-8 | U-9 | Configuration (4-2) |
| I-8 | U-9 | Configuration (5-2) |
| I-8 | U-10 | Configuration (1-1) |
| I-8 | U-10 | Configuration (2-1) |
| I-8 | U-10 | Configuration (3-1) |
| I-8 | U-10 | Configuration (4-1) |
| I-8 | U-10 | Configuration (5-1) |
| I-8 | U-10 | Configuration (1-2) |
| I-8 | U-10 | Configuration (2-2) |
| I-8 | U-10 | Configuration (3-2) |
| I-8 | U-10 | Configuration (4-2) |
| I-8 | U-10 | Configuration (5-2) |
| I-8 | U-23 | Configuration (1-1) |
| I-8 | U-23 | Configuration (2-1) |
| I-8 | U-23 | Configuration (3-1) |
| I-8 | U-23 | Configuration (4-1) |
| I-8 | U-23 | Configuration (5-1) |
| I-8 | U-23 | Configuration (1-2) |
| I-8 | U-23 | Configuration (2-2) |
| I-8 | U-23 | Configuration (3-2) |
| I-8 | U-23 | Configuration (4-2) |
| I-8 | U-23 | Configuration (5-2) |

Among the embodiments I-1 to I-8 cited, preference is given to the following: I-1, I-4, I5, I-6, I-7, I-8.

Among the embodiments I-1 to I-8 cited, particular preference is given to the following: I-6, I-7, I-8.

Among the embodiments I-1 to I-8 cited, very particular preference is given to the following: I-7, I-8.

Compounds according to the invention which are likewise preferred are the compounds of the general formula (I) shown in Table 1.

The radical definitions or elucidations given above in general terms or within areas of preference apply to all compounds of the formula (I) and correspondingly to the starting materials and intermediates. These radical definitions can be combined with one another as desired, i.e. including combinations between the respective ranges of preference.

Preference is given in accordance with the invention to compounds of the formula (I) in which a combination of the definitions given above as preferred is present, and every embodiment described above as preferred constitutes an independent combination, in particular a combination as described in Embodiment 2-1 or in Embodiment 2-2.

Further preference is given in accordance with the invention to compounds of the formula (I) in which a combination of the definitions given above as further preferred is present, and every embodiment described above as further preferred constitutes an independent combination, in particular a combination as described in Embodiment 3-1 or in Embodiment 3-2.

Particular preference is given in accordance with the invention to compounds of the formula (I) in which a combination of the definitions given above as particularly preferred is present, and every embodiment described above as particularly preferred constitutes an independent combination, in particular a combination as described in Embodiment 4-1 or in Embodiment 4-2.

Very particular preference is given in accordance with the invention to compounds of the formula (I) in which a combination of the definitions given above as very particularly preferred is present, and every embodiment described above as very particularly preferred constitutes an independent combination, in particular a combination as described in Embodiment 5-1 or in Embodiment 5-2.

Optionally substituted radicals may be mono- or polysubstituted, where the substituents in the case of polysubstitutions may be the same or different.

The compounds of the formula (I) are mesoionic internal salts. Internal salts, also known as zwitterions, are electrically uncharged molecules which formally bear positive and negative charges on different atoms. The compounds of the formula (I) can be formally represented by various structures which bear the positive and negative charges on different atoms. The figure which follows shows 4 possible representation forms without excluding further possible representation forms. All structural representations are equivalent. For reasons of simplification, just one possible structural representation in each case is chosen here. This representation should be understood in each case as being representative of all valence bond structural representations.

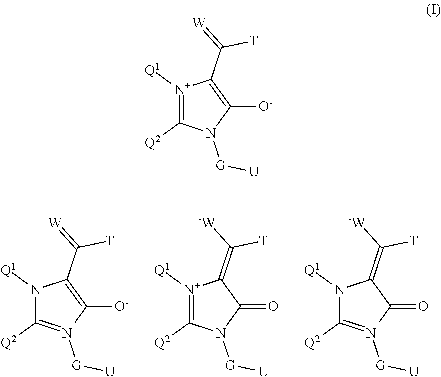

The compounds of the formula (I) may possibly also, depending on the nature of the substituents, be in the form of stereoisomers, i.e. in the form of geometric and/or optical isomers or isomer mixtures of varying composition. This invention provides both the pure stereoisomers and any desired mixtures of these isomers, even though it is generally only compounds of the formula (I) that are discussed here.

However, preference is given in accordance with the invention to using the optically active, stereoisomeric forms of the compounds of the formula (I) and salts thereof.

The invention therefore relates both to the pure enantiomers and diastereomers and to mixtures thereof for controlling animal pests, including arthropods and particularly insects.

If appropriate, the compounds of the formula (I) may be present in various polymorphic forms or as a mixture of various polymorphic forms. Both the pure polymorphs and the polymorph mixtures are provided by the invention and can be used in accordance with the invention.

In the context of the present invention, unless defined differently elsewhere, the term “alkyl”, either on its own or else in combination with further terms, for example haloalkyl, is understood to mean a radical of a saturated aliphatic hydrocarbon group which has 1 to 12 carbon atoms and may be branched or unbranched. Examples of C₁-C₁₂-alkyl radicals are methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1-ethylpropyl, 1,2-dimethylpropyl, hexyl, n-heptyl, n-octyl, n-nonyl, n-decyl, n-undecyl and n-dodecyl. Among these alkyl radicals, particular preference is given to C₁-C₆-alkyl radicals. Special preference is given to C₁-C₄-alkyl radicals.

According to the invention, unless defined differently elsewhere, the term “alkenyl”, either on its own or else in combination with further terms, is understood to mean a straight-chain or branched C2-C12-alkenyl radical which has at least one double bond, for example vinyl, allyl, 1-propenyl, isopropenyl, 1-butenyl, 2-butenyl, 3-butenyl, 1,3-butadienyl, 1-pentenyl, 2-pentenyl, 3-pentenyl, 4-pentenyl, 1,3-pentadienyl, 1-hexenyl, 2-hexenyl, 3-hexenyl, 4-hexenyl, 5-hexenyl and 1,4-hexadienyl. Among these, preference is given to C2-C6-alkenyl radicals and particular preference is C2-C4-alkenyl radicals.

According to the invention, unless defined differently elsewhere, the term “alkynyl”, either on its own or else in combination with further terms, is understood to mean a straight-chain or branched C2-C12-alkynyl radical which has at least one triple bond, for example ethynyl, 1-propynyl and propargyl. Among these, preference is given to C3-C6-alkynyl radicals and particular preference to C3-C4-alkynyl radicals. The alkynyl radical may also contain at least one double bond.

According to the invention, unless defined differently elsewhere, the term “cycloalkyl”, either on its own or else in combination with further terms, is understood to mean a C3-C8-cycloalkyl radical, for example cyclopropyl, cyclobutyl, cyclopentyl, cyclohexyl, cycloheptyl and cyclooctyl. Among these, preference is given to C3-C6-cycloalkyl radicals.

According to the invention, unless defined differently elsewhere, the term “aryl” is understood to mean an aromatic radical having 6 to 14 carbon atoms, preferably phenyl, naphthyl, anthryl or phenanthrenyl, more preferably phenyl.

Unless defined differently elsewhere, the term “arylalkyl” is understood to mean a combination of the radicals “aryl” and “alkyl” defined according to the invention, where the radical is generally attached via the alkyl group. Examples of these are benzyl, phenylethyl or α-methylbenzyl, benzyl being particularly preferred.

Unless defined differently elsewhere, “hetaryl” denotes a mono-, bi- or tricyclic heterocyclic group of carbon atoms and at least one heteroatom, where at least one cycle is aromatic. Preferably, the hetaryl group contains 3, 4, 5 or 6 carbon atoms selected from the group of furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl.

Unless defined differently elsewhere, “heterocyclyl” denotes a monocyclic, saturated or partially saturated 4-, 5-, 6- or 7-membered ring of carbon atoms and at least one heteroatom in the ring.

Preferably, the heterocyclyl group contains 3, 4, 5 or 6 carbon atoms and 1 or 2 heteroatoms from the group consisting of oxygen, sulphur and nitrogen. Examples of heterocyclyl are azetidinyl, azolidinyl, azinanyl, oxetanyl, oxolanyl, oxanyl, dioxanyl, thietanyl, thiolanyl, thianyl and tetrahydrofuryl.

Unless defined differently elsewhere, “oxoheterocyclyl” and “dioxoheterocyclyl” denote a heterocyclyl which contains, in at least one position in the ring, a ring atom substituted, respectively, by one and two (═O) groups. Preferably, a heteroatom, for example sulphur, is substituted by one or two (═O) groups, resulting respectively in the —S(═O)— and —S(═O)₂— groups, where the sulphur atom is a constituent of the ring.

In the context of the present invention, halogen-substituted radicals, for example “haloalkyl”, are understood to mean radicals which are mono- or polysubstituted by halogen up to the maximum possible number of substituents. In the case of polyhalogenation, the halogen atoms may be identical or different. “Halogen” here is fluorine, chlorine, bromine or iodine, preferably fluorine or chlorine.

The term “alkoxy”, either on its own or else in combination with further terms, for example haloalkoxy, is understood in the present case to mean an O-alkyl radical, where the term “alkyl” is as defined above.

### Description of the Processes and Intermediates

The compounds of the formula (I) can be synthesized, for example, by the methods specified in Schemes 1 to 10. In this context, the R¹, R², R³, R⁴, R⁵, R⁶, R⁷, R⁸, R⁵¹, R⁵³, R⁵⁴, R⁵⁵, G and U radicals specified in the formulae, unless stated otherwise, each have the definition given in Configurations (1-1) to (5-1).

Compounds of the formula (Ia) can be synthesized are shown in Scheme 1, where
L¹ is Br, Cl or —O—C(═O)CH₂—Hal and
Hal is Br or Cl,
   by reacting compounds of the formula (II) with 2 equivalents of a halo ketone or a halo ketone anhydride of the formula (III) to give compounds of the formula (Ia) (see, for example, Canadian Journal of Chemistry (1971), 49(4), 668-671).

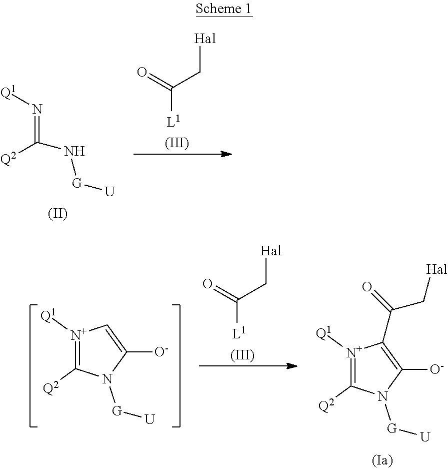

Compounds of the formula II are known from the literature (cf., for example, WO2009099929, WO2012092115, WO2011057022, WO2009099929, WO2011017342) or can be obtained in analogy to methods known from the literature. Compounds of the formula (III) are available for sale.

Compounds of the formula (Ib) in which ALK is C₁-C₄ alkyl can, as shown in Scheme 2, be obtained from amines of the formula (II) by reaction with 2-chloromalonic acid monochloride monoesters of the formula (IV) in the presence of a base, for example triethylamine.

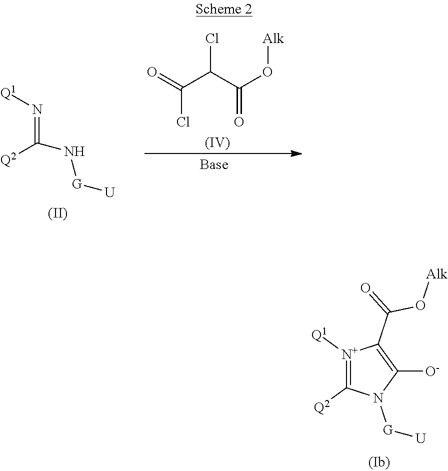

Compounds of the formula (IV) are known in the literature or can be prepared in analogy to known methods (cf. Tetrahedron 1993, 49, 9447-9452).

Compounds of the formula (Ic) can be obtained as shown in Scheme 3, by reacting esters of the formula (Ib) with amino compounds of the formula (V) by commonly known methods. For the reaction with particular amines, it is optionally possible to use activating reagents, for example trimethylaluminium.

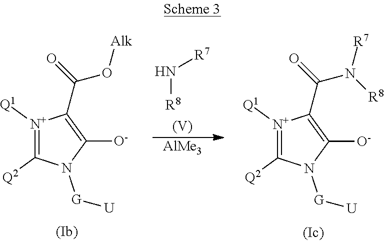

Amines of the formula (V) are available for sale or known in the literature.

Compounds of the formula (Id) can, as shown in Scheme 4, be obtained from compounds of the formula (Ia) by commonly known methods, by reactions with alkoxides or alcohols in the presence of a base, for example sodium hydride. Compounds of the formula (Ie) can be obtained proceeding from compounds of the formula (Id; R⁵¹═H) by commonly known methods, by reaction with compounds of the formula (VII) where L¹ is Cl, Br or —O(═O)R⁵⁵ in the presence of a base, for example triethylamine.

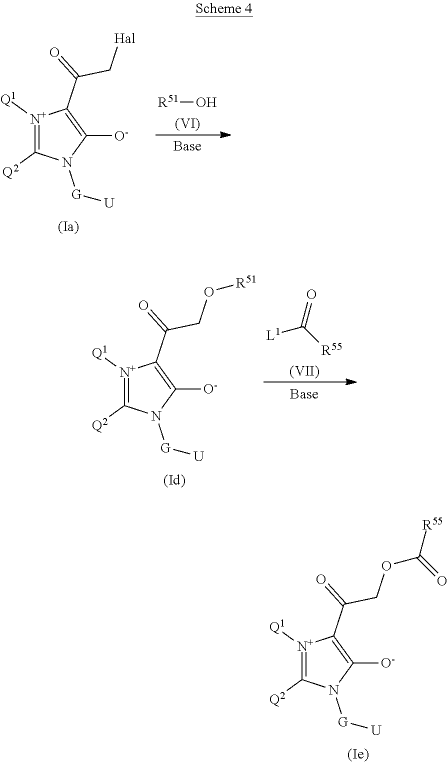

Compounds of the formulae (VI) and (VII) are available for sale or known in the literature.

Compounds of the formula (If) can be obtained by commonly known methods from compounds of the formula (Ia) by reactions with thiolates or thiols in the presence of a base, for example sodium hydride, as shown in Scheme 5. Compounds of the formula (If) can then be oxidized by commonly known methods with an oxidizing agent, for example meta-chloroperbenzoic acid, to give sulphoxides and sulphones of the formula (Ig).

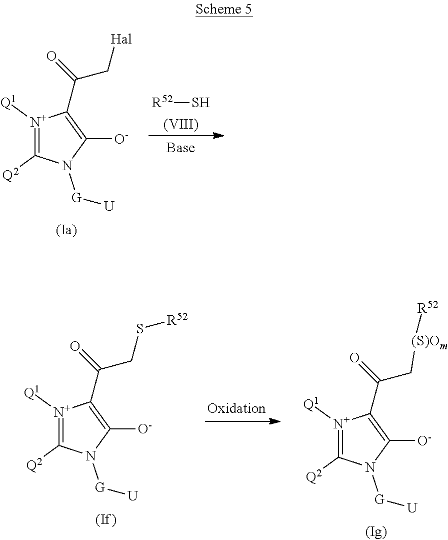

Compounds of the formula (VIII) are available for sale or known in the literature.

Compounds of the formula (Ih) can be obtained from compounds of the formula (Ia) by reactions with amino compounds of the formula (IX) by commonly known methods in the presence of a base, for example triethylamine, as shown in Scheme 6. In the same way, it is also possible to convert other amino compounds, for example hydrazines. Compounds of the formula (Ih, R⁵³═H) in which L¹ is Cl, Br or —O(═O)R⁵⁵ can then be reacted further with carbonyl halides or carboxylic anhydrides of the formula (VII) in the presence of a base, for example triethylamine, to give compounds of the formula (Ii).

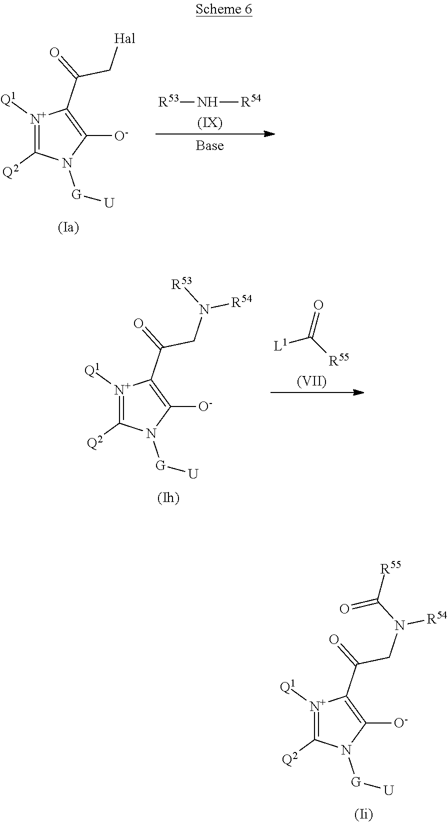

Compounds of the formulae (VII) and (IX) are available for sale or known in the literature.

Compounds of the formula (Ij) can, as shown in Scheme 7, be obtained by dehalogenation of compounds of the formula (Ia) with activated zinc (cf., for example, Dyes and Pigments 2013, 96, 7-15).

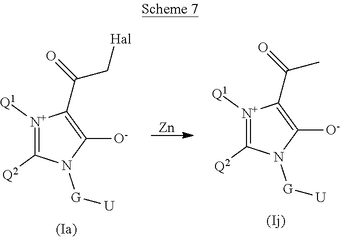

Compounds of the formula (Im) can, as shown in Scheme 8, be obtained in analogy to commonly known methods by reacting amides of the formula (Ic) with a thionating reagent, for example P₂S₅ or Lawesson's reagent.

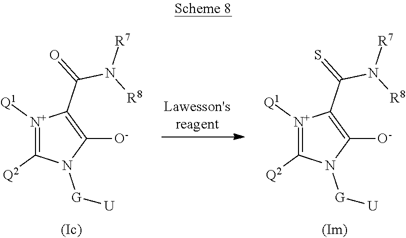

Compounds of the formula (Ik) can be obtained as shown in Scheme 9, wherein carboxylic esters of the formula (Ib) are first reacted in analogy to the method specified in Scheme 3 with N,O-dimethylaminohydroxylamine to give a “Weinreb amide” of the formula (II). The latter is subsequently reacted in analogy to known methods [cf., for example, WO201154844, WO2005100301] with an organometallic compound of the formula (XI), for example an alkyl- or aryllithium compound or a Grignard compound, to give compounds of the formula (Ik).

Compounds of the formulae (XI) and (XII) are available for sale or known in the literature.

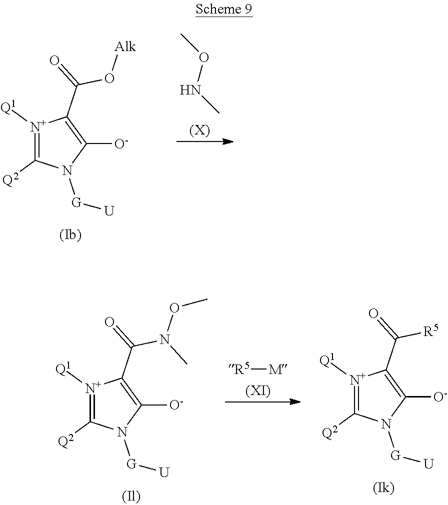

Compounds of the formula (II) can also be obtained as shown in Scheme 10, wherein amines of the formula (II) are reacted in analogy to the method specified in Scheme 2 with acid chlorides of the formula (XII) in the presence of a base.

Compounds of the formula (XII) are either known as acid chlorides in the literature or can be obtained from the corresponding carboxylic acids in analogy to known methods. [cf., for example, EP 37015, DE4301356, JP2006232788].

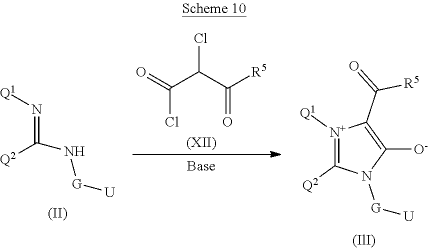

In general, compounds of the formula (I) can be prepared by the processes described above. If individual compounds cannot be prepared by the processes described above, synthesis is possible by derivatization of other compounds of the formula (I), or by individual modifications to the processes described. For example, it may have advantages to prepare certain compounds of the formula (I) from other compounds of the formula (I), for example by hydrolysis, esterification, amide formation, reduction, etherification, oxidation, olefination, halogenation, acylation, alkylation and the like.

The processes according to the invention for preparation of the novel compounds of the formula (I) are preferably performed using a diluent. Useful diluents for performance of the processes according to the invention are, as well as water, all inert solvents. Examples include: halohydrocarbons (e.g. hydrochlorocarbons such as tetraethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene), alcohols (e.g. methanol, ethanol, isopropanol, butanol), ethers (e.g. ethyl propyl ether, methyl tert-butyl ether, anisole, phenetole, cyclohexyl methyl ether, dimethyl ether, diethyl ether, dipropyl ether, diisopropyl ether, di-n-butyl ether, diisobutyl ether, diisoamyl ether, ethylene glycol dimethyl ether, tetrahydrofuran, 1,4-dioxane, dichlorodiethyl ether and polyethers of ethylene oxide and/or propylene oxide), amines (e.g. trimethyl-, triethyl-, tripropyl- and tributylamine, N-methylmorpholine, pyridine and tetramethylenediamine), nitrohydrocarbons (e.g. nitromethane, nitroethane, nitropropane, nitrobenzene, chloronitrobenzene, o-nitrotoluene); nitriles (for example acetonitrile, propionitrile, butyronitrile, isobutyronitrile, benzonitrile, m-chlorobenzonitrile), tetrahydrothiophene dioxide, dimethyl sulphoxide, tetramethylene sulphoxide, dipropyl sulphoxide, benzyl methyl sulphoxide, diisobutyl sulphoxide, dibutyl sulphoxide, diisoamyl sulphoxide, sulphones (e.g. dimethyl, diethyl, dipropyl, dibutyl, diphenyl, dihexyl, methyl ethyl, ethyl propyl, ethyl isobutyl and pentamethylene sulphone), aliphatic, cycloaliphatic or aromatic hydrocarbons (e.g. pentane, hexane, heptane, octane, nonane and technical grade hydrocarbons), and also what are called “white spirits” with components having boiling points in the range of, for example, 40° C. to 250° C., cymene, benzine fractions within a boiling range of 70° C. to 190° C., cyclohexane, methylcyclohexane, petroleum ether, ligroin, octane, benzene, toluene, chlorobenzene, bromobenzene, nitrobenzene, xylene, esters (e.g. methyl, ethyl, butyl and isobutyl acetate, dimethyl, dibutyl and ethylene carbonate); amides (e.g. hexamethylenephosphoramide, formamide, N-methylformamide, N,N-dimethylformamide, N,N-dipropylformamide, N,N-dibutylformamide, N-methylpyrrolidine, N-methylcaprolactam, 1,3-dimethyl-3,4,5,6-tetrahydro-2(1H)-pyrimidine, octylpyrrolidone, octylcaprolactam, 1,3-dimethyl-2-imidazolinedione, N-formylpiperidine, N,N′-diformylpiperazine) and ketones (e.g. acetone, acetophenone, methyl ethyl ketone, methyl butyl ketone).

It is of course also possible to perform the process according to the invention in mixtures of the solvents and diluents mentioned.

When performing the processes according to the invention, the reaction temperatures can be varied within a relatively wide range. In general, the process is carried out at temperatures between −30° C. and +150° C., preferably between −10° C. and +100° C.

The processes according to the invention are generally carried out under standard pressure. However, it is also possible to perform the process according to the invention under elevated or reduced pressure—generally at absolute pressures between 0.1 bar and 15 bar.

To perform the process according to the invention, the starting materials are generally used in approximately equimolar amounts. However, it is also possible to use one of the components in a relatively large excess. The reaction is generally performed in a suitable diluent in the presence of a reaction auxiliary, optionally also under a protective gas atmosphere (for example under nitrogen, argon or helium) and the reaction mixture is generally stirred at the temperature required for several hours. The workup is performed by customary methods (cf. the preparation examples).

The basic reaction auxiliaries used to perform the processes according to the invention may be all suitable acid binders. Examples include: alkaline earth metal or alkali metal compounds (e.g. hydroxides, hydrides, oxides and carbonates of lithium, sodium, potassium, magnesium, calcium and barium), amidine bases or guanidine bases (e.g. 7-methyl-1,5,7-triazabicyclo[4.4.0]dec-5-ene (MTBD); diazabicyclo[4.3.0]nonene (DBN), diazabicyclo[2.2.2]octane (DABCO), 1,8-diazabicyclo[5.4.0]undecene (DBU), cyclohexyltetrabutylguanidine (CyTBG), cyclohexyltetramethylguanidine (CyTMG), N,N,N,N-tetramethyl-1,8-naphthalenediamine, pentamethylpiperidine) and amines, especially tertiary amines (e.g. triethylamine, trimethylamine, tribenzylamine, triisopropylamine, tributylamine, tricyclohexylamine, triamylamine, trihexylamine, N,N-dimethylaniline, N,N-dimethyltoluidine, N,N-dimethyl-p-aminopyridine, N-methylpyrrolidine, N-methylpiperidine, N-methylimidazole, N-methylpyrazole, N-methylmorpholine, N-methylhexamethylenediamine, pyridine, 4-pyrrolidinopyridine, 4-dimethylaminopyridine, quinoline, α-picoline, β-picoline, pyrimidine, acridine, N,N,N′,N′-tetramethylenediamine, N,N,N′,N′-tetraethylenediamine, quinoxaline, N-propyldiisopropylamine, N-ethyldiisopropylamine, N,N′-dimethylcyclohexylamine, 2,6-lutidine, 2,4-lutidine or triethyldiamine).

The acidic reaction auxiliaries which may be used to perform the processes according to the invention include all mineral acids (e.g. hydrohalic acids such as hydrofluoric acid, hydrochloric acid, hydrobromic acid or hydriodic acid, and also sulphuric acid, phosphoric acid, phosphorous acid, nitric acid), Lewis acids (e.g. aluminium(III) chloride, boron trifluoride or its etherate, titanium(IV) chloride, tin(IV) chloride) and organic acids (e.g. formic acid, acetic acid, propionic acid, malonic acid, lactic acid, oxalic acid, fumaric acid, adipic acid, stearic acid, tartaric acid, oleic acid, methanesulphonic acid, benzoic acid, benzenesulphonic acid or para-toluenesulphonic acid).

### Isomers

Depending on the nature of the substituents, the compounds of the formula (I) may be in the form of geometric and/or optically active isomers or corresponding isomer mixtures in different compositions. These stereoisomers are, for example, enantiomers, diastereomers, atropisomers or geometric isomers. The invention therefore encompasses both pure stereoisomers and any desired mixtures of these isomers.

### Methods and Uses

The invention also relates to methods for controlling animal pests, in which compounds of the formula (I) are allowed to act on animal pests and/or their habitat. The control of the animal pests is preferably conducted in agriculture and forestry, and in material protection. This preferably excludes methods for surgical or therapeutic treatment of the human or animal body and diagnostic methods carried out on the human or animal body.

The invention further relates to the use of the compounds of the formula (I) as pesticides, especially crop protection agents.

In the context of the present application, the term “pesticide” in each case also always encompasses the term “crop protection agent”.

The compounds of the formula (I), given good plant tolerance, favourable homeotherm toxicity and good environmental compatibility, are suitable for protecting plants and plant organs against biotic and abiotic stress factors, for increasing harvest yields, for improving the quality of the harvested material and for controlling animal pests, especially insects, arachnids, helminths, especially nematodes and molluscs, which are encountered in agriculture, in horticulture, in animal husbandry, in aquatic cultures, in forests, in gardens and leisure facilities, in the protection of stored products and of materials, and in the hygiene sector.

In the context of the present patent application, the term “hygiene” should be understood to mean any and all measures, provisions and procedures which have the aim of preventing diseases, especially infection diseases, and which serve to protect the health of humans and animals and/or protect the environment and/or maintain cleanliness. According to the invention, this especially includes measures for cleaning, disinfection and sterilization, for example of textiles or hard surfaces, especially surfaces made of glass, wood, cement, porcelain, ceramic, plastic or else metal(s), in order to ensure that these are free of hygiene pests and/or their secretions. The scope of protection of the invention in this regard preferably excludes surgical or therapeutic treatment procedures to be applied to the human body or the bodies of animals, and diagnostic procedures which are conducted on the human body or the bodies of animals.

The term “hygiene sector” covers all areas, technical fields and industrial applications in which these hygiene measures, provisions and procedures are important, for example with regard to hygiene in kitchens, bakeries, airports, bathrooms, swimming pools, department stores, hotels, hospitals, stalls, animal keeping, etc.

The term “hygiene pest” should therefore be understood to mean one or more animal pests whose presence in the hygiene sector is problematic, especially for reasons of health. A main aim is therefore that of avoiding, or limiting to a minimum degree, the presence of hygiene pests and/or the exposure to these in the hygiene sector. This can especially be achieved through the use of a pesticide which can be used both for prevention of infestation and for prevention of an existing infestation. It is also possible to use formulations which prevent or reduce exposure to pests. Hygiene pests include, for example, the organisms mentioned below.

The term “hygiene protection” thus covers all acts by which these hygiene measures, provisions and procedures are maintained and/or improved.

The compounds of the formula (I) can preferably be used as pesticides. They are active against normally sensitive and resistant species and also against all or some stages of development. The abovementioned pests include:

pests from the phylum of the Arthropoda, especially from the class of the Arachnida, for example Acarus spp., e.g. Acarus siro, Aceria kuko, Aceria sheldoni, Aculops spp., Aculus spp., e.g. Aculus fockeui, Aculus schlechtendali, Amblyomma spp., Amphitetranychus viennensis, Argas spp., Boophilus spp., Brevipalpus spp., e.g. Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., e.g. Eotetranychus hicoriae, Epitrimerus pyri, Eutetranychus spp., e.g. Eutetranychus banksi, Eriophyes spp., e.g. Eriophyes pyri, Glycyphagus domesticus, Halotydeus destructor, Hemitarsonemus spp., e.g. Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., e.g. Oligonychus coffeae, Oligonychus coniferarum, Oligonychus ilicis, Oligonychus indicus, Oligonychus mangiferus, Oligonychus pratensis, Oligonychus punicae, Oligonychus yothersi, Ornithodorus spp., Ornithonyssus spp., Panonychus spp., e.g. Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., e.g. Tarsonemus confusus, Tarsonemus pallidus, Tetranychus spp., e.g. Tetranychus canadensis, Tetranychus cinnabarinus, Tetranychus turkestani, Tetranychus urticae, Trombicula alfreddugesi, Vaejovis spp., Vasates lycopersici;
from the class of the Chilopoda, e.g. Geophilus spp., Scutigera spp.;
from the order or the class of the Collembola, e.g. Onychiurus armatus; Sminthurus viridis;
from the class of the Diplopoda, e.g. Blaniulus guttulatus;
from the class of the Insecta, for example from the order of the Blattodea, e.g. Blatta orientalis, Blattella asahinai, Blattella germanica, Leucophaea maderae, Loboptera decipiens, Neostylopyga rhombifolia, Panchlora spp., Parcoblatta spp., Periplaneta spp., e.g. Periplaneta americana, Periplaneta australasiae, Pycnoscelus surinamensis, Supella longipalpa;
from the order of the Coleoptera for example Acalymma vittatum, Acanthoscelides obtectus, Adoretus spp., Aethina tumida, Agelastica alni, Agriotes spp., e.g. Agriotes linneatus, Agriotes mancus, Alphitobius diaperinus, Amphimallon solstitialis, Anobium punctatum, Anoplophora spp., Anthonomus spp., e.g. Anthonomus grandis, Anthrenus spp., Apion spp., Apogonia spp., Atomaria spp., e.g. Atomaria linearis, Attagenus spp., Baris caerulescens, Bruchidius obtectus, Bruchus spp., e.g. Bruchus pisorum, Bruchus rufimanus, Cassida spp., Cerotoma trifurcata, Ceutorrhynchus spp., e.g. Ceutorrhynchus assimilis, Ceutorrhynchus quadridens, Ceutorrhynchus rapae, Chaetocnema spp., e.g. Chaetocnema confinis, Chaetocnema denticulata, Chaetocnema ectypa, Cleonus mendicus, Conoderus spp., Cosmopolites spp., e.g. Cosmopolites sordidus, Costelytra zealandica, Ctenicera spp., Curculio spp., e.g. Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus furnissi, Dermestes spp., Diabrotica spp., e.g. Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epicaerus spp., Epilachna spp., e.g. Epilachna borealis, Epilachna varivestis, Epitrix spp., e.g. Epitrix cucumeris, Epitrix fuscula, Epitrix hirtipennis, Epitrix subcrinita, Epitrix tuberis, Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., e.g. Hypothenemus hampei, Hypothenemus obscurus, Hypothenemus pubescens, Lachnosterna consanguinea, Lasioderma serricorne, Latheticus oryzae, Lathridius spp., Lema spp., Leptinotarsa decemlineata, Leucoptera spp., e.g. Leucoptera coffeella, Lissorhoptrus oryzophilus, Listronotus (=Hyperodes) spp., Lixus spp., Luperodes spp., Luperomorpha xanthodera, Lyctus spp., Megascelis spp., Melanotus spp., e.g. Melanotus longulus oregonensis, Meligethes aeneus, Melolontha spp., e.g. Melolontha melolontha, Migdolus spp., Monochamus spp., Naupactus xanthographus, Necrobia spp., Neogalerucella spp., Niptus hololeucus, Oryctes rhinoceros, Oryzaephilus surinamensis, Oryzaphagus oryzae, Otiorhynchus spp., e.g. Otiorhynchus cribricollis, Otiorhynchus ligustici, Otiorhynchus ovatus, Otiorhynchus rugosostriarus, Otiorhynchus sulcatus, Oulema spp., e.g. Oulema melanopus, Oulema oryzae, Oxycetonia jucunda, Phaedon cochleariae, Phyllophaga spp., Phyllophaga helleri, Phyllotreta spp., e.g. Phyllotreta armoraciae, Phyllotreta pusilla, Phyllotreta ramosa, Phyllotreta striolata, Popillia japonica, Premnotrypes spp., Prostephanus truncatus, Psylliodes spp., e.g. Psylliodes affinis, Psylliodes chrysocephala, Psylliodes punctulata, Ptinus spp., Rhizobius ventralis, Rhizopertha dominica, Rhynchophorus spp., Rhynchophorus ferrugineus, Rhynchophorus palmarum, Sinoxylon perforans, Sitophilus spp., e.g. Sitophilus granarius, Sitophilus linearis, Sitophilus oryzae, Sitophilus zeamais, Sphenophorus spp., Stegobium paniceum, Sternechus spp., e.g. Sternechus paludatus, Symphyletes spp., Tanymecus spp., e.g. Tanymecus dilaticollis, Tanymecus indicus, Tanymecus palliatus, Tenebrio molitor, Tenebrioides mauretanicus, Tribolium spp., e.g. Tribolium audax, Tribolium castaneum, Tribolium confusum, Trogoderma spp., Tychius spp., Xylotrechus spp., Zabrus spp., e.g. Zabrus tenebrioides;
from the order of the Dermaptera, for example Anisolabis maritime, Forficula auricularia, Labidura riparia;
from the order of the Diptera, for example Aedes spp., e.g. Aedes aegypti, Aedes albopictus, Aedes sticticus, Aedes vexans, Agromyza spp., e.g. Agromyza frontella, Agromyza parvicornis, Anastrepha spp., Anopheles spp., e.g. Anopheles quadrimaculatus, Anopheles gambiae, Asphondylia spp., Bactrocera spp., e.g. Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., e.g. Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., e.g. Culex pipiens, Culex quinquefasciatus, Culicoides spp., Culiseta spp., Cuterebra spp., Dacus oleae, Dasineura spp., e.g. Dasineura brassicae, Delia spp., e.g. Delia antiqua, Delia coarctata, Delia florilega, Delia platura, Delia radicum, Dermatobia hominis, Drosophila spp., e.g. Drosphila melanogaster, Drosophila suzukii, Echinocnemus spp., Euleia heraclei, Fannia spp., Gasterophilus spp., Glossina spp., Haematopota spp., Hydrellia spp., Hydrellia griseola, Hylemya spp., Hippobosca spp., Hypoderma spp., Liriomyza spp., e.g. Liriomyza brassicae, Liriomyza huidobrensis, Liriomyza sativae, Lucilia spp., e.g. Lucilia cuprina, Lutzomyia spp., Mansonia spp., Musca spp., e.g. Musca domestica, Musca domestica vicina, Oestrus spp., Oscinella frit, Paratanytarsus spp., Paralauterbomiella subcincta, Pegomya or Pegomyia spp., e.g. Pegomya betae, Pegomya hyoscyami, Pegomya rubivora, Phlebotomus spp., Phorbia spp., Phormia spp., Piophila casei, Platyparea poeciloptera, Prodiplosis spp., Psila rosae, Rhagoletis spp., e.g. Rhagoletis cingulata, Rhagoletis completa, Rhagoletis fausta, Rhagoletis indifferens, Rhagoletis mendax, Rhagoletis pomonella, Sarcophaga spp., Simulium spp., e.g. Simulium meridionale, Stomoxys spp., Tabanus spp., Tetanops spp., Tipula spp., e.g. Tipula paludosa, Tipula simplex, Toxotrypana curvicauda;
from the order of the Hemiptera, for example Acizzia acaciaebaileyanae, Acizzia dodonaeae, Acizzia uncatoides, Acrida turrita, Acyrthosipon spp., e.g. Acyrthosiphon pisum, Acrogonia spp., Aeneolamia spp., Agonoscena spp., Aleurocanthus spp., Aleyrodes proletella, Aleurolobus barodensis, Aleurothrixus floccosus, Allocaridara malayensis, Amrasca spp., e.g. Amrasca bigutulla, Amrasca devastans, Anuraphis cardui, Aonidiella spp., e.g. Aonidiella aurantii, Aonidiella citrina, Aonidiella inomata, Aphanostigma piri, Aphis spp., e.g. Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis vibumiphila, Arboridia apicalis, Arytainilla spp., Aspidiella spp., Aspidiotus spp., e.g. Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., e.g. Cacopsylla pyricola, Calligypona marginata, Capulinia spp., Cameocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus aonidum, Chrysomphalus ficus, Cicadulina mbila, Coccomytilus halli, Coccus spp., e.g. Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes chittendeni, Dialeurodes citri, Diaphorina citri, Diaspis spp., Diuraphis spp., Doralis spp., Drosicha spp., Dysaphis spp., e.g. Dysaphis apiifolia, Dysaphis plantaginea, Dysaphis tulipae, Dysmicoccus spp., Empoasca spp., e.g. Empoasca abrupta, Empoasca fabae, Empoasca maligna, Empoasca solana, Empoasca stevensi, Eriosoma spp., e.g. Eriosoma americanum, Eriosoma lanigerum, Eriosoma pyricola, Erythroneura spp., Eucalyptolyma spp., Euphyllura spp., Euscelis bilobatus, Ferrisia spp., Fiorinia spp., Furcaspis oceanica, Geococcus coffeae, Glycaspis spp., Heteropsylla cubana, Heteropsylla spinulosa, Homalodisca coagulata, Hyalopterus arundinis, Hyalopterus pruni, Icerya spp., e.g. Icerya purchasi, Idiocerus spp., Idioscopus spp., Laodelphax striatellus, Lecanium spp., e.g. Lecanium corni (=Parthenolecanium corni), Lepidosaphes spp., e.g. Lepidosaphes ulmi, Lipaphis erysimi, Lopholeucaspis japonica, Lycorma delicatula, Macrosiphum spp., e.g. Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., e.g. Myzus ascalonicus, Myzus cerasi, Myzus ligustri, Myzus ornatus, Myzus persicae, Myzus nicotianae, Nasonovia ribisnigri, Neomaskellia spp., Nephotettix spp., e.g. Nephotettix cincticeps, Nephotettix nigropictus, Nettigoniclla spectra, Nilaparvata lugens, Oncometopia spp., Orthezia praelonga, Oxya chinensis, Pachypsylla spp., Parabemisia myricae, Paratrioza spp., e.g. Paratrioza cockerelli, Parlatoria spp., Pemphigus spp., e.g. Pemphigus bursarius, Pemphigus populivenae, Peregrinus maidis, Perkinsiella spp., Phenacoccus spp., e.g. Phenacoccus madeirensis, Phloeomyzus passerinii, Phorodon humuli, Phylloxera spp., e.g. Phylloxera devastatrix, Phylloxera notabilis, Pinnaspis aspidistrae, Planococcus spp., e.g. Planococcus citri, Prosopidopsylla flava, Protopulvinaria pyriformis, Pseudaulacaspis pentagona, Pseudococcus spp., e.g. Pseudococcus calceolariae, Pseudococcus comstocki, Pseudococcus longispinus, Pseudococcus maritimus, Pseudococcus viburni, Psyllopsis spp., Psylla spp., e.g. Psylla buxi, Psylla mali, Psylla pyri, Pteromalus spp., Pulvinaria spp., Pyrilla spp., Quadraspidiotus spp., e.g. Quadraspidiotus juglansregiae, Quadraspidiotus ostreaeformis, Quadraspidiotus perniciosus, Quesada gigas, Rastrococcus spp., Rhopalosiphum spp., e.g. Rhopalosiphum maidis, Rhopalosiphum oxyacanthae, Rhopalosiphum padi, Rhopalosiphum rufiabdominale, Saissetia spp., e.g. Saissetia coffeae, Saissetia miranda, Saissetia neglecta, Saissetia oleae, Scaphoideus titanus, Schizaphis graminum, Selenaspidus articulatus, Sipha flava, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., e.g. Toxoptera aurantii, Toxoptera citricidus, Trialeurodes vaporariorum, Trioza spp., e.g. Trioza diospyri, Typhlocyba spp., Unaspis spp., Viteus vitifolii, Zygina spp.; from the suborder of the Heteroptera, for example Aelia spp., Anasa tristis, Antestiopsis spp., Boisea spp., Blissus spp., Calocoris spp., Campylomma livida, Cavelerius spp., Cimex spp., e.g. Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., e.g. Euschistus heros, Euschistus servus, Euschistus tristigmus, Euschistus variolarius, Eurydema spp., Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., e.g. Lygocoris pabulinus, Lygus spp., e.g. Lygus elisus, Lygus hesperus, Lygus lineolaris, Macropes excavatus, Megacopta cribraria, Miridae, Monalonion atratum, Nezara spp., e.g. Nezara viridula, Nysius spp., Oebalus spp., Pentomidae, Piesma quadrata, Piezodorus spp., e.g. Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;
from the order of the Hymenoptera, for example Acromyrmex spp., Athalia spp., e.g. Athalia rosae, Atta spp., Camponotus spp., Dolichovespula spp., Diprion spp., e.g. Diprion similis, Hoplocampa spp., e.g. Hoplocampa cookei, Hoplocampa testudinea, Lasius spp., Linepithema (Iridiomyrmex) humile, Monomorium pharaonis, Paratrechina spp., Paravespula spp., Plagiolepis spp., Sirex spp., Solenopsis invicta, Tapinoma spp., Technomyrmex albipes, Urocerus spp., Vespa spp., e.g. Vespa crabro, Wasmannia auropunctata, Xeris spp.;
from the order of the Isopoda, for example Armadillidium vulgare, Oniscus asellus, Porcellio scaber;
from the order of the Isoptera, for example Coptotermes spp., e.g. Coptotermes formosanus, Cornitermes cumulans, Cryptotermes spp., Incisitermes spp., Kalotermes spp., Microtermes obesi, Nasutitermes spp., Odontotermes spp., Porotermes spp., Reticulitermes spp., e.g. Reticulitermes flavipes, Reticulitermes hesperus;
from the order of the Lepidoptera, for example Achroia grisella, Acronicta major, Adoxophyes spp., e.g. Adoxophyes orana, Aedia leucomelas, Agrotis spp., e.g. Agrotis segetum, Agrotis ipsilon, Alabama spp., e.g. Alabama argillacea, Amyelois transitella, Anarsia spp., Anticarsia spp., e.g. Anticarsia gemmatalis, Argyroploce spp., Autographa spp., Barathra brassicae, Blastodacna atra, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., e.g. Chilo plejadellus, Chilo suppressalis, Choreutis pariana, Choristoneura spp., Chrysodeixis chalcites, Clysia ambiguella, Cnaphalocerus spp., Cnaphalocrocis medinalis, Cnephasia spp., Conopomorpha spp., Conotrachelus spp., Copitarsia spp., Cydia spp., e.g. Cydia nigricana, Cydia pomonella, Dalaca noctuides, Diaphania spp., Diparopsis spp., Diatraea saccharalis, Earias spp., Ecdytolopha aurantium, Elasmopalpus lignosellus, Eldana saccharina, Ephestia spp., e.g. Ephestia elutella, Ephestia kuehniella, Epinotia spp., Epiphyas postvittana, Erannis spp., Erschoviella musculana, Etiella spp., Eudocima spp., Eulia spp., Eupoecilia ambiguella, Euproctis spp., e.g. Euproctis chrysorrhoea, Euxoa spp., Feltia spp., Galleria mellonella, Gracillaria spp., Grapholitha spp., e.g. Grapholita molesta, Grapholita prunivora, Hedylepta spp., Helicoverpa spp., e.g. Helicoverpa armigera, Helicoverpa zea, Heliothis spp., e.g. Heliothis virescens Hofmannophila pseudospretella, Homoeosoma spp., Homona spp., Hyponomeuta padella, Kakivoria flavofasciata, Lampides spp., Laphygma spp., Laspeyresia molesta, Leucinodes orbonalis, Leucoptera spp., e.g. Leucoptera coffeella, Lithocolletis spp., e.g. Lithocolletis blancardella, Lithophane antennata, Lobesia spp., e.g. Lobesia botrana, Loxagrotis albicosta, Lymantria spp., e.g. Lymantria dispar, Lyonetia spp., e.g. Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Omphisa spp., Operophtera spp., Oria spp., Orthaga spp., Ostrinia spp., e.g. Ostrinia nubilalis, Panolis flammea, Parnara spp., Pectinophora spp., e.g. Pectinophora gossypiella, Perileucoptera spp., Phthorimaea spp., e.g. Phthorimaea operculella, Phyllocnistis citrella, Phyllonorycter spp., e.g. Phyllonorycter blancardella, Phyllonorycter crataegella, Pieris spp., e.g. Pieris rapae, Platynota stultana, Plodia interpunctella, Plusia spp., Plutella xylostella (=Plutella maculipennis), Prays spp., Prodenia spp., Protoparce spp., Pseudaletia spp., e.g. Pseudaletia unipuncta, Pseudoplusia includens, Pyrausta nubilalis, Rachiplusia nu, Schoenobius spp., e.g. Schoenobius bipunctifer, Scirpophaga spp., e.g. Scirpophaga innotata, Scotia segetum, Sesamia spp., e.g. Sesamia inferens, Sparganothis spp., Spodoptera spp., e.g. Spodoptera eradiana, Spodoptera exigua, Spodoptera frugiperda, Spodoptera praefica, Stathmopoda spp., Stenoma spp., Stomopteryx subsecivella, Synanthedon spp., Tecia solanivora, Thaumetopoea spp., Thermesia gemmatalis, Tinea cloacella, Tinea pellionella, Tineola bisselliella, Tortrix spp., Trichophaga tapetzella, Trichoplusia spp., e.g. Trichoplusia ni, Tryporyza incertulas, Tuta absoluta, Virachola spp.;
from the order of the Orthoptera or Saltatoria, for example Acheta domesticus, Dichroplus spp., Gryllotalpa spp., e.g. Gryllotalpa gryllotalpa, Hieroglyphus spp., Locusta spp., e.g. Locusta migratoria, Melanoplus spp., e.g. Melanoplus devastator, Paratlanticus ussuriensis, Schistocerca gregaria;
from the order of the Phthiraptera, for example Damalinia spp., Haematopinus spp., Linognathus spp., Pediculus spp., Phylloxera vastatrix, Phthirus pubis, Trichodectes spp.;
from the order of the Psocoptera, for example Lepinotus spp., Liposcelis spp.;
from the order of the Siphonaptera, for example Ceratophyllus spp., Ctenocephalides spp., e.g. Ctenocephalides canis, Ctenocephalides felis, Pulex irritans, Tunga penetrans, Xenopsylla cheopis;
from the order of the Thysanoptera, for example Anaphothrips obscurus, Baliothrips biformis, Chaetanaphothrips leeuweni, Drepanothrips reuteri, Enneothrips flavens, Frankliniella spp., e.g. Frankliniella fusca, Frankliniella occidentalis, Frankliniella schultzei, Frankliniella tritici, Frankliniella vaccinii, Frankliniella williamsi, Haplothrips spp., Heliothrips spp., Hercinothrips femoralis, Kakothrips spp., Rhipiphorothrips cruentatus, Scirtothrips spp., Taeniothrips cardamomi, Thrips spp., e.g. Thrips palmi, Thrips tabaci;
from the order of the Zygentoma (=Thysanura), for example Ctenolepisma spp., Lepisma saccharina, Lepismodes inquilinus, Thermobia domestica;
from the class of the Symphyla, for example Scutigerella spp., e.g. Scutigerella immaculata;
pests from the phylum of the Mollusca, for example from the class of the Bivalvia, e.g. Dreissena spp.;
and also from the class of the Gastropoda, for example Arion spp., e.g. Arion ater rufus, Biomphalaria spp., Bulinus spp., Deroceras spp., e.g. Deroceras laeve, Galba spp., Lymnaea spp., Oncomelania spp., Pomacea spp., Succinea spp.;
plant pests from the phylum of the Nematoda, i.e. plant-parasitic nematodes, in particular Aglenchus spp., e.g. Aglenchus agricola, Anguina spp., e.g. Anguina tritici, Aphelenchoides spp., e.g. Aphelenchoides arachidis, Aphelenchoides fragariae, Belonolaimus spp., e.g. Belonolaimus gracilis, Belonolaimus longicaudatus, Belonolaimus nortoni, Bursaphelenchus spp., e.g. Bursaphelenchus cocophilus, Bursaphelenchus eremus, Bursaphelenchus xylophilus, Cacopaurus spp., e.g. Cacopaurus pestis, Criconemella spp., e.g. Criconemella curvata, Criconemella onoensis, Criconemella ornata, Criconemella rusium, Criconemella xenoplax (=Mesocriconema xenoplax), Criconemoides spp., e.g. Criconemoides ferniae, Criconemoides onoense, Criconemoides ornatum, Ditylenchus spp., e.g. Ditylenchus dipsaci, Dolichodorus spp., Globodera spp., e.g. Globodera pallida, Globodera rostochiensis, Helicotylenchus spp., e.g. Helicotylenchus dihystera, Hemicriconemoides spp., Hemicycliophora spp., Heterodera spp., e.g. Heterodera avenae, Heterodera glycines, Heterodera schachtii, Hirschmaniella spp., Hoplolaimus spp., Longidorus spp., e.g. Longidorus africanus, Meloidogyne spp., e.g. Meloidogyne chitwoodi, Meloidogyne fallax, Meloidogyne hapla, Meloidogyne incognita, Meloinema spp., Nacobbus spp., Neotylenchus spp., Paralongidorus spp., Paraphelenchus spp., Paratrichodorus spp., e.g. Paratrichodorus minor, Paratylenchus spp., Pratylenchus spp., e.g. Pratylenchus penetrans, Pseudohalenchus spp., Psilenchus spp., Punctodera spp., Quinisulcius spp., Radopholus spp., e.g. Radopholus citrophilus, Radopholus similis, Rotylenchulus spp., Rotylenchus spp., Scutellonema spp., Subanguina spp., Trichodorus spp., e.g. Trichodorus obtusus, Trichodorus primitivus, Tylenchorhynchus spp., e.g. Tylenchorhynchus annulatus, Tylenchulus spp., e.g.

The compounds of the formula (I) can optionally, at certain concentrations or application rates, also be used as herbicides, safeners, growth regulators or agents to improve plant properties, as microbicides or gametocides, for example as fungicides, antimycotics, bactericides, virucides (including agents against viroids) or as agents against MLO (mycoplasma-like organisms) and RLO (rickettsia-like organisms). They can, as the case may be, also be used as intermediates or precursors for the synthesis of other active ingredients.

### Formulations

The present invention further relates to formulations and use forms prepared therefrom as pesticides, for example drench, drip and spray liquors, comprising at least one compound of the formula (I). Optionally, the use forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropylguar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more compounds of the formula (I), optionally comprise further active agrochemical ingredients.

Preference is given to formulations or use forms comprising auxiliaries, for example extenders, solvents, spontaneity promoters, carriers, emulsifiers, dispersants, frost protection agents, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are produced in a known manner, for example by mixing the compounds of the formula (I) with auxiliaries, for example extenders, solvents and/or solid carriers and/or other auxiliaries, for example surfactants. The formulations are produced either in suitable facilities or else before or during application.

The auxiliaries used may be substances suitable for imparting special properties, such as certain physical, technical and/or biological properties, to the formulation of the compounds of the formula (I), or to the use forms prepared from these formulations (for example ready-to-use pesticides such as spray liquors or seed-dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the simple and substituted amines, amides, lactams (such as N-alkylpyrrolidones) and lactones, the sulphones and sulphoxides (such as dimethyl sulphoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and also water.

In principle, it is possible to use all suitable solvents. Examples of suitable solvents are aromatic hydrocarbons, for example xylene, toluene or alkylnaphthalenes, chlorinated aromatic or chlorinated aliphatic hydrocarbons, for example chlorobenzene, chloroethylene or methylene chloride, aliphatic hydrocarbons, for example cyclohexane, paraffins, petroleum fractions, mineral and vegetable oils, alcohols, for example methanol, ethanol, isopropanol, butanol or glycol and their ethers and esters, ketones, for example acetone, methyl ethyl ketone, methyl isobutyl ketone or cyclohexanone, strongly polar solvents, for example dimethyl sulphoxide, and water.

In principle, it is possible to use all suitable carriers. Useful carriers especially include, for example, ammonium salts and natural, finely ground rocks, such as kaolins, aluminas, talc, chalk, quartz, attapulgite, montmorillonite or diatomaceous earth, and synthetic, finely ground rocks, such as highly disperse silica, aluminium oxide and natural or synthetic silicates, resins, waxes and/or solid fertilizers.

It is likewise possible to use mixtures of such carriers. Useful carriers for granules include: for example crushed and fractionated natural rocks such as calcite, marble, pumice, sepiolite, dolomite, and synthetic granules of inorganic and organic flours, and also granules of organic material such as sawdust, paper, coconut shells, maize cobs and tobacco stalks.

It is also possible to use liquefied gaseous extenders or solvents. Especially suitable extenders or carriers are those which are gaseous at standard temperature and under atmospheric pressure, for example aerosol propellants such as halogenated hydrocarbons, and also butane, propane, nitrogen and carbon dioxide.

Examples of emulsifiers and/or foam formers, dispersants or wetting agents having ionic or nonionic properties or mixtures of these surface-active substances are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), phosphoric esters of polyethoxylated alcohols or phenols, fatty acid esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is advantageous if one of the compounds of the formula (I) and/or one of the inert carriers is insoluble in water and when the application takes place in water.

Further auxiliaries which may be present in the formulations and the use forms derived therefrom include dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components which may be present are stabilizers, such as cold stabilizers, preservatives, antioxidants, light stabilizers, or other agents which improve chemical and/or physical stability. Foam generators or antifoams may also be present.

In addition, the formulations and the use forms derived therefrom may also comprise, as additional auxiliaries, stickers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids. Further auxiliaries may be mineral and vegetable oils.

It is possible if appropriate for still further auxiliaries to be present in the formulations and the use forms derived therefrom. Examples of such additives are fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce dynamic surface tension, for example dioctyl sulphosuccinate, or increase viscoelasticity, for example hydroxypropylguar polymers.

Suitable penetrants in the present context are all those substances which are usually used for improving the penetration of active agrochemical ingredients into plants. Penetrants are defined in this context by their ability to penetrate from the (generally aqueous) application liquor and/or from the spray coating into the cuticle of the plant and hence increase the mobility of the active ingredients in the cuticle. The method described in the literature (Baur et al., 1997, Pesticide Science 51, 131-152) can be used for determining this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably comprise between 0.00000001% and 98% by weight of the compound of the formula (I), more preferably between 0.01% and 95% by weight of the compound of the formula (I), most preferably between 0.5% and 90% by weight of the compound of the formula (I), based on the weight of the formulation.

The content of the compound of the formula (I) in the use forms prepared from the formulations (in particular pesticides) may vary within wide ranges. The concentration of the compound of the formula (I) in the use forms may typically be between 0.00000001% and 95% by weight of the compound of the formula (I), preferably between 0.00001% and 1% by weight, based on the weight of the use form. Application is accomplished in a customary manner appropriate for the use forms.

### Mixtures

The compounds of the formula (I) can also be used in a mixture with one or more suitable fungicides, bactericides, acaricides, molluscicides, nematicides, insecticides, microbiological agents, beneficial organisms, herbicides, fertilizers, bird repellents, phytotonics, sterilants, safeners, semiochemicals and/or plant growth regulators, in order thus, for example, to broaden the spectrum of action, prolong the period of action, enhance the rate of action, prevent repellency or prevent evolution of resistance. In addition, active ingredient combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

In addition, the compounds of the formula (I) may be present in a mixture with other active ingredients or semiochemicals such as attractants and/or bird repellents and/or plant activators and/or growth regulators and/or fertilizers. Likewise, the compounds of the formula (I) can be used to improve plant properties, for example growth, yield and quality of the harvested material.

In a particular embodiment according to the invention, the compounds of the formula (I) are present in formulations or in the use forms prepared from these formulations in a mixture with further compounds, preferably those as described below.

If one of the compounds mentioned below can occur in different tautomeric forms, these forms are also included even if not explicitly mentioned in each case. All the mixing components mentioned, as the case may be, may also form salts with suitable bases or acids if they are capable of doing so on the basis of their functional groups.

### Insecticides/Acaricides/Nematicides

The active ingredients specified here with their common names are known and are described for example in “The Pesticide Manual”, 16th ed., British Crop Protection Council 2012, or can be searched for on the Internet (e.g. http://www.alanwood.net/pesticides). The classification is based on the IRAC Mode of Action Classification Scheme applicable at the time of filing of this patent application.

(1) Acetylcholinesterase (AChE) inhibitors, for example carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, e.g. acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel blockers, for example cyclodiene-organochlorines, e.g. chlordane and endosulfan or phenylpyrazoles (fiproles), e.g. ethiprole and fipronil.
(3) Sodium channel modulators, for example pyrethroids, e.g. acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomer], deltamethrin, empenthrin [(EZ)-(1R) isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans isomer], prallethrin, pyrethrins (pyrethrum), resmethrin, silafluofen, tefluthrin, tetramethrin, tetramethrin [(1R) isomer], tralomethrin and transfluthrin or DDT or methoxychlor.
(4) Nicotinic acetylcholine receptor (nAChR) competitive modulators, for example neonicotinoids, e.g. acetamiprid, clothianidin, dinotefuran, imidacloprid, nitenpyram, thiacloprid and thiamethoxam or nicotine or sulfoxaflor or flupyradifurone.
(5) Nicotinic acetylcholine receptor (nAChR) allosteric modulators, for example spinosyns, e.g. spinetoram and spinosad.
(6) Glutamate-gated chloride channel (GluCl) allosteric modulators, for example avermectins/milbemycins, e.g. abamectin, emamectin benzoate, lepimectin and milbemectin.
(7) Juvenile hormone mimetics, for example juvenile hormone analogues, e.g. hydroprene, kinoprene and methoprene or fenoxycarb or pyriproxyfen.
(8) Miscellaneous non-specific (multisite) inhibitors, for example alkyl halides, e.g. methyl bromide and other alkyl halides; or chloropicrin or sulphuryl fluoride or borax or tartar emetic or methyl isocyanate generator, e.g. diazomet and metam.
(9) Chordotonal organ modulators, e.g. pymetrozine or flonicamide.
(10) Mite growth inhibitors, for example clofentezine, hexythiazox and diflovidazin or etoxazole.
(11) Microbial disruptors of the insect midgut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis and B.t. plant proteins: Cry1Ab, Cry1Ac, Cry1Fa, Cry1A.105, Cry2Ab, VIP3A, mCry3A, Cry3Ab, Cry3Bb, Cry34Ab1/35Ab1.
(12) Inhibitors of mitochondrial ATP synthase, such as ATP disruptors, for example diafenthiuron or organotin compounds, e.g. azocyclotin, cyhexatin and fenbutatin oxide or propargite or tetradifon.
(13) Uncouplers of oxidative phosphorylation via disruption of the proton gradient, for example chlorfenapyr, DNOC and sulfluramid.
(14) Nicotinic acetylcholine receptor channel blockers, for example bensultap, cartap hydrochloride, thiocyclam, and thiosultap-sodium.
(15) Inhibitors of chitin biosynthesis, type 0, for example bistrifluron, chlorfluazuron, diflubenzuron, flucycloxuron, flufenoxuron, hexaflumuron, lufenuron, novaluron, noviflumuron, teflubenzuron and triflumuron.
(16) Inhibitors of chitin biosynthesis, type 1, for example buprofezin.
(17) Moulting disruptors (especially in the case of Diptera), for example cyromazine.
(18) Ecdysone receptor agonists, for example chromafenozide, halofenozide, methoxyfenozide and tebufenozide.
(19) Octopamine receptor agonists, for example amitraz.
(20) Mitochondrial complex III electron transport inhibitors, for example hydramethylnon or acequinocyl or fluacrypyrim.
(21) Mitochondrial complex I electron transport inhibitors, for example METI acaricides, e.g. fenazaquin, fenpyroximate, pyrimidifen, pyridaben, tebufenpyrad and tolfenpyrad or rotenone (Derris).
(22) Voltage-dependent sodium channel blockers, for example indoxacarb or metaflumizone.
(23) Inhibitors of acetyl CoA carboxylase, for example tetronic and tetramic acid derivatives, e.g. spirodiclofen, spiromesifen and spirotetramat.
(24) Mitochondrial complex IV electron transport inhibitors, for example phosphines, e.g. aluminium phosphide, calcium phosphide, phosphine and zinc phosphide, or cyanides, calcium cyanide, potassium cyanide and sodium cyanide.
(25) Mitochondrial complex II electron transport inhibitors, for example beta-keto nitrile derivatives, e.g. cyenopyrafen and cyflumetofen and carboxanilides, for example pyflubumide.
(28) Ryanodine receptor modulators, for example diamides, e.g. chlorantraniliprole, cyantraniliprole and flubendiamide,
further active ingredients, for example afidopyropen, afoxolaner, azadirachtin, benclothiaz, benzoximate, bifenazate, broflanilide, bromopropylate, chinomethionat, chloroprallethrin, cryolite, cyclaniliprole, cycloxaprid, cyhalodiamide, dicloromezotiaz, dicofol, epsilon metofluthrin, epsilon momfluthrin, flometoquin, fluazaindolizine, fluensulfone, flufenerim, flufenoxystrobin, flufiprole, fluhexafon, fluopyram, fluralaner, fluxametamide, fufenozide, guadipyr, heptafluthrin, imidaclothiz, iprodione, kappa bifenthrin, kappa tefluthrin, lotilaner, meperfluthrin, paichongding, pyridalyl, pyrifluquinazon, pyriminostrobin, spirobudiclofen, tetramethylfluthrin, tetraniliprole, tetrachlorantraniliprole, tioxazafen, thiofluoximate, triflumezopyrim and iodomethane; additionally preparations based on Bacillus firmus (1-1582, BioNeem, Votivo), and the following compounds: 1-{2-fluoro-4-methyl-5-[(2,2,2-trifluoroethyl)sulphinyl]phenyl}-3-(trifluoromethyl)-1H-1,2,4-triazole-5-amine (known from WO2006/043635) (CAS 885026-50-6), {1′-[(2E)-3-(4-chlorophenyl)prop-2-en-1-yl]-5-fluorospiro[indole-3,4′-piperidine]-1 (2H)-yl}(2-chloropyridin-4-yl)methanone (known from WO2003/106457) (CAS 637360-23-7), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethylcarbonate (known from EP 2647626) (CAS-1440516-42-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS Reg. No. 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoropropan-2-one (known from WO2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)benzamide, 4-[5-(3,5-dichlorophenyl)-4,5-dihydro-5-(trifluoromethyl)-3-isoxazolyl]-2-methyl-N-(trans-1-oxido-3-thietanyl)benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3,3,3-trifluoropropyl)sulphinyl]propanamide, (+)-N-[3-chloro-1-(3-pyridinyl)-1H-pyrazol-4-yl]-N-ethyl-3-[(3,3,3-trifluoropropyl)sulphinyl]propanamide and (−)-N-[3-chloro-1-(3-pyridinyl)-1H-pyrazol-4-yl]-N-ethyl-3-[(3,3,3-trifluoropropyl)sulphinyl]propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulphinyl]-1H-pyrazole-3-carbonitrile (known from CN 101337937 A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide, (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]hydrazinecarboxamide (known from CN 101715774 A) (CAS 1232543-85-9); cyclopropanecarboxylic acid 3-(2,2-dichloroethenyl)-2,2-dimethyl-4-(1H-benzimidazol-2-yl)phenyl ester (known from CN 103524422 A) (CAS 1542271-46-4); (4aS)-7-chloro-2,5-dihydro-2-[[(methoxy carbonyl) [4-[(trifluoromethyl)thio]phenyl]amino]carbonyl]indeno[1,2-e][1,3,4]oxadiazine-4a(3H)-carboxylic acid methyl ester (known from CN 102391261 A) (CAS 1370358-69-2); 6-deoxy-3-O-ethyl-2,4-di-O-methyl-1-[N-[4-[1-[4-(1,1,2,2,2-pentafluoroethoxy)phenyl]-1H-1,2,4-triazole-3-yl]phenyl]carbamate]-α-L-mannopyranose (known from US 2014/0275503 A1) (CAS 1181213-14-8); 8-(2-cyclopropylmethoxy-4-trifluoromethylphenoxy)-3-(6-trifluoromethylpyridazin-3-yl)-3-azabicyclo[3.2.1]octane (CAS 1253850-56-4), (8-anti)-8-(2-cyclopropylmethoxy-4-trifluoromethylphenoxy)-3-(6-trifluoromethylpyridazin-3-yl)-3-azabicyclo[3.2.1]octane (CAS 933798-27-7), (8-syn)-8-(2-cyclopropylmethoxy-4-trifluoromethylphenoxy)-3-(6-trifluoromethylpyridazin-3-yl)-3-azabicyclo[3.2.1]octane (known from WO 2007040280 A1, WO 2007040282 A1) (CAS 934001-66-8) and N-[3-chloro-1-(3-pyridinyl)-1H-pyrazol-4-yl]-N-ethyl-3-[(3,3,3-trifluoropropyl)thio]propanamide (known from WO 2015/058021 A1, WO 2015/058028 A1) (CAS 1477919-27-9).

### Fungicides

The active ingredients specified here by their “common names” are known and are described, for example, in the “Pesticide Manual” (16th ed., British Crop Protection Council) or can be searched for on the Internet (for example: http://www.alanwood.net/pesticides).

All the mixing components mentioned in classes (1) to (15), as the case may be, may form salts with suitable bases or acids if they are capable of doing so on the basis of their functional groups. All the fungicidal mixing components of classes (1) to (15) mentioned, as the case may be, may include tautomeric forms.
1) Ergosterol biosynthesis inhibitors, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-2,4-triazol-1-yl)butan-2-ol, (1.033) (2S)-2-[4-(4-chlorophenoxy)-2-(trifluoromethyl)phenyl]-1-(1H-1,2,4-triazol 1-y)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulphanyl)-1-{[3-(2-chlorophenyl)-2-(2,4-difluorophenyl)oxiran-2-yl]methyl}-1H-1,2,4-triazole, (1.061) 5-(allylsulphanyl)-1-{[rel(2R,3R)-3-(2-chlorophenyl)-2-(2,4-difluorophenyl)oxiran-2-yl]methyl}-1H-1,2,4-triazole, (1.062) 5-(allylsulphanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulphanyl}phenyl)-N-ethyl-N-methylimidoformamide, (1.064) N′-(2,5-dimethyl-4-{[3-(2,2,2-trifluoroethoxy)phenyl]sulphanyl}phenyl)-N-ethyl-N-methylimidoformamide, (1.065) N′-(2,5-dimethyl-4-{[3-(2,2,3,3-tetrafluoropropoxy)phenyl]sulphanyl}phenyl)-N-ethyl-N-methylimidoformamide, (1.066) N′-(2,5-dimethyl-4-{[3-(pentafluoroethoxy)phenyl]sulphanyl}phenyl)-N-ethyl-N-methylimidoformamide, (1.067) N′-(2,5-dimethyl-4-{3-[(1,1,2,2-tetrafluoroethyl)sulphanyl]phenoxy}phenyl)-N-ethyl-N-methylimidoformamide, (1.068) N′-(2,5-dimethyl-4-{3-[(2,2,2-trifluoroethyl)sulphanyl]phenoxy}phenyl)-N-ethyl-N-methylimidoformamide, (1.069) N′-(2,5-dimethyl-4-{3-[(2,2,3,3-tetrafluoropropyl)sulphanyl]phenoxy}phenyl)-N-ethyl-N-methylimidoformamide, (1.070) N′-(2,5-dimethyl-4-{3-[(pentafluoroethyl)sulphanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulphanyl}-2,5-dimethylphenyl)-N-ethyl-N-methylimidoformamide, (1.073) N′-(4-{3-[(difluoromethyl)sulphanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1 S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide.
2) Inhibitors of the respiratory chain in complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of the syn-epimeric racemate 1RS,4SR,9RS and the anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034) N-(2-cyclopentyl-5-fluorobenzyl)-N-cyclopropyl-3-(difluoromethyl)-5-fluoro-1-methyl-1H-pyrazole-4-carboxamide, (2.035) N-(2-tert-butyl-5-methylbenzyl)-N-cyclopropyl-3-(difluoromethyl)-5-fluoro-1-methyl-1H-pyrazole-4-carboxamide, (2.036) N-(2-tert-butylbenzyl)-N-cyclopropyl-3-(difluoromethyl)-5-fluoro-1-methyl-1H-pyrazole-4-carboxamide, (2.037) N-(5-chloro-2-ethylbenzyl)-N-cyclopropyl-3-(difluoromethyl)-5-fluoro-1-methyl-1H-pyrazole-4-carboxamide, (2.038) N-(5-chloro-2-isopropylbenzyl)-N-cyclopropyl-3-(difluoromethyl)-5-fluoro-1-methyl-1H-pyrazole-4-carboxamide, (2.039) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041) N-[1-(2,4-dichlorophenyl)-1-methoxypropan-2-yl]-3-(difluoromethyl)-1-methyl-1H-pyrazole-4-carboxamide, (2.042) N-[2-chloro-6-(trifluoromethyl)benzyl]-N-cyclopropyl-3-(difluoromethyl)-5-fluoro-1-methyl-1H-pyrazole-4-carboxamide, (2.043) N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044) N-[5-chloro-2-(trifluoromethyl)benzyl]-N-cyclopropyl-3-(difluoromethyl)-5-fluoro-1-methyl-1H-pyrazole-4-carboxamide, (2.045) N-cyclopropyl-3-(difluoromethyl)-5-fluoro-1-methyl-N-[5-methyl-2-(trifluoromethyl)benzyl]-1H-pyrazole-4-carboxamide, (2.046) N-cyclopropyl-3-(difluoromethyl)-5-fluoro-N-(2-fluoro-6-isopropylbenzyl)-1-methyl-1H-pyrazole-4-carboxamide, (2.047) N-cyclopropyl-3-(difluoromethyl)-5-fluoro-N-(2-isopropyl-5-methylbenzyl)-1-methyl-1H-pyrazole-4-carboxamide, (2.048) N-cyclopropyl-3-(difluoromethyl)-5-fluoro-N-(2-isopropylbenzyl)-1-methyl-1H-pyrazole-4-carbothioamide, (2.049) N-cyclopropyl-3-(difluoromethyl)-5-fluoro-N-(2-isopropylbenzyl)-1-methyl-1H-pyrazole-4-carboxamide, (2.050) N-cyclopropyl-3-(difluoromethyl)-5-fluoro-N-(5-fluoro-2-isopropylbenzyl)-1-methyl-1H-pyrazole-4-carboxamide, (2.051) N-cyclopropyl-3-(difluoromethyl)-N-(2-ethyl-4,5-dimethylbenzyl)-5-fluoro-1-methyl-1H-pyrazole-4-carboxamide, (2.052) N-cyclopropyl-3-(difluoromethyl)-N-(2-ethyl-5-fluorobenzyl)-5-fluoro-1-methyl-1H-pyrazole-4-carboxamide, (2.053) N-cyclopropyl-3-(difluoromethyl)-N-(2-ethyl-5-methylbenzyl)-5-fluoro-1-methyl-1H-pyrazole-4-carboxamide, (2.054) N-cyclopropyl-N-(2-cyclopropyl-5-fluorobenzyl)-3-(difluoromethyl)-5-fluoro-1-methyl-1H-pyrazole-4-carboxamide, (2.055) N-cyclopropyl-N-(2-cyclopropyl-5-methylbenzyl)-3-(difluoromethyl)-5-fluoro-1-methyl-1H-pyrazole-4-carboxamide, (2.056) N-cyclopropyl-N-(2-cyclopropylbenzyl)-3-(difluoromethyl)-5-fluoro-1-methyl-1H-pyrazole-4-carboxamide.
3) Inhibitors of the respiratory chain in complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadon, (3.010) fenamidon, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)methyl]phenyl}-2-methoxy-N-methylacetamide, (3.027) N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide.
4) Mitosis and cell division inhibitors, for example (4.001) carbendazim, (4.002) diethofencarb, (4.003) ethaboxam, (4.004) fluopicolid, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023) N-(2-bromo-6-fluorophenyl)-4-(2-chloro-4-fluorophenyl)-1,3-dimethyl-1H-pyrazol-5-amine, (4.024) N-(2-bromophenyl)-4-(2-chloro-4-fluorophenyl)-1,3-dimethyl-1H-pyrazol-5-amine, (4.025) N-(4-chloro-2,6-difluorophenyl)-4-(2-chloro-4-fluorophenyl)-1,3-dimethyl-1H-pyrazol-5-amine.
5) Compounds having capacity for multisite activity, for example (5.001) Bordeaux mixture, (5.002) captafol, (5.003) captan, (5.004) chlorthalonil, (5.005) copper hydroxide, (5.006) copper naphthenate, (5.007) copper oxide, (5.008) copper oxychloride, (5.009) copper(2+) sulphate, (5.010) dithianon, (5.011) dodin, (5.012) folpet, (5.013) mancozeb, (5.014) maneb, (5.015) metiram, (5.016) zinc metiram, (5.017) copper oxine, (5.018) propineb, (5.019) sulphur and sulphur preparations including calcium polysulphide, (5.020) thiram, (5.021) zineb, (5.022) ziram.
6) Compounds capable of triggering host defence, for example (6.001) acibenzolar-S-methyl, (6.002) isotianil, (6.003) probenazole, (6.004) tiadinil.
7) Amino acid and/or protein biosynthesis inhibitors, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.
(8) ATP production inhibitors, for example (8.001) silthiofam.
9) Cell wall synthesis inhibitors, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.
10) Lipid and membrane synthesis inhibitors, for example (10.001) propamocarb, (10.002) propamocarb hydrochloride, (10.003) tolclofos-methyl.
11) Melanin biosynthesis inhibitors, for example (11.001) tricyclazole, (11.002) 2,2,2-trifluoroethyl {3-methyl-1-[(4-methylbenzoyl)amino]butan-2-yl}carbamate.
12) Nucleic acid synthesis inhibitors, for example (12.001) benalaxyl, (12.002) benalaxyl-M (kiralaxyl), (12.003) metalaxyl, (12.004) metalaxyl-M (mefenoxam).
13) Signal transduction inhibitors, for example (13.001) fludioxonil, (13.002) iprodione, (13.003) procymidone, (13.004) proquinazid, (13.005) quinoxyfen, (13.006) vinclozolin.
14) Compounds that can act as decouplers, for example (14.001) fluazinam, (14.002) meptyldinocap.
15) Further compounds, for example (15.001) abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenon, (15.017) mildiomycin, (15.018) natamycin, (15.019) nickel dimethyldithiocarbamate, (15.020) nitrothal-isopropyl, (15.021) oxamocarb, (15.022) oxathiapiprolin, (15.023) oxyfenthiin, (15.024) pentachlorophenol and salts, (15.025) phosphonic acid and salts thereof, (15.026) propamocarb-fosetylate, (15.027) pyriofenone (chlazafenone) (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulphonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulphonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulphonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulphonate, (15.045) 2-phenylphenol and salts thereof, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butyric acid, (15.050) 5-amino-1,3,4-thiadiazole-2-thiol, (15.051) 5-chloro-N′-phenyl-N’-(prop-2-yn-1-yl)thiophene 2-sulphonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulphate (2:1), (15.061) tert-butyl {6-[({[(1-methyl-1H-tetrazol-5-yl)(phenyl)methylene]amino}oxy)methyl]pyridin-2-yl}carbamate.

### Biological Pesticides as Mixing Components

The compounds of the formula (I) can be combined with biological pesticides.

Biological pesticides especially include bacteria, fungi, yeasts, plant extracts and products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which are used or can be used as biological pesticides are:

Bacillus amyloliquefaciens, strain FZB42 (DSM 231179), or Bacillus cereus, especially B. cereus strain CNCM 1-1562 or Bacillus firmus, strain 1-1582 (Accession number CNCM 1-1582) or Bacillus pumilus, especially strain GB34 (Accession No. ATCC 700814) and strain QST2808 (Accession No. NRRL B-30087), or Bacillus subtilis, especially strain GB03 (Accession No. ATCC SD-1397), or Bacillus subtilis strain QST713 (Accession No. NRRL B-21661) or Bacillus subtilis strain OST 30002 (Accession No. NRRL B-50421), Bacillus thuringiensis, especially B. thuringiensis subspecies israelensis (serotype H-14), strain AM65-52 (Accession No. ATCC 1276), or B. thuringiensis subsp. aizawai, especially strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are used or can be used as biological pesticides are:

Beauveria bassiana, especially strain ATCC 74040, Coniothyrium minitans, especially strain CON/M/91-8 (Accession No. DSM-9660), Lecanicillium spp., especially strain HRO LEC 12, Lecanicillium lecanii (formerly known as Verticillium lecanii), especially strain KV01, Metarhizium anisopliae, especially strain F52 (DSM3884/ATCC 90448), Metschnikowia fructicola, especially strain NRRL Y-30752, Paecilomyces fumosoroseus (new: Isaria fumosorosea), especially strain IFPC 200613, or strain Apopka 97 (Accession No. ATCC 20874), Paecilomyces lilacinus, especially P. lilacinus strain 251 (AGAL 89/030550), Talaromyces flavus, especially strain V117b, Trichoderma atroviride, especially strain SC1 (Accession Number CBS 122089), Trichoderma harzianum, especially T. harzianum rifai T39 (Accession Number CNCM 1-952).

Examples of viruses which are used or can be used as biological pesticides are:

Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples include:

Agrobacterium spp., Azorhizobium caulinodans, Azospirillum spp., Azotobacter spp., Bradyrhizobium spp., Burkholderia spp., especially Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., especially Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are:

Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), pyrethrum/pyrethrins, Quassia amara, Quercus, Quillaja, Regalia, “Requiem Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder.

### Safeners as Mixing Components

The compounds of the formula (I) can be combined with safeners, for example benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulphonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

### Plants and Plant Parts

All plants and plant parts can be treated in accordance with the invention. Plants are understood here to mean all plants and populations of plants, such as desirable and undesirable wild plants or crop plants (including naturally occurring crop plants), for example cereals (wheat, rice, triticale, barley, rye, oats), maize, soya beans, potatoes, sugar beet, sugar cane, tomatoes, bell peppers, cucumbers, melons, carrots, water melons, onions, lettuce, spinach, leeks, beans, Brassica oleracea (e.g. cabbage) and other vegetable species, cotton, tobacco, oilseed rape, and also fruit plants (the fruits being apples, pears, citrus fruits and grapes). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights. Plants shall be understood to mean all development stages such as seed, seedlings, young (immature) plants, up to and including mature plants. Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested plants or harvested plant parts and vegetative and generative propagation material, for example cuttings, tubers, rhizomes, slips and seeds.

The inventive treatment of the plants and parts of plants with the compounds of the formula (I) is effected directly or by allowing the compounds to act on the surroundings, the habitat or the storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible to treat all plants and their parts in accordance with the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) and which have been obtained by conventional breeding, by mutagenesis or by recombinant DNA techniques. They may be cultivars, varieties, biotypes or genotypes.

### Transgenic Plants, Seed Treatment and Integration Events

The preferred transgenic plants or plant cultivars (those obtained by genetic engineering) which are to be treated in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher harvest yields, higher quality and/or higher nutritional value of the harvested products, better storage life and/or processability of the harvested products. Further and particularly emphasized examples of such properties are increased resistance of the plants against animal and microbial pests, such as insects, arachnids, nematodes, mites, slugs and snails, owing, for example, to toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof), and also increased resistance of the plants against phytopathogenic fungi, bacteria and/or viruses caused, for example, by systemic acquired resistance (SAR), systemin, phytoalexins, elicitors and resistance genes and correspondingly expressed proteins and toxins, and also increased tolerance of the plants to certain herbicidally active ingredients, for example imidazolinones, sulphonylureas, glyphosate or phosphinothricin (for example the “PAT” gene). The genes which impart the desired properties (“traits”) in question may also be present in combinations with one another in the transgenic plants. Examples of transgenic plants mentioned include the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (the fruits being apples, pears, citrus fruits and grapevines), particular emphasis being given to maize, soya beans, wheat, rice, potatoes, cotton, sugar cane, tobacco and oilseed rape. Properties (“traits”) which are particularly emphasized are the increased resistance of the plants to insects, arachnids, nematodes and slugs and snails.

### Crop Protection—Types of Treatment

The treatment of the plants and plant parts with the compounds of the formula (I) is effected directly or by action on their surroundings, habitat or storage space by the customary treatment methods, for example by dipping, spraying, atomizing, irrigating, evaporating, dusting, fogging, broadcasting, foaming, painting, spreading-on, injecting, watering (drenching), drip irrigating and, in the case of propagation material, especially in the case of seed, also by dry seed treatment, wet seed treatment, slurry treatment, incrustation, coating with one or more coats, etc. It is also possible to deploy the compounds of the formula (I) by the ultra-low volume method or to inject the use form or the compound of the formula (I) itself into the soil.

A preferred direct treatment of the plants is foliar application, meaning that the compounds of the formula (I) are applied to the foliage, in which case the treatment frequency and the application rate should be adjusted according to the level of infestation with the pest in question.

In the case of systemically active ingredients, the compounds of the formula (I) also access the plants via the root system. The plants are then treated by the action of the compounds of the formula (I) on the habitat of the plant. This can be accomplished, for example, by drenching, or by mixing into the soil or the nutrient solution, meaning that the locus of the plant (e.g. soil or hydroponic systems) is impregnated with a liquid form of the compounds of the formula (I), or by soil application, meaning that the inventive compounds of the formula (I) are introduced in solid form (e.g. in the form of granules) into the locus of the plants. In the case of paddy rice crops, this can also be accomplished by metering the compound of the formula (I) in a solid application form (for example as granules) into a flooded paddy field.

### Seed Treatment

The control of animal pests by the treatment of the seed of plants has long been known and is the subject of constant improvements. Nevertheless, the treatment of seed entails a series of problems which cannot always be solved in a satisfactory manner. Thus, it is desirable to develop methods for protecting the seed and the germinating plant which dispense with, or at least reduce considerably, the additional application of pesticides during storage, after sowing or after emergence of the plants. It is additionally desirable to optimize the amount of active ingredient used so as to provide optimum protection for the seed and the germinating plant from attack by animal pests, but without damage to the plant itself by the active ingredient used. In particular, methods for the treatment of seed should also take account of the intrinsic insecticidal or nematicidal properties of pest-resistant or -tolerant transgenic plants in order to achieve optimal protection of the seed and also the germinating plant with a minimum expenditure on pesticides.

The present invention therefore in particular also relates to a method for the protection of seed and germinating plants from attack by pests, by treating the seed with one of the compounds of the formula (I). The method according to the invention for protecting seed and germinating plants against attack by pests further comprises a method in which the seed is treated simultaneously in one operation or sequentially with a compound of the formula (I) and a mixing component. It further also comprises a method where the seed is treated at different times with a compound of the formula (I) and a mixing component.

The invention likewise relates to the use of the compounds of the formula (I) for the treatment of seed for protecting the seed and the resulting plant from animal pests.

The invention further relates to seed which has been treated with a compound of the formula (I) according to the invention for protection from animal pests. The invention also relates to seed which has been treated simultaneously with a compound of the formula (I) and a mixing component. The invention further relates to seed which has been treated at different times with a compound of the formula (I) and a mixing component. In the case of seed which has been treated at different times with a compound of the formula (I) and a mixing component, the individual substances may be present on the seed in different layers. In this case, the layers comprising a compound of the formula (I) and mixing components may optionally be separated by an intermediate layer. The invention also relates to seed in which a compound of the formula (I) and a mixing component have been applied as part of a coating or as a further layer or further layers in addition to a coating.

The invention further relates to seed which, after the treatment with a compound of the formula (I), is subjected to a film-coating process to prevent dust abrasion on the seed.

One of the advantages that occur when a compound of the formula (I) acts systemically is that the treatment of the seed protects not only the seed itself but also the plants resulting therefrom, after emergence, from animal pests. In this way, the immediate treatment of the crop at the time of sowing or shortly thereafter can be dispensed with.

A further advantage is that the treatment of the seed with a compound of the formula (I) can enhance germination and emergence of the treated seed.

It is likewise considered to be advantageous that compounds of the formula (I) can especially also be used for transgenic seed.

Furthermore, compounds of the formula (I) can be employed in combination with compositions of signalling technology, leading to better colonization by symbionts such as, for example, rhizobia, mycorrhizae and/or endophytic bacteria or fungi, and/or to optimized nitrogen fixation.

The compounds of the formula (I) are suitable for protection of seed of any plant variety which is used in agriculture, in the greenhouse, in forests or in horticulture. More particularly, this is the seed of cereals (for example wheat, barley, rye, millet and oats), maize, cotton, soya beans, rice, potatoes, sunflowers, coffee, tobacco, canola, oilseed rape, beets (for example sugar beets and fodder beets), peanuts, vegetables (for example tomatoes, cucumbers, beans, cruciferous vegetables, onions and lettuce), fruit plants, lawns and ornamental plants. Of particular significance is the treatment of the seed of cereals (such as wheat, barley, rye and oats), maize, soya beans, cotton, canola, oilseed rape, vegetables and rice.

As already mentioned above, the treatment of transgenic seed with a compound of the formula (I) is also of particular importance. This involves the seed of plants which generally contain at least one heterologous gene which controls the expression of a polypeptide having insecticidal and/or nematicidal properties in particular. The heterologous genes in transgenic seed may originate from microorganisms such as Bacillus, Rhizobium, Pseudomonas, Serratia, Trichoderma, Clavibacter, Glomus or Gliocladium. The present invention is particularly suitable for treatment of transgenic seed which comprises at least one heterologous gene originating from Bacillus sp. The heterologous gene is more preferably derived from Bacillus thuringiensis.

In the context of the present invention, the compound of the formula (I) is applied to the seed. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content which allows storage. Alternatively, it is also possible to use seed which, after drying, has been treated with, for example, water and then dried again, for example priming. In the case of rice seed, it is also possible to use seed which has been soaked, for example in water, until it reaches a certain stage of the rice embryo (“pigeon breast stage”) which results in stimulation of germination and more uniform emergence.

When treating the seed, care must generally be taken that the amount of the compound of the formula (I) applied to the seed and/or the amount of further additives is chosen in such a way that the germination of the seed is not adversely affected, or that the resulting plant is not damaged. This has to be ensured particularly in the case of active ingredients which can exhibit phytotoxic effects at certain application rates.

In general, the compounds of the formula (I) are applied to the seed in the form of a suitable formulation. Suitable formulations and processes for seed treatment are known to the person skilled in the art.

The compounds of the formula (I) can be converted to the customary seed-dressing formulations, such as solutions, emulsions, suspensions, powders, foams, slurries or other coating compositions for seed, and also ULV formulations.

These formulations are prepared in a known manner, by mixing the compounds of the formula (I) with customary additives, for example customary extenders and solvents or diluents, dyes, wetting agents, dispersants, emulsifiers, antifoams, preservatives, secondary thickeners, adhesives, gibberellins, and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of active agrochemical ingredients. Usable with preference are alkyl naphthalenesulphonates, such as diisopropyl or diisobutyl naphthalenesulphonates.

Suitable dispersants and/or emulsifiers which may be present in the seed-dressing formulations usable in accordance with the invention are all nonionic, anionic and cationic dispersants customary for the formulation of active agrochemical ingredients. Nonionic or anionic dispersants or mixtures of nonionic or anionic dispersants can be used with preference. Suitable nonionic dispersants especially include ethylene oxide/propylene oxide block polymers, alkylphenol polyglycol ethers and tristyrylphenol polyglycol ethers, and the phosphated or sulphated derivatives thereof. Suitable anionic dispersants are especially lignosulphonates, polyacrylic acid salts and arylsulphonate-formaldehyde condensates.

Antifoams which may be present in the seed-dressing formulations usable in accordance with the invention are all foam-inhibiting substances customary for the formulation of active agrochemical ingredients. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances which can be used for such purposes in agrochemical compositions.

Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

Gibberellins which may be present in the seed-dressing formulations usable in accordance with the invention are preferably the gibberellins A1, A3 (=gibberellic acid), A4 and A7; particular preference is given to using gibberellic acid. The gibberellins are known (cf. R. Wegler “Chemie der Pflanzenschutz-und Schadlingsbekaimpfungsmittel” [Chemistry of Crop Protection Compositions and Pesticides], vol. 2, Springer Verlag, 1970, p. 401-412).

The seed-dressing formulations usable in accordance with the invention can be used to treat a wide variety of different kinds of seed, either directly or after prior dilution with water. For instance, the concentrates or the preparations obtainable therefrom by dilution with water can be used to dress the seed of cereals, such as wheat, barley, rye, oats and triticale, and also the seed of maize, rice, oilseed rape, peas, beans, cotton, sunflowers, soya beans and beets, or else a wide variety of different vegetable seed. The seed-dressing formulations usable in accordance with the invention, or the dilute use forms thereof, can also be used to dress seed of transgenic plants.

For the treatment of seed with the seed-dressing formulations usable in accordance with the invention, or the use forms prepared therefrom by adding water, all mixing units usable customarily for the seed dressing are useful. Specifically, the procedure in seed dressing is to place the seed into a mixer in batchwise or continuous operation, to add the particular desired amount of seed-dressing formulations, either as such or after prior dilution with water, and to mix until the formulation is distributed homogeneously on the seed. If appropriate, this is followed by a drying operation.

The application rate of the seed-dressing formulations usable in accordance with the invention can be varied within a relatively wide range. It is guided by the particular content of the compounds of the formula (I) in the formulations and by the seed. The application rates of the compound of the formula (I) are generally between 0.001 and 50 g per kilogram of seed, preferably between 0.01 and 15 g per kilogram of seed.

### Animal Health

In the animal health field, i.e. the field of veterinary medicine, the compounds of the formula (I) are active against animal parasites, in particular ectoparasites or endoparasites. The term “endoparasite” includes especially helminths and protozoa, such as coccidia. Ectoparasites are typically and preferably arthropods, especially insects or acarids.

In the field of veterinary medicine, the compounds of the formula (I) having favourable homeotherm toxicity are suitable for controlling parasites which occur in animal breeding and animal husbandry in livestock, breeding animals, zoo animals, laboratory animals, experimental animals and domestic animals. They are active against all or specific stages of development of the parasites.

Agricultural livestock include, for example, mammals, such as sheep, goats, horses, donkeys, camels, buffalo, rabbits, reindeer, fallow deer and especially cattle and pigs; or poultry such as turkeys, ducks, geese and especially chickens; or fish or crustaceans, for example in aquaculture; or, as the case may be, insects such as bees.

Domestic animals include, for example, mammals, such as hamsters, guinea pigs, rats, mice, chinchillas, ferrets, and particularly dogs, cats, caged birds, reptiles, amphibians or aquarium fish.

In a specific embodiment, the compounds of the formula (I) are administered to mammals.

In another specific embodiment, the compounds of the formula (I) are administered to birds, namely caged birds or particularly poultry.

Use of the compounds of the formula (I) for the control of animal parasites is intended to reduce or prevent illness, cases of death and reductions in performance (in the case of meat, milk, wool, hides, eggs, honey and the like), such that more economical and simpler animal husbandry is enabled and better animal well-being is achievable.

In relation to the field of animal health, the term “control” or “controlling” in the present context means that the compounds of the formula (I) are effective in reducing the incidence of the particular parasite in an animal infected with such parasites to an innocuous degree. More specifically, “controlling” in the present context means that the compounds of the formula (I) kill the respective parasite, inhibit its growth, or inhibit its proliferation.

The arthropods include, but are not limited to,

from the order of Anoplurida, for example, Haematopinus spp., Linognathus spp., Pediculus spp., Phtirus spp. and Solenopotes spp.;
from the order of Mallophagida and the suborders Amblycerina and Ischnocerina, for example, Bovicola spp., Damalina spp., Felicola spp.; Lepikentron spp., Menopon spp., Trichodectes spp., Trimenopon spp., Trinoton spp., Werneckiella spp;
from the order of Diptera and the suborders Nematocerina and Brachycerina, for example, Aedes spp., Anopheles spp., Atylotus spp., Braula spp., Calliphora spp., Chrysomyia spp., Chrysops spp., Culex spp., Culicoides spp., Eusimulium spp., Fannia spp., Gasterophilus spp., Glossina spp., Haematobia spp., Haematopota spp., Hippobosca spp., Hybomitra spp., Hydrotaea spp., Hypoderma spp., Lipoptena spp., Lucilia spp., Lutzomyia spp., Melophagus spp., Morellia spp., Musca spp., Odagmia spp., Oestrus spp., Philipomyia spp., Phlebotomus spp., Rhinoestrus spp., Sarcophaga spp., Simulium spp., Stomoxys spp., Tabanus spp., Tipula spp., Wilhelmia spp., Wohlfahrtia spp.;
from the order of Siphonapterida, for example, Ceratophyllus spp., Ctenocephalides spp., Pulex spp., Tunga spp., Xenopsylla spp.;
from the order of Heteropterida, for example, Cimex spp., Panstrongylus spp., Rhodnius spp., Triatoma spp.; and also nuisance and hygiene pests from the order Blattarida.

In addition, in the case of the arthropods, mention should be made by way of example, without limitation, of the following Acari:

from the subclass of Acari (Acarina) and the order of Metastigmata, for example from the family of Argasidae such as Argas spp., Omithodorus spp., Otobius spp., from the family of Ixodidae such as Amblyomma spp., Dermacentor spp., Haemaphysalis spp., Hyalomma spp., Ixodes spp., Rhipicephalus (Boophilus) spp., Rhipicephalus spp. (the original genus of multi-host ticks); from the order of Mesostigmata such as Dermanyssus spp., Ornithonyssus spp., Pneumonyssus spp., Raillietia spp., Sternostoma spp., Tropilaelaps spp., Varroa spp.; from the order of the Actinedida (Prostigmata), for example, Acarapis spp., Cheyletiella spp., Demodex spp., Listrophorus spp., Myobia spp., Neotrombicula spp., Omithocheyletia spp., Psorergates spp., Trombicula spp.; and from the order of the Acaridida (Astigmata), for example, Acarus spp., Caloglyphus spp., Chorioptes spp., Cytodites spp., Hypodectes spp., Knemidocoptes spp., Laminosioptes spp., Notoedres spp., Otodectes spp., Psoroptes spp., Pterolichus spp., Sarcoptes spp., Trixacarus spp., Tyrophagus spp.

Examples of parasitic protozoa include, but are not limited to:

Mastigophora (Flagellata), such as:
Metamonada: from the order of Diplomonadida, for example Giardia spp., Spironucleus spp.
Parabasala: from the order of Trichomonadida, for example Histomonas spp., Pentatrichomonas spp., Tetratrichomonas spp., Trichomonas spp., Tritrichomonas spp.
Euglenozoa: from the order of Trypanosomatida, for example Leishmania spp., Trypanosoma spp.
Sarcomastigophora (Rhizopoda) such as Entamoebidae, for example Entamoeba spp., Centramoebidae, for example Acanthamoeba sp., Euamoebidae, e.g. Hartmanella sp.
Alveolata such as Apicomplexa (Sporozoa): e.g. Cryptosporidium spp.; from the order of Eimeriida, for example, Besnoitia spp., Cystoisospora spp., Eimeria spp., Hammondia spp., Isospora spp., Neospora spp., Sarcocystis spp., Toxoplasma spp.; from the order of Adeleida, for example, Hepatozoon spp., Klossiella spp.; from the order of Haemosporida, for example, Leucocytozoon spp., Plasmodium spp.; from the order of Piroplasmida, for example, Babesia spp., Ciliophora spp., Echinozoon spp., Theileria spp.; from the order of Vesibuliferida, for example, Balantidium spp., Buxtonella spp.
Microspora such as Encephalitozoon spp., Enterocytozoon spp., Globidium spp., Nosema spp., and also, for example, Myxozoa spp.

The helminths that are pathogenic to humans or animals include, for example, Acanthocephala, Nematoden, Pentastoma and Platyhelminthes (e.g. Monogenea, cestodes and trematodes).

Illustrative helminths include, but are not limited to:

Monogenea: for example: Dactylogyrus spp., Gyrodactylus spp., Microbothrium spp., Polystoma spp., Troglecephalus spp.;
Cestodes: from the order of Pseudophyllidea, for example: Bothridium spp., Diphyllobothrium spp., Diplogonoporus spp. Ichthyobothrium spp., Ligula spp., Schistocephalus spp., Spirometra spp.
From the order of Cyclophyllida, for example: Andyra spp., Anoplocephala spp., Avitellina spp., Bertiella spp., Cittotaenia spp., Davainea spp., Diorchis spp., Diplopylidium spp., Dipylidium spp., Echinococcus spp., Echinocotyle spp., Echinolepis spp., Hydatigera spp., Hymenolepis spp., Joyeuxiella spp., Mesocestoides spp., Moniezia spp., Paranoplocephala spp., Raillietina spp., Stilesia spp., Taenia spp., Thysaniezia spp., Thysanosoma spp.
Trematodes: from the class of Digenea, for example: Austrobilharzia spp., Brachylaima spp., Calicophoron spp., Catatropis spp., Clonorchis spp. Collyriclum spp., Cotylophoron spp., Cyclocoelum spp., Dicrocoelium spp., Diplostomum spp., Echinochasmus spp., Echinoparyphium spp., Echinostoma spp., Eurytrema spp., Fasciola spp., Fasciolides spp., Fasciolopsis spp., Fischoederius spp., Gastrothylacus spp., Gigantobilharzia spp., Gigantocotyle spp., Heterophyes spp., Hypoderaeum spp., Leucochloridium spp., Metagonimus spp., Metorchis spp., Nanophyetus spp., Notocotylus spp., Opisthorchis spp., Omithobilharzia spp., Paragonimus spp., Paramphistomum spp., Plagiorchis spp., Posthodiplostomum spp., Prosthogonimus spp., Schistosoma spp., Trichobilharzia spp., Troglotrema spp., Typhlocoelum spp.
Nematodes: from the order of Trichinellida, for example: Capillaria spp., Trichinella spp., Trichomosoides spp., Trichuris spp.
From the order of Tylenchida, for example: Micronema spp., Parastrangyloides spp., Strongyloides spp.
From the order of Rhabditida, for example: Aelurostrongylus spp., Amidostomum spp., Ancylostoma spp., Angiostrongylus spp., Bronchonema spp., Bunostomum spp., Chabertia spp., Cooperia spp., Cooperioides spp., Crenosoma spp., Cyathostomum spp., Cyclococercus spp., Cyclodontostomum spp., Cylicocyclus spp., Cylicostephanus spp., Cylindropharynx spp., Cystocaulus spp., Dictyocaulus spp., Elaphostrongylus spp., Filaroides spp., Globocephalus spp., Graphidium spp., Gyalocephalus spp., Haemonchus spp., Heligmosomoides spp., Hyostrongylus spp., Marshallagia spp., Metastrongylus spp., Muellerius spp., Necator spp., Nematodirus spp., Neostrongylus spp., Nippostrongylus spp., Obeliscoides spp., Oesophagodontus spp., Oesophagostomum spp., Ollulanus spp.; Omithostrongylus spp., Oslerus spp., Ostertagia spp., Paracooperia spp., Paracrenosoma spp., Parafilaroides spp., Parelaphostrongylus spp., Pneumocaulus spp., Pneumostrongylus spp., Poteriostomum spp., Protostrongylus spp., Spicocaulus spp., Stephanurus spp., Strongylus spp., Syngamus spp., Teladorsagia spp., Trichonema spp., Trichostrongylus spp., Triodontophorus spp., Troglostrongylus spp., Uncinaria spp.

From the order of Spirurida, for example: Acanthocheilonema spp., Anisakis spp., Ascaridia spp.; Ascaris spp., Ascarops spp., Aspiculuris spp., Baylisascaris spp., Brugia spp., Cercopithifilaria spp., Crassicauda spp., Dipetalonema spp., Dirofilaria spp., Dracunculus spp.; Draschia spp., Enterobius spp., Filaria spp., Gnathostoma spp., Gongylonema spp., Habronema spp., Heterakis spp.; Litomosoides spp., Loa spp., Onchocerca spp., Oxyuris spp., Parabronema spp., Parafilaria spp., Parascaris spp., Passalurus spp., Physaloptera spp., Probstmayria spp., Pseudofilaria spp., Setaria spp., Skjrabinema spp., Spirocerca spp., Stephanofilaria spp., Strongyluris spp., Syphacia spp., Thelazia spp., Toxascaris spp., Toxocara spp., Wuchereria spp.

Acanthocephala: from the order of Oligacanthorhynchida, for example: Macracanthorhynchus spp., Prosthenorchis spp.; from the order of Moniliformida, for example: Moniliformis spp.
From the order of Polymorphida, for example: Filicollis spp.; from the order of Echinorhynchida, for example Acanthocephalus spp., Echinorhynchus spp., Leptorhynchoides spp.
Pentastoma: from the order of Porocephalida, for example, Linguatula spp.

In the veterinary field and in animal husbandry, the compounds of the formula (I) are administered by methods generally known in the art, such as via the enteral, parenteral, dermal or nasal route in the form of suitable preparations. Administration may be prophylactic, metaphylactic or therapeutic.

Thus, one embodiment of the present invention refers to the compounds of the formula (I) for use as a medicament.

A further aspect relates to the compounds of the formula (I) for use as an antiendoparasitic agent.

A further specific aspect of the invention relates to the compounds of the formula (I) for use as an antihelminthic agent, especially for use as a nematicide, platyhelminthicide, acanthocephalizide or pentastomicide.

A further specific aspect of the invention relates to the compounds of the formula (I) for use as an antiprotozoic agent.

A further aspect relates to the compounds of the formula (I) for use as an antiectoparasitic agent, especially an arthropodicide, very particularly an insecticide or an acaricide.

Further aspects of the invention are veterinary medicine formulations comprising an effective amount of at least one compound of the formula (I) and at least one of the following: a pharmaceutically acceptable excipient (e.g. solid or liquid diluents), a pharmaceutically acceptable auxiliary (e.g. surfactants), especially a pharmaceutically acceptable excipient used conventionally in veterinary medicine formulations and/or a pharmaceutically acceptable auxiliary conventionally used in veterinary medicine formulations.

A related aspect of the invention is a method for production of a veterinary medicine formulation as described here, which comprises the step of mixing at least one compound of the formula (I) with pharmaceutically acceptable excipients and/or auxiliaries, especially with pharmaceutically acceptable excipients used conventionally in veterinary medicine formulations and/or auxiliaries used conventionally in veterinary medicine formulations.

Another specific aspect of the invention is veterinary medicine formulations selected from the group of ectoparasiticidal and endoparasiticidal formulations, especially selected from the group of anthelmintic, antiprotozoic and arthropodicidal formulations, very particularly selected from the group of nematicidal, platyhelminthicidal, acanthocephalidicidal, pentastomicidal, insecticidal and acaricidal formulations, according to the aspects mentioned, and methods for production thereof.

Another aspect relates to a method for treatment of a parasitic infection, especially an infection caused by a parasite selected from the group of the ectoparasites and endoparasites mentioned here, by use of an effective amount of a compound of the formula (I) in an animal, especially a nonhuman animal, having a need therefor.

Another aspect relates to a method for treatment of a parasitic infection, especially an infection caused by a parasite selected from the group of the ectoparasites and endoparasites mentioned here, by use of a veterinary medicine formulation as defined here in an animal, especially a nonhuman animal, having a need therefor.

Another aspect relates to the use of the compounds of the formula (I) in the treatment of a parasite infection, especially an infection caused by a parasite selected from the group of the ectoparasites and endoparasites mentioned here, in an animal, especially a nonhuman animal.

In the present context of animal health or veterinary medicine, the term “treatment” includes prophylactic, metaphylactic and therapeutic treatment.

In a particular embodiment, in this way, mixtures of at least one compound of the formula (I) with other active ingredients, especially with endo- and ectoparasiticides, are provided for the field of veterinary medicine.

In the field of animal health, “mixture” means not just that two (or more) different active ingredients are formulated in a common formulation and correspondingly employed together, but also relates to products comprising formulations separated for each active ingredient. Accordingly, when more than two active ingredients are to be employed, all active ingredients can be formulated in a common formulation or all active ingredients can be formulated in separate formulations; likewise conceivable are mixed forms in which some of the active ingredients are formulated together and some of the active ingredients are formulated separately. Separate formulations allow the separate or successive application of the active ingredients in question.

The active ingredients specified here by their “common names” are known and are described, for example, in the “Pesticide Manual” (see above) or can be searched for on the Internet (e.g.: http://www.alanwood.net/pesticides).

Illustrative active ingredients from the group of the ectoparasiticides as mixing components, without any intention that this should constitute a restriction, include the insecticides and acaricides listed in detail above. Further usable active ingredients are listed below in accordance with the abovementioned classification based on the current IRAC Mode of Action Classification Scheme: (1) acetylcholinesterase (AChE) inhibitors; (2) GABA-gated chloride channel blockers; (3) sodium channel modulators; (4) nicotinic acetylcholine receptor (nAChR) competitive modulators; (5) nicotinic acetylcholine receptor (nAChR) allosteric modulators; (6) glutamate-gated chloride channel (GluC1) allosteric modulators; (7) juvenile hormone mimetics; (8) miscellaneous non-specific (multi-site) inhibitors; (9) chordotonal organ modulators; (10) mite growth inhibitors; (12) inhibitors of mitochondrial ATP synthase, such as ATP disruptors; (13) uncouplers of oxidative phosphorylation via disruption of the proton gradient; (14) nicotinic acetylcholine receptor channel blockers; (15) inhibitors of chitin biosynthesis, type 0; (16) inhibitors of chitin biosynthesis, type 1; (17) moulting disruptors (especially in Diptera); (18) ecdysone receptor agonists; (19) octopamine receptor agonists; (21) mitochondrial complex I electron transport inhibitors; (25) mitochondrial complex II electron transport inhibitors; (20) mitochondrial complex III electron transport inhibitors; (22) voltage-dependent sodium channel blockers; (23) inhibitors of acetyl CoA carboxylase; (28) ryanodine receptor modulators; active ingredients having unknown or non-specific mechanisms of action, e.g. fentrifanil, fenoxacrim, cycloprene, chlorobenzilate, chlordimeform, flubenzimin, dicyclanil, amidoflumet, quinomethionat, triarathene, clothiazoben, tetrasul, potassium oleate, petroleum, metoxadiazone, gossyplur, flutenzine, brompropylate, cryolite;

compounds from other classes, for example butacarb, dimetilan, cloethocarb, phosphocarb, pirimiphos(-ethyl), parathion(-ethyl), methacrifos, isopropyl o-salicylate, trichlorfon, sulprofos, propaphos, sebufos, pyridathion, prothoate, dichlofenthion, demeton-S-methyl sulfone, isazofos, cyanofenphos, dialifos, carbophenothion, autathiofos, aromfenvinfos(-methyl), azinphos(-ethyl), chlorpyrifos(-ethyl), fosmethilan, iodofenphos, dioxabenzofos, formothion, fonofos, flupyrazofos, fensulfothion, etrimfos; organochlorine compounds, for example camphechlor, lindane, heptachlor; or phenylpyrazoles, e.g. acetoprole, pyrafluprole, pyriprole, vaniliprole, sisapronil; or isoxazolines, e.g. sarolaner, afoxolaner, lotilaner, fluralaner;
pyrethroids, e.g. (cis-, trans-)metofluthrin, profluthrin, flufenprox, flubrocythrinate, fubfenprox, fenfluthrin, protrifenbut, pyresmethrin, RU15525, terallethrin, cis-resmethrin, heptafluthrin, bioethanomethrin, biopermethrin, fenpyrithrin, cis-cypermethrin, cis-permethrin, clocythrin, cyhalothrin (lambda-), chlovaporthrin, or halogenated hydrocarbon compounds (HCHs),
neonicotinoids, e.g. nithiazine
dicloromezotiaz, triflumezopyrim
macrocyclic lactones, e.g. nemadectin, ivermectin, latidectin, moxidectin, selamectin, eprinomectin, doramectin, emamectin benzoate; milbemycin oxime
triprene, epofenonane, diofenolan;
biologicals, hormones or pheromones, for example natural products, e.g. thuringiensin, codlemone or neem components
dinitrophenols, e.g. dinocap, dinobuton, binapacryl;
benzoylureas, e.g. fluazuron, penfluron,
amidine derivatives, e.g. chlormebuform, cymiazole, demiditraz
beehive varroa acaricides, for example organic acids, e.g. formic acid, oxalic acid.

Illustrative active ingredients from the group of the endoparasiticides, as mixing components, include but are not limited to active anthelmintic ingredients and active antiprotozoic ingredients.

The active anthelmintic ingredients include but are not limited to the following active nematicidal, trematicidal and/or cestocidal ingredients:

from the class of the macrocyclic lactones, for example: eprinomectin, abamectin, nemadectin, moxidectin, doramectin, selamectin, lepimectin, latidectin, milbemectin, ivermectin, emamectin, milbemycin;
from the class of the benzimidazoles and probenzimidazoles, for example: oxibendazole, mebendazole, triclabendazole, thiophanate, parbendazole, oxfendazole, netobimin, fenbendazole, febantel, thiabendazole, cyclobendazole, cambendazole, albendazole sulfoxide, albendazole, flubendazole;
from the class of the depsipeptides, preferably cyclic depsipetides, especially 24-membered cyclic depsipeptides, for example: emodepside, PF1022A;
from the class of the tetrahydropyrimidines, for example: morantel, pyrantel, oxantel;
from the class of the imidazothiazoles, for example: butamisole, levamisole, tetramisole;
from the class of the aminophenylamidines, for example: amidantel, deacylated amidantel (dAMD), tribendimidine;
from the class of the aminoacetonitriles, for example: monepantel;
from the class of the paraherquamides, for example: paraherquamide, derquantel;
from the class of the salicylanilides, for example: tribromsalan, bromoxanide, brotianide, clioxanide, closantel, niclosamide, oxyclozanide, rafoxanide;
from the class of the substituted phenols, for example: nitroxynil, bithionol, disophenol, hexachlorophen, niclofolan, meniclopholan;
from the class of the organophosphates, for example: trichlorfon, naphthalofos, dichlorvos/DDVP, crufomate, coumaphos, haloxon;
from the class of the piperazinones/quinolines, for example: praziquantel, epsiprantel;
from the class of the piperazines, for example: piperazine, hydroxyzine;
from the class of the tetracyclines, for example: tetracycline, chlorotetracycline, doxycycline, oxytetracycline, rolitetracycline;
from various other classes, for example: bunamidine, niridazole, resorantel, omphalotin, oltipraz, nitroscanate, nitroxynil, oxamniquin, mirasan, miracil, lucanthon, hycanthon, hetolin, emetin, diethylcarbamazine, dichlorophen, diamfenetide, clonazepam, bephenium, amoscanate, clorsulon.

Active antiprotozoic ingredients include but are not limited to the following active ingredients: from the class of the triazines, for example: diclazuril, ponazuril, letrazuril, toltrazuril; from the class of polyether ionophores, for example: monensin, salinomycin, maduramicin, narasin; from the class of the macrocyclic lactones, for example: milbemycin, erythromycin;

from the class of the quinolones, for example: enrofloxacin, pradofloxacin;
from the class of the quinines, for example: chloroquin;
from the class of the pyrimidines, for example: pyrimethamine;
from the class of the sulphonamides, for example: sulfaquinoxaline, trimethoprim, sulfaclozin;
from the class of the thiamines, for example: amprolium;
from the class of the lincosamides, for example: clindamycin;
from the class of the carbanilides, for example: imidocarb;
from the class of the nitrofurans, for example: nifurtimox;
from the class of the quinazolinone alkaloids, for example: halofuginone;
from various other classes, for example: oxamniquin, paromomycin;
from the class of the vaccines or antigens from microorganisms, for example: Babesia canis rossi, Eimeria tenella, Eimeria praecox, Eimeria necatrix, Eimeria mitis, Eimeria maxima, Eimeria brunetti, Eimeria acervulina, Babesia canis vogeli, Leishmania infantum, Babesia canis canis, Dictyocaulus viviparus.

All the mixing components mentioned, as the case may be, may also form salts with suitable bases or acids if they are capable of doing so on the basis of their functional groups.

### Vector Control

The compounds of the formula (I) can also be used in vector control. In the context of the present invention, a vector is an arthropod, especially an insect or arachnid, capable of transmitting pathogens, for example viruses, worms, single-cell organisms and bacteria, from a reservoir (plant, animal, human, etc.) to a host. The pathogens can be transmitted into a host either mechanically (for example trachoma by non-stinging flies) to a host or after injection (for example malaria parasites by mosquitoes).

Examples of vectors and the diseases or pathogens they transmit are:

1) mosquitoes
Anopheles: malaria, filariasis;
Culex: Japanese encephalitis, filariasis, other viral diseases, transmission of other worms;
Aedes: yellow fever, dengue fever, further viral disorders, filariasis;
Simuliidae: transmission of worms, especially Onchocerca volvulus;
Psychodidae: transmission of leishmaniasis
   2) Lice: skin infections, epidemic typhus;
   3) Fleas: plague, endemic typhus, tapeworms;
   4) Flies: sleeping sickness (trypanosomiasis); cholera, other bacterial diseases;
   5) Mites: acariosis, epidemic typhus, rickettsialpox, tularaemia, Saint Louis encephalitis, tick-borne encephalitis (TBE), Crimean-Congo haemorrhagic fever, borreliosis;
   6) Ticks: borellioses such as Borrelia bungdorferi sensu lato., Borrelia duttoni, tick-borne encephalitis, Q fever (Coxiella burnetii), babesioses (Babesia canis canis), ehrlichiosis.

Examples of vectors in the context of the present invention are insects, for example aphids, flies, leafhoppers or thrips, which can transmit plant viruses to plants. Other vectors capable of transmitting plant viruses are spider mites, lice, beetles and nematodes.

Further examples of vectors in the context of the present invention are insects and arachnids such as mosquitoes, especially of the genera Aedes, Anopheles, for example A. gambiae, A. arabiensis, A. funestus, A. dirus (malaria) and Culex, Psychodidae such as Phlebotomus, Lutzomyia, lice, fleas, flies, mites and ticks, which can transmit pathogens to animals and/or humans.

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of the formula (I) for vector control, for example in agriculture, in horticulture, in forests, in gardens and in leisure facilities, and also in the protection of materials and stored products.

### Protection of Industrial Materials

The compounds of the formula (I) are suitable for protecting industrial materials against attack or destruction by insects, for example from the orders of Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protection of wood is particularly preferred.

In a further embodiment, the compounds of the formula (I) are used together with at least one further insecticide and/or at least one fungicide.

In a further embodiment, the compounds of the formula (I) take the form of a ready-to-use pesticide, meaning that they can be applied to the material in question without further modifications. Useful further insecticides or fungicides especially include those mentioned above.

Surprisingly, it has also been found that the compounds of the formula (I) can be employed for protecting objects which come into contact with saltwater or brackish water, in particular hulls, screens, nets, buildings, moorings and signalling systems, against fouling. It is equally possible to use the compounds of the formula (I), alone or in combinations with other active ingredients, as antifouling agents.

### Control of Animal Pests in the Hygiene Sector

The compounds of the formula (I) are suitable for controlling animal pests in the hygiene sector. More particularly, the invention can be used in the domestic protection sector, in the hygiene protection sector and in the protection of stored products, particularly for control of insects, arachnids, ticks and mites encountered in enclosed spaces, for example dwellings, factory halls, offices, vehicle cabins, animal breeding facilities. For controlling animal pests, the compounds of the formula (I) are used alone or in combination with other active ingredients and/or auxiliaries. They are preferably used in domestic insecticide products. The compounds of the formula (I) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or bait stations.

The preparation and use examples which follow illustrate the invention without limiting it.

### PREPARATION EXAMPLES

### Synthesis Example No. 1

### 3-(Chloroacetyl)-1-[(2-chloro-1,3-thiazol-5-yl)methyl]-1H-imidazo[1,2-a]pyridin-4-ium-2-olate (Compound No. I-1-002)

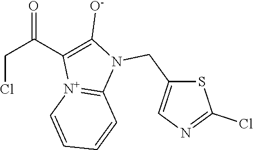 [SMILES: O=C(CCl)c1c([O-])n(Cc2cnc(Cl)s2)c2cccc[n+]12]

To a solution of N-[(2-chloro-1,3-thiazol-5-yl)methyl]pyridin-2-amine [known from WO2009099929](5.00 g, 22.1 mmol) in dioxane were added chloroacetic anhydride (7.58 g, 44.3 mmol) and chloroacetic acid (4.19 g, 44.3 mmol), and then the mixture was heated under reflux for 16 hours. After cooling, the reaction mixture was poured cautiously into a mixture of 200 ml of 10% sodium carbonate solution and 400 ml of ice-water. The precipitated solids were filtered off with suction and taken up in dichloromethane. The organic phase was dried over magnesium sulphate, and the solvent was removed under reduced pressure. 6.48 g (82.9% of theory) of 3-(chloroacetyl)-1-[(2-chloro-1,3-thiazol-5-yl)methyl]-1H-imidazo[1,2-a]pyridin-4-ium-2-olate were obtained. HPLC-MS: log P=1.69; Mass (m/z): 341.9; ¹HNMR (D₆-DMSO): δ 4.79 (s, 2H), 5.42 (s, 2H), 7.42, (m, 1H), 7.86 (s, 1H), 7.92 (m, 2H), 9.82 (m, 1H).

### Synthesis Example No. 2

### 1-[(2-Chloro-1,3-thiazol-5-yl)methyl]-3-(methoxyacetyl)-1H-imidazo[1,2-a]pyridin-4-ium-2-olate (Compound No. I-1-008)

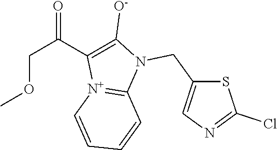 [SMILES: COCC(=O)c1c([O-])n(Cc2cnc(Cl)s2)c2cccc[n+]12]

3-(Chloroacetyl)-1-[(2-chloro-1,3-thiazol-5-yl)methyl]-1H-imidazo[1,2-a]pyridin-4-ium-2-olate (Compound No. I-1-002) (250 mg, 0.73 mmol) was initially charged in methanol/dichloromethane (1:1) (12 ml), and sodium methoxide (59.2 mg, 1.09 mmol) was added. The reaction mixture was then stirred at room temperature for 16 hours. After diluting with dichloromethane, the mixture was washed with water, the organic phase was dried over magnesium sulphate and the solvent was distilled off under reduced pressure. The residue was adsorbed on silica gel and chromatographed with dichloromethane/2-propanol (92:8) as eluent. 69.0 mg (26% of theory) of 1-[(2-chloro-1,3-thiazol-5-yl)methyl]-3-(methoxyacetyl)-1H-imidazo[1,2-a]pyridin-4-ium-2-olate were obtained. HPLC-MS: log P=1.23; Mass (m/z): 338.0; ¹HNMR (D₆-DMSO): δ 4.53 (s, 2H), 5.39 (s, 2H), 7.38, (m, 1H), 7.88 (m, 3H), 9.88 (m, 1H).

### Synthesis Example No. 3

### 1-[(2-Chloro-1,3-thiazol-5-yl)methyl]-3-(isopropoxyacetyl)-1H-imidazo[,2-a]pyridin-4-ium-2-olate (Compound No. I-1-026)

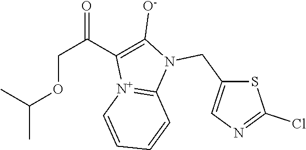 [SMILES: CC(C)OCC(=O)c1c([O-])n(Cc2cnc(Cl)s2)c2cccc[n+]12]

To 2-propanol (2.5 ml) was added NaH, 60% in oil dispersion (21 mg, 0.87 mmol), and the mixture was stirred at room temperature for 30 min. This solution is slowly added dropwise to a solution of 3-(chloroacetyl)-1-[(2-chloro-1,3-thiazol-5-yl)methyl]-1H-imidazo[1,2-a]pyridin-4-ium-2-olate (200 mg, 0.58 mmol) (Compound No. I-1-002) in 2-propanol/dichloromethane (1:1) (10.0 ml) and then stirred at room temperature for 16 hours. After diluting with dichloromethane, the mixture was washed with water, the organic phase was dried over magnesium sulphate and the solvent was distilled off under reduced pressure. The residue was adsorbed on silica gel and chromatographed with dichloromethane/2-propanol (92:8) as eluent. 129.0 mg (60% of theory) of 1-[(2-chloro-1,3-thiazol-5-yl)methyl]-3-(isopropoxyacetyl)-1H-imidazo[1,2-a]pyridin-4-ium-2-olate were obtained. HPLC-MS: log P=1.73; Mass (m/z): 365.9; ¹HNMR (D₆-DMSO): δ 1.14 (d, 6H), 3.68 (m, 1H), 4.58 (s, 2H), 5.40 (s, 2H), 7.37, (m, 1H), 7.85 (m, 3H), 9.88 (m, 1H).

### Synthesis Example No. 4

### 1-[(2-Chloro-1,3-thiazol-5-yl)methyl]-3-[(ethylsulphanyl)acetyl]-1H-imidazo[1,2-a]pyridin-4-ium-2-olate (Compound No. I-1-028)

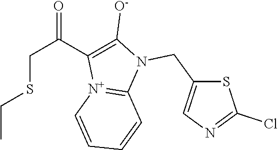 [SMILES: CCSCC(=O)c1c([O-])n(Cc2cnc(Cl)s2)c2cccc[n+]12]

3-(Chloroacetyl)-1-[(2-chloro-1,3-thiazol-5-yl)methyl]-1H-imidazo[1,2-a]pyridin-4-ium-2-olate (Compound No. I-1-002) (255 mg, 0.74 mmol) was initially charged in DMF (10 ml), and a solution of sodium ethanethiolate (93.8 mg, 1.11 mmol) in 2 ml of DMF was added at 0° C. The reaction mixture was then stirred at room temperature for 16 hours and the DMF was subsequently distilled off under reduced pressure. The residue was taken up in dichloromethane/water, and the organic phase was removed and dried over magnesium sulphate. After the solvent had been distilled off under reduced pressure, the residue was adsorbed on silica gel and chromatographed with dichloromethane/2-propanol (92:8) as eluent. 205 mg (61.4% of theory) of 1-[(2-chloro-1,3-thiazol-5-yl)methyl]-3-[(ethylsulphanyl)acetyl]-1H-imidazo[1,2-a]pyridin-4-ium-2-olate were obtained. HPLC-MS: log P=1.99; Mass (m/z): 368.0; ¹HNMR (D₆-DMSO): δ 1.20 (t, 3H), 2.50 (q, 2H), 3.86 (s, 2H), 5.41 (s, 2H), 7.37, (m, 1H), 7.89 (m, 3H), 9.88 (m, 1H).

### Synthesis Example No. 5

### 1-[(2-Chloro-1,3-thiazol-5-yl)methyl]-3-[(ethylsulphanyl)acetyl]-1H-imidazo[1,2-a]pyridin-4-ium-2-olate and 1-[(2-chloro-1,3-thiazol-5-yl)methyl]-3-[(ethylsulphonyl) acetyl]-1H-imidazo[1,2-a]pyridin-4-ium-2-olate (Compound No. I-1-037 and I-1-042)

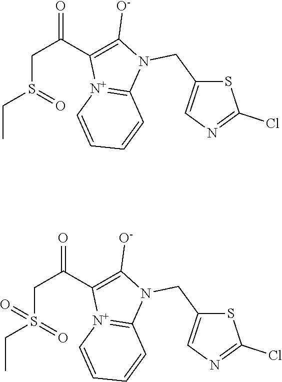 [SMILES: CCS(=O)CC(=O)c1c([O-])n(Cc2cnc(Cl)s2)c2cccc[n+]12 ; CCS(=O)(=O)CC(=O)c1c([O-])n(Cc2cnc(Cl)s2)c2cccc[n+]12]

1-[(2-Chloro-1,3-thiazol-5-yl)methyl]-3-[(ethylsulphanyl)acetyl]-1H-imidazo[1,2-a]pyridin-4-ium-2-olate (Compound No. I-1-028) (161 mg, 0.43 mmol) was initially charged in dichloromethane (10 ml), and meta-chloroperbenzoic acid, 70% (146 mg, 0.59 mmol) was added at 0° C. The reaction mixture was stirred while it warmed to room temperature for 16 hours, then the reaction mixture was taken up in dichloromethane/water and washed with saturated sodium hydrogencarbonate solution, and the organic phase was removed and dried over magnesium sulphate. After the solvent had been distilled off under reduced pressure, the residue was adsorbed on silica gel and chromatographed with dichloromethane/2-propanol (92:8) as eluent. 82 mg (45% of theory) of 1-[(2-chloro-1,3-thiazol-5-yl)methyl]-3-[(ethylsulphanyl)acetyl]-1H-imidazo[1,2-a]pyridin-4-ium-2-olate and 15 mg (7% of theory) of 1-[(2-chloro-1,3-thiazol-5-yl)methyl]-3-[(ethylsulphonyl)acetyl]-1H-imidazo[1,2-a]pyridin-4-ium-2-olate were obtained. 1-[(2-Chloro-1,3-thiazol-5-yl)methyl]-3-[(ethylsulphanyl)acetyl]-1H-imidazo[1,2-a]pyridin-4-ium-2-olate: HPLC-MS: log P=1.13; Mass (m/z): 384.0; ¹HNMR (D₆-DMSO): δ 1.24 (t, 3H), 2.78 (m, 1H), 2.92 (m, 1H), 4.40 (q, 2H), 5.41 (s, 2H), 7.40, (m, 1H), 7.86 (s, 1H), 7.92 (m, 2H), 9.84 (m, 1H); 1-[(2-Chloro-1,3-thiazol-5-yl)methyl]-3-[(ethylsulphonyl)acetyl]-1H-imidazo[1,2-a]pyridin-4-ium-2-olate: HPLC-MS: log P=1.38; Mass (m/z): 400.0; ¹HNMR (D₆-DMSO): δ 1.28 (t, 3H), 3.1 (m, 2H), 4.93 (s, 2H), 5.42 (s, 2H), 7.42, (m, 1H), 7.86 (s, 1H), 7.97 (m, 2H), 9.83 (m, 1H);

### Synthesis Example No. 6

### 1-[(2-Chloro-1,3-thiazol-5-yl)methyl]-3-(1H-pyrazol-1-ylacetyl)-1H-imidazo[1,2-a]pyridin-4-ium-2-olate (Compound No. I-1-009)

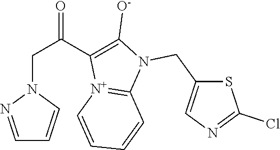 [SMILES: O=C(Cn1cccn1)c1c([O-])n(Cc2cnc(Cl)s2)c2cccc[n+]12]

3-(Chloroacetyl)-1-[(2-chloro-1,3-thiazol-5-yl)methyl]-1H-imidazo[1,2-a]pyridin-4-ium-2-olate (Compound No. I-1-002) (100 mg, 0.74 mmol) was added to a suspension of caesium carbonate (190 mg, 0.58 mmol) and pyrazole (30 mg, 0.43 mmol) in DMF (5 ml), and then the mixture was stirred at room temperature for 16 hours. The reaction mixture was taken up in dichloromethane/water, and the organic phase was removed and dried over magnesium sulphate. After the solvent had been distilled off under reduced pressure, the residue was adsorbed on silica gel and chromatographed with dichloromethane/2-propanol (92:8) as eluent. 64 mg (50.9% of theory) of 1-[(2-chloro-1,3-thiazol-5-yl)methyl]-3-(1H-pyrazol-1-ylacetyl)-1H-imidazo[1,2-a]pyridin-4-ium-2-olate were obtained. HPLC-MS: log P=1.47; Mass (m/z): 374.0; ¹HNMR (D₆-DMSO): δ 5.49 (s, 2H), 5.52 (s, 2H), 6.27 (s, 1H), 7.38, (m, 2H), 7.73 (s, 1H), 7.87 (m, 3H), 9.76 (m, 1H).

### Synthesis Example No. 7

### 1-[(2-Chloro-1,3-thiazol-5-yl)methyl]-3-(N-ethylglycyl)-1H-imidazo[1,2-a]pyridin-4-ium-2-olate (Compound No. I-1-021)

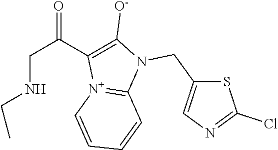 [SMILES: CCNCC(=O)c1c([O-])n(Cc2cnc(Cl)s2)c2cccc[n+]12]

3-(Chloroacetyl)-1-[(2-chloro-1,3-thiazol-5-yl)methyl]-1H-imidazo[1,2-a]pyridin-4-ium-2-olate (Compound No. I-1-002) (200 mg, 0.58 mmol) was initially charged in THF (10 ml), and ethylamine (66.0 mg, 1.46 mmol) in 1 ml of THF was added. The reaction mixture was then stirred at 50° C. for 16 hours. The reaction mixture was taken up in dichloromethane/water, and the organic phase was removed and dried over magnesium sulphate. After the solvent had been distilled off under reduced pressure, the residue was adsorbed on silica gel and chromatographed with dichloromethane/2-propanol (92:8) as eluent. 132 mg (60.5% of theory) of 1-[(2-chloro-1,3-thiazol-5-yl)methyl]-3-(N-ethylglycyl)-1H-imidazo[1,2-a]pyridin-4-ium-2-olate were obtained. HPLC-MS: log P=0.57; Mass (m/z): 350.9; ¹HNMR (D₆-DMSO): δ 1.15 (m, 3H), 3.02 (m, 2H), 4.27 (s, 2H), 5.43 (s, 2H), 7.45, (m, 1H), 7.87 (s, 1H), 7.96 (m, 2H), 9.76 (m, 1H).

### Synthesis Example No. 8

### 3-(N-Acetyl-N-ethylglycyl)-1-[(2-chloro-1,3-thiazol-5-yl)methyl]-1H-imidazo[1,2-a]pyridin-4-ium-2-olate (Compound No. I-1-040)

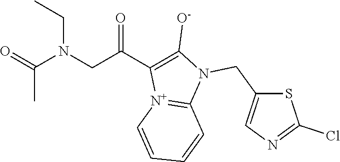 [SMILES: CCN(CC(=O)c1c([O-])n(Cc2cnc(Cl)s2)c2cccc[n+]12)C(C)=O]

1-[(2-Chloro-1,3-thiazol-5-yl)methyl]-3-(N-ethylglycyl)-1H-imidazo[1,2-a]pyridin-4-ium-2-olate (Compound No. I-1-021) (56.0 mg, 0.16 mmol) was initially charged in dichloromethane (5 ml), and triethylamine (19.5 mg, 0.19 mmol) and then acetyl chloride (15.1 mg, 0.19 mmol) were added. The reaction mixture was stirred at room temperature for 2 hours and then the solvent was distilled off under reduced pressure. The residue was adsorbed on Isolute and chromatographed using alumina with dichloromethane/2-propanol (92:8) as eluent. 30.0 mg (46.9% of theory) of 3-(N-acetyl-N-ethylglycyl)-1-[(2-chloro-1,3-thiazol-5-yl)methyl]-1H-imidazo[1,2-a]pyridin-4-ium-2-olate were obtained. HPLC-MS: log P=1.49; Mass (m/z): 392.9; ¹HNMR (D₆-DMSO): δ 1.01 (t, 3H), 2.07 (s, 3H), 3.85 (m, 2H), 4.57 (s, 1H), 4.68 (s, 1H), 5.42 (s, 2H), 7.38, (m, 1H), 7.90 (m, 3H), 9.84 (m, 1H).

### Synthesis Example No. 9

### 3-Acetyl-1-[(2-chloro-1,3-thiazol-5-yl)methyl]-1H-imidazo[1,2-a]pyridin-4-ium-2-olate (Compound No. I-1-043)

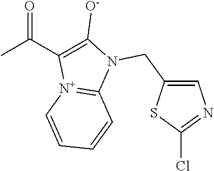 [SMILES: CC(=O)c1c([O-])n(Cc2cnc(Cl)s2)c2cccc[n+]12]

3-(Chloroacetyl)-1-[(2-chloro-1,3-thiazol-5-yl)methyl]-1H-imidazo[1,2-a]pyridin-4-ium-2-olate (Compound No. I-1-002) (1.53 g, 4.46 mmol) was initially charged in THF (20 ml), and activated zinc powder (875 mg, 13.3 mmol) was added. The reaction mixture was then stirred at room temperature for 5 hours and filtered through kieselguhr, and the residue was washed through with dichloromethane and washed successively with water and saturated sodium hydrogencarbonate solution. After drying over magnesium sulphate and distillative removal of the solvent under reduced pressure, the residue was adsorbed on silica gel and chromatographed with dichloromethane/2-propanol (92:8) as eluent. 1.02 g (71.7% of theory) of 3-acetyl-1-[(2-chloro-1,3-thiazol-5-yl)methyl]-1H-imidazo[1,2-a]pyridin-4-ium-2-olate were obtained. HPLC-MS: log P=1.29; Mass (m/z): 308.1; ¹HNMR (D₆-DMSO): δ 2.44 (s, 3H), 5.40 (s, 2H), 7.34, (m, 1H), 7.85 (m, 3H), 9.92 (m, 1H).

### Synthesis Example No. 10

### 1-[(2-Chloro-1,3-thiazol-5-yl)methyl]-3-(methoxycarbonyl)-1H-imidazo[1,2-a]pyridin-4-ium-2-olate (Compound No. I-1-085)

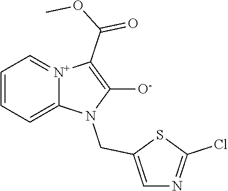 [SMILES: COC(=O)c1c([O-])n(Cc2cnc(Cl)s2)c2cccc[n+]12]

Methyl 2,3-dichloro-3-oxopropanoate (known from Tetrahedron 1993, 49, 9447-9452) (2.85 g, 16.6 mmol) dissolved in 20 ml of dichloromethane was slowly added dropwise to a solution of N-[(2-chloro-1,3-thiazol-5-yl)methyl]pyridin-2-amine (3.14 g, 13.8 mmol) and triethylamine (1.69 g, 16.6 mmol) in dichloromethane (50 ml), and then the mixture was stirred at room temperature for 3 hours. The reaction mixture was diluted with dichloromethane and washed with water, and the organic phase was removed and dried over magnesium sulphate. After the solvent had been distilled off under reduced pressure, the residue was adsorbed on silica gel and chromatographed with dichloromethane/2-propanol (92:8) as eluent. 2.48 g (54.5% of theory) of 1-[(2-chloro-1,3-thiazol-5-yl)methyl]-3-(methoxycarbonyl)-1H-imidazo[1,2-a]pyridin-4-ium-2-olate were obtained. HPLC-MS: log P=1.23; Mass (m/z): 324.0; ¹HNMR (D₆-DMSO): δ 3.74 (s, 3H), 5.38 (s, 2H), 7.34, (m, 1H), 7.76 (m, 1H), 7.83 (m, 2H), 9.46 (s, 1H).

### Synthesis Example No. 11

### 1-[(2-Chloro-1,3-thiazol-5-yl)methyl]-3-(cyclopropylcarbamoyl)-1H-imidazo[1,2-a]pyridin-4-ium-2-olate (Compound No. I-1-111)

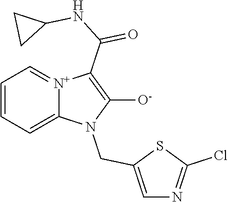 [SMILES: O=C(NC1CC1)c1c([O-])n(Cc2cnc(Cl)s2)c2cccc[n+]12]

Cyclopropylamine (50.0 mg, 0.87 mmol) was initially charged in THF, a 2 M trimethylaluminium solution in toluene (56.4 mg, 0.78 mmol) was added dropwise at 0° C., and then the mixture was stirred for 20 minutes. Thereafter, a solution of 1-[(2-chloro-1,3-thiazol-5-yl)methyl]-3-(methoxycarbonyl)-1H-imidazo[1,2-a]pyridin-4-ium-2-olate (Compound No. I-1-085) (101.3 mg, 0.31 mmol) in dichloromethane (5 ml) was added, and the mixture was stirred at 40° C. for four hours. For workup, the reaction mixture was first stirred with 15 ml of 10% hydrochloric acid and then repeatedly extracted with dichloromethane. The organic phase was washed with water and dried over magnesium sulphate. After the solvent had been distilled off under reduced pressure, the residue was adsorbed on silica gel and chromatographed with dichloromethane/2-propanol (92:8) as eluent. 99 mg (40.8% of theory) of 1-[(2-chloro-1,3-thiazol-5-yl)methyl]-3-(cyclopropylcarbamoyl)-1H-imidazo[1,2-a]pyridin-4-ium-2-olate were obtained. HPLC-MS: log P=1.63; Mass (m/z): 349.0; ¹HNMR (D₆-DMSO): δ 0.48 (m, 2H), 0.71 (m, 2H), 2.76 (m, 1H), 5.43 (s, 2H), 7.32, (m, 1H), 7.71 (m, 1H), 7.84 (m, 1H), 7.94 (m, 1H), 9.63 (m, 1H).

### Synthesis Example No. 12

### 1-[(6-Chloropyridin-3-yl)methyl]-3-(methoxycarbonyl)-1H-imidazo[1,2-a]pyrimidin-4-ium-2-olate (Compound No. I-4-001)

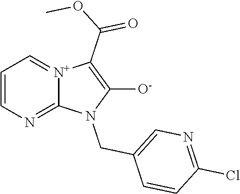 [SMILES: COC(=O)c1c([O-])n(Cc2ccc(Cl)nc2)c2nccc[n+]12]

### 1. Synthesis of N-[(6-chloropyridin-3-yl)methyl]pyrimidin-2-amine

To a solution of 2-bromopyrimidine (5.00 g, 31.5 mmol), palladium acetate (0.71 mg, 3.1 mmol), 4,5-bis(diphenylphosphino)-9,9-dimethylxanthene (3.64 g, 6.3 mmol) and potassium carbonate (34.77 g, 251.6 mmol) in dioxane (80 ml) was added 1-(6-chloropyridin-3-yl)methanamine (6.73 g, 47.2 mmol), and then the mixture was heated to 100° C. The reaction mixture was filtered through Celite and taken up in dichloromethane. The organic phase was dried over magnesium sulphate, and the solvent was removed under reduced pressure. The residue was adsorbed on silica gel and chromatographed. 1.750 g (23.9% of theory) of N-[(6-chloropyridin-3-yl)methyl]pyrimidin-2-amine were obtained. HPLC-MS: log P=1.36; Mass (m/z): 221.1; ¹HNMR (D6-DMSO): δ 8.32 (m, 4H), 7.76 (m, 2H), 7.46 (m, 1H), 6.60 (m, 1H), 4.49 (d, 2H).

### 2. Synthesis of 1-[(6-chloropyridin-3-yl)methyl]-3-(methoxycarbonyl)-1H-imidazo[1,2-a]pyrimidin-4-ium-2-olate

To a solution of N-[(6-chloropyridin-3-yl)methyl]pyrimidin-2-amine (0.50 g, 2.3 mmol) in dioxane, 4-dimethylaminopyridine (1 spatula-tip) and triethylamine (0.46 g, 4.5 mmol) was added, at 0° C., methyl 3-chloro-3-oxopropanoate (0.93 g, 6.8 mmol), and then the mixture was stirred at 25° C. overnight. The reaction mixture was poured into an ammonium chloride solution and then taken up in ethyl acetate. The organic phase was dried over magnesium sulphate, and the solvent was removed under reduced pressure. The residue was adsorbed on silica gel, chromatographed rapidly and converted further.

To a solution of methyl 3-{[(6-chloropyridin-3-yl)methyl](pyrimidin-2-yl)amino}-3-oxopropanoate (0.29 g, 0.9 mmol) in dioxane was added, at 25° C., N-bromosuccinimide (0.32 g, 1.8 mmol), and then the mixture was stirred at 50° C. for 2 hours. The reaction mixture was poured into an ammonium chloride solution and then taken up in ethyl acetate. The organic phase was dried over magnesium sulphate, and the solvent was removed under reduced pressure. The residue was adsorbed on silica gel and chromatographed. 97 mg (29% of theory) of 1-[(6-chloropyridin-3-yl)methyl]-3-(methoxycarbonyl)-1H-imidazo[1,2-a]pyrimidin-4-ium-2-olate were obtained. HPLC-MS: log P=1.15; Mass (m/z): 319.0; ¹HNMR (D6-DMSO): δ 9.62 (m, 1H), 8.55 (m, 1H), 8.45 (m, 1H), 7.83 (m, 1H), 7.49 (m, 2H), 5.18 (s, 2H), 3.75 (s, 3H).

### Synthesis Example No. 13

### 5-(Chloroacetyl)-7-[(6-chloropyridin-3-yl)methyl]-7H-imidazo[2,1-b][1,3]thiazol-4-ium-6-olate (Compound No. I-5-001)

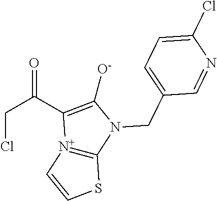 [SMILES: O=C(CCl)c1c([O-])n(Cc2ccc(Cl)nc2)c2scc[n+]12]

To a solution of N-[(2-chloro-1,3-thiazol-5-yl)methyl]pyridin-2-amine [known from WO2009099929](500 mg, 2.21 mmol) in dioxane were added chloroacetic anhydride (757 mg, 4.43 mmol) and chloroacetic acid (418 mg, 4.43 mmol), and then the mixture was heated under reflux for 40 hours. After cooling, the reaction mixture was poured cautiously into 150 ml of 10% cold sodium carbonate solution and extracted repeatedly with dichloromethane. The organic phase was dried over magnesium sulphate, and the solvent was removed under reduced pressure. The residue was adsorbed on silica gel and chromatographed with dichloromethane/2-propanol (92:8) as eluent. 30.0 mg (3.76% of theory) of 5-(chloroacetyl)-7-[(6-chloropyridin-3-yl)methyl]-7H-imidazo[2,1-b][1,3]thiazol-4-ium-6-olate were obtained. HPLC-MS: log P=1.33; Mass (m/z): 341.9; ¹HNMR (D₆-DMSO): δ 4.65 (s, 2H), 5.17 (s, 2H), 7.47, (m, 1H), 7.55 (m, 1H), 7.92 (m, 1H), 8.52 (m, 2H).

The inventive compounds of the formula (I-1), (1-4) and (1-5) described in Tables 1-3 below are likewise preferred inventive compounds of the formula (I) which are obtained according to or analogously to the Synthesis Examples described above.

TABLE 1
(I-1)

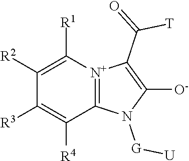

| No. | R¹ | R² | R³ | R⁴ | G-U | T | log p^{a)} |
|---|---|---|---|---|---|---|---|
| I-1-001 | H | H | H | H | 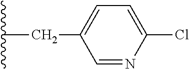 [SMILES: *Cc1ccc(Cl)nc1] | 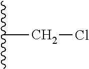 [SMILES: *CCl] | 1.62 |
| I-1-002 | H | H | H | H | 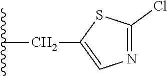 [SMILES: *Cc1cnc(Cl)s1] | 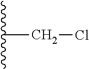 [SMILES: *CCl] | 1.69 |
| I-1-003 | H | H | H | H | 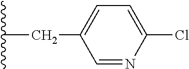 [SMILES: *Cc1ccc(Cl)nc1] | 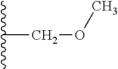 [SMILES: *COC] | 1.18 |
| I-1-004 | H | H | H | H | 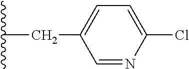 [SMILES: *Cc1ccc(Cl)nc1] | 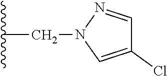 [SMILES: *Cn1cc(Cl)cn1] | 1.96 |
| I-1-005 | H | H | H | H | 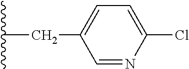 [SMILES: *Cc1ccc(Cl)nc1] | 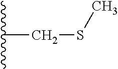 [SMILES: *CSC] | 1.62 |
| I-1-006 | H | H | H | H | 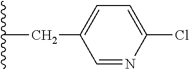 [SMILES: *Cc1ccc(Cl)nc1] | 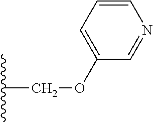 [SMILES: *COc1cccnc1] | 0.93 |
| I-1-007 | H | H | H | H | 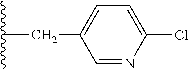 [SMILES: *Cc1ccc(Cl)nc1] | 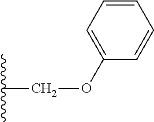 [SMILES: *COc1ccccc1] | 2.2 |
| I-1-008 | H | H | H | H | 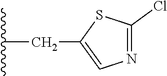 [SMILES: *Cc1cnc(Cl)s1] | 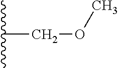 [SMILES: *COC] | 1.23 |
| I-1-009 | H | H | H | H | 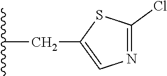 [SMILES: *Cc1cnc(Cl)s1] | 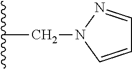 [SMILES: *Cn1cccn1] | 1.47 |
| I-1-010 | H | H | H | H | 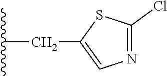 [SMILES: *Cc1cnc(Cl)s1] | 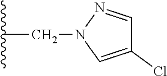 [SMILES: *Cn1cc(Cl)cn1] | 2.09 |
| I-1-011 | H | H | H | H | 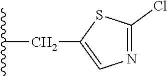 [SMILES: *Cc1cnc(Cl)s1] | 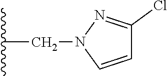 [SMILES: *Cn1ccc(Cl)n1] | 2.03 |
| I-1-012 | H | H | H | H | 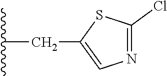 [SMILES: *Cc1cnc(Cl)s1] | 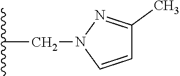 [SMILES: *Cn1ccc(C)n1] | 1.63 |
| I-1-013 | H | H | H | H | 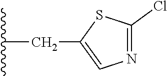 [SMILES: *Cc1cnc(Cl)s1] | 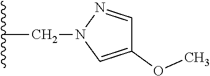 [SMILES: *Cn1cc(OC)cn1] | 1.58 |
| I-1-014 | H | H | H | H | 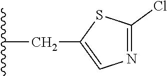 [SMILES: *Cc1cnc(Cl)s1] | 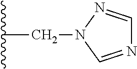 [SMILES: *Cn1cncn1] | 1.1 |
| I-1-015 | H | H | H | H | 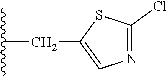 [SMILES: *Cc1cnc(Cl)s1] | 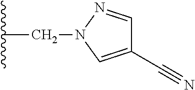 [SMILES: *Cn1cc(C#N)cn1] | 1.68 |
| I-1-016 | H | H | H | H | 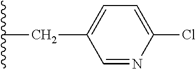 [SMILES: *Cc1ccc(Cl)nc1] | 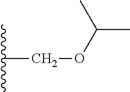 [SMILES: *COC(C)C] | 1.67 |
| I-1-017 | H | H | H | H | 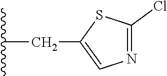 [SMILES: *Cc1cnc(Cl)s1] | 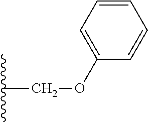 [SMILES: *COc1ccccc1] | 2.34 |
| I-1-018 | H | H | H | H | 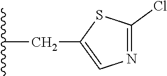 [SMILES: *Cc1cnc(Cl)s1] | 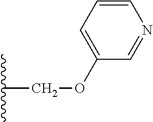 [SMILES: *COc1cccnc1] | 1 |
| I-1-019 | H | H | H | H | 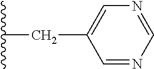 [SMILES: *Cc1cncnc1] | 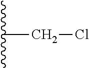 [SMILES: *CCl] | 0.77 |
| I-1-020 | H | H | H | H | 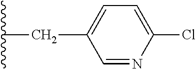 [SMILES: *Cc1ccc(Cl)nc1] | 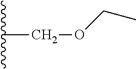 [SMILES: *COCC] | 1.39 |
| I-1-021 | H | H | H | H | 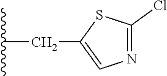 [SMILES: *Cc1cnc(Cl)s1] | 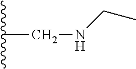 [SMILES: *CNCC] | 0.57 |
| I-1-022 | H | H | H | H | 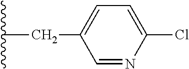 [SMILES: *Cc1ccc(Cl)nc1] | 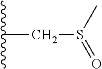 [SMILES: *CS(C)=O] | 0.91 |
| I-1-023 | H | H | H | H | 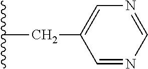 [SMILES: *Cc1cncnc1] | 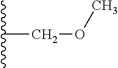 [SMILES: *COC] | 0.4 |
| I-1-024 | H | H | H | H | 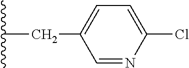 [SMILES: *Cc1ccc(Cl)nc1] | 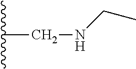 [SMILES: *CNCC] | 0.42 |
| I-1-025 | H | H | H | H | 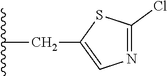 [SMILES: *Cc1cnc(Cl)s1] | 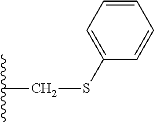 [SMILES: *CSc1ccccc1] | 2.67 |
| I-1-026 | H | H | H | H | 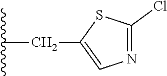 [SMILES: *Cc1cnc(Cl)s1] | 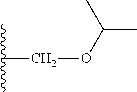 [SMILES: *COC(C)C] | 1.8 |
| I-1-027 | H | H | H | H | 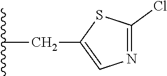 [SMILES: *Cc1cnc(Cl)s1] | 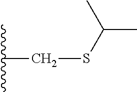 [SMILES: *CSC(C)C] | 2.27 |
| I-1-028 | H | H | H | H | 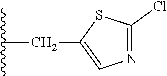 [SMILES: *Cc1cnc(Cl)s1] | 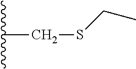 [SMILES: *CSCC] | 1.99 |
| I-1-029 | H | H | H | H | 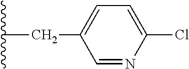 [SMILES: *Cc1ccc(Cl)nc1] | 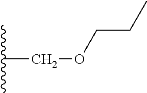 [SMILES: *COCCC] | 1.72 |
| I-1-030 | H | H | H | H | 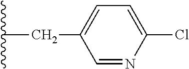 [SMILES: *Cc1ccc(Cl)nc1] | 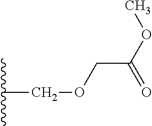 [SMILES: *COCC(=O)OC] | 1.31 |
| I-1-031 | H | H | H | H | 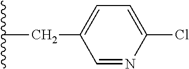 [SMILES: *Cc1ccc(Cl)nc1] | 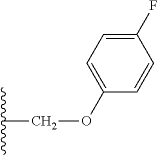 [SMILES: *COc1ccc(F)cc1] | 2.27 |
| I-1-032 | H | H | H | H | 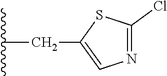 [SMILES: *Cc1cnc(Cl)s1] | 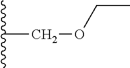 [SMILES: *COCC] | 1.57 |
| I-1-033 | H | H | H | H | 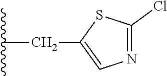 [SMILES: *Cc1cnc(Cl)s1] | 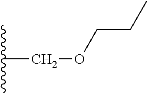 [SMILES: *COCCC] | 1.92 |
| I-1-034 | H | H | H | H | 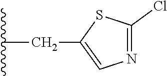 [SMILES: *Cc1cnc(Cl)s1] | 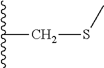 [SMILES: *CSC] | 1.73 |
| I-1-035 | H | H | H | H | 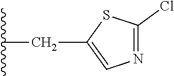 [SMILES: *Cc1cnc(Cl)s1] | 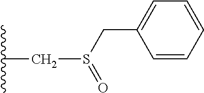 [SMILES: *CS(=O)Cc1ccccc1] | 1.72 |
| I-1-036 | H | H | H | H | 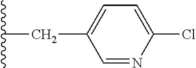 [SMILES: *Cc1ccc(Cl)nc1] | 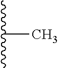 | 1.2 |
| I-1-037 | H | H | H | H | 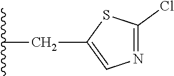 [SMILES: *Cc1cnc(Cl)s1] | 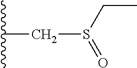 [SMILES: *CS(=O)CC] | 1.13 |
| I-1-038 | H | H | H | H | 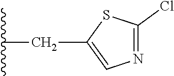 [SMILES: *Cc1cnc(Cl)s1] | 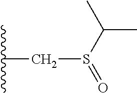 [SMILES: *CS(=O)C(C)C] | 1.32 |
| I-1-039 | H | H | H | H | 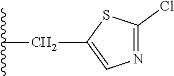 [SMILES: *Cc1cnc(Cl)s1] | 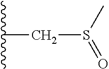 [SMILES: *CS(C)=O] | 1.08 |
| I-1-040 | H | H | H | H | 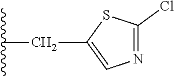 [SMILES: *Cc1cnc(Cl)s1] | 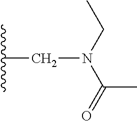 [SMILES: *CN(CC)C(C)=O] | 1.49 |
| I-1-041 | H | H | H | H | 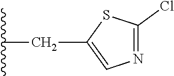 [SMILES: *Cc1cnc(Cl)s1] | 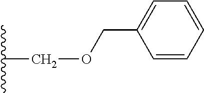 [SMILES: *COCc1ccccc1] | 2.35 |
| I-1-042 | H | H | H | H | 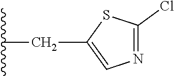 [SMILES: *Cc1cnc(Cl)s1] | 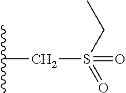 [SMILES: *CS(=O)(=O)CC] | 1.38 |
| I-1-043 | H | H | H | H | 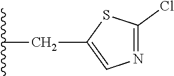 [SMILES: *Cc1cnc(Cl)s1] | 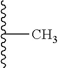 | 1.29 |
| I-1-044 | H | H | H | H | 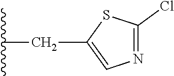 [SMILES: *Cc1cnc(Cl)s1] | 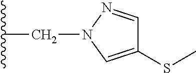 [SMILES: *Cn1cc(SC)cn1] | 1.97 |
| I-1-045 | H | H | H | H | 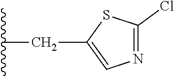 [SMILES: *Cc1cnc(Cl)s1] | 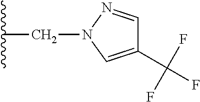 [SMILES: *Cn1cc(C(F)(F)F)cn1] | 2.37 |
| I-1-046 | H | H | H | H | 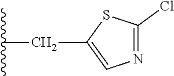 [SMILES: *Cc1cnc(Cl)s1] | 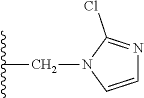 [SMILES: *Cn1ccnc1Cl] | 1.44 |
| I-1-047 | H | H | H | H | 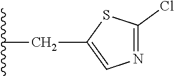 [SMILES: *Cc1cnc(Cl)s1] | 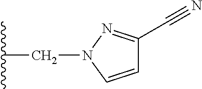 [SMILES: *Cn1ccc(C#N)n1] | 1.83 |
| I-1-048 | H | H | H | H | 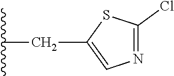 [SMILES: *Cc1cnc(Cl)s1] | 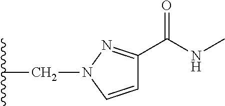 [SMILES: *Cn1ccc(C(=O)NC)n1] | 1.27 |
| I-1-049 | H | H | H | H | 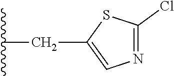 [SMILES: *Cc1cnc(Cl)s1] | 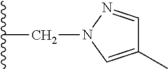 [SMILES: *Cn1cc(C)cn1] | 1.72 |
| I-1-050 | H | H | H | H | 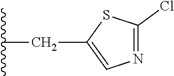 [SMILES: *Cc1cnc(Cl)s1] | 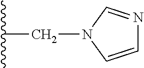 [SMILES: *Cn1ccnc1] | 0.46 |
| I-1-051 | H | H | H | H | 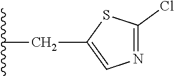 [SMILES: *Cc1cnc(Cl)s1] | 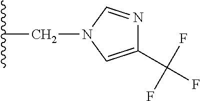 [SMILES: *Cn1cnc(C(F)(F)F)c1] | 2 |
| I-1-052 | H | H | H | H | 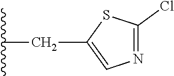 [SMILES: *Cc1cnc(Cl)s1] | 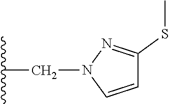 [SMILES: *Cn1ccc(SC)n1] | 1.94 |
| I-1-053 | H | H | H | H | 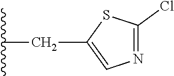 [SMILES: *Cc1cnc(Cl)s1] | 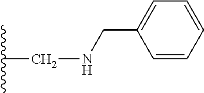 [SMILES: *CNCc1ccccc1] | 1 |
| I-1-054 | H | H | H | H | 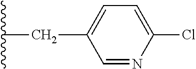 [SMILES: *Cc1ccc(Cl)nc1] | 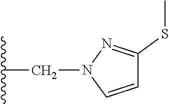 [SMILES: *Cn1ccc(SC)n1] | 1.35 |
| I-1-055 | H | H | H | H | 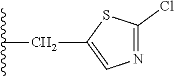 [SMILES: *Cc1cnc(Cl)s1] | 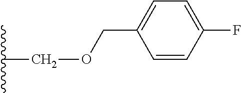 [SMILES: *COCc1ccc(F)cc1] | 2.41 |
| I-1-056 | H | H | H | H | 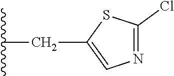 [SMILES: *Cc1cnc(Cl)s1] | 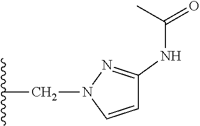 [SMILES: *Cn1ccc(NC(C)=O)n1] | 1.21 |
| I-1-057 | H | H | H | H | 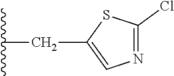 [SMILES: *Cc1cnc(Cl)s1] | 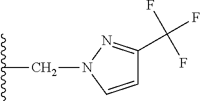 [SMILES: *Cn1ccc(C(F)(F)F)n1] | 2.44 |
| I-1-058 | H | H | H | H | 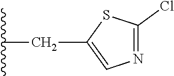 [SMILES: *Cc1cnc(Cl)s1] | 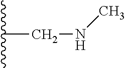 [SMILES: *CNC] | 0.31 |
| I-1-059 | H | H | H | H | 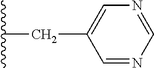 [SMILES: *Cc1cncnc1] | 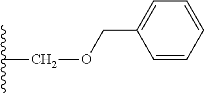 [SMILES: *COCc1ccccc1] | 1.44 |
| I-1-060 | H | H | H | H | 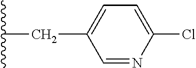 [SMILES: *Cc1ccc(Cl)nc1] | 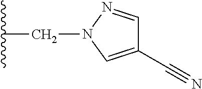 [SMILES: *Cn1cc(C#N)cn1] | 1.55 |
| I-1-061 | H | H | H | H | 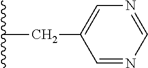 [SMILES: *Cc1cncnc1] | 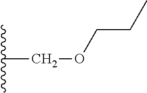 [SMILES: *COCCC] | 1.08 |
| I-1-062 | H | H | H | H | 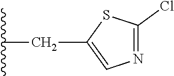 [SMILES: *Cc1cnc(Cl)s1] | 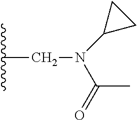 [SMILES: *CN(C(C)=O)C1CC1] | 1.58 |
| I-1-063 | H | H | CF₃ | H | 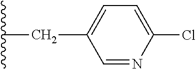 [SMILES: *Cc1ccc(Cl)nc1] | 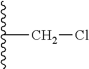 [SMILES: *CCl] | 2.52 |
| I-1-064 | H | H | H | H | 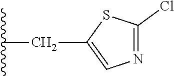 [SMILES: *Cc1cnc(Cl)s1] | 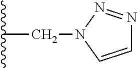 [SMILES: *Cn1ccnn1] | 1.18 |
| I-1-065 | H | H | H | H | 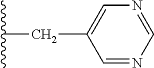 [SMILES: *Cc1cncnc1] | 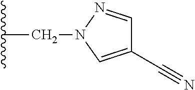 [SMILES: *Cn1cc(C#N)cn1] | 0.73 |
| I-1-066 | H | H | H | H | 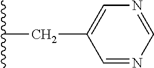 [SMILES: *Cc1cncnc1] | 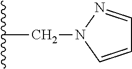 [SMILES: *Cn1cccn1] | 0.56 |
| I-1-067 | H | H | CN | H | 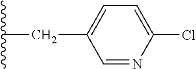 [SMILES: *Cc1ccc(Cl)nc1] | 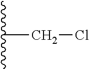 [SMILES: *CCl] | 1.83 |
| I-1-068 | H | H | H | H | 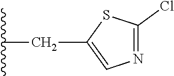 [SMILES: *Cc1cnc(Cl)s1] | 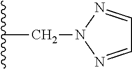 [SMILES: *Cn1nccn1] | 1.41 |
| I-1-069 | H | H | CF₃ | H | 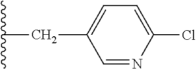 [SMILES: *Cc1ccc(Cl)nc1] | 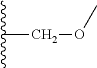 [SMILES: *COC] | 1.99 |
| I-1-070 | H | H | H | H | 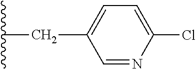 [SMILES: *Cc1ccc(Cl)nc1] | 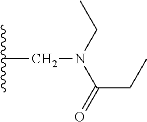 [SMILES: *CN(CC)C(=O)CC] | 1.64 |
| I-1-071 | H | H | H | H | 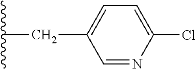 [SMILES: *Cc1ccc(Cl)nc1] | 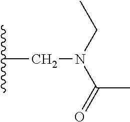 [SMILES: *CN(CC)C(C)=O] | 1.4 |
| I-1-072 | H | H | H | H | 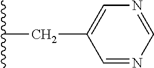 [SMILES: *Cc1cncnc1] | 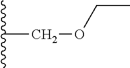 [SMILES: *COCC] | 0.59 |
| I-1-073 | H | H | H | H | 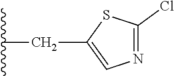 [SMILES: *Cc1cnc(Cl)s1] | 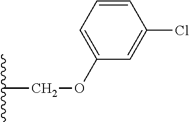 [SMILES: *COc1cccc(Cl)c1] | 2.87 |
| I-1-074 | H | H | H | H | 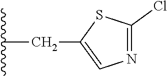 [SMILES: *Cc1cnc(Cl)s1] | 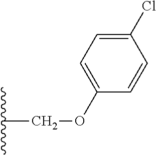 [SMILES: *COc1ccc(Cl)cc1] | 2.84 |
| I-1-075 | H | H | H | H | 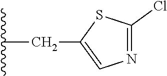 [SMILES: *Cc1cnc(Cl)s1] | 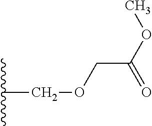 [SMILES: *COCC(=O)OC] | 1.41 |
| I-1-076 | H | H | H | H | 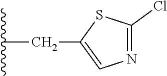 [SMILES: *Cc1cnc(Cl)s1] | 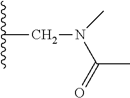 [SMILES: *CN(C)C(C)=O] | 1.18 |
| I-1-077 | H | H | H | H | 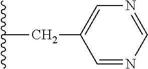 [SMILES: *Cc1cncnc1] | 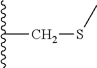 [SMILES: *CSC] | 0.79 |
| I-1-078 | H | H | H | H | 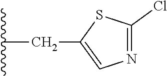 [SMILES: *Cc1cnc(Cl)s1] | 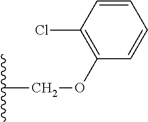 [SMILES: *COc1ccccc1Cl] | 2.77 |
| I-1-079 | H | H | CF₃ | H | 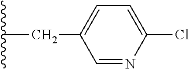 [SMILES: *Cc1ccc(Cl)nc1] | 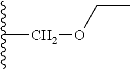 [SMILES: *COCC] | 2.17 |
| I-1-080 | CH₃ | H | H | H | 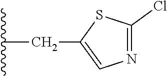 [SMILES: *Cc1cnc(Cl)s1] | 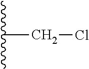 [SMILES: *CCl] | 1.81 |
| I-1-081 | H | H | H | CH₃ | 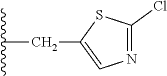 [SMILES: *Cc1cnc(Cl)s1] | 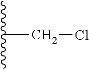 [SMILES: *CCl] | 1.98 |
| I-1-082 | H | H | H | CH₃ | 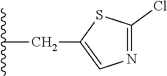 [SMILES: *Cc1cnc(Cl)s1] | 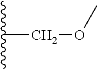 [SMILES: *COC] | 1.46 |
| I-1-083 | H | H | H | CH₃ | 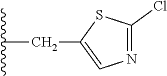 [SMILES: *Cc1cnc(Cl)s1] | 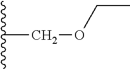 [SMILES: *COCC] | 1.72 |
| I-1-084 | H | H | H | H | 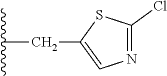 [SMILES: *Cc1cnc(Cl)s1] | 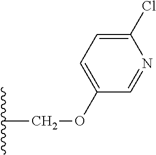 [SMILES: *COc1ccc(Cl)nc1] | 2.17 |
| I-1-085 | H | H | H | H | 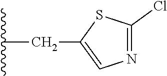 [SMILES: *Cc1cnc(Cl)s1] | 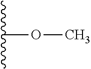 [SMILES: *OC] | 1.23 |
| I-1-086 | H | H | H | H | 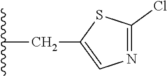 [SMILES: *Cc1cnc(Cl)s1] | 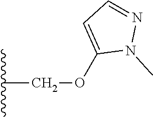 [SMILES: *COc1ccnn1C] | 1.53 |
| I-1-087 | H | H | H | H | 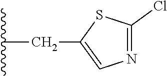 [SMILES: *Cc1cnc(Cl)s1] | 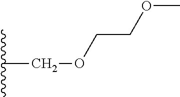 [SMILES: *COCCOC] | 1.38 |
| I-1-088 | H | H | H | H | 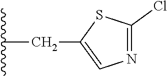 [SMILES: *Cc1cnc(Cl)s1] | 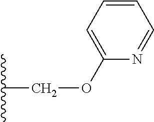 [SMILES: *COc1ccccn1] | 1.32 |
| I-1-089 | H | H | H | H | 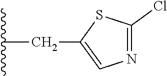 [SMILES: *Cc1cnc(Cl)s1] | 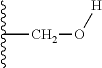 [SMILES: *CO] | 1.14 |
| I-1-090 | H | H | H | Cl | 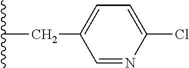 [SMILES: *Cc1ccc(Cl)nc1] | 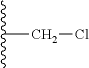 [SMILES: *CCl] | 1.99 |
| I-1-091 | H | CH3 | H | H | 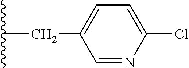 [SMILES: *Cc1ccc(Cl)nc1] | 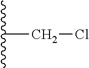 [SMILES: *CCl] | 1.87 |
| I-1-092 | H | H | H | CH₃ | 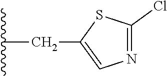 [SMILES: *Cc1cnc(Cl)s1] | 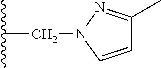 [SMILES: *Cn1ccc(C)n1] | 1.84 |
| I-1-093 | H | H | H | CH₃ | 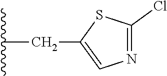 [SMILES: *Cc1cnc(Cl)s1] | 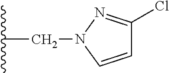 [SMILES: *Cn1ccc(Cl)n1] | 2.23 |
| I-1-094 | H | H | H | CH₃ | 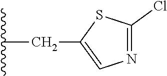 [SMILES: *Cc1cnc(Cl)s1] | 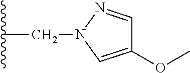 [SMILES: *Cn1cc(OC)cn1] | 1.75 |
| I-1-095 | H | H | H | CH₃ | 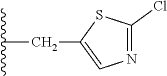 [SMILES: *Cc1cnc(Cl)s1] | 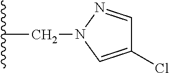 [SMILES: *Cn1cc(Cl)cn1] | 2.27 |
| I-1-096 | H | H | H | CH₃ | 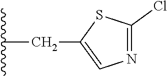 [SMILES: *Cc1cnc(Cl)s1] | 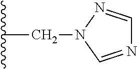 [SMILES: *Cn1cncn1] | 1.31 |
| I-1-097 | H | H | H | CH₃ | 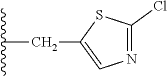 [SMILES: *Cc1cnc(Cl)s1] | 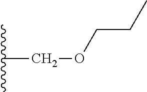 [SMILES: *COCCC] | 2.11 |
| I-1-098 | CH₃ | H | H | H | 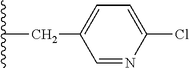 [SMILES: *Cc1ccc(Cl)nc1] | 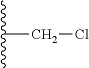 [SMILES: *CCl] | 1.74 |
| I-1-099 | H | F | H | H | 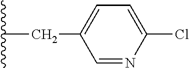 [SMILES: *Cc1ccc(Cl)nc1] | 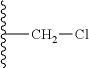 [SMILES: *CCl] | 1.78 |
| I-1-100 | H | H | H | F | 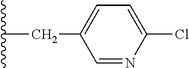 [SMILES: *Cc1ccc(Cl)nc1] | 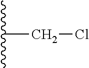 [SMILES: *CCl] | 1.81 |
| I-1-101 | H | H | H | H | 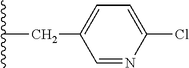 [SMILES: *Cc1ccc(Cl)nc1] | 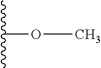 [SMILES: *OC] | 1.18 |
| I-1-102 | H | H | CF₃ | H | 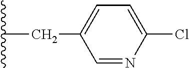 [SMILES: *Cc1ccc(Cl)nc1] | 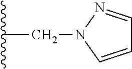 [SMILES: *Cn1cccn1] | 2.08 |
| I-1-103 | CH₃ | H | H | H | 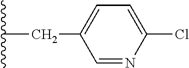 [SMILES: *Cc1ccc(Cl)nc1] | 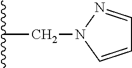 [SMILES: *Cn1cccn1] | 1.47 |
| I-1-104 | H | CH₃ | H | H | 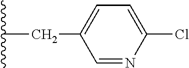 [SMILES: *Cc1ccc(Cl)nc1] | 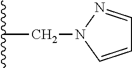 [SMILES: *Cn1cccn1] | 1.63 |
| I-1-105 | H | H | H | F | 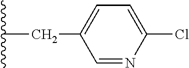 [SMILES: *Cc1ccc(Cl)nc1] | 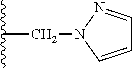 [SMILES: *Cn1cccn1] | 1.55 |
| I-1-106 | H | F | H | H | 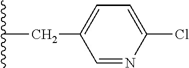 [SMILES: *Cc1ccc(Cl)nc1] | 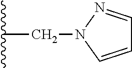 [SMILES: *Cn1cccn1] | 1.53 |
| I-1-107 | H | H | H | H | 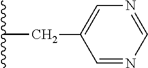 [SMILES: *Cc1cncnc1] | 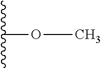 [SMILES: *OC] | 0.42 |
| I-1-108 | H | Cl | H | H | 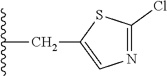 [SMILES: *Cc1cnc(Cl)s1] | 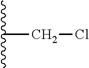 [SMILES: *CCl] | 2.2 |
| I-1-109 | H | H | H | H | 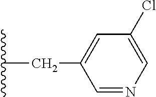 [SMILES: *Cc1cncc(Cl)c1] | 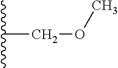 [SMILES: *COC] | 1.15 |
| I-1-110 | H | F | H | F | 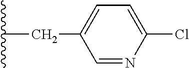 [SMILES: *Cc1ccc(Cl)nc1] | 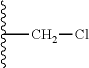 [SMILES: *CCl] | 1.96 |
| I-1-111 | H | H | H | H | 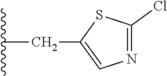 [SMILES: *Cc1cnc(Cl)s1] | 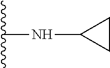 [SMILES: *NC1CC1] | 1.63 |
| I-1-112 | H | F | H | H | 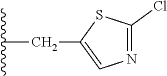 [SMILES: *Cc1cnc(Cl)s1] | 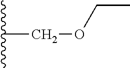 [SMILES: *COCC] | 1.58 |
| I-1-113 | H | CH₃ | H | H | 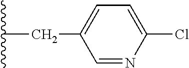 [SMILES: *Cc1ccc(Cl)nc1] | 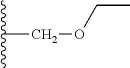 [SMILES: *COCC] | 1.66 |
| I-1-114 | CH₃ | H | H | H | 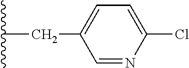 [SMILES: *Cc1ccc(Cl)nc1] | 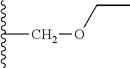 [SMILES: *COCC] | 1.47 |
| I-1-115 | H | H | H | 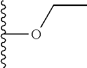 [SMILES: *OCC] | 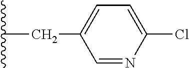 [SMILES: *Cc1ccc(Cl)nc1] | 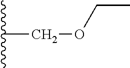 [SMILES: *COCC] | 1.9 |
| I-1-116 | H | H | H | Cl | 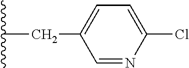 [SMILES: *Cc1ccc(Cl)nc1] | 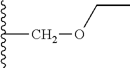 [SMILES: *COCC] | 1.78 |
| I-1-117 | H | Cl | H | H | 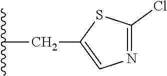 [SMILES: *Cc1cnc(Cl)s1] | 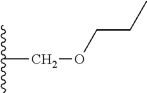 [SMILES: *COCCC] | 2.41 |
| I-1-118 | H | Cl | H | H | 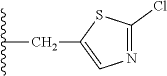 [SMILES: *Cc1cnc(Cl)s1] | 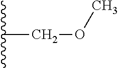 [SMILES: *COC] | 1.69 |
| I-1-119 | H | Cl | H | H | 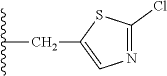 [SMILES: *Cc1cnc(Cl)s1] | 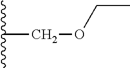 [SMILES: *COCC] | 1.96 |
| I-1-120 | H | H | Cl | H | 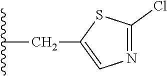 [SMILES: *Cc1cnc(Cl)s1] | 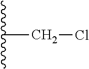 [SMILES: *CCl] | 2.17 |
| I-1-121 | Cl | H | H | H | 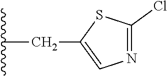 [SMILES: *Cc1cnc(Cl)s1] | 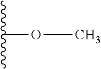 [SMILES: *OC] | 1.32 |
| I-1-122 | CH₃ | H | H | H | 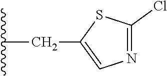 [SMILES: *Cc1cnc(Cl)s1] | 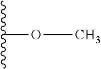 [SMILES: *OC] | 1.32 |
| I-1-123 | H | H | Cl | H | 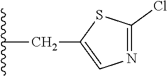 [SMILES: *Cc1cnc(Cl)s1] | 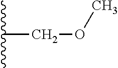 [SMILES: *COC] | 1.90 |
| I-1-124 | H | H | Cl | H | 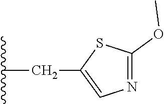 [SMILES: *Cc1cnc(OC)s1] | 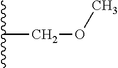 [SMILES: *COC] | 1.51 |
| I-1-125 | H | CH₃ | H | H | 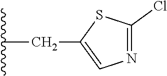 [SMILES: *Cc1cnc(Cl)s1] | 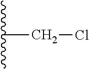 [SMILES: *CCl] | 1.99 |
| I-1-126 | H | H | CH₃ | H | 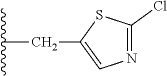 [SMILES: *Cc1cnc(Cl)s1] | 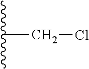 [SMILES: *CCl] | 1.96 |
| I-1-127 | H | H | H | F | 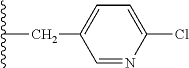 [SMILES: *Cc1ccc(Cl)nc1] | 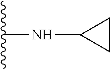 [SMILES: *NC1CC1] | 1.82 |
| I-1-128 | H | H | H | H | 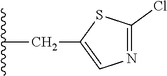 [SMILES: *Cc1cnc(Cl)s1] | 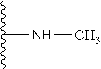 [SMILES: *NC] | 1.31 |
| I-1-129 | H | H | H | H | 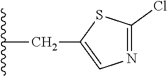 [SMILES: *Cc1cnc(Cl)s1] | 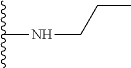 [SMILES: *NCCC] | 1.98 |
| I-1-130 | H | H | H | Cl | 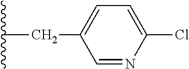 [SMILES: *Cc1ccc(Cl)nc1] | 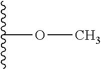 [SMILES: *OC] | 1.56 |
| I-1-131 | H | H | H | H | 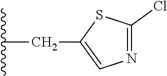 [SMILES: *Cc1cnc(Cl)s1] | 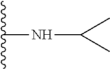 [SMILES: *NC(C)C] | 1.95 |
| I-1-132 | H | H | CF₃ | H | 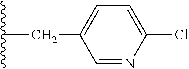 [SMILES: *Cc1ccc(Cl)nc1] | 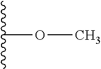 [SMILES: *OC] | 1.93 |
| I-1-133 | H | H | H | H | 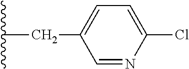 [SMILES: *Cc1ccc(Cl)nc1] | 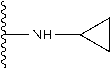 [SMILES: *NC1CC1] | 1.85 |
| I-1-134 | H | H | H | H | 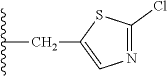 [SMILES: *Cc1cnc(Cl)s1] | 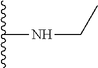 [SMILES: *NCC] | 1.58 |
| I-1-135 | H | H | H | H | 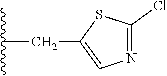 [SMILES: *Cc1cnc(Cl)s1] | 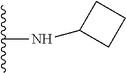 [SMILES: *NC1CCC1] | 2.11 |
| I-1-136 | H | H | H | H | 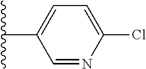 [SMILES: *c1ccc(Cl)nc1] | 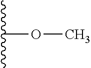 [SMILES: *OC] | 1.11 |
| I-1-137 | H | H | H | H | 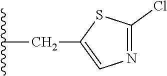 [SMILES: *Cc1cnc(Cl)s1] | 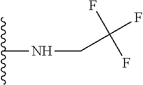 [SMILES: *NCC(F)(F)F] | 2.08 |
| I-1-138 | H | H | H | H | 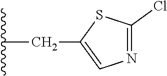 [SMILES: *Cc1cnc(Cl)s1] | 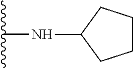 [SMILES: *NC1CCCC1] | 2.35 |
| I-1-139 | H | H | H | H | 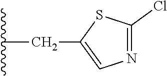 [SMILES: *Cc1cnc(Cl)s1] | 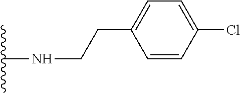 [SMILES: *NCCc1ccc(Cl)cc1] | 3 |
| I-1-140 | H | H | H | Cl | 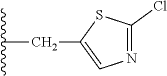 [SMILES: *Cc1cnc(Cl)s1] | 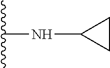 [SMILES: *NC1CC1] | 2.01 |
| I-1-141 | H | H | CF₃ | H | 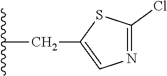 [SMILES: *Cc1cnc(Cl)s1] | 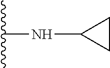 [SMILES: *NC1CC1] | 2.38 |
| I-1-142 | H | H | H | H | 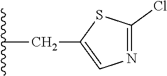 [SMILES: *Cc1cnc(Cl)s1] | 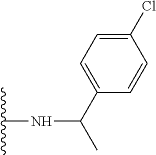 [SMILES: *NC(C)c1ccc(Cl)cc1] | 3.13 |
| I-1-143 | H | H | H | H | 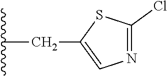 [SMILES: *Cc1cnc(Cl)s1] | 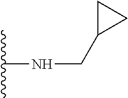 [SMILES: *NCC1CC1] | 1.99 |
| I-1-144 | H | H | H | H | 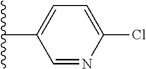 [SMILES: *c1ccc(Cl)nc1] | 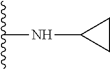 [SMILES: *NC1CC1] | 1.47 |
| I-1-145 | H | H | H | H | 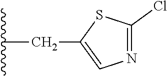 [SMILES: *Cc1cnc(Cl)s1] | 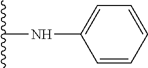 [SMILES: *Nc1ccccc1] | 2.73 |
| I-1-146 | H | H | H | H | 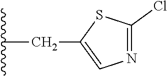 [SMILES: *Cc1cnc(Cl)s1] | 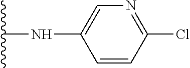 [SMILES: *Nc1ccc(Cl)nc1] | 2.55 |
| I-1-147 | H | H | H | H | 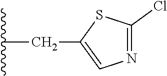 [SMILES: *Cc1cnc(Cl)s1] | 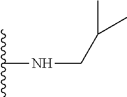 [SMILES: *NCC(C)C] | 2.29 |
| I-1-148 | H | H | H | H | 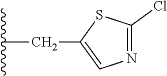 [SMILES: *Cc1cnc(Cl)s1] | 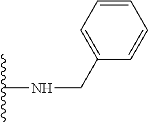 [SMILES: *NCc1ccccc1] | 2.41 |
| I-1-149 | H | H | H | H | 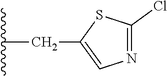 [SMILES: *Cc1cnc(Cl)s1] | 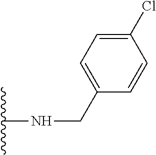 [SMILES: *NCc1ccc(Cl)cc1] | 2.86 |
| I-1-150 | H | H | H | H | 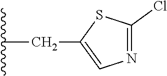 [SMILES: *Cc1cnc(Cl)s1] | 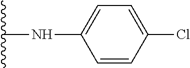 [SMILES: *Nc1ccc(Cl)cc1] | 3.39 |
| I-1-151 | H | H | H | H | 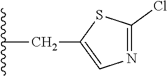 [SMILES: *Cc1cnc(Cl)s1] | 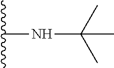 [SMILES: *NC(C)(C)C] | 2.39 |
| I-1-152 | H | H | H | H | 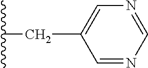 [SMILES: *Cc1cncnc1] | 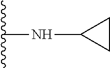 [SMILES: *NC1CC1] | 0.84 |
| I-1-153 | H | H | H | H | 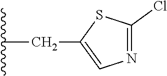 [SMILES: *Cc1cnc(Cl)s1] | 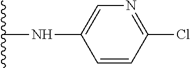 [SMILES: *Nc1ccc(Cl)nc1] | 3.06 |
| I-1-154 | H | H | H | H | 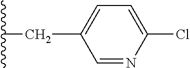 [SMILES: *Cc1ccc(Cl)nc1] | 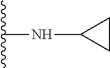 [SMILES: *NC1CC1] | 1.59 |
| I-1-155 | H | F | H | H | 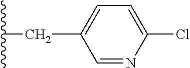 [SMILES: *Cc1ccc(Cl)nc1] | 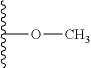 [SMILES: *OC] | 1.43 |
| I-1-156 | H | F | H | F | 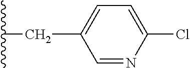 [SMILES: *Cc1ccc(Cl)nc1] | 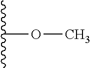 [SMILES: *OC] | 1.56 |
| I-1-157 | CH₃ | H | H | H | 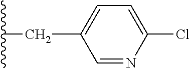 [SMILES: *Cc1ccc(Cl)nc1] | 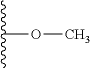 [SMILES: *OC] | 1.27 |
| I-1-158 | H | H | H | H | 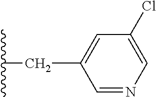 [SMILES: *Cc1cncc(Cl)c1] | 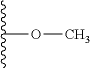 [SMILES: *OC] | 1.15 |
| I-1-159 | H | H | H | CH₃ | 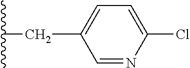 [SMILES: *Cc1ccc(Cl)nc1] | 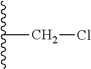 [SMILES: *CCl] | 1.78 |
| I-1-160 | H | H | H | CH₃ | 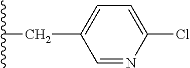 [SMILES: *Cc1ccc(Cl)nc1] | 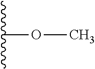 [SMILES: *OC] | 1.39 |
| I-1-161 | H | H | H | H | 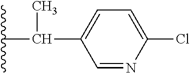 [SMILES: *C(C)c1ccc(Cl)nc1] | 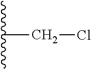 [SMILES: *CCl] | 1.75 |
| I-1-162 | H | H | H | H | 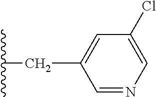 [SMILES: *Cc1cncc(Cl)c1] | 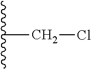 [SMILES: *CCl] | 1.53 |
| I-1-163 | H | H | H | H | 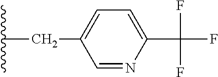 [SMILES: *Cc1ccc(C(F)(F)F)nc1] | 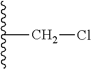 [SMILES: *CCl] | 1.9 |
| I-1-164 | H | H | H | Cl | 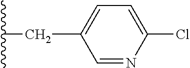 [SMILES: *Cc1ccc(Cl)nc1] | 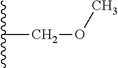 [SMILES: *COC] | 1.59 |
| I-1-165 | H | F | H | H | 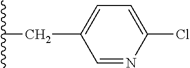 [SMILES: *Cc1ccc(Cl)nc1] | 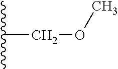 [SMILES: *COC] | 1.39 |
| I-1-166 | CH₃ | H | H | H | 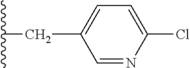 [SMILES: *Cc1ccc(Cl)nc1] | 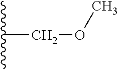 [SMILES: *COC] | 1.33 |
| I-1-167 | H | CH₃ | H | H | 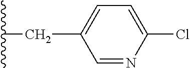 [SMILES: *Cc1ccc(Cl)nc1] | 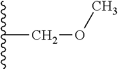 [SMILES: *COC] | 1.48 |
| I-1-168 | H | H | H | H | 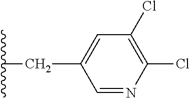 [SMILES: *Cc1cnc(Cl)c(Cl)c1] | 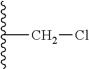 [SMILES: *CCl] | 2.11 |
| I-1-169 | H | H | H | H | 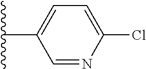 [SMILES: *c1ccc(Cl)nc1] | 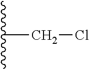 [SMILES: *CCl] | 1.54 |
| I-1-170 | H | F | H | H | 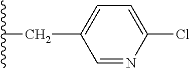 [SMILES: *Cc1ccc(Cl)nc1] | 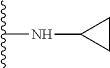 [SMILES: *NC1CC1] | 1.79 |
| I-1-171 | CH₃ | H | H | H | 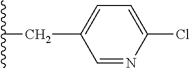 [SMILES: *Cc1ccc(Cl)nc1] | 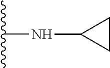 [SMILES: *NC1CC1] | 1.05 |
| I-1-172 | H | H | H | CN | 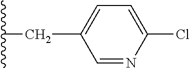 [SMILES: *Cc1ccc(Cl)nc1] | 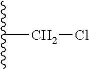 [SMILES: *CCl] | 1.78 |
| I-1-173 | H | CH₃ | H | H | 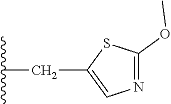 [SMILES: *Cc1cnc(OC)s1] | 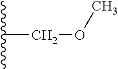 [SMILES: *COC] | 1.35 |
| I-1-174 | H | CH₃ | H | H | 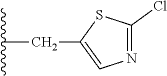 [SMILES: *Cc1cnc(Cl)s1] | 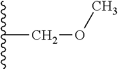 [SMILES: *COC] | 1.52 |
| I-1-175 | H | CH₃ | H | H | 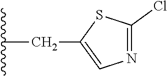 [SMILES: *Cc1cnc(Cl)s1] | 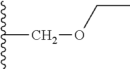 [SMILES: *COCC] | 1.78 |
| I-1-176 | H | H | H | CH₃ | 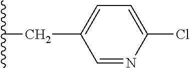 [SMILES: *Cc1ccc(Cl)nc1] | 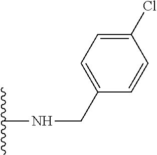 [SMILES: *NCc1ccc(Cl)cc1] | 2.92 |
| I-1-177 | H | H | H | H | 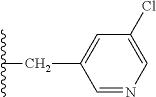 [SMILES: *Cc1cncc(Cl)c1] | 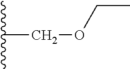 [SMILES: *COCC] | 1.37 |
| I-1-178 | H | H | H | H | 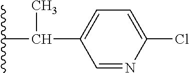 [SMILES: *C(C)c1ccc(Cl)nc1] | 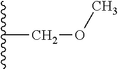 [SMILES: *COC] | 1.35 |
| I-1-179 | H | H | H | H | 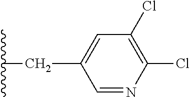 [SMILES: *Cc1cnc(Cl)c(Cl)c1] | 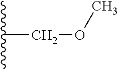 [SMILES: *COC] | 1.66 |
| I-1-180 | H | H | H | H | 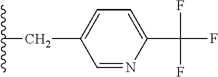 [SMILES: *Cc1ccc(C(F)(F)F)nc1] | 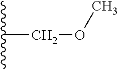 [SMILES: *COC] | 1.49 |
| 1-1-181 | H | H | H | CH₃ | 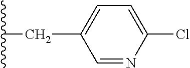 [SMILES: *Cc1ccc(Cl)nc1] | 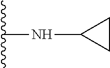 [SMILES: *NC1CC1] | 1.75 |
| I-1-182 | H | H | H | CH₃ | 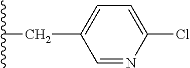 [SMILES: *Cc1ccc(Cl)nc1] | 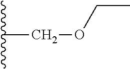 [SMILES: *COCC] | 1.6 |
| I-1-183 | H | H | H | CH₃ | 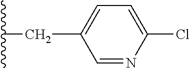 [SMILES: *Cc1ccc(Cl)nc1] | 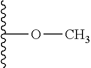 [SMILES: *OC] | 1.66 |
| I-1-184 | H | H | Cl | H | 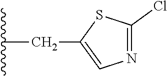 [SMILES: *Cc1cnc(Cl)s1] | 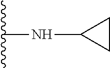 [SMILES: *NC1CC1] | 2.14 |
| I-1-185 | H | H | H | H | 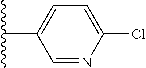 [SMILES: *c1ccc(Cl)nc1] | 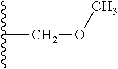 [SMILES: *COC] | 1.17 |
| I-1-186 | H | H | H | H | 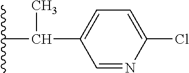 [SMILES: *C(C)c1ccc(Cl)nc1] | 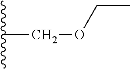 [SMILES: *COCC] | 1.58 |
| I-1-187 | H | H | H | CH₃ | 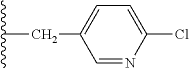 [SMILES: *Cc1ccc(Cl)nc1] | 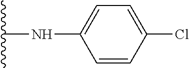 [SMILES: *Nc1ccc(Cl)cc1] | 3.41 |
| I-1-188 | H | H | H | H | 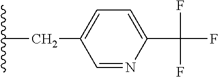 [SMILES: *Cc1ccc(C(F)(F)F)nc1] | 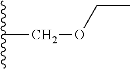 [SMILES: *COCC] | 1.72 |
| I-1-189 | H | H | H | OCF₃ | 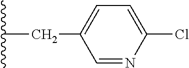 [SMILES: *Cc1ccc(Cl)nc1] | 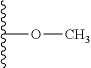 [SMILES: *OC] | 1.84 |
| I-1-190 | CH₃ | H | H | H | 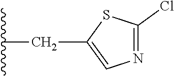 [SMILES: *Cc1cnc(Cl)s1] | 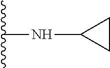 [SMILES: *NC1CC1] | 1.16 |
| I-1-191 | H | H | H | CH₃ | 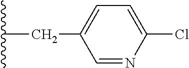 [SMILES: *Cc1ccc(Cl)nc1] | 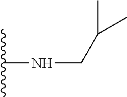 [SMILES: *NCC(C)C] | 2.33 |
| I-1-192 | H | H | H | H | 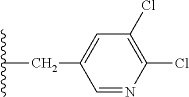 [SMILES: *Cc1cnc(Cl)c(Cl)c1] | 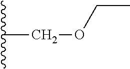 [SMILES: *COCC] | 1.93 |
| I-1-193 | H | H | H | CH₃ | 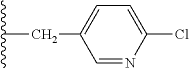 [SMILES: *Cc1ccc(Cl)nc1] | 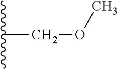 [SMILES: *COC] | 1.38 |
| I-1-194 | H | H | H | SCH₃ | 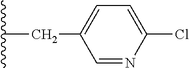 [SMILES: *Cc1ccc(Cl)nc1] | 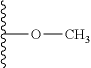 [SMILES: *OC] | 1.64 |
| I-1-195 | H | H | H | H | 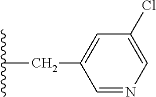 [SMILES: *Cc1cncc(Cl)c1] | 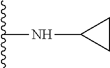 [SMILES: *NC1CC1] | 1.52 |
| I-1-196 | H | H | H | OCF₃ | 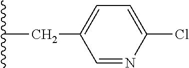 [SMILES: *Cc1ccc(Cl)nc1] | 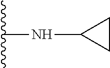 [SMILES: *NC1CC1] | 2.33 |
| I-1-197 | H | F | H | F | 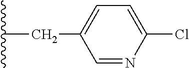 [SMILES: *Cc1ccc(Cl)nc1] | 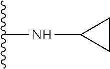 [SMILES: *NC1CC1] | 1.99 |
| I-1-198 | H | H | H | H | 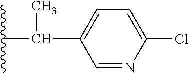 [SMILES: *C(C)c1ccc(Cl)nc1] | 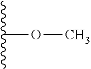 [SMILES: *OC] | 1.32 |
| I-1-199 | H | H | H | H | 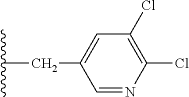 [SMILES: *Cc1cnc(Cl)c(Cl)c1] | 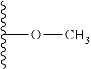 [SMILES: *OC] | 1.66 |
| I-1-200 | H | H | H | H | 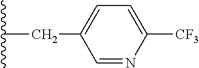 [SMILES: *Cc1ccc(C(F)(F)F)nc1] | 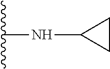 [SMILES: *NC1CC1] | 1.87 |
| I-1-201 | H | H | H | H | 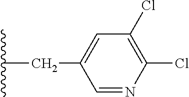 [SMILES: *Cc1cnc(Cl)c(Cl)c1] | 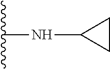 [SMILES: *NC1CC1] | 2.08 |
| I-1-202 | H | H | H | H | 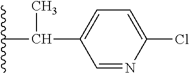 [SMILES: *C(C)c1ccc(Cl)nc1] | 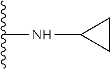 [SMILES: *NC1CC1] | 1.75 |
| I-1-203 | H | H | H | CH₃ | 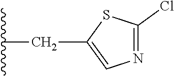 [SMILES: *Cc1cnc(Cl)s1] | 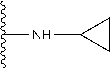 [SMILES: *NC1CC1] | 1.93 |
| I-1-204 | H | H | H | SCH₃ | 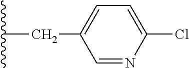 [SMILES: *Cc1ccc(Cl)nc1] | 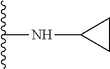 [SMILES: *NC1CC1] | 2.05 |
| I-1-205 | H | H | H | F | 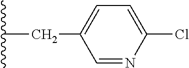 [SMILES: *Cc1ccc(Cl)nc1] | 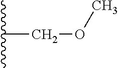 [SMILES: *COC] | 1.44 |
| I-1-206 | H | H | H | CH₃ | 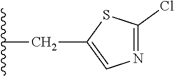 [SMILES: *Cc1cnc(Cl)s1] | 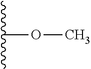 [SMILES: *OC] | 1.46 |
| I-1-207 | H | H | H | H | 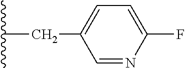 [SMILES: *Cc1ccc(F)nc1] | 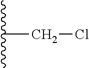 [SMILES: *CCl] | 1.38 |
| I-1-208 | H | H | H | CH₃ | 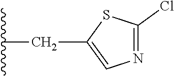 [SMILES: *Cc1cnc(Cl)s1] | 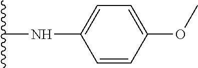 [SMILES: *Nc1ccc(OC)cc1] | 2.91 |
| I-1-209 | H | H | H | H | 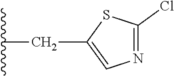 [SMILES: *Cc1cnc(Cl)s1] | 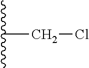 [SMILES: *CCl] | 1.24 |
| I-1-210 | H | H | H | CH₃ | 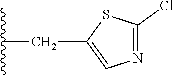 [SMILES: *Cc1cnc(Cl)s1] | 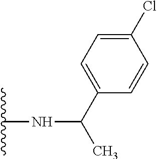 [SMILES: *NC(C)c1ccc(Cl)cc1] | 3.46 |
| I-1-211 | H | H | H | CH₃ | 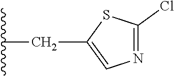 [SMILES: *Cc1cnc(Cl)s1] | 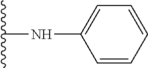 [SMILES: *Nc1ccccc1] | 3.1 |
| I-1-212 | H | H | H | CH₃ | 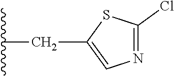 [SMILES: *Cc1cnc(Cl)s1] | 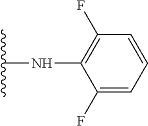 [SMILES: *Nc1c(F)cccc1F] | 2.73 |
| I-1-213 | H | H | H | CH₃ | 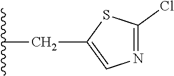 [SMILES: *Cc1cnc(Cl)s1] | 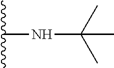 [SMILES: *NC(C)(C)C] | 2.69 |
| I-1-214 | H | H | H | CH₃ | 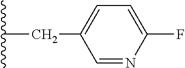 [SMILES: *Cc1ccc(F)nc1] | 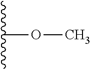 [SMILES: *OC] | 1.21 |
| I-1-215 | H | H | H | H | 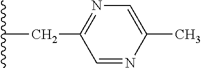 [SMILES: *Cc1cnc(C)cn1] | 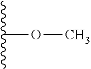 [SMILES: *OC] | 0.80 |
| I-1-216 | H | H | H | CH₃ | 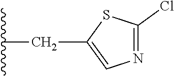 [SMILES: *Cc1cnc(Cl)s1] | 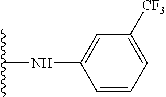 [SMILES: *Nc1cccc(C(F)(F)F)c1] | 4.06 |
| I-1-217 | H | H | H | H | 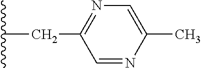 [SMILES: *Cc1cnc(C)cn1] | 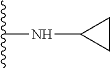 [SMILES: *NC1CC1] | 1.13 |
| I-1-218 | H | H | H | H | 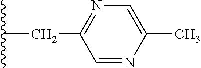 [SMILES: *Cc1cnc(C)cn1] | 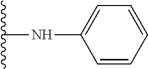 [SMILES: *Nc1ccccc1] | 2.05 |
| I-1-219 | H | H | H | CH₃ | 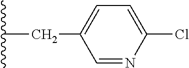 [SMILES: *Cc1ccc(Cl)nc1] | 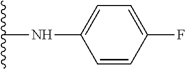 [SMILES: *Nc1ccc(F)cc1] | 2.9 |
| I-1-220 | H | H | H | CH₃ | 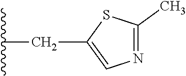 [SMILES: *Cc1cnc(C)s1] | 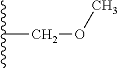 [SMILES: *COC] | 1.13 |
| I-1-221 | H | H | H | H | 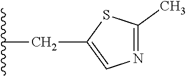 [SMILES: *Cc1cnc(C)s1] | 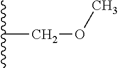 [SMILES: *COC] | 0.9 |
| I-1-222 | H | H | H | CH₃ | 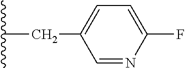 [SMILES: *Cc1ccc(F)nc1] | 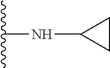 [SMILES: *NC1CC1] | 1.58 |
| I-1-223 | H | H | H | CH₃ | 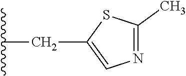 [SMILES: *Cc1cnc(C)s1] | 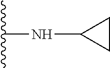 [SMILES: *NC1CC1] | 1.45 |
| I-1-224 | H | H | H | H | 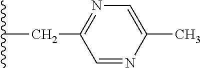 [SMILES: *Cc1cnc(C)cn1] | 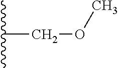 [SMILES: *COC] | 0.83 |
| I-1-225 | H | H | H | CH₃ | 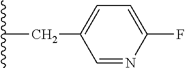 [SMILES: *Cc1ccc(F)nc1] | 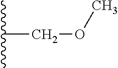 [SMILES: *COC] | 1.24 |
| I-1-226 | H | H | H | H | 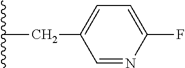 [SMILES: *Cc1ccc(F)nc1] | 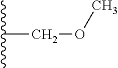 [SMILES: *COC] | 0.97 |
| I-1-227 | H | H | H | H | 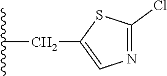 [SMILES: *Cc1cnc(Cl)s1] | 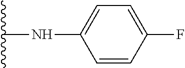 [SMILES: *Nc1ccc(F)cc1] | 2.82 |
| I-1-228 | H | H | H | H | 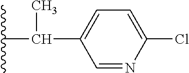 [SMILES: *C(C)c1ccc(Cl)nc1] | 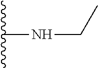 [SMILES: *NCC] | 1.72 |
| I-1-229 | H | H | H | H | 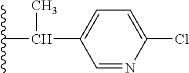 [SMILES: *C(C)c1ccc(Cl)nc1] | 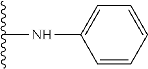 [SMILES: *Nc1ccccc1] | 2.8 |
| I-1-230 | H | H | H | H | 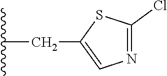 [SMILES: *Cc1cnc(Cl)s1] | 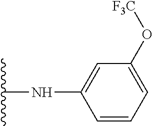 [SMILES: *Nc1cccc(OC(F)(F)F)c1] | 3.87 |
| I-1-231 | H | H | H | H | 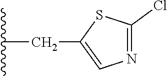 [SMILES: *Cc1cnc(Cl)s1] | 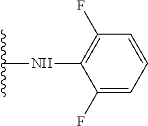 [SMILES: *Nc1c(F)cccc1F] | 2.43 |
| I-1-232 | H | H | H | H | 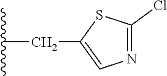 [SMILES: *Cc1cnc(Cl)s1] | 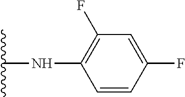 [SMILES: *Nc1ccc(F)cc1F] | 3.13 |
| I-1-233 | H | H | H | H | 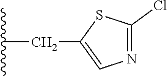 [SMILES: *Cc1cnc(Cl)s1] | 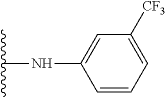 [SMILES: *Nc1cccc(C(F)(F)F)c1] | 3.76 |
| I-1-234 | H | H | H | H | 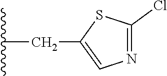 [SMILES: *Cc1cnc(Cl)s1] | 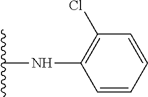 [SMILES: *Nc1ccccc1Cl] | 3.29 |
| I-1-235 | H | H | H | H | 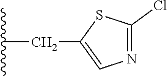 [SMILES: *Cc1cnc(Cl)s1] | 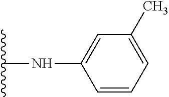 [SMILES: *Nc1cccc(C)c1] | 3.21 |
| I-1-236 | H | H | H | H | 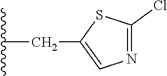 [SMILES: *Cc1cnc(Cl)s1] | 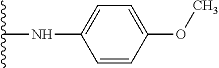 [SMILES: *Nc1ccc(OC)cc1] | 2.62 |
| I-1-237 | H | H | H | H | 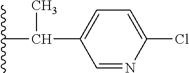 [SMILES: *C(C)c1ccc(Cl)nc1] | 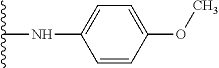 [SMILES: *Nc1ccc(OC)cc1] | 2.66 |
| I-1-238 | H | H | H | H | 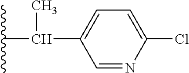 [SMILES: *C(C)c1ccc(Cl)nc1] | 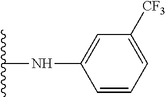 [SMILES: *Nc1cccc(C(F)(F)F)c1] | 3.72 |
| I-1-239 | H | H | H | H | 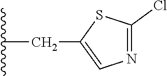 [SMILES: *Cc1cnc(Cl)s1] | 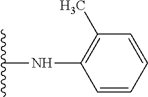 [SMILES: *Nc1ccccc1C] | 3.03 |
| I-1-240 | H | H | H | OCH₃ | 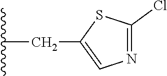 [SMILES: *Cc1cnc(Cl)s1] | 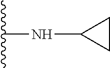 [SMILES: *NC1CC1] | 1.96 |
| I-1-241 | H | H | H | H | 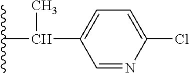 [SMILES: *C(C)c1ccc(Cl)nc1] | 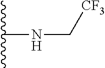 [SMILES: *NCC(F)(F)F] | 2.2 |
| I-1-242 | H | H | H | H | 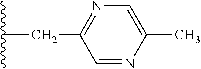 [SMILES: *Cc1cnc(C)cn1] | 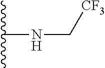 [SMILES: *NCC(F)(F)F] | 1.57 |
| I-1-243 | H | H | H | H | 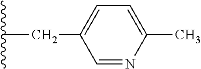 [SMILES: *Cc1ccc(C)nc1] | 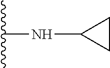 [SMILES: *NC1CC1] | 0.81 |
| I-1-244 | H | H | H | H | 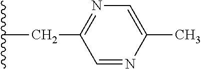 [SMILES: *Cc1cnc(C)cn1] | 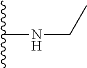 [SMILES: *NCC] | 1.07 |
| I-1-245 | H | H | H | H | 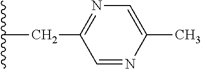 [SMILES: *Cc1cnc(C)cn1] | 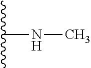 [SMILES: *NC] | 0.76 |
| I-1-246 | H | H | H | H | 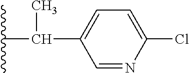 [SMILES: *C(C)c1ccc(Cl)nc1] | 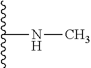 [SMILES: *NC] | 1.41 |
| I-1-247 | H | H | H | H | 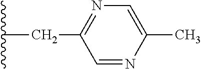 [SMILES: *Cc1cnc(C)cn1] | 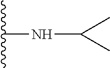 [SMILES: *NC(C)C] | 1.38 |
| I-1-248 | H | H | H | CH₃ | 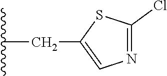 [SMILES: *Cc1cnc(Cl)s1] | 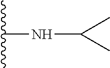 [SMILES: *NC(C)C] | 2.2 |
| I-1-249 | H | H | H | H | 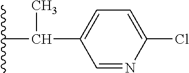 [SMILES: *C(C)c1ccc(Cl)nc1] | 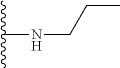 [SMILES: *NCCC] | 2.02 |
| I-1-250 | H | H | H | H | 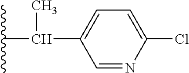 [SMILES: *C(C)c1ccc(Cl)nc1] | 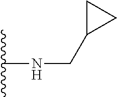 [SMILES: *NCC1CC1] | 2.12 |
| I-1-251 | H | H | H | CH₃ | 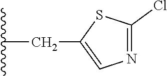 [SMILES: *Cc1cnc(Cl)s1] | 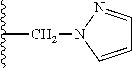 [SMILES: *Cn1cccn1] | 1.69 |
| I-1-252 | H | H | H | CH₃ | 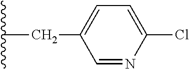 [SMILES: *Cc1ccc(Cl)nc1] | 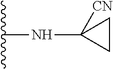 [SMILES: *NC1(C#N)CC1] | 1.78 |
| I-1-253 | H | H | H | CH₃ | 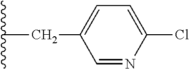 [SMILES: *Cc1ccc(Cl)nc1] | 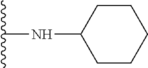 [SMILES: *NC1CCCCC1] | 2.72 |
| I-1-254 | H | H | H | H | 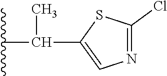 [SMILES: *C(C)c1cnc(Cl)s1] | 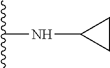 [SMILES: *NC1CC1] | 1.94 |
| I-1-255 | H | H | H | CH3 | 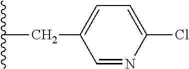 [SMILES: *Cc1ccc(Cl)nc1] | 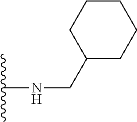 [SMILES: *NCC1CCCCC1] | 3.21 |
| I-1-256 | H | H | H | CH₃ | 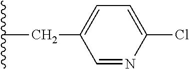 [SMILES: *Cc1ccc(Cl)nc1] | 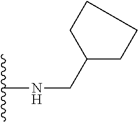 [SMILES: *NCC1CCCC1] | 2.8 |
| I-1-257 | H | H | H | CH₃ | 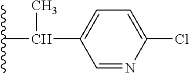 [SMILES: *C(C)c1ccc(Cl)nc1] | 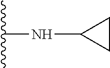 [SMILES: *NC1CC1] | 1.52 |
| I-1-258 | H | H | H | CH₃ | 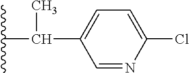 [SMILES: *C(C)c1ccc(Cl)nc1] | 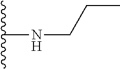 [SMILES: *NCCC] | 2.23 |
| I-1-259 | H | H | H | CH₃ | 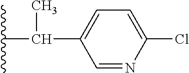 [SMILES: *C(C)c1ccc(Cl)nc1] | 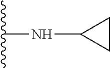 [SMILES: *NC1CC1] | 1.96 |
| I-1-262 | H | H | H | H | 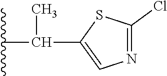 [SMILES: *C(C)c1cnc(Cl)s1] | 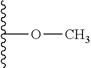 [SMILES: *OC] | 1.45 |
| I-1-263 | H | H | H | CH₃ | 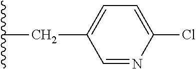 [SMILES: *Cc1ccc(Cl)nc1] | 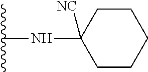 [SMILES: *NC1(C#N)CCCCC1] | 2.62 |
| I-1-264 | H | H | H | CH₃ | 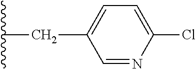 [SMILES: *Cc1ccc(Cl)nc1] | 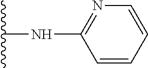 [SMILES: *Nc1ccccn1] | 1.68 |
| I-1-265 | H | H | H | CH₃ | 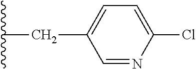 [SMILES: *Cc1ccc(Cl)nc1] | 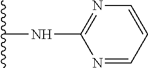 [SMILES: *Nc1ncccn1] | 1.51 |
| I-1-267 | H | H | H | CH₃ | 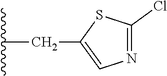 [SMILES: *Cc1cnc(Cl)s1] | 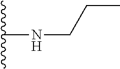 [SMILES: *NCCC] | 2.26 |
| I-1-269 | H | H | H | H | 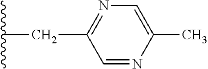 [SMILES: *Cc1cnc(C)cn1] | 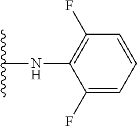 [SMILES: *Nc1c(F)cccc1F] | 1.75 |
| I-1-270 | H | H | H | H | 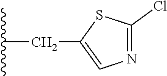 [SMILES: *Cc1cnc(Cl)s1] | 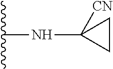 [SMILES: *NC1(C#N)CC1] | 1.58 |
| I-1-271 | H | H | H | H | 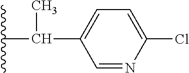 [SMILES: *C(C)c1ccc(Cl)nc1] | 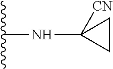 [SMILES: *NC1(C#N)CC1] | 1.69 |
| I-1-272 | H | H | H | H | 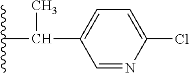 [SMILES: *C(C)c1ccc(Cl)nc1] | 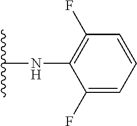 [SMILES: *Nc1c(F)cccc1F] | 2.46 |
| I-1-273 | H | H | H | H | 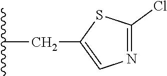 [SMILES: *Cc1cnc(Cl)s1] | 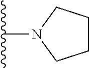 [SMILES: *N1CCCC1] | 1.52 |
| I-1-274 | H | H | H | CH₃ | 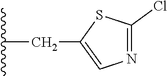 [SMILES: *Cc1cnc(Cl)s1] | 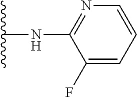 [SMILES: *Nc1ncccc1F] | 1.78 |
| I-1-275 | H | H | H | CH₃ | 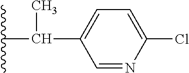 [SMILES: *C(C)c1ccc(Cl)nc1] | 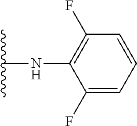 [SMILES: *Nc1c(F)cccc1F] | 2.67 |
| I-1-276 | H | H | H | CH₃ | 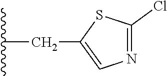 [SMILES: *Cc1cnc(Cl)s1] | 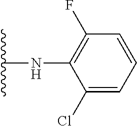 [SMILES: *Nc1c(F)cccc1Cl] | 2.93 |
| I-1-277 | H | H | H | CH₃ | 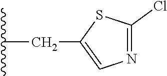 [SMILES: *Cc1cnc(Cl)s1] | 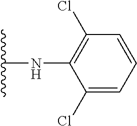 [SMILES: *Nc1c(Cl)cccc1Cl] | 3.11 |
| I-1-278 | H | H | H | H | 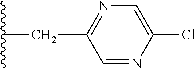 [SMILES: *Cc1cnc(Cl)cn1] | 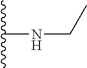 [SMILES: *NCC] | 1.51 |
| I-1-279 | H | H | H | H | 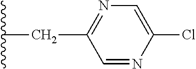 [SMILES: *Cc1cnc(Cl)cn1] | 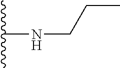 [SMILES: *NCCC] | 1.81 |
| I-1-280 | H | H | H | H | 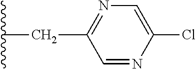 [SMILES: *Cc1cnc(Cl)cn1] | 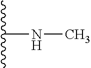 [SMILES: *NC] | 1.15 |
| I-1-281 | H | H | H | CH₃ | 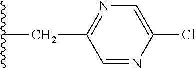 [SMILES: *Cc1cnc(Cl)cn1] | 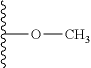 [SMILES: *OC] | 1.47 |
| I-1-282 | H | H | H | H | 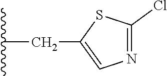 [SMILES: *Cc1cnc(Cl)s1] | 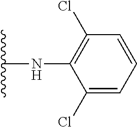 [SMILES: *Nc1c(Cl)cccc1Cl] | 2.8 |
| I-1-283 | H | H | H | H | 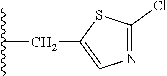 [SMILES: *Cc1cnc(Cl)s1] | 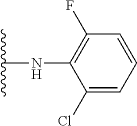 [SMILES: *Nc1c(F)cccc1Cl] | 2.57 |
| I-1-284 | H | H | H | H | 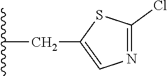 [SMILES: *Cc1cnc(Cl)s1] | 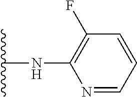 [SMILES: *Nc1ncccc1F] | 1.68 |
| I-1-285 | H | H | H | H | 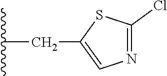 [SMILES: *Cc1cnc(Cl)s1] | 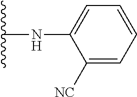 [SMILES: *Nc1ccccc1C#N] | 2.71 |
| I-1-286 | H | H | H | H | 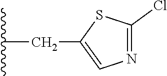 [SMILES: *Cc1cnc(Cl)s1] | 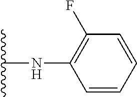 [SMILES: *Nc1ccccc1F] | 2.95 |
| I-1-287 | H | H | H | CH₃ | 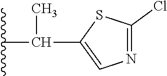 [SMILES: *C(C)c1cnc(Cl)s1] | 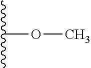 [SMILES: *OC] | 1.68 |
| I-1-288 | H | H | H | CH₃ | 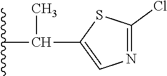 [SMILES: *C(C)c1cnc(Cl)s1] | 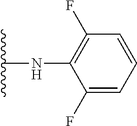 [SMILES: *Nc1c(F)cccc1F] | 2.76 |
| I-1-289 | H | H | H | H | 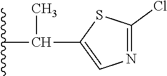 [SMILES: *C(C)c1cnc(Cl)s1] | 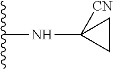 [SMILES: *NC1(C#N)CC1] | 1.86 |
| I-1-290 | H | H | H | CH₃ | 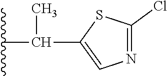 [SMILES: *C(C)c1cnc(Cl)s1] | 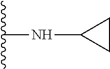 [SMILES: *NC1CC1] | 2.2 |
| I-1-291 | H | H | H | CH₃ | 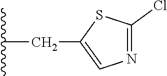 [SMILES: *Cc1cnc(Cl)s1] | 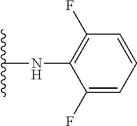 [SMILES: *Nc1c(F)cccc1F] | 3.07 |
| I-1-292 | H | H | H | CH₃ | 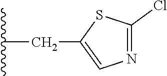 [SMILES: *Cc1cnc(Cl)s1] | 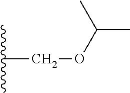 [SMILES: *COC(C)C] | 1.97 |
| I-1-293 | H | H | H | CH₃ | 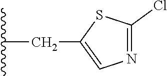 [SMILES: *Cc1cnc(Cl)s1] | 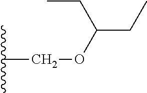 [SMILES: *COC(CC)CC] | 2.76 |
| I-1-294 | H | H | H | CH₃ | 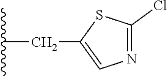 [SMILES: *Cc1cnc(Cl)s1] | 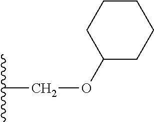 [SMILES: *COC1CCCCC1] | 2.77 |
| I-1-295 | H | H | H | H | 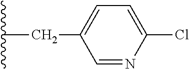 [SMILES: *Cc1ccc(Cl)nc1] | 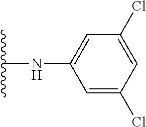 [SMILES: *Nc1cc(Cl)cc(Cl)c1] | 4.1 |
| I-1-296 | H | H | H | CH₃ | 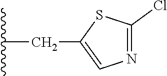 [SMILES: *Cc1cnc(Cl)s1] | 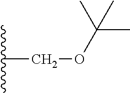 [SMILES: *COC(C)(C)C] | 2.12 |
| I-1-297 | H | H | H | CH₃ | 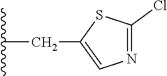 [SMILES: *Cc1cnc(Cl)s1] | 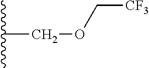 [SMILES: *COCC(F)(F)F] | 2.34 |
| I-1-298 | H | H | H | H | 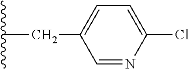 [SMILES: *Cc1ccc(Cl)nc1] | 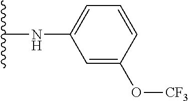 [SMILES: *Nc1cccc(OC(F)(F)F)c1] | 3.55 |
| I-1-299 | H | H | H | H | 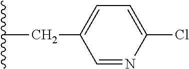 [SMILES: *Cc1ccc(Cl)nc1] | 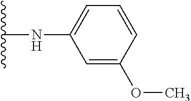 [SMILES: *Nc1cccc(OC)c1] | 2.55 |
| I-1-300 | H | H | H | H | 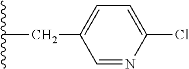 [SMILES: *Cc1ccc(Cl)nc1] | 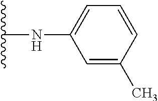 [SMILES: *Nc1cccc(C)c1] | 1.41 |
| I-1-301 | H | H | H | H | 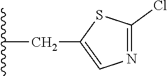 [SMILES: *Cc1cnc(Cl)s1] | 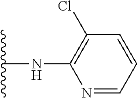 [SMILES: *Nc1ncccc1Cl] | 1.63 |
| I-1-302 | H | H | H | CH₃ | 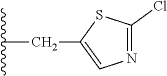 [SMILES: *Cc1cnc(Cl)s1] | 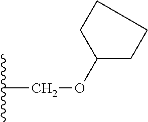 [SMILES: *COC1CCCC1] | 2.45 |
| I-1-303 | H | H | H | H | 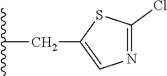 [SMILES: *Cc1cnc(Cl)s1] | 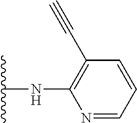 [SMILES: *Nc1ncccc1C#C] | 1.3 |
| I-1-304 | H | H | H | H | 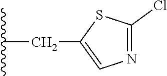 [SMILES: *Cc1cnc(Cl)s1] | 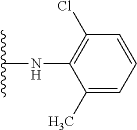 [SMILES: *Nc1c(C)cccc1Cl] | 2.8 |
| I-1-305 | H | H | H | H | 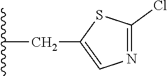 [SMILES: *Cc1cnc(Cl)s1] | 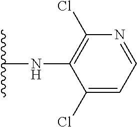 [SMILES: *Nc1c(Cl)ccnc1Cl] | 2.14 |
| I-1-306 | H | H | H | H | 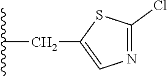 [SMILES: *Cc1cnc(Cl)s1] | 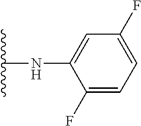 [SMILES: *Nc1cc(F)ccc1F] | 3.23 |
| I-1-307 | H | H | H | H | 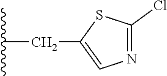 [SMILES: *Cc1cnc(Cl)s1] | 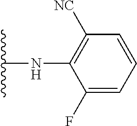 [SMILES: *Nc1c(F)cccc1C#N] | 2.2 |
| I-1-308 | H | H | H | H | 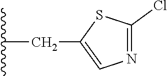 [SMILES: *Cc1cnc(Cl)s1] | 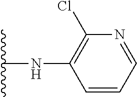 [SMILES: *Nc1cccnc1Cl] | 2.4 |
| I-1-309 | H | H | H | H | 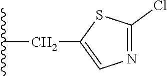 [SMILES: *Cc1cnc(Cl)s1] | 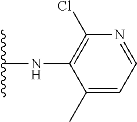 [SMILES: *Nc1c(C)ccnc1Cl] | 2.03 |
| I-1-310 | H | H | H | H | 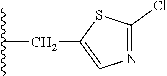 [SMILES: *Cc1cnc(Cl)s1] | 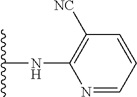 [SMILES: *Nc1ncccc1C#N] | 1.75 |
| I-1-311 | H | H | H | H | 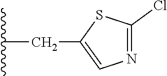 [SMILES: *Cc1cnc(Cl)s1] | 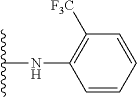 [SMILES: *Nc1ccccc1C(F)(F)F] | 3.22 |
| I-1-312 | H | H | H | H | 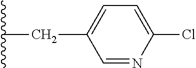 [SMILES: *Cc1ccc(Cl)nc1] | 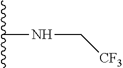 [SMILES: *NCC(F)(F)F] | 1.98 |
| I-1-313 | H | H | H | H | 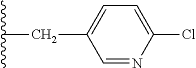 [SMILES: *Cc1ccc(Cl)nc1] | 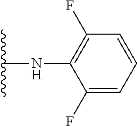 [SMILES: *Nc1c(F)cccc1F] | 2.27 |
| I-1-314 | H | H | H | H | 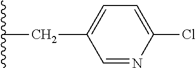 [SMILES: *Cc1ccc(Cl)nc1] | 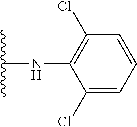 [SMILES: *Nc1c(Cl)cccc1Cl] | 2.59 |
| I-1-315 | H | H | H | H | 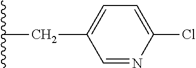 [SMILES: *Cc1ccc(Cl)nc1] | 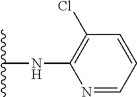 [SMILES: *Nc1ncccc1Cl] | 1.46 |
| I-1-316 | H | H | H | H | 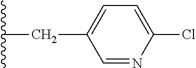 [SMILES: *Cc1ccc(Cl)nc1] | 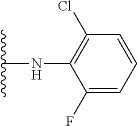 [SMILES: *Nc1c(F)cccc1Cl] | 2.44 |
| I-1-317 | H | H | H | H | 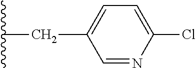 [SMILES: *Cc1ccc(Cl)nc1] | 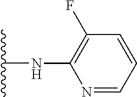 [SMILES: *Nc1ncccc1F] | 1.49 |
| I-1-318 | H | H | H | H | 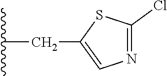 [SMILES: *Cc1cnc(Cl)s1] | 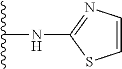 [SMILES: *Nc1nccs1] | 2.05 |
| I-1-319 | H | H | H | H | 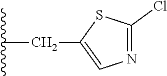 [SMILES: *Cc1cnc(Cl)s1] | 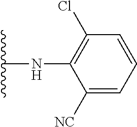 [SMILES: *Nc1c(Cl)cccc1C#N] | 2.43 |
| I-1-320 | H | H | H | H | 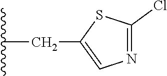 [SMILES: *Cc1cnc(Cl)s1] | 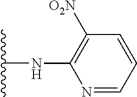 [SMILES: *Nc1ncccc1[N+](=O)[O-]] | 1.91 |
| I-1-321 | H | H | H | H | 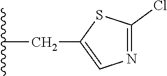 [SMILES: *Cc1cnc(Cl)s1] | 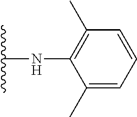 [SMILES: *Nc1c(C)cccc1C] | 2.76 |
| I-1-322 | H | H | H | H | 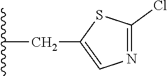 [SMILES: *Cc1cnc(Cl)s1] | 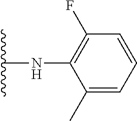 [SMILES: *Nc1c(C)cccc1F] | 3.1 |
| I-1-323 | H | H | H | H | 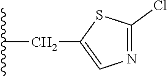 [SMILES: *Cc1cnc(Cl)s1] | 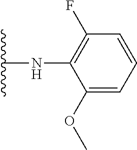 [SMILES: *Nc1c(F)cccc1OC] | 2.27 |
| I-1-324 | H | H | H | H | 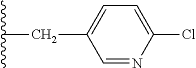 [SMILES: *Cc1ccc(Cl)nc1] | 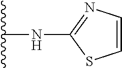 [SMILES: *Nc1nccs1] | 1.93 |
| I-1-325 | H | H | H | H | 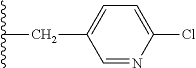 [SMILES: *Cc1ccc(Cl)nc1] | 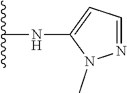 [SMILES: *Nc1ccnn1C] | 1.49 |
| I-1-326 | H | H | H | H | 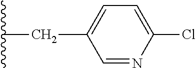 [SMILES: *Cc1ccc(Cl)nc1] | 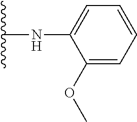 [SMILES: *Nc1ccccc1OC] | 2.55 |
| I-1-327 | H | H | H | H | 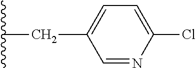 [SMILES: *Cc1ccc(Cl)nc1] | 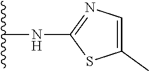 [SMILES: *Nc1ncc(C)s1] | 2.17 |
| I-1-328 | H | H | H | H | 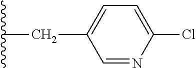 [SMILES: *Cc1ccc(Cl)nc1] | 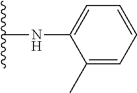 [SMILES: *Nc1ccccc1C] | 2.73 |
| I-1-329 | H | H | H | H | 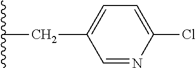 [SMILES: *Cc1ccc(Cl)nc1] | 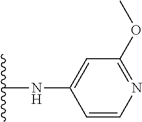 [SMILES: *Nc1ccnc(OC)c1] | 1.41 |
| I-1-330 | H | H | H | H | 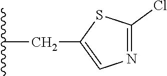 [SMILES: *Cc1cnc(Cl)s1] | 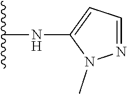 [SMILES: *Nc1ccnn1C] | 1.6 |
| I-1-331 | H | H | H | H | 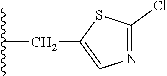 [SMILES: *Cc1cnc(Cl)s1] | 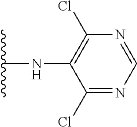 [SMILES: *Nc1c(Cl)ncnc1Cl] | 2.17 |
| I-1-332 | H | H | H | H | 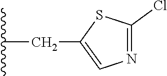 [SMILES: *Cc1cnc(Cl)s1] | 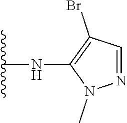 [SMILES: *Nc1c(Br)cnn1C] | 1.99 |
| I-1-334 | H | H | H | H | 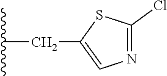 [SMILES: *Cc1cnc(Cl)s1] | 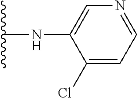 [SMILES: *Nc1cnccc1Cl] | 1.81 |
| I-1-335 | H | H | H | H | 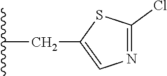 [SMILES: *Cc1cnc(Cl)s1] | 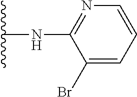 [SMILES: *Nc1ncccc1Br] | 1.58 |
| I-1-336 | H | H | H | H | 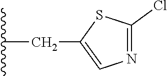 [SMILES: *Cc1cnc(Cl)s1] | 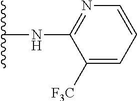 [SMILES: *Nc1ncccc1C(F)(F)F] | 1.99 |
| I-1-337 | H | H | H | H | 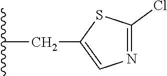 [SMILES: *Cc1cnc(Cl)s1] | 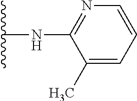 [SMILES: *Nc1ncccc1C] | 0.91 |
| I-1-338 | H | H | H | H | 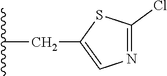 [SMILES: *Cc1cnc(Cl)s1] | 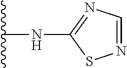 [SMILES: *Nc1ncns1] | 1.98 |

TABLE 2
(I-4)

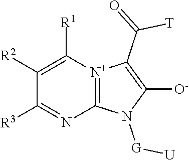

| No. | R¹ | R² | R³ | G-U | T | log p^{a)} |
|---|---|---|---|---|---|---|
| I-4-001 | H | H | H | 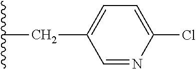 [SMILES: *Cc1ccc(Cl)nc1] | 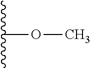 [SMILES: *OC] | 1.15 |

TABLE 3
(I-5)

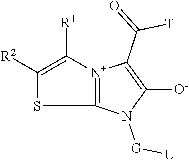

| No. | R¹ | R² | R³ | G-U | T | log p^{a)} |
|---|---|---|---|---|---|---|
| I-5-001 | H | H | H | 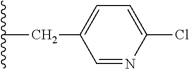 [SMILES: *Cc1ccc(Cl)nc1] | 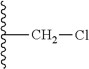 [SMILES: *CCl] | 1.33 |
| I-5-002 | H | H | H | 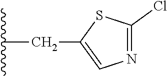 [SMILES: *Cc1cnc(Cl)s1] | 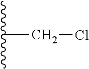 [SMILES: *CCl] | 1.46 |

1H NMR Data^{b)} of the Compounds According to Tables 1, 2 and 3

### Example I-1-001

¹H-NMR (601.6 MHz, d₆-DMSO): δ=9.855 (2.1); 9.844 (2.1); 8.5171 (2.5); 8.514 (2.5); 7.894 (1.0); 7.892 (1.0); 7.882 (1.3); 7.880 (2.3); 7.878 (1.5); 7.867 (1.4); 7.865 (1.4); 7.832 (1.6); 7.828 (1.6); 7.819 (1.8); 7.814 (1.7); 7.762 (2.5); 7.748 (2.0); 7.503 (3.0); 7.489 (2.7); 7.413 (1.2); 7.411 (1.2); 7.400 (2.2); 7.390 (1.2); 7.388 (1.2); 5.262 (8.5); 4.807 (16.0); 3.320 (17.8); 2.523 (0.4); 2.520 (0.4); 2.511 (5.8); 2.508 (11.4); 2.505 (15.2); 2.502 (11.1); 2.499 (5.3)

### Example I-1-002

¹H-NMR (400.0 MHz, d₆-DMSO): δ=9.842 (2.7); 9.826 (2.7); 7.956 (0.4); 7.934 (2.7); 7.926 (3.5); 7.921 (5.9); 7.906 (0.6); 7.854 (6.1); 7.433 (1.2); 7.426 (1.1); 7.411 (1.8); 7.410 (1.7); 7.402 (1.1); 7.394 (1.1); 5.755 (1.2); 5.414 (10.1); 4.794 (16.0); 3.320 (45.3); 2.672 (0.5); 2.507 (57.3); 2.503 (72.0); 2.499 (54.9); 2.329 (0.5); 1.046 (0.6); 1.031 (0.6); 0.000 (3.3)

### Example I-1-003

¹H-NMR (601.6 MHz, d₆-DMSO): δ=9.900 (2.8); 9.889 (2.8); 8.509 (3.4); 8.505 (3.3); 7.826 (1.3); 7.824 (1.3); 7.812 (4.7); 7.798 (3.8); 17.719 (3.2); 7.704 (2.5); 17.495 (3.8); 7.481 (3.4); 7.373 (1.6); 7.361 (2.9); 7.350 (1.5); 5.238 (11.0); 4.542 (16.0); 3.818 (0.4); 3.802 (0.4); 3.364 (29.5); 3.311 (20.5); 2.614 (0.5); 2.505 (51.1); 2.502 (65.3); 2.499 (47.8); 2.381 (0.4); 0.000 (51.4)

### Example I-1-004

¹H-NMR (400.0 MHz, d₆-DMSO): δ=9.766 (3.4); 9.750 (3.5); 8.539 (4.3); 8.533 (4.4); 8.387 (0.4); 8.179 (0.5); 7.959 (9.1); 7.901 (0.5); 7.886 (1.3); 7.884 (1.4); 7.862 (4.6); 7.855 (2.7); 7.846 (2.5); 7.841 (3.4); 7.834 (2.9); 7.776 (3.8); 7.755 (2.5); 7.533 (8.9); 7.521 (5.1); 7.500 (4.5); 7.398 (1.8); 7.395 (1.9); 7.379 (3.3); 7.363 (1.6); 7.360 (1.6); 5.548 (0.6); 5.497 (16.0); 5.289 (11.5); 5.259 (0.5); 4.806 (0.7); 3.320 (147.5); 2.891 (0.8); 2.732 (0.7); 2.676 (0.6); 2.671 (0.8); 2.667 (0.6); 2.507 (98.9); 2.502 (129.1); 2.498 (94.6); 2.333 (0.6); 2.329 (0.8); 2.325 (0.6); 0.000 (4.7)

### Example I-1-005

¹H-NMR (400.0 MHz, d₆-DMSO): δ=9.909 (1.4); 9.893 (1.5); 9.859 (0.3); 9.842 (0.4); 8.513 (2.2); 8.507 (1.9); 7.879 (0.3); 7.849 (0.6); 7.846 (0.6); 7.827 (1.7); 7.824 (2.0); 7.817 (1.3); 7.809 (1.4); 7.803 (1.5); 7.797 (1.2); 7.765 (0.5); 7.740 (1.8); 7.719 (1.1); 7.506 (2.5); 7.485 (2.2); 7.400 (0.4); 7.380 (0.9); 7.377 (0.8); 7.361 (1.4); 7.345 (0.7); 7.342 (0.7); 5.256 (5.7); 4.806 (2.3); 3.819 (8.3); 3.322 (22.3); 2.512 (14.6); 2.508 (29.3); 2.504 (38.3); 2.499 (27.8); 2.495 (13.5); 2.335 (0.4); 2.143 (16.0); 1.047 (0.3); 0.008 (0.5); 0.000 (14.5); −0.008 (0.5)

### Example I-1-006

¹H-NMR (400.0 MHz, d₆-DMSO): δ=9.838 (0.4); 9.811 (3.2); 9.794 (3.2); 8.539 (4.0); 8.533 (4.1); 8.313 (0.4); 8.289 (4.0); 8.282 (4.2); 8.151 (3.0); 8.148 (3.3); 8.141 (3.1); 8.137 (3.1); 8.125 (0.3); 7.874 (1.4); 7.871 (1.7); 7.867 (2.6); 7.861 (2.5); 7.853 (3.2); 7.847 (3.3); 7.840 (2.8); 7.834 (2.3); 7.831 (2.1); 7.763 (3.7); 7.742 (2.4); 7.523 (4.6); 7.502 (4.1); 7.389 (1.7); 7.386 (1.7); 7.370 (3.1); 7.354 (1.6); 7.351 (1.5); 7.336 (0.7); 7.332 (1.0); 7.325 (0.8); 7.315 (2.7); 7.311 (3.0); 7.308 (3.2); 7.304 (2.9); 7.300 (3.6); 7.289 (3.2); 7.279 (0.9); 7.268 (1.1); 5.754 (7.7); 5.332 (16.0); 5.283 (11.1); 3.319 (95.1); 2.676 (0.7); 2.671 (0.9); 2.667 (0.7); 2.507 (120.4); 2.502 (154.8); 2.498 (111.4); 2.333 (0.7); 2.329 (0.9); 2.325 (0.7); 1.352 (0.3); 1.234 (0.9); 1.046 (0.5); 1.030 (0.5); 0.008 (2.6); 0.000 (56.6); −0.009 (2.0)

### Example I-1-007

¹H-NMR (400.0 MHz, d₆-DMSO): δ=9.840 (3.0); 9.823 (3.1); 8.541 (3.9); 8.536 (3.8); 8.364 (0.3); 7.867 (3.3); 7.861 (2.4); 7.847 (5.4); 7.840 (2.9); 7.828 (2.1); 7.825 (2.0); 7.761 (3.7); 7.739 (2.3); 7.522 (4.5); 7.501 (4.0); 7.456 (0.4); 7.435 (0.4); 7.391 (1.6); 7.388 (1.6); 7.372 (3.0); 7.362 (0.4); 7.356 (1.6); 7.353 (1.5); 7.286 (0.4); 7.279 (3.4); 7.273 (1.0); 7.260 (4.9); 7.257 (4.5); 7.247 (0.9); 7.239 (4.0); 7.232 (0.4); 6.931 (1.9); 6.929 (1.8); 6.915 (7.0); 6.912 (6.4); 6.895 (8.8); 6.527 (0.4); 6.506 (0.4); 6.503 (0.3); 5.754 (2.9); 5.282 (10.6); 5.247 (16.0); 4.484 (0.6); 4.469 (0.6); 3.322 (56.4); 2.676 (0.4); 2.672 (0.5); 2.667 (0.4); 2.525 (1.3); 2.512 (32.1); 2.507 (64.9); 2.503 (85.0); 2.498 (61.1); 2.494 (29.1); 2.334 (0.4); 2.330 (0.5); 2.325 (0.4); 1.046 (1.4); 1.031 (1.4); 0.008 (1.2); 0.000 (31.7); −0.009 (1.0)

### Example I-1-008

¹H-NMR (601.6 MHz, d₆-DMSO): δ=9.885 (2.8); 9.884 (1.7); 9.874 (2.7); 9.873 (1.8); 7.886 (0.5); 7.876 (4.8); 7.873 (3.1); 7.872 (2.6); 7.866 (2.4); 7.864 (2.2); 7.852 (0.5); 7.850 (0.6); 7.844 (6.7); 7.396 (0.4); 7.388 (1.3); 7.384 (1.2); 7.378 (1.9); 7.374 (2.0); 7.367 (1.3); 7.363 (1.4); 5.753 (1.1); 5.392 (8.6); 5.259 (0.5); 4.532 (16.0); 3.949 (1.8); 3.367 (36.0); 3.312 (52.7); 2.615 (0.3); 2.524 (0.6); 2.521 (0.7); 2.518 (0.7); 2.509 (19.2); 2.506 (43.3); 2.503 (61.1); 2.500 (43.7); 2.497 (19.6); 2.387 (0.3); 2.185 (0.3); 1.357 (2.8); 0.097 (0.4); 0.005 (3.5); 0.000 (127.8); −0.006 (3.9); −0.100 (0.5)

### Example I-1-009

¹H-NMR (400.0 MHz, d₆-DMSO): δ=9.782 (3.2); 9.765 (3.3); 8.462 (0.5); 8.455 (0.5); 7.940 (0.9); 7.922 (6.5); 7.907 (2.9); 7.905 (2.7); 7.877 (8.0); 7.842 (0.6); 7.833 (0.9); 7.743 (5.0); 7.738 (4.6); 7.428 (5.3); 7.425 (5.3); 7.403 (1.5); 7.396 (1.5); 7.386 (2.4); 7.380 (2.7); 7.370 (1.3); 7.364 (1.4); 6.616 (0.4); 6.609 (0.4); 6.605 (0.4); 6.273 (3.5); 6.268 (5.8); 6.263 (3.5); 5.544 (1.7); 5.522 (16.0); 5.466 (1.3); 5.449 (10.8); 3.321 (49.3); 2.892 (0.3); 2.672 (0.4); 2.508 (50.4); 2.503 (66.5); 2.499 (48.9); 2.330 (0.4); 1.233 (0.6); 1.047 (0.4); 1.031 (0.4); 0.008 (1.1); 0.000 (31.9); −0.008 (1.3)

### Example I-1-010

¹H-NMR (400.0 MHz, d₆-DMSO): δ=9.752 (3.5); 9.735 (3.5); 7.964 (9.1); 7.944 (0.7); 7.929 (7.0); 7.923 (4.0); 7.916 (3.2); 7.891 (0.5); 7.872 (8.3); 7.534 (8.9); 7.412 (1.5); 7.404 (1.4); 7.396 (2.1); 7.389 (2.1); 7.380 (1.3); 7.373 (1.4); 5.487 (16.0); 5.445 (11.3); 4.342 (0.5); 4.331 (0.5); 3.322 (43.1); 2.893 (0.8); 2.733 (0.7); 2.673 (0.3); 2.508 (43.7); 2.504 (57.2); 2.499 (41.4); 2.331 (0.3); 1.047 (4.3); 1.031 (4.3); 0.008 (1.1); 0.000 (28.7); −0.008 (1.0)

### Example I-1-011

¹H-NMR (400.0 MHz, d₆-DMSO): δ=9.761 (3.1); 9.745 (3.1); 7.954 (2.6); 7.929 (6.5); 7.924 (4.0); 7.917 (3.3); 7.892 (0.6); 7.874 (7.7); 7.818 (5.5); 7.812 (5.4); 7.771 (0.3); 7.573 (0.4); 7.411 (1.4); 7.404 (1.5); 7.395 (1.9); 7.389 (2.1); 7.380 (1.3); 7.373 (1.3); 6.338 (5.7); 6.332 (5.7); 5.499 (0.5); 5.4); 5.421 (0.5); 5.414 (0.4); 4.583 (0.3); 3.321 (45.4); 2.893 (16.0); 2.872 (2.2); 2.746 (0.5); 2.733 (14.5); 2.673 (0.4); 2.508 (53.2); 2.504 (70.4); 2.499 (52.1); 2.330 (0.4); 2.326 (0.4); 1.338 (0.4); 1.226 (0.5); 0.008 (0.5); 0.000 (11.7); −0.008 (0.5)

### Example I-1-012

¹H-NMR (400.0 MHz, d₆-DMSO): δ=9.790 (2.0); 9.773 (2.2); 9.754 (0.3); 8.313 (0.4); 7.934 (0.7); 7.918 (4.5); 7.903 (2.0); 7.901 (1.9); 7.875 (5.3); 7.783 (0.3); 7.596 (3.0); 7.591 (2.9); 7.402 (0.9); 7.395 (1.0); 7.386 (1.4); 7.379 (1.7); 7.370 (0.8); 7.363 (1.0); 7.281 (0.6); 7.277 (0.6); 6.043 (0.6); 6.026 (3.0); 6.021 (3.1); 5.443 (7.1); 5.410 (9.3); 3.325 (29.0); 2.891 (0.3); 2.676 (0.4); 2.671 (0.6); 2.667 (0.4); 2.524 (1.4); 2.511 (36.6); 2.507 (75.7); 2.502 (101.0); 2.498 (74.2); 2.493 (36.6); 2.333 (0.4); 2.329 (0.6); 2.324 (0.4); 2.244 (1.2); 2.215 (2.5); 2.135 (16.0); 1.234 (0.5); 1.046 (1.3); 1.030 (1.3); 0.008 (1.7); 0.000 (50.3); −0.008 (1.8)

### Example I-1-013

¹H-NMR (400.0 MHz, d₆-DMSO): δ=9.785 (1.4); 9.768 (1.4); 7.936 (0.4); 7.921 (2.8); 7.906 (1.3); 7.904 (1.2); 7.881 (0.3); 7.872 (3.5); 7.477 (3.3); 7.475 (3.3); 7.405 (0.6); 7.398 (0.6); 7.389 (1.0); 7.382 (1.1); 7.373 (0.5); 7.366 (0.6); 7.192 (3.4); 7.191 (3.4); 5.442 (4.6); 5.393 (0.6); 5.376 (6.7); 3.765 (1.1); 3.672 (16.0); 3.318 (81.0); 2.675 (0.5); 2.671 (0.6); 2.666 (0.4); 2.541 (0.4); 2.524 (1.6); 2.510 (39.2); 2.506 (78.2); 2.502 (102.0); 2.497 (73.7); 2.493 (35.6); 2.333 (0.4); 2.328 (0.6); 2.324 (0.4); 1.045 (0.7); 1.030 (0.7); 0.008 (1.9); 0.000 (48.3); −0.009 (1.7)

### Example I-1-014

¹H-NMR (400.0 MHz, d₆-DMSO): δ=9.743 (3.6); 9.726 (3.5); 8.518 (10.5); 7.960 (10.9); 7.952 (0.8); 7.938 (6.6); 7.936 (7.0); 7.930 (3.9); 7.925 (3.5); 7.922 (3.1); 7.903 (0.5); 7.900 (0.5); 7.877 (8.7); 7.412 (1.5); 7.404 (1.5); 7.398 (1.7); 7.396 (1.9); 7.390 (1.9); 7.388 (1.9); 7.381 (1.4); 7.374 (1.4); 5.755 (2.6); 5.618 (16.0); 5.451 (11.2); 3.321 (35.7); 2.893 (0.7); 2.733 (0.6); 2.673 (0.3); 2.526 (0.8); 2.513 (20.0); 2.508 (41.6); 2.504 (55.1); 2.499 (39.6); 2.495 (18.9); 1.233 (0.5); 0.008 (1.0); 0.000 (30.8); −0.009 (1.1)

### Example I-1-015

¹H-NMR (400.0 MHz, d₆-DMSO): δ=9.727 (3.8); 9.710 (3.7); 8.561 (9.9); 8.313 (0.4); 8.064 (9.9); 7.950 (0.8); 7.937 (7.9); 7.928 (5.3); 7.901 (0.5); 7.872 (8.9); 7.415 (1.6); 7.406 (1.7); 7.398 (2.1); 7.391 (2.1); 7.385 (1.5); 7.377 (1.4); 5.602 (16.0); 5.446 (12.3); 3.320 (212.8); 2.671 (1.1); 2.506 (150.8); 2.502 (196.3); 2.498 (146.9); 2.329 (1.1); 1.226 (0.3); 0.146 (0.3); 0.000 (73.0); −0.150 (0.3)

### Example I-1-016

¹H-NMR (400.0 MHz, d₆-DMSO): δ=9.953 (0.4); 9.936 (0.4); 9.914 (1.6); 9.898 (1.7); 8.511 (2.6); 8.505 (2.2); 7.821 (1.6); 7.815 (1.6); 7.807 (1.7); 7.801 (2.0); 7.794 (1.9); 7.788 (1.4); 7.719 (1.9); 7.706 (0.6); 7.698 (1.3); 7.500 (2.9); 7.480 (2.6); 7.373 (0.9); 7.371 (0.9); 7.355 (1.6); 7.338 (0.8); 7.336 (0.8); 5.754 (4.1); 5.250 (1.8); 5.239 (5.9); 4.589 (8.8); 3.709 (0.4); 3.694 (1.1); 3.679 (1.5); 3.664 (1.1); 3.649 (0.5); 3.319 (59.5); 2.672 (0.5); 2.667 (0.4); 2.507 (70.7); 2.502 (91.8); 2.498 (67.0); 2.446 (3.3); 2.329 (0.5); 2.325 (0.4); 1.234 (0.6); 1.143 (16.0); 1.128 (15.8); 0.000 (32.4); −0.008 (1.3)

### Example I-1-017

¹H-NMR (400.0 MHz, d₆-DMSO): δ=9.826 (3.3); 9.810 (3.3); 7.955 (0.5); 7.933 (0.7); 7.916 (6.5); 7.901 (3.1); 7.873 (8.0); 7.531 (0.4); 7.406 (1.4); 7.400 (1.4); 7.390 (2.4); 7.384 (2.5); 7.374 (1.3); 7.368 (1.4); 7.331 (0.4); 7.310 (0.3); 7.286 (0.4); 7.280 (3.4); 7.274 (1.1); 7.259 (4.9); 7.257 (4.7); 7.246 (1.1); 7.239 (4.0); 6.933 (1.9); 6.917 (8.0); 6.914 (6.6); 6.896 (9.3); 6.880 (0.3); 5.755 (0.5); 5.439 (10.9); 5.331 (0.5); 5.239 (16.0); 5.215 (0.8); 3.322 (44.7); 2.893 (3.1); 2.734 (2.7); 2.673 (0.4); 2.526 (0.9); 2.508 (46.2); 2.504 (60.4); 2.500 (43.6); 2.331 (0.4); 1.047 (0.5); 1.032 (0.5); 0.008 (0.8); 0.000 (22.9); −0.008 (0.8)

### Example I-1-018

¹H-NMR (400.0 MHz, d₆-DMSO): δ=9.843 (0.5); 9.797 (3.3); 9.781 (3.3); 8.313 (0.4); 8.287 (3.8); 8.281 (3.9); 8.138 (3.0); 8.126 (0.5); 8.119 (0.4); 7.934 (0.6); 7.919 (6.5); 7.914 (3.8); 7.906 (3.0); 7.903 (2.7); 7.884 (0.7;); 7.881 (06); 7.869 (8.0); 7.403 (1.4); 7.396 (1.4); 7.388 (2.0); 7.381 (2.1); 7.372 (1.3); 7.365 (1.4); 7.337 (0.7); 7.333 (0.9); 7.330 (0.7); 7.326 (0.8); 7.316 (2.5); 7.312 (2.8); 7.309 (3.0); 7.305 (2.6); 7.299 (3.3); 7.288 (3.0); 7.278 (0.9); 7.267 (1.1); 5.754 (0.7); 5.438 (10.7); 5.323 (16.0); 3.322 (97.3); 2.676 (0.5); 2.672 (0.8); 2.667 (0.5); 2.525 (1.9); 2.511 (47.8); 2.507 (96.1); 2.503 (125.7); 2.498 (90.9); 2.494 (44.2); 2.334 (0.6); 2.329 (0.8); 2.325 (0.6); 1.352 (0.3); 1.234 (1.0); 0.008 (1.8); 0.000 (47.4); −0.009 (1.7)

### Example I-1-019

¹H-NMR (400.0 MHz, d₆-DMSO): δ=9.859 (2.2); 9.843 (2.2); 9.123 (5.2); 8.850 (11.6); 7.915 (0.8); 7.912 (0.8); 7.893 (2.1); 7.890 (1.6); 7.875 (1.7); 7.872 (1.7); 7.841 (2.7); 7.819 (1.5); 7.423 (1.2); 7.420 (1.2); 7.405 (2.1); 7.403 (2.0); 7.389 (1.2); 7.385 (1.2); 5.289 (8.2); 4.802 (16.0); 4.748 (1.1); 4.410 (0.7); 4.408 (0.7); 4.358 (0.5); 4.348 (0.6); 4.266 (0.9); 4.016 (0.4); 3.324 (22.5); 3.177 (1.2); 3.164 (1.2); 2.526 (0.5); 2.512 (18.2); 2.508 (36.9); 2.504 (48.7); 2.499 (32.512 (188); 2495 (17.9); 0008 (0.7); 0000 (190.7); −0.009 (0.8)

### Example I-1-020

¹H-NMR (400.0 MHz, d₆-DMSO): δ=9.905 (2.9); 9.888 (2.9); 8.509 (3.7); 8.503 (3.7); 7.829 (1.3); 7.819 (2.3); 7.811 (3.9); 7.798 (2.6); 7.792 (4.1); 7.720 (3.4); 7.698 (2.2); 7.498 (4.3); 7.478 (3.8); 7.376 (1.6); 7.359 (2.8); 7.343 (1.3); 7.340 (1.4); 5.754 (3.7); 5.237 (10.6); 4.580 (16.0); 3.583 (2.2); 3.565 (7.0); 3.548 (7.1); 3.530 (2.2); 3.320 (139.8); 2.671 (0.7); 2.667 (0.5); 2.507 (84.5); 2.502 (112.3); 2.498 (84.1); 2.329 (0.7); 2.325 (0.5); 1.174 (7.1); 1.156 (14.4); 1.139 (6.9); 0.000 (12.0); −0.009 (0.5)

### Example I-1-021

¹H-NMR (400.0 MHz, d₆-DMSO): δ=9.777 (3.9); 9.760 (3.9); 8.324 (0.9); 8.002 (1.2); 7.980 (7.3); 7.964 (3.1); 7.962 (3.0); 7.942 (1.0); 7.940 (1.0); 7.874 (9.9); 7.858 (0.4); 7.472 (1.7); 7.467 (1.7); 7.456 (3.1); 7.450 (3.2); 7.439 (1.6); 7.434 (1.6); 5.434 (12.3); 5.407 (0.4); 4.350 (0.6); 4.341 (0.6); 4.274 (14.6); 4.094 (0.3); 3.780 (0.9); 3.769 (0.3); 3.753 (0.3); 3.449 (0.6); 3.412 (0.4); 3.326 (51.1); 3.173 (2.2); 3.164 (2.1); 3.037 (1.9); 3.019 (6.3); 3.001 (6.5); 2.983 (2.0); 2.839 (1.1); 2.821 (3.4); 2.803 (3.6); 2.785 (1.2); 2.677 (0.9); 2.673 (1.2); 2.668 (0.9); 2.526 (2.7); 2.512 (73.7); 2.508 (152.5); 2.503 (203.7); 2.499 (149.8); 2.335 (0.9); 2.330 (1.2); 2.326 (0.9); 1.352 (0.5); 1.260 (0.4); 1.243 (7.7); 1.235 (3.8); 1.225 (16.0); 1.207 (6.9); 1.166 (3.7); 1.148 (7.3); 1.137 (0.6); 1.129 (3.5); 1.119 (0.8); 1.101 (0.4); 1.059 (0.5); 1.045 (10.4); 1.030 (10.3); 1.012 (0.5); 0.992 (0.5); 0.008 (2.5); 0.000 (75.3); −0.008 (2.8); −0.150 (0.3)

### Example I-1-022

¹H-NMR (601.6 MHz, d₆-DMSO): δ=9.868 (1.4); 9.857 (1.5); 8.516 (1.6); 8.512 (1.7); 7.893 (0.7); 7.891 (0.7); 7.878 (1.5); 7.876 (1.0); 7.866 (0.9); 7.864 (0.9); 7.833 (1.1); 7.829 (1.1); 7.819 (1.2); 7.815 (1.2); 7.766 (1.7); 7.751 (1.3); 7.505 (2.1); 7.491 (1.9); 7.401 (0.8); 7.399 (0.8); 7.389 (1.5); 7.378 (0.8); 7.376 (0.8); 5.751 (0.4); 5.264 (5.6); 4.547 (2.2); 4.526 (2.8); 4.385 (3.1); 4.364 (2.4); 3.312 (56.1); 2.677 (16.0); 2.523 (0.4); 2.520 (0.5); 2.517 (0.5); 2.508 (10.1); 2.505 (22.1); 2.502 (31.0); 2.499 (22.4); 20.496 (10.4); 0.005 (2.1); 0.000 (69.5); −0.006 (2.3)

### Example I-1-023

¹H-NMR (601.6 MHz, d₆-DMSO): δ=9.902 (2.3); 9.891 (2.5); 9.118 (6.0); 8.838 (13.6); 7.841 (0.9); 7.839 (0.9); 7.829 (1.0); 7.827 (2.3); 7.825 (1.9); 7.815 (2.0); 7.813 (2.0); 7.794 (2.8); 7.780 (1.3); 7.378 (1.3); 7.376 (1.3); 7.366 (2.0); 7.365 (1.9); 7.355 (1.3); 7.353 (1.3); 5.752 (0.5); 5.266 (8.8); 4.539 (16.0); 4.481 (1.0); 3.362 (34.2); 3.347 (2.0); 3.311 (44.3); 3.175 (1.1); 3.166 (1.2); 2.614 (0.4); 2.523 (0.6); 2.520 (0.7); 2.517 (0.8); 2.508 (21.5); 2.505 (47.7); 2.502 (67.4); 2.499 (47.8); 2.496 (21.5); 2.387 (0.4); 0.097 (0.5); 0.005 (3.8); 0.000 (128.0); −0.006 (3.9); −0.100 (0.4)

### Example I-1-024

¹H-NMR (601.6 MHz, d₆-DMSO): δ=9.785 (3.1); 9.776 (2.3); 9.774 (3.2); 9.772 (2.2); 8.638 (0.4); 8.518 (3.7); 8.515 (3.7); 7.931 (1.5); 7.929 (1.6); 7.919 (1.8); 7.917 (3.5); 7.914 (2.2); 7.904 (2.4); 7.902 (2.4); 7.855 (2.7); 7.851 (2.6); 7.841 (2.9); 7.837 (3.0); 7.832 (3.8); 7.817 (2.8); 7.518 (4.6); 7.504 (4.2); 7.455 (1.9); 7.453 (1.9); 7.442 (3.2); 7.431 (1.9); 7.429 (1.9); 5.273 (11.2); 4.290 (14.3); 3.317 (12.7); 3.031 (1.8); 3.019 (6.2); 3.007 (6.3); 2.995 (1.9); 2.892 (0.8); 2.732 (0.6); 2.617 (0.4); 2.614 (0.5); 2.611 (0.4); 2.524 (0.8); 2.521 (1.0); 2.517 (0.9); 2.509 (22.3); 2.506 (50.8); 2.503 (71.7); 2.500 (51.5); 2.497 (23.3); 2.390 (0.4); 2.387 (0.5); 2.384 (0.4); 1.238 (7.2); 1.226 (16.0); 1.214 (7.0); 1.043 (0.5); 1.033 (0.4); 0.097 (0.7); 0.005 (5.5); 0.000 (200.9); −0.006 (6.3); −0.100 (0.7)

### Example I-1-025

¹H-NMR (400.0 MHz, d₆-DMSO): δ=9.848 (2.9); 9.831 (2.9); 7.925 (0.6); 7.909 (5.6); 7.894 (2.5); 7.891 (2.4); 7.872 (0.6); 7.869 (0.7); 7.863 (7.4); 7.573 (0.5); 7.428 (3.3); 7.424 (4.7); 7.420 (1.3); 7.406 (5.5); 7.404 (5.4); 7.399 (0.7); 7.392 (1.5); 7.385 (1.4); 7.376 (2.1); 7.370 (2.2); 7.360 (1.2); 7.354 (1.3); 7.296 (3.4); 7.291 (1.1); 7.277 (5.9); 7.261 (1.4); 7.257 (3.5); 7.175 (1.2); 7.172 (2.1); 7.169 (1.2); 7.158 (1.0); 7.154 (3.1); 7.138 (0.7); 7.135 (1.2); 5.755 (0.4); 5.429 (9.7); 4.584 (0.4); 4.569 (0.4); 4.417 (16.0); 3.326 (29.4); 3.321 (33.1); 2.673 (0.4); 2.526 (0.9); 2.521 (1.3); 2.513 (21.3); 2.508 (44.6); 2.504 (59.6); 2.499 (43.1); 2.495 (20.5); 2.330 (0.4); 1.047 (2.8); 1.032 (2.8); 0.146 (0.4); 0.008 (2.9); 0.000 (85.9); −0.009 (2.9); −0.150 (0.4)

### Example I-1-026

¹H-NMR (400.0 MHz, d₆-DMSO): δ=9.901 (1.5); 9.884 (1.5); 7.893 (0.3); 7.891 (0.3); 7.877 (2.7); 7.875 (2.7); 7.871 (1.8); 7.869 (1.6); 7.862 (1.4); 7.859 (1.3); 7.849 (3.8); 7.840 (0.3); 7.390 (0.7); 7.384 (0.7); 7.374 (1.0); 7.368 (1.0); 7.358 (0.6); 7.352 (0.7); 5.756 (0.8); 5.395 (4.6); 4.579 (8.3); 3.715 (0.4); 3.700 (1.1); 3.684 (1.5); 3.669 (1.1); 3.654 (0.4); 3.326 (14.6); 3.323 (14.8); 2.523 (0.5); 2.515 (7.7); 2.510 (16.2); 2.506 (21.7); 2.501 (15.7); 2.496 (7.6); 1.233 (0.4); 1.147 (16.0); 1.132 (15.7); 0.008 (1.3); 0.000 (36.5); −0.009 (1.3)

### Example I-1-027

¹H-NMR (400.0 MHz, d₆-DMSO): δ=9.895 (1.6); 9.878 (1.6); 7.958 (1.0); 7.913 (0.3); 7.897 (3.1); 7.882 (1.4); 7.879 (1.3); 7.861 (4.1); 7.392 (0.7); 7.385 (0.7); 7.375 (1.1); 7.369 (1.2); 7.359 (0.6); 7.353 (0.7); 5.415 (5.3); 4.343 (0.6); 4.333 (0.7); 3.909 (8.5); 3.325 (11.9); 3.126 (0.4); 3.109 (1.1); 3.092 (1.5); 3.076 (1.1); 3.059 (0.4); 2.896 (7.6); 2.737 (6.4); 2.517 (5.3); 2.512 (10.9); 2.508 (14.5); 2.503 (10.4); 2.499 (4.9); 1.236 (16.0); 1.219 (15.5); 1.049 (5.1); 1.034 (5.1); 0.008 (0.6); 0.000 (16.6); −0.009 (0.6)

### Example I-1-028

¹H-NMR (400.0 MHz, d₆-DMSO): δ=9.891 (3.1); 9.875 (3.1); 7.956 (1.4); 7.911 (0.6); 7.895 (5.6); 7.879 (2.7); 7.869 (1.7); 7.858 (7.3); 7.392 (1.1); 7.386 (1.2); 7.377 (2.0); 7.370 (2.1); 7.360 (1.2); 7.354 (1.2); 5.412 (9.9); 5.388 (1.7); 4.341 (2.0); 4.331 (2.1); 3.861 (14.4); 3.796 (0.6); 3.785 (0.7); 3.780 (0.8); 3.770 (0.8); 3.765 (0.7); 3.755 (0.6); 3.321 (28.3); 3.165 (0.9); 3.147 (1.0); 3.129 (0.3); 2.894 (8.3); 2.735 (8.0); 2.645 (2.1); 2.627 (6.6); 2.609 (6.8); 2.590 (2.3); 2.505 (46.9); 1.320 (1.0); 1.302 (2.0); 1.284 (1.0); 1.222 (6.9); 1.204 (13.8); 1.185 (6.6); 1.047 (16.0); 1.032 (15.8); 0.008 (1.5); 0.001 (30.0); 0.000 (36.7)

### Example I-1-029

¹H-NMR (400.0 MHz, d₆-DMSO): δ=9.911 (2.8); 9.894 (2.9); 8.510 (3.5); 8.504 (3.5); 7.832 (1.2); 7.829 (1.2); 7.820 (2.3); 7.814 (3.6); 7.811 (3.2); 7.808 (2.1); 7.799 (2.6); 7.793 (3.8); 7.720 (3.3); 7.699 (2.1); 7.499 (4.1); 7.479 (3.7); 7.376 (1.5); 7.373 (1.5); 7.358 (2.7); 7.341 (1.4); 7.338 (1.3); 5.239 (10.1); 4.585 (16.0); 3.482 (4.6); 3.465 (9.6); 3.449 (4.8); 3.318 (28.6); 2.672 (0.4); 2.512 (22.1); 2.508 (44.1); 2.503 (57.8); 2.499 (42.2); 2.494 (20.8); 2.330 (0.4); 1.603 (0.5); 1.584 (2.2); 1.567 (4.4); 1.549 (4.6); 1.532 (2.4); 1.514 (0.6); 1.046 (1.2); 1.031 (1.2); 0.914 (7.7); 0.895 (15.3); 0.877 (6.9); 0.008 (2.1); 0.000 (53.1); −0.008 (2.1)

### Example I-1-030

¹H-NMR (400.0 MHz, d₆-DMSO): δ=9.871 (2.0); 9.854 (2.0); 8.504 (2.4); 8.498 (2.6); 7.840 (0.8); 7.818 (2.8); 7.812 (1.6); 7.798 (2.3); 7.791 (1.6); 7.727 (2.3); 7.706 (1.6); 7.498 (2.8); 7.477 (2.4); 7.384 (1.1); 7.367 (2.0); 7.349 (1.0); 5.754 (0.5); 5.234 (7.1); 4.695 (9.0); 4.280 (10.1); 3.664 (16.0); 3.316 (31.7); 2.671 (0.4); 2.502 (62.7); 2.328 (0.4); 0.000 (36.7); −0.002 (34.9)

### Example I-1-031

¹H-NMR (400.0 MHz, d₆-DMSO): δ=9.832 (3.0); 9.816 (3.1); 8.537 (3.8); 8.531 (3.8); 7.870 (1.3); 7.867 (1.6); 7.863 (2.5); 7.857 (2.5); 7.851 (1.8); 7.848 (3.1); 7.843 (3.2); 7.836 (2.8); 7.830 (2.2); 7.827 (2.1); 7.761 (3.6); 7.739 (2.3); 7.521 (4.6); 7.500 (4.1); 7.390 (1.6); 7.388 (1.7); 7.372 (2.9); 7.356 (1.6); 7.353 (1.5); 7.105 (3.6); 7.099 (1.2); 7.094 (0.5); 7.088 (1.6); 7.083 (6.5); 7.072 (0.7); 7.067 (1.4); 7.061 (4.8); 7.051 (0.4); 6.945 (0.4); 6.935 (4.8); 6.929 (1.6); 6.924 (4.9); 6.918 (2.4); 6.912 (3.6); 6.907 (1.4); 6.901 (3.5); 5.755 (0.8); 5.277 (10.5); 5.229 (16.0); 3.789 (0.5); 3.774 (0.6); 3.759 (0.4); 3.320 (20.6); 3.171 (0.4); 2.672 (0.4); 2.525 (1.0); 2.512 (23.2); 2.508 (47.9); 2.503 (63.9); 2.498 (46.3); 2.494 (22.4); 2.330 (0.4); 1.046 (11.2); 1.031 (11.1); 0.008 (2.0); 0.000 (57.0); −0.009 (2.0)

### Example I-1-032

¹H-NMR (400.0 MHz, d₆-DMSO): δ=9.890 (2.9); 9.873 (2.9); 7.891 (0.4); 7.889 (0.5); 7.877 (5.3); 7.875 (5.6); 7.870 (3.0); 7.868 (2.9); 7.863 (2.7); 7.860 (2.5); 7.845 (6.9); 7.838 (0.7); 7.394 (1.2); 7.386 (1.2); 7.378 (1.9); 7.372 (1.5); 7.363 (1.1); 7.355 (1.2); 5.390 (9.0); 5.251 (0.5); 4.569 (16.0); 4.348 (0.4); 4.330 (0.4); 3.585 (2.2); 3.568 (7.4); 3.550 (7.5); 3.533 (2.3); 3.324 (46.2); 3.320 (50.0); 2.672 (0.4); 2.668 (0.3); 2.526 (1.1); 2.521 (1.6); 2.512 (25.9); 2.508 (54.0); 2.503 (72.2); 2.499 (52.2); 2.494 (25.1); 2.330 (0.5); 2.325 (0.3); 1.316 (0.4); 1.299 (0.8); 1.281 (0.4); 1.176 (7.4); 1.158 (15.1); 1.141 (7.2); 1.046 (1.0); 1.031 (1.0); 0.146 (0.5); 0.008 (3.6); 0.000 (108.7); −0.009 (3.6); −0.150 (0.5)

### Example I-1-033

¹H-NMR (400.0 MHz, d₆-DMSO): δ=9.897 (3.1); 9.895 (1.9); 9.881 (3.0); 9.878 (1.9); 7.894 (0.5); 7.892 (0.5); 7.880 (5.3); 7.877 (5.6); 7.873 (3.1); 7.871 (2.9); 7.865 (2.7); 7.862 (2.5); 7.848 (7.2); 7.840 (0.7); 7.393 (1.3); 7.386 (1.3); 7.377 (1.8); 7.370 (1.7); 7.362 (1.2); 7.355 (1.2); 5.394 (8.9); 4.576 (16.0); 4.343 (0.8); 4.332 (0.8); 4.246 (0.3); 3.797 (0.4); 3.787 (0.4); 3.782 (0.6); 3.771 (0.5); 3.767 (0.4); 3.756 (0.4); 3.486 (4.8); 3.470 (10.0); 3.453 (4.9); 3.327 (13.3); 3.324 (15.4); 2.896 (0.7); 2.736 (0.5); 2.529 (0.4); 2.525 (0.5); 2.516 (10.1); 2.512 (21.5); 2.507 (28.9); 2.502 (20.9); 2.498 (10.0); 1.606 (0.5); 1.588 (2.1); 1.571 (4.2); 1.552 (4.5); 1.535 (2.3); 1.517 (0.6); 1.049 (11.9); 1.034 (11.8); 0.923 (0.5); 0.917 (8.0); 0.905 (1.0); 0.899 (16.0); 0.886 (0.6); 0.880 (7.2); 0.008 (1.7); 0.000 (55.4); −0.009 (1.9)

### Example I-1-034

¹H-NMR (400.0 MHz, d₆-DMSO): δ=9.894 (1.6); 9.878 (1.6); 9.843 (0.4); 9.826 (0.4); 7.934 (0.4); 7.926 (0.4); 7.919 (0.8); 7.908 (0.5); 7.893 (3.2); 7.879 (1.5); 7.876 (1.5); 7.855 (4.8); 7.394 (0.9); 7.388 (0.7); 7.378 (1.1); 7.372 (1.2); 7.362 (0.6); 7.356 (0.7); 5.409 (6.0); 4.794 (2.3); 3.807 (8.6); 3.321 (72.2); 2.893 (0.5); 2.734 (0.4); 2.526 (0.9); 2.508 (33.2); 2.504 (44.7); 2.499 (35.0); 2.335 (0.4); 2.141 (16.0); 1.047 (0.6); 1.032 (0.6); 0.008 (2.0); 0.000 (48.0); −0.008 (2.4)

### Example I-1-035

¹H-NMR (400.0 MHz, d₆-DMSO): δ=9.847 (6.4); 9.830 (6.8); 7.968 (1.1); 7.965 (1.2); 7.946 (5.0); 7.943 (5.7); 7.928 (13.8); 7.911 (1.5); 7.839 (16.0); 7.757 (7.6); 7.753 (9.8); 7.737 (11.6); 7.734 (10.8); 7.593 (2.9); 7.588 (3.9); 7.584 (2.3); 7.572 (11.9); 7.553 (10.1); 7.549 (5.9); 7.545 (7.6); 7.541 (4.6); 7.535 (2.2); 7.527 (4.7); 7.518 (0.9); 7.513 (0.9); 7.509 (1.3); 7.441 (2.9); 7.435 (2.7); 7.425 (5.0); 7.419 (4.8); 7.409 (2.8); 7.402 (2.7); 5.755 (0.7); 5.441 (0.7); 5.398 (16.0); 5.355 (0.7); 4.564 (6.8); 4.532 (11.8); 4.459 (11.9); 4.426 (6.9); 3.321 (77.8); 3.178 (0.3); 2.678 (0.5); 2.673 (0.6); 2.669 (0.5); 2.526 (2.0); 2.513 (38.1); 2.509 (76.5); 2.504 (101.1); 2.500 (76.3); 2.495 (39.7); 2.335 (0.5); 2.331 (0.6); 2.326 (0.5); 1.232 (0.5); 1.047 (1.0); 1.032 (1.0); 0.146 (0.4); 0.008 (4.2); 0.000 (89.9); −0.008 (4.8); −0.150 (0.4)

### Example I-1-036

¹H-NMR (400.0 MHz, d₆-DMSO): δ=9.953 (1.8); 9.936 (1.8); 8.517 (2.2); 8.511 (2.3); 7.821 (1.3); 7.814 (1.8); 7.800 (1.6); 7.793 (2.6); 7.771 (1.2); 7.706 (2.1); 7.685 (1.4); 7.499 (2.6); 7.479 (2.3); 7.346 (1.0); 7.329 (1.8); 7.311 (0.9); 5.754 (1.0); 5.250 (7.3); 3.318 (41.9); 2.676 (0.3); 2.672 (0.4); 2.507 (52.3); 2.502 (69.0); 2.498 (52.6); 2.447 (16.0); 2.329 (0.4); 0.008 (2.2); 0.000 (48.6)

### Example I-1-037

¹H-NMR (400.0 MHz, d₆-DMSO): δ=9.858 (3.2); 9.842 (3.4); 7.960 (0.3); 7.960 (057 (0.3); 7.938 (2.8); 7.935 (3.4); 7.931 (3.6); 7.924 (6.7); 7.922 (6.4); 7.909 (0.6); 7.871 (0.6); 7.858 (7.7); 7.422 (1.3); 7.414 (1.3); 7.408 (1.6); 7.406 (1.7); 7.400 (1.6); 7.397 (1.6); 7.392 (1.4); 7.384 (1.3); 5.419 (10.2); 5.395 (0.7); 4.490 (3.8); 4.458 (6.0); 4.364 (6.2); 4.340 (0.4); 4.332 (4.0); 4.094 (0.4); 4.081 (0.4); 3.319 (79.6); 3.177 (1.9); 3.168 (0.6); 3.164 (1.8); 3.150 (0.5); 2.981 (0.4); 2.962 (1.5); 2.947 (0.9); 2.943 (1.7); 2.929 (2.4); 2.910 (2.3); 2.891 (0.8); 2.824 (0.7); 2.805 (2.3); 2.787 (2.5); 2.772 (1.7); 2.769 (1.1); 2.754 (1.6); 2.735 (0.5); 2.676 (0.4); 2.672 (0.5); 2.668 (0.4); 2.525 (1.1); 2.512 (29.9); 2.508 (61.2); 2.503 (80.6); 2.498 (58.5); 2.494 (28.3); 2.334 (0.4); 2.330 (0.5); 2.325 (0.4); 1.322 (0.5); 1.304 (1.1); 1.285 (0.5); 1.259 (7.6); 1.240 (16.0); 1.221 (7.3); 1.046 (2.2); 1.031 (2.2); 0.008 (2.1); 0.000 (61.1); −0.008 (2.3)

### Example I-1-038

¹H-NMR (400.0 MHz, d₆-DMSO): δ=9.870 (2.9); 9.854 (3.0); 7.957 (0.4); 7.935 (3.2); 7.930 (3.5); 7.923 (6.3); 7.922 (6.3); 7.909 (0.5); 7.857 (7.4); 7.424 (1.2); 7.416 (1.2); 7.408 (1.6); 7.402 (1.5); 7.394 (1.2); 7.386 (1.2); 5.419 (9.5); 4.390 (2.7); 4.358 (6.1); 4.317 (5.7); 4.285 (2.4); 4.093 (0.7); 4.080 (0.7); 3.318 (167.0); 3.176 (2.3); 3.163 (2.3); 3.003 (0.7); 2.986 (1.8); 2.969 (2.5); 2.952 (1.9); 2.935 (0.7); 2.676 (0.6); 2.671 (0.8); 2.667 (0.6); 2.524 (2.0); 2.506 (100.5); 2.502 (132.5); 2.498 (97.8); 2.333 (0.6); 2.329 (0.8); 2.324 (0.6); 1.248 (13.0); 1.236 (16.0); 1.231 (14.7); 1.219 (12.9); 0.146 (0.4); 0.008 (2.9); 0.000 (86.5); −0.008 (3.8); −0.150 (0.4)

### Example I-1-039

¹H-NMR (400.0 MHz, d₆-DMSO): δ=9.855 (1.9); 9.839 (2.0); 7.937 (3.8); 7.927 (4.1); 7.861 (5.0); 7.833 (0.4); 7.814 (0.4); 7.422 (1.0); 7.411 (1.4); 7.406 (1.1); 7.401 (1.0); 7.395 (1.4); 7.384 (0.9); 7.364 (0.4); 5.754 (2.2); 5.421 (6.8); 4.543 (2.0); 4.511 (3.0); 4.388 (3.1); 4.356 (2.1); 3.319 (60.4); 3.175 (0.5); 3.163 (0.5); 2.771 (0.3); 2.679 (16.0); 2.541 (0.7); 2.506 (95.8); 2.502 (128.9); 2.498 (105.6); 2.329 (0.7); 1.235 (5.6); 0.854 (0.5); 0.146 (0.4); 0.000 (82.4); −0.150 (0.4)

### Example I-1-040

¹H-NMR (400.0 MHz, d₆-DMSO): δ=9.859 (2.0); 9.842 (2.1); 9.833 (1.7); 9.816 (1.7); 7.920 (0.4); 7.907 (4.8); 7.899 (2.8); 7.896 (2.6); 7.893 (2.3); 7.885 (2.3); 7.880 (2.9); 7.860 (9.6); 7.841 (0.5); 7.406 (0.8); 7.398 (0.8); 7.390 (1.1); 7.381 (1.6); 7.376 (1.5); 7.364 (1.5); 7.359 (1.4); 7.347 (0.7); 7.343 (0.7); 5.756 (4.0); 5.423 (7.8); 5.418 (7.0); 4.680 (8.1); 4.573 (6.7); 3.395 (0.8); 3.377 (2.5); 3.359 (2.6); 3.341 (1.2); 3.325 (39.2); 3.323 (31.8); 3.311 (3.3); 3.293 (3.1); 3.275 (1.0); 2.510 (32.4); 2.506 (43.3); 2.502 (32.9); 2.070 (12.7); 1.893 (16.0); 1.138 (2.9); 1.120 (6.3); 1.102 (2.8); 1.010 (3.6); 0.993 (7.7); 0.975 (3.5); 0.006 (1.4); 0.000 (33.2); −0.008 (1.6)

### Example I-1-041

¹H-NMR (400.0 MHz, d₆-DMSO): δ=9.909 (3.5); 9.893 (3.4); 7.938 (0.5); 7.935 (0.6); 7.921 (0.6); 7.918 (0.8); 7.912 (0.6); 7.905 (0.5); 7.895 (0.7); 7.883 (6.0); 7.881 (6.5); 7.875 (3.6); 7.873 (3.7); 7.870 (3.7); 7.867 (3.1); 7.855 (0.8); 7.843 (8.8); 7.438 (0.4); 7.433 (0.4); 7.420 (0.8); 7.416 (1.0); 7.401 (4.3); 7.394 (4.1); 7.385 (7.9); 7.381 (7.8); 7.378 (7.8); 7.359 (6.4); 7.354 (2.3); 7.339 (2.5); 7.321 (0.7); 7.315 (1.6); 7.309 (3.0); 7.300 (0.9); 7.293 (2.1); 7.286 (0.6); 7.280 (0.5); 7.276 (0.8); 7.271 (0.4); 5.754 (4.0); 5.412 (0.5); 5.387 (10.1); 5.374 (1.1); 5.018 (0.9); 5.013 (0.9); 4.794 (0.4); 4.657 (16.0); 4.617 (14.5); 4.498 (0.5); 4.484 (0.5); 4.146 (0.8); 4.142 (0.8); 3.331 (87.0); 3.324 (72.7); 2.677 (0.6); 2.672 (0.8); 2.668 (0.6); 2.525 (1.8); 2.512 (47.0); 2.508 (97.5); 2.503 (130.2); 2.499 (94.3); 2.494 (45.8); 2.440 (0.9); 2.334 (0.6); 2.330 (0.8); 2.325 (0.6); 1.336 (1.2); 1.299 (1.3); 1.259 (1.9); 1.250 (1.7); 1.233 (1.7); 0.146 (0.4); 0.008 (2.8); 0.000 (87.3); −0.009 (2.9); −0.150 (0.4)

### Example I-1-042

¹H-NMR (400.0 MHz, d₆-DMSO): δ=9.851 (3.1); 9.835 (3.2); 8.186 (0.5); 7.995 (0.5); 7.992 (0.5); 7.973 (2.3); 7.970 (2.5); 7.958 (5.1); 7.955 (6.0); 7.937 (0.8); 7.876 (0.6); 7.862 (7.3); 7.442 (1.4); 7.436 (1.4); 7.431 (0.6); 7.426 (2.4); 7.419 (2.3); 7.410 (1.4); 7.403 (1.2); 5.754 (0.7); 5.538 (0.7); 5.427 (9.6); 5.403 (0.8); 4.931 (12.1); 4.714 (0.4); 4.699 (0.4); 3.317 (82.4); 3.307 (6.4); 3.288 (5.8); 3.270 (1.9); 3.169 (0.6); 3.151 (0.6); 2.676 (0.5); 2.671 (0.6); 2.667 (0.5); 2.524 (1.4); 2.520 (2.2); 2.511 (36.6); 2.507 (76.4); 2.502 (102.4); 2.498 (73.9); 2.493 (35.5); 2.333 (0.4); 2.329 (0.6); 2.324 (0.4); 1.322 (0.7); 1.300 (7.5); 1.282 (16.0); 1.263 (7.2); 1.235 (1.0); 1.110 (0.6); 1.107 (0.6); 1.092 (1.2); 1.089 (1.3); 1.074 (0.6); 1.071 (0.6); 1.046 (0.6); 1.030 (0.6); 0.146 (0.4); 0.008 (2.4); 0.000 (77.6); −0.008 (2.8); −0.150 (0.4)

### Example I-1-043

¹H-NMR (400.0 MHz, d₆-DMSO): δ=9.937 (1.6); 9.920 (1.6); 7.881 (0.4); 7.880 (0.4); 7.862 (2.8); 7.859 (3.3); 7.854 (4.2); 7.846 (1.4); 7.843 (1.3); 7.824 (0.4); 7.821 (0.3); 7.573 (0.4); 7.364 (0.7); 7.358 (0.7); 7.348 (1.3); 7.342 (1.4); 7.331 (0.7); 7.326 (0.7); 5.755 (0.4); 5.403 (5.7); 3.322 (21.4); 2.528 (0.4); 2.523 (0.6); 2.514 (8.6); 2.510 (18.0); 2.505 (24.1); 2.501 (17.2); 2.496 (8.2); 2.441 (16.0); 0.008 (0.3); 0.000 (10.0); −0.009 (0.3)

### Example I-1-044

¹H-NMR (400.0 MHz, d₆-DMSO): δ=9.769 (1.3); 9.753 (1.3); 7.924 (2.7); 7.919 (1.5); 7.911 (1.2); 7.908 (1.1); 7.873 (3.4); 7.822 (3.7); 7.479 (3.1); 7.478 (3.6); 7.405 (0.6); 7.398 (0.6); 7.389 (0.8); 7.382 (0.9); 7.374 (0.5); 7.367 (0.6); 5.478 (6.1); 5.443 (4.2); 3.318 (37.7); 2.671 (0.4); 2.524 (1.0); 2.511 (22.3); 2.506 (47.2); 2.502 (63.9); 2.497 (46.9); 2.493 (23.0); 2.333 (0.4); 2.321 (16.0); 2.298 (0.5); 1.234 (0.4); 1.045 (0.5); 1.030 (0.5); 0.000 (4.0)

### Example I-1-045

¹H-NMR (400.0 MHz, d₆-DMSO): δ=9.747 (3.8); 9.731 (3.8); 8.377 (6.6); 7.950 (0.7); 7.935 (7.7); 7.930 (4.2); 7.923 (3.5); 7.921 (3.4); 7.899 (0.7); 7.886 (8.1); 7.876 (9.6); 7.411 (1.6); 7.404 (1.6); 7.395 (2.2); 7.388 (2.2); 7.380 (1.4); 7.373 (1.5); 5.585 (16.0); 5.449 (11.9); 3.319 (87.6); 2.676 (0.5); 2.672 (0.7); 2.667 (0.5); 2.525 (1.6); 2.511 (41.4); 2.507 (88.5); 2.503 (120.0); 2.498 (87.1); 2.494 (42.0); 2.334 (0.5); 2.329 (0.7); 2.325 (0.5); 1.234 (0.6); 1.046 (0.8); 1.031 (0.8); 0.000 (8.3)

### Example I-1-046

¹H-NMR (400.0 MHz, d₆-DMSO): δ=9.739 (3.7); 9.723 (3.6); 7.947 (0.7); 7.934 (6.6); 7.932 (6.9); 7.928 (4.0); 7.920 (3.4); 7.917 (3.1); 7.898 (0.7); 7.895 (0.6); 7.874 (8.9); 7.413 (1.7); 7.406 (1.7); 7.397 (2.2); 7.390 (2.3); 7.382 (1.5); 7.375 (1.6); 7.295 (7.2); 7.291 (7.3); 6.894 (7.4); 6.890 (7.4); 5.755 (1.1); 5.449 (10.9); 5.350 (16.0); 3.320 (87.2); 2.892 (1.2); 2.732 (1.0); 2.676 (0.5); 2.672 (0.7); 2.667 (0.5); 2.525 (1.7); 2.520 (2.7); 2.512 (39.6); 2.507 (83.6); 2.503 (112.2); 2.498 (80.9); 2.494 (38.6); 2.334 (0.5); 2.330 (0.6); 2.325 (0.5); 1.235 (0.8); 0.000 (8.2)

### Example I-1-047

¹H-NMR (400.0 MHz, d₆-DMSO): δ=9.728 (3.5); 9.711 (3.7); 9.698 (0.3); 8.046 (7.2); 8.040 (7.3); 7.952 (0.6); 7.940 (7.2); 7.938 (7.7); 7.930 (4.5); 7.928 (4.5); 7.925 (3.3); 7.905 (0.4); 7.902 (0.4); 7.874 (9.8); 7.748 (0.7); 7.743 (0.7); 7.414 (1.6); 7.406 (1.6); 7.401 (1.7); 7.398 (1.8); 7.393 (1.7); 7.390 (1.9); 7.384 (1.6); 7.376 (1.5); 7.212 (0.8); 7.207 (0.7); 7.007 (7.7); 7.001 (7.7); 5.754 (5.5); 5.719 (1.5); 5.677 (0.5); 5.653 (16.0); 5.449 (11.4); 3.320 (67.4); 2.892 (0.6); 2.732 (0.5); 2.677 (0.4); 2.672 (0.6); 2.667 (0.5); 2.525 (1.3); 2.521 (2.0); 2.512 (34.9); 2.507 (75.2); 2.503 (102.0); 2.498 (72.7); 2.494 (34.1); 2.334 (0.4); 2.330 (0.6); 2.325 (0.4); 1.234 (0.4); 1.046 (0.7); 1.031 (0.7); 0.000 (8.5)

### Example I-1-048

¹H-NMR (400.0 MHz, d₆-DMSO): δ=9.762 (3.9); 9.745 (3.9); 8.313 (0.6); 8.002 (2.2); 7.990 (2.1); 7.979 (0.8); 7.953 (0.8); 7.936 (7.5); 7.921 (3.3); 7.919 (3.1); 7.900 (0.8); 7.897 (0.8); 7.879 (10.2); 7.814 (7.5); 7.809 (7.7); 7.412 (1.7); 7.406 (1.7); 7.396 (2.8); 7.390 (3.0); 7.380 (1.5); 7.374 (1.7); 6.638 (7.8); 6.632 (7.9); 5.754 (0.8); 5.592 (16.0); 5.452 (11.8); 3.318 (215.3); 3.176 (0.4); 3.163 (0.4); 3.163 (0.3); 2.737 (0.6); 2.725 (0.7); 2.714 (15.6); 2.702 (15.6); 2.680 (0.6); 2.675 (1.1); 2.671 (1.4); 2.666 (1.1); 2.524 (3.3); 2.519 (5.2); 2.511 (85.6); 2.506 (182.0); 2.502 (245.7); 2.497 (176.5); 2.493 (83.9); 2.333 (1.1); 2.328 (1.5); 2.324 (1.1); 2.320 (0.5); 1.352 (2.4); 1.336 (0.6); 1.299 (0.3); 1.259 (0.6); 1.250 (0.8); 1.235 (3.2); 0.854 (0.4); 0.008 (0.5); 0.000 (15.5); −0.009 (0.5)

### Example I-1-049

¹H-NMR (400.0 MHz, d₆-DMSO): δ=9.779 (2.5); 9.762 (2.5); 8.313 (0.4); 7.932 (0.6); 7.917 (4.5); 7.913 (3.0); 7.903 (2.2); 7.900 (2.0); 7.881 (0.6); 7.872 (6.4); 7.489 (4.9); 7.403 (1.2); 7.396 (1.2); 7.387 (1.7); 7.380 (1.9); 7.371 (1.0); 7.364 (1.2); 7.213 (5.4); 5.754 (0.3); 5.520 (0.5); 5.442 (7.9); 5.421 (12.4); 3.317 (133.0); 2.675 (0.7); 2.671 (0.9); 2.666 (0.7); 2.662 (0.3); 2.524 (2.1); 2.519 (3.2); 2.511 (54.0); 2.506 (118.0); 2.502 (161.4); 2.497 (116.7); 2.492 (55.4); 2.333 (0.7); 2.328 (1.0); 2.324 (0.7); 2.074 (0.7); 2.029 (16.0); 1.235 (0.7); 1.045 (0.4); 1.030 (0.4); 0.000 (1.3)

### Example I-1-050

¹H-NMR (400.0 MHz, d₆-DMSO): δ=9.784 (3.6); 9.768 (3.6); 7.944 (0.9); 7.926 (7.7); 7.911 (3.5); 7.889 (0.9); 7.872 (8.5); 7.643 (0.3); 7.619 (5.9); 7.409 (1.4); 7.404 (1.4); 7.393 (2.4); 7.387 (2.6); 7.377 (1.3); 7.371 (1.4); 7.130 (6.0); 7.015 (0.5); 6.887 (6.0); 5.450 (12.9); 5.361 (16.0); 3.323 (27.6); 3.175 (0.8); 3.164 (0.8); 2.671 (0.5); 2.503 (81.9); 2.328 (0.5); 1.352 (2.4); 1.336 (0.6); 1.299 (0.3); 1.257 (0.6); 1.249 (0.8); 1.232 (3.5); 0.852 (0.4); 0.000 (4.1)

### Example I-1-051

¹H-NMR (400.0 MHz, d₆-DMSO): δ=9.755 (3.7); 9.739 (3.6); 8.313 (0.4); 7.958 (0.8); 7.942 (7.0); 7.939 (6.2); 7.926 (3.0); 7.923 (2.8); 7.904 (0.7); 7.901 (0.7); 7.872 (9.4); 7.824 (6.0); 7.780 (4.0); 7.777 (5.3); 7.420 (1.6); 7.414 (1.6); 7.404 (2.7); 7.398 (2.9); 7.388 (1.4); 7.382 (1.5); 5.456 (12.3); 5.448 (16.0); 3.319 (110.8); 2.675 (0.8); 2.671 (1.1); 2.667 (0.8); 2.662 (0.4); 2.524 (2.7); 2.520 (4.2); 2.511 (65.7); 2.507 (138.3); 2.502 (184.8); 2.497 (132.4); 2.493 (62.7); 2.338 (0.4); 2.333 (0.8); 2.329 (1.1); 2.324 (0.8); 1.352 (0.8); 1.250 (0.3); 1.235 (1.3); 1.046 (0.3); 0.008 (0.3); 0.000 (12.2); −0.008 (0.4)

### Example I-1-052

¹H-NMR (400.0 MHz, d₆-DMSO): δ=9.777 (1.2); 9.761 (1.2); 7.924 (2.1); 7.922 (2.3); 7.917 (1.2); 7.909 (1.1); 7.906 (1.0); 7.874 (3.0); 7.748 (2.3); 7.742 (2.4); 7.407 (0.6); 7.400 (0.5); 7.391 (0.7); 7.384 (0.7); 7.376 (0.5); 7.369 (0.5); 6.255 (2.6); 6.249 (2.6); 5.463 (5.6); 5.444 (3.6); 3.320 (47.2); 2.671 (0.4); 2.529 (0.5); 2.524 (1.0); 2.520 (1.4); 2.511 (24.4); 2.507 (52.9); 2.502 (71.8); 2.497 (51.0); 2.493 (23.8); 2.406 (16.0); 2.329 (0.4); 2.324 (0.3); 0.000 (0.6)

### Example I-1-053

¹H-NMR (400.0 MHz, d₆-DMSO): δ=9.762 (5.7); 9.746 (5.8); 9.474 (3.3); 8.326 (0.3); 8.005 (1.1); 8.003 (1.2); 7.987 (10.7); 7.984 (9.0); 7.972 (4.7); 7.969 (4.5); 7.950 (1.2); 7.947 (1.2); 7.869 (150.2); 7.577 (5.5); 7.571 (5.0); 7.557 (8.0); 7.553 (7.3); 7.499 (0.4); 7.477 (2.8); 7.471 (2.7); 7.461 (4.9); 7.455 (6.6); 7.444 (9.7); 7.439 (7.7); 7.435 (5.4); 7.429 (13.0); 7.425 (13.5); 7.417 (3.1); 7.413 (1.6); 7.401 (0.8); 7.396 (0.7); 7.298 (0.4); 5.756 (4.1); 5.424 (16.0); 4.295 (6.0); 4.251 (5.9); 4.024 (0.4); 4.010 (0.4); 3.360 (46.0); 2.970 (0.7); 2.678 (0.5); 2.673 (0.7); 2.669 (0.5); 2.526 (1.6); 2.522 (2.4); 2.513 (41.3); 2.509 (88.9); 2.504 (120.1); 2.499 (86.2); 2.495 (40.7); 2.335 (0.5); 2.331 (0.7); 2.326 (0.5); 1.235 (0.8); 0.146 (0.6); 0.008 (4.4); 0.000 (139.0); −0.009 (4.5); −0.150 (0.6)

### Example I-1-054

¹H-NMR (400.0 MHz, d₆-DMSO): δ=9.796 (3.0); 9.779 (3.0); 8.546 (3.8); 8.540 (3.8); 8.314 (1.3); 7.877 (1.3); 7.874 (1.4); 7.868 (2.4); 7.861 (2.8); 7.856 (3.1); 7.853 (2.3); 7.847 (2.8); 7.841 (2.9); 7.837 (2.5); 7.834 (2.2); 7.772 (3.6); 7.750 (2.5); 7.741 (4.7); 7.736 (4.7); 7.522 (4.5); 7.502 (4.1); 7.429 (4.8); 7.426 (4.7); 7.387 (1.6); 7.384 (1.7); 7.368 (3.0); 7.352 (1.5); 7.349 (1.5); 6.275 (3.3); 6.269 (5.4); 6.264 (3.2); 5.533 (16.0); 5.296 (10.4); 3.329 (37.4); 2.527 (0.5); 2.513 (11.6); 2.509 (23.8); 2.505 (31.6); 20.500 (22.9); 2.496 (11.1); 0.008 (0.6); 0.000 (17.9); −0.009 (0.7)

### Example I-1-055

¹H-NMR (400.0 MHz, d₆-DMSO): δ=9.820 (3.3); 9.803 (3.2); 7.932 (0.6); 7.930 (0.7); 7.917 (5.9); 7.915 (6.2); 7.911 (3.7); 7.909 (3.4); 7.902 (2.9); 7.899 (2.8); 7.880 (0.6); 7.877 (0.7); 7.868 (8.2); 7.855 (0.4); 7.573 (0.7); 7.406 (1.6); 7.399 (1.5); 7.390 (2.2); 7.383 (2.3); 7.374 (1.3); 7.368 (1.4); 7.105 (3.4); 7.099 (1.2); 7.094 (0.5); 7.088 (1.4); 7.083 (6.4); 7.072 (0.7); 7.067 (1.3); 7.061 (4.6); 7.051 (0.5); 6.947 (0.4); 6.937 (4.7); 6.931 (1.5); 6.926 (4.8); 6.920 (2.3); 6.914 (3.6); 6.909 (1.4); 6.903 (3.5); 6.522 (0.4); 6.501 (0.3); 5.433 (10.2); 5.433 (1 (0.0.); 5.416 (0.7); 220 (16.0); 4.795 (0.9); 4.584 (0.7); 4.569 (0.6); 4.040 (0.4); 4.022 (0.4); 3.322 (33.9); 2.673 (0.3); 2.527 (0.7); 2.522 (1.1); 2.513 (18.9); 2.509 (40.4); 2.504 (55.0); 2.500 (39.9); 2.495 (19.5); 2.331 (0.3); 1.990 (1.7); 1.232 (0.4); 1.195 (0.5); 1.177 (0.9); 1.159 (0.5); 0.008 (2.0); 0.000 (60.8); −0.009 (2.3)

### Example I-1-056

¹H-NMR (400.0 MHz, d₆-DMSO): δ=10.322 (2.7); 9.784 (1.9); 9.768 (1.9); 7.942 (0.5); 7.925 (3.9); 7.922 (3.7); 7.909 (1.7); 7.907 (1.7); 7.888 (0.6); 7.885 (0.6); 7.875 (5.2); 7.604 (2.9); 7.598 (2.9); 7.407 (0.9); 7.401 (0.9); 7.390 (1.5); 7.385 (1.7); 7.374 (0.8); 7.368 (0.9); 6.472 (3.1); 6.466 (3.2); 5.442 (6.2); 5.386 (7.5); 4.343 (1.0); 4.332 (1.1); 3.794 (00.4); 3.783 (0.4); 3.779 (0.5); 3.763 (0.4); 3.753 (0.3); 3.321 (66.9); 2.676 (0.4); 2.672 (0.5); 2.667 (0.4); 2.525 (1.3); 2.520 (2.0); 2.511 (30.0); 2.507 (63.4); 2.502 (85.6); 2.498 (62.4); 2.494 (30.5); 2.334 (0.4); 2.329 (0.5); 2.325 (0.4); 1.981 (0.5); 1.967 (16.0); 1.235 (3.8); 1.046 (9.9); 1.030 (9.8); 0.854 (0.4); 0.000 (5.6)

### Example I-1-057

¹H-NMR (400.0 MHz, d₆-DMSO): δ=9.753 (3.9); 9.737 (3.9); 7.985 (4.1); 7.982 (4.3); 7.949 (0.6); 7.935 (7.9); 7.927 (4.3); 7.924 (4.0); 7.921 (3.5); 7.902 (0.5); 7.899 (0.5); 7.876 (9.6); 7.411 (1.7); 7.403 (1.6); 7.397 (1.8); 7.395 (2.0); 7.389 (1.9); 7.387 (2.0); 7.380 (1.5); 7.373 (1.6); 6.747 (4.8); 6.742 (4.9); 5.659 (0.5); 5.633 (16.0); 5.453 (12.2); 3.320 (65.3); 2.677 (0.4); 2.672 (0.6); 2.668 (0.4); 2.525 (1.4); 2.521 (2.0); 2.512 (33.8); 2.508 (72.4); 2.503 (97.9); 2.498 (70.3); 2.494 (33.3); 2.334 (0.4); 2.330 (0.6); 2.325 (0.4); 1.235 (0.6); 0.000 (7.6)

### Example I-1-058

¹H-NMR (400.0 MHz, d₆-DMSO): δ=9.757 (1.9); 9.740 (1.9); 8.009 (0.5); 7.987 (3.4); 7.985 (3.3); 7.971 (1.5); 7.968 (1.5); 7.949 (0.5); 7.946 (0.6); 7.875 (4.8); 7.861 (0.5); 7.475 (0.8); 7.470 (0.9); 7.458 (1.5); 7.453 (1.6); 7.442 (0.8); 7.437 (0.8); 5.756 (1.1); 5.436 (5.8); 5.411 (0.5); 4.312 (7.5); 3.324 (23.6); 2.673 (0.4); 2.668 (0.4); 2.653 (16.0); 2.526 (0.9); 2.521 (1.2); 2.513 (18.3); 2.508 (38.8); 2.504 (52.2); 2.499 (37.8); 2.495 (18.2); 2.356 (3.8); 2.330 (0.3); 1.046 (3.5); 1.031 (3.5); 0.008 (1.4); 0.000 (45.5); −0.009 (1.7)

### Example I-1-059

¹H-NMR (400.0 MHz, d₆-DMSO): δ=9.842 (2.7); 9.825 (2.8); 9.136 (7.1); 8.875 (16.0); 7.882 (0.7); 7.879 (0.8); 7.865 (0.7); 7.861 (2.1); 7.858 (2.1); 7.843 (2.4); 7.840 (2.6); 7.832 (2.9); 7.830 (3.2); 7.828 (2.8); 7.811 (1.1); 7.396 (1.4); 7.392 (1.4); 7.379 (2.5); 7.375 (2.5); 7.362 (1.4); 7.358 (1.3); 7.283 (0.4); 7.276 (3.2); 7.272 (1.1); 7.267 (0.5); 7.256 (40.3); 7.254 (3.8); 7.236 (4.0); 7.229 (0.5); 6.928 (1.6); 6.926 (2.1); 6.919 (4.7); 6.917 (4.8); 6.912 (3.6); 6.909 (4.3); 6.902 (1.7); 6.895 (5.4); 5.310 (9.2); 5.243 (15.1); 3.320 (75.9); 3.177 (0.8); 3.163 (0.7); 2.891 (1.1); 2.732 (1.0); 2.676 (0.4); 2.671 (0.5); 2.667 (0.4); 2.525 (1.1); 2.520 (1.7); 2.511 (28.4); 2.507 (61.5); 2.502 (83.6); 2.498 (59.9); 2.493 (28.2); 2.334 (0.4); 2.329 (0.5); 2.324 (0.4); 1.234 (2.1); 0.008 (0.4); 0.000 (16.8); −0.009 (0.5)

### Example I-1-060

¹H-NMR (400.0 MHz, d₆-DMSO): δ=9.740 (4.2); 9.724 (4.2); 8.853 (0.7); 8.560 (10.0); 8.538 (6.0); 8.534 (6.1); 8.313 (0.6); 8.151 (0.7); 8.063 (9.9); 7.952 (0.8); 7.895 (1.9); 7.876 (4.1); 7.857 (5.9); 7.842 (3.7); 7.836 (3.6); 7.784 (5.1); 7.762 (3.5); 7.522 (5.2); 7.501 (4.8); 7.399 (2.5); 7.382 (4.2); 7.364 (2.3); 6.570 (0.7); 5.612 (16.0); 5.290 (13.8); 3.318 (265.9); 2.891 (3.5); 2.731 (3.3); 2.671 (2.3); 2.502 (384.2); 2.328 (2.4); 1.234 (0.7); 0.146 (0.6); 0.000 (127.2); −0.150 (0.7)

### Example I-1-061

¹H-NMR (400.0 MHz, d₆-DMSO): δ=9.913 (2.6); 9.897 (2.7); 9.120 (7.1); 9.042 (0.4); 8.851 (0.7); 8.841 (15.6); 8.750 (0.9); 7.849 (0.8); 7.846 (0.9); 7.832 (0.7); 7.828 (2.2); 7.824 (2.2); 7.810 (2.5); 7.807 (2.6); 7.800 (2.9); 7.798 (3.2); 7.796 (2.8); 7.778 (1.0); 7.383 (1.4); 7.378 (1.4); 7.366 (2.5); 7.362 (2.4); 7.349 (1.3); 7.345 (1.3); 5.756 (2.9); 5.291 (00.4); 5.269 (9.5); 4.803 (0.7); 4.582 (16.0); 4.501 (0.4); 4.486 (0.4); 3.482 (4.5); 3.465 (9.4); 3.449 (4.6); 3.337 (23.8); 3.329 (19.1); 2.528 (0.4); 2.523 (0.7); 2.515 (11.9); 2.510 (25.7); 2.506 (34.9); 2.501 (25.2); 2.497 (12.1); 1.602 (0.5); 1.583 (2.1); 1.566 (4.2); 1.548 (4.4); 1.531 (2.2); 1.513 (0.6); 1.354 (0.8); 1.232 (1.1); 1.226 (1.0); 0.912 (7.7); 0.894 (15.4); 0.875 (6.9); 0.000 (9.9); −0.009 (0.3)

### Example I-1-062

¹H-NMR (400.0 MHz, d₆-DMSO): δ=9.818 (1.9); 9.802 (1.9); 7.900 (0.6); 7.895 (0.6); 7.891 (0.4); 7.878 (3.6); 7.875 (3.7); 7.859 (7.0); 7.840 (0.4); 7.837 (0.4); 7.370 (0.9); 7.364 (0.9); 7.354 (1.5); 7.348 (1.6); 7.337 (0.8); 7.332 (0.8); 5.411 (6.3); 4.641 (0.6); 4.574 (7.1); 4.341 (0.5); 4.330 (0.5); 3.319 (54.1); 2.892 (0.4); 2.886 (0.6); 2.879 (0.7); 2.869 (1.2); 2.859 (0.7); 2.853 (0.6); 2.672 (0.4); 2.525 (0.8); 2.521 (1.2); 2.512 (19.9); 2.508 (42.5); 2.503 (57.7); 2.498 (41.9); 2.494 (20.2); 2.330 (0.3); 2.184 (16.0); 1.871 (1.1); 1.352 (0.8); 1.234 (1.1); 1.046 (3.3); 1.031 (3.3); 0.786 (1.8); 0.777 (1.4); 0.768 (1.9); 0.754 (1.5); 0.743 (2.5); 0.008 (0.8); 0.000 (27.5); −0.009 (1.0)

### Example I-1-063

¹H-NMR (400.0 MHz, d₆-DMSO): δ=9.998 (2.1); 9.980 (2.2); 8.515 (2.6); 8.509 (2.6); 8.343 (2.8); 7.866 (1.6); 7.859 (1.6); 7.845 (1.8); 7.839 (1.8); 7.728 (1.6); 7.724 (1.6); 7.711 (1.5); 7.707 (1.6); 7.520 (3.1); 7.499 (2.8); 5.754 (1.1); 5.360 (7.3); 4.825 (16.0); 4.269 (1.6); 4.094 (0.4); 4.081 (0.5); 3.568 (0.8); 3.320 (62.5); 3.176 (2.7); 3.163 (2.6); 2.671 (0.4); 2.525 (0.9); 2.520 (1.5); 2.511 (23.5); 2.507 (50.0); 2.502 (67.4); 2.498 (48.6); 2.493 (23.1); 2.329 (0.4); 0.008 (1.2); 0.000 (41.0); −0.009 (1.5)

### Example I-1-064

¹H-NMR (400.0 MHz, d₆-DMSO): δ=9.734 (3.2); 9.718 (3.2); 8.110 (6.9); 8.109 (7.5); 7.956 (0.6); 7.941 (6.5); 7.936 (3.6); 7.929 (3.0); 7.926 (2.8); 7.907 (0.5); 7.904 (0.5); 7.880 (8.0); 7.755 (6.8); 7.753 (7.2); 7.416 (1.4); 7.408 (1.4); 7.400 (1.9); 7.393 (2.0); 7.385 (1.3); 7.378 (1.4); 5.812 (16.0); 5.754 (1.5); 5.460 (10.6); 3.316 (125.0); 3.175 (0.7); 3.162 (0.6); 2.675 (0.7); 2.670 (1.0); 2.666 (0.8); 2.524 (2.1); 2.519 (3.3); 2.5101 (168.8); 2.497 (123.4); 2.492 (59.9); 2.337 (0.3); 2.332 (0.7); 2.328 (1.0); 2.323 (0.8); 1.234 (0.6); −0.001 (1.2)

### Example I-1-065

¹H-NMR (400.0 MHz, d₆-DMSO): δ=9.742 (2.9); 9.726 (2.9); 9.135 (7.1); 8.875 (16.0); 8.855 (1.4); 8.557 (8.0); 8.313 (0.6); 8.183 (0.6); 8.151 (1.3); 8.061 (7.7); 7.953 (0.4); 7.912 (0.9); 7.909 (0.9); 7.891 (2.3); 7.888 (2.1); 7.873 (2.4); 7.870 (2.4); 7.856 (3.5); 7.836 (1.3); 7.405 (1.4); 7.401 (1.4); 7.388 (2.5); 7.384 (2.4); 7.371 (1.3); 7.367 (1.3); 6.573 (10.4); 5.611 (13.4); 5.320 (9.6); 3.323 (10.8); 2.892 (2.2); 2.732 (1.9); 2.672 (0.4); 2.525 (0.8); 2.512 (21.0); 2.507 (44.5); 2.503 (60.3); 2.498 (44.3); 2.494 (22.0); 2.330 (0.4); 0.008 (0.5); 0.000 (20.1); −0.009 (1.0)

### Example I-1-066

¹H-NMR (400.0 MHz, d₆-DMSO): δ=9.797 (2.8); 9.781 (2.9); 9.136 (7.2); 8.877 (16.0); 8.313 (5.6); 7.892 (0.7); 7.889 (0.8); 7.870 (2.2); 7.867 (2.3); 7.853 (2.6); 7.850 (3.0); 7.844 (3.2); 7.842 (3.5); 7.823 (1.1); 7.735 (4.3); 7.729 (4.3); 7.598 (0.4); 7.425 (4.4); 7.420 (4.3); 7.393 (1.4); 7.388 (1.4); 7.376 (2.6); 7.371 (2.5); 7.359 (1.3); 7.355 (1.3); 6.271 (3.2); 6.265 (5.3); 6.260 (3.2); 6.252 (0.6); 6.247 (0.3); 5.527 (15.7); 5.323 (9.9); 3.322 (37.9); 3.299 (1.5); 2.891 (0.8); 2.732 (0.7); 2.672 (0.3); 2.525 (0.7); 2.511 (19.6); 2.507 (42.1); 2.503 (57.2); 2.498 (41.6); 2.494 (20.1); 2.329 (0.3); 0.008 (0.5); 0.000 (18.1); −0.009 (0.7)

### Example I-1-067

¹H-NMR (400.0 MHz, d₆-DMSO): δ=9.903 (3.3); 9.886 (3.4); 8.531 (3.1); 8.526 (3.2); 8.506 (3.9); 8.504 (3.9); 7.885 (1.8); 7.879 (1.7); 7.864 (2.0); 7.858 (2.0); 7.771 (2.3); 7.767 (2.3); 7.753 (2.2); 7.749 (2.2); 7.521 (3.7); 7.500 (3.3); 5.272 (8.6); 4.829 (16.0); 3.318 (49.9); 2.671 (0.4); 2.507 (47.0); 2.502 (61.6); 2.498 (44.8); 2.329 (0.4); 0.008 (0.5); 0.000 (13.3)

### Example I-1-068

¹H-NMR (400.0 MHz, d₆-DMSO): δ=9.716 (2.0); 9.714 (1.2); 9.700 (1.9); 7.936 (3.4); 7.933 (3.9); 7.928 (2.0); 7.926 (2.0); 7.922 (2); 7.919 (1.8); 7.878 (4.8); 7.818 (16.0); 7.408 (0.8); 7.399 (0.8); 7.394 (0.9); 7.391 (1.0); 7.386 (0.9); 7.383 (1.0); 7.377 (0.8); 7.369 (0.8); 5.828 (10.7); 5.755 (4.8); 5.451 (5.8); 3.321 (27.6); 2.526 (0.4); 2.521 (0.7); 2.513 (11.5); 2.508 (25.0); 2.504 (34.1); 2.499 (24.6); 2.494 (11.6); 1.353 (0.6); 1.233 (1.0); 0.000 (0.3)

### Example I-1-069

¹H-NMR (400.0 MHz, d₆-DMSO): δ=10.039 (2.9); 10.021 (3.0); 8.510 (3.6); 8.504 (3.7); 8.295 (3.8); 7.852 (2.3); 7.846 (2.3); 7.832 (2.6); 7.825 (2.6); 7.691 (2.2); 7.686 (2.2); 7.673 (2.1); 7.669 (20.1); 7.514 (4.4); 7.493 (3.9); 5.341 (9.7); 4.556 (16.0); 3.931 (0.5); 3.813 (0.5); 3.370 (39.8); 3.323 (35.9); 3.292 (1.1); 2.673 (0.4); 2.526 (1.0); 2.521 (1.5); 2.513 (22.5); 2.508 (47.5); 2.503 (63.7); 2.499 (45.2); 2.494 (21.1); 2.330 (0.4); 1.234 (0.4); 1.046 (6.0); 1.031 (5.9); 0.000 (0.5)

### Example I-1-070

¹H-NMR (400.0 MHz, d₆-DMSO): δ=9.868 (2.8); 9.853 (3.3); 9.842 (2.3); 8.530 (5.9); 8.321 (0.5); 7.864 (1.3); 7.853 (2.3); 7.845 (4.7); 7.833 (3.6); 7.825 (4.6); 7.815 (3.2); 7.794 (1.4); 7.751 (3.3); 7.741 (2.8); 7.730 (2.3); 7.719 (1.6); 7.523 (4.3); 7.502 (3.9); 7.387 (1.5); 7.370 (2.9); 7.349 (2.5); 7.331 (1.0); 5.761 (1.8); 5.273 (16.0); 4.693 (10.5); 4.599 (8.1); 4.351 (0.4); 4.339 (0.4); 4.104 (0.6); 4.091 (0.6); 3.397 (1.2); 3.380 (3.4); 3.362 (4.4); 3.330 (328.6); 3.299 (2.4); 3.183 (3.2); 3.170 (3.1); 2.678 (1.7); 2.509 (291.4); 2.506 (223.7); 2.432 (1.4); 2.414 (3.8); 2.395 (3.9); 2.377 (1.3); 2.336 (1.7); 2.203 (1.5); 2.185 (4.8); 2.166 (4.9); 2.148 (1.6); 1.359 (0.4); 1.242 (1.1); 1.131 (3.6); 1.114 (7.6); 1.096 (3.5); 1.053 (3.6); 1.038 (7.5); 1.019 (11.3); 1.000 (13.3); 0.982 (4.9); 0.966 (5.4); 0.948 (10.6); 0.929 (4.9); 0.007 (1.0)

### Example I-1-071

¹H-NMR (400.0 MHz, d₆-DMSO): δ=9.871 (1.6); 9.855 (1.7); 9.845 (1.4); 9.829 (1.3); 8.526 (3.3); 8.520 (3.4); 8.311 (0.4); 7.863 (0.7); 7.860 (0.7); 7.847 (1.6); 7.845 (1.5); 7.841 (2.9); 7.838 (2.2); 7.830 (1.8); 7.826 (2.3); 7.822 (1.9); 7.819 (2.8); 7.816 (1.6); 7.809 (2.2); 7.805 (1.1); 7.790 (1.0); 7.786 (0.9); 7.747 (1.9); 7.734 (1.7); 7.726 (1.4); 7.712 (1.0); 7.514 (2.5); 7.507 (2.1); 7.494 (2.3); 7.487 (1.9); 7.390 (0.9); 7.387 (0.9); 7.371 (1.6); 7.365 (0.9); 7.362 (0.8); 7.355 (1.0); 7.352 (1.0); 7.346 (1.3); 7.330 (0.7); 7.327 (0.6); 5.752 (1.2); 5.268 (6.8); 4.685 (7.0); 4.585 (5.5); 4.522 (0.4); 3.391 (0.7); 3.373 (2.2); 3.356 (2.7); 3.328 (116.3); 3.309 (3.3); 3.291 (2.7); 3.273 (0.9); 2.677 (0.4); 2.673 (0.5); 2.668 (0.4); 2.526 (1.2); 2.521 (2.0); 2.513 (29.9); 2.508 (63.4); 2.504 (85.2); 2.499 (60.6); 2.495 (28.4); 2.335 (0.4); 2.331 (0.5); 2.326 (0.4); 2.078 (1.6); 2.069 (12.0); 1.891 (16.0); 1.234 (0.5); 1.134 (2.5); 1.116 (5.7); 1.098 (2.4); 1.008 (3.3); 0.990 (7.5); 0.972 (3.2); 0.000 (0.8)

### Example I-1-072

¹H-NMR (400.0 MHz, d₆-DMSO): δ=9.907 (2.5); 9.891 (2.6); 9.119 (6.5); 8.838 (14.6); 8.742 (0.6); 7.847 (0.7); 7.844 (0.8); 7.830 (0.7); 7.826 (2.0); 7.822 (2.0); 7.808 (2.3); 7.805 (2.3); 7.796 (2.7); 7.794 (3.0); 7.792 (2.6); 7.774 (1.0); 7.383 (1.3); 7.379 (1.3); 7.366 (2.4); 7.362 (2.3); 7.349 (1.3); 7.345 (1.2); 5.755 (1.5); 5.265 (9.0); 4.576 (16.0); 4.565 (0.4); 4.519 (1.3); 3.582 (2.1); 3.564 (7.3); 3.547 (7.5); 3.529 (2.4); 3.318 (38.0); 2.676 (0.3); 2.672 (0.5); 2.667 (0.4); 2.525 (1.0); 2.520 (1.5); 2.512 (27.6); 2.507 (59.4); 2.503 (80.6); 2.498 (57.4); 2.493 (26.9); 2.334 (0.3); 2.329 (0.5); 2.325 (0.3); 1.234 (0.9); 1.172 (7.4); 1.166 (0.9); 1.154 (15.2); 1.148 (1.7); 1.137 (7.2); 1.131 (0.8); 0.008 (0.4); 0.000 (12.3); −0.009 (0.4)

### Example I-1-073

¹H-NMR (400.0 MHz, d₆-DMSO): δ=9.805 (3.4); 9.788 (3.4); 7.936 (0.8); 7.921 (7.2); 7.906 (3.0); 7.884 (0.7); 7.882 (0.7); 7.870 (8.5); 7.853 (0.5); 7.407 (1.6); 7.400 (1.5); 7.391 (2.4); 7.384 (2.5); 7.375 (1.3); 7.368 (1.4); 7.301 (2.2); 7.294 (0.5); 7.280 (4.9); 7.269 (0.7); 7.259 (2.9); 6.986 (6.6); 6.981 (5.3); 6.976 (3.6); 6.971 (3.1); 6.908 (2.3); 6.903 (2.1); 6.888 (2.1); 6.882 (1.9); 5.755 (0.5); 5.437 (11.4); 5.414 (0.7); 5.276 (16.0); 4.794 (1.1); 3.320 (73.4); 2.676 (0.5); 2.672 (0.6); 2.668 (0.5); 2.525 (1.8); 2.507 (84.6); 2.503 (110.9); 2.498 (79.8); 2.334 (0.5); 2.330 (0.7); 2.325 (0.5); 1.234 (0.9); 1.046 (1.3); 1.031 (1.3); 0.146 (0.6); 0.008 (5.2); 0.000 (128.3); −0.008 (4.9); −0.150 (0.6)

### Example I-1-074

¹H-NMR (400.0 MHz, d₆-DMSO): δ=9.803 (3.4); 9.786 (3.4); 9.649 (0.4); 7.932 (0.7); 7.917 (6.5); 7.913 (4.0); 7.903 (3.0); 7.901 (2.8); 7.881 (0.7); 7.879 (0.7); 7.868 (8.6); 7.406 (1.6); 7.399 (1.5); 7.390 (2.2); 7.383 (2.4); 7.374 (1.3); 7.368 (1.5); 7.312 (0.7); 7.303 (8.3); 7.298 (2.6); 7.286 (2.7); 7.281 (9.5); 7.272 (0.9); 7.197 (0.4); 7.175 (0.4); 6.950 (0.9); 6.942 (9.1); 6.936 (2.8); 6.925 (2.5); 6.919 (8.1); 6.910 (0.7); 6.773 (0.4); 6.751 (0.4); 5.755 (10.0); 5.433 (10.8); 5.244 (16.0); 3.320 (51.2); 2.676 (0.4); 2.672 (0.6); 2.667 (0.5); 2.525 (1.5); 2.512 (37.6); 2.507 (78.5); 2.503 (104.9); 2.498 (75.2); 2.494 (35.6); 2.334 (0.4); 2.329 (0.6); 2.325 (0.5); 1.234 (0.8); 1.046 (1.5); 1.031 (1.5); 0.146 (0.6); 0.008 (5.5); 0.000 (151.9); −0.009 (5.5); −0.150 (0.7)

### Example I-1-075

¹H-NMR (400.0 MHz, d₆-DMSO): δ=9.858 (1.7); 9.841 (1.7); 7.887 (3.0); 7.885 (3.0); 7.879 (1.8); 7.874 (1.7); 7.871 (1.5); 7.853 (0.3); 7.842 (3.9); 7.402 (0.7); 7.394 (0.7); 7.388 (0.8); 7.386 (0.8); 7.380 (0.9); 7.372 (0.7); 7.364 (0.9); 5.390 (5.1); 4.991 (0.5); 4.764 (0.4); 4.711 (0.4); 4.688 (8.1); 4.401 (0.4); 4.285 (9.1); 3.694 (0.8); 3.671 (16.0); 3.322 (27.0); 2.527 (0.5); 2.522 (0.7); 2.513 (11.8); 2.509 (25.0); 2.504 (33.7); 2.500 (24.2); 2.495 (11.6); 0.008 (1.8); 0.000 (52.4); −0.009 (1.9)

### Example I-1-076

¹H-NMR (400.0 MHz, d₆-DMSO): δ=9.851 (1.8); 9.834 (1.9); 9.832 (1.5); 9.827 (2.0); 9.810 (1.7); 8.314 (0.4); 7.953 (0.4); 7.906 (3.4); 7.903 (3.7); 7.897 (2.1); 7.894 (2.7); 7.890 (1.9); 7.878 (3.1); 7.865 (1.6); 7.862 (1.9); 7.856 (7.2); 7.855 (7.2); 7.843 (0.6); 7.840 (0.7); 7.823 (0.5); 7.803 (0.4); 7.407 (0.8); 7.398 (0.9); 7.394 (0.8); 7.390 (0.9); 7.384 (1.6); 7.381 (1.3); 7.378 (1.4); 7.368 (2.1); 7.362 (1.8); 7.356 (1.4); 7.351 (0.9); 7.346 (1.0); 5.417 (6.9); 5.414 (6.9); 5.236 (1.0); 5.231 (1.0); 4.671 (7.4); 4.591 (7.6); 3.318 (101.6); 3.022 (15.8); 2.994 (0.6); 2.952 (9.3); 2.891 (2.8); 2.818 (14.8); 2.732 (2.3); 2.680 (0.4); 2.676 (0.8); 2.671 (1.1); 2.667 (0.8); 2.662 (0.4); 2.525 (2.7); 2.520 (4.1); 2.511 (64.6); 2.507 (137.1); 2.502 (184.3); 2.498 (131.2); 2.493 (61.3); 2.338 (0.3); 2.333 (0.8); 2.329 (1.1); 2.324 (0.8); 2.058 (16.0); 2.010 (0.5); 1.909 (0.9); 1.898 (15.3); 1.234 (0.6); 1.196 (0.5); 1.178 (1.0); 1.159 (0.5); 0.000 (2.6)

### Example I-1-077

¹H-NMR (400.0 MHz, d₆-DMSO): δ=9.910 (1.3); 9.893 (1.3); 9.122 (3.3); 8.843 (7.3); 7.865 (0.4); 7.862 (0.4); 7.848 (0.4); 7.844 (1.0); 7.841 (1.0); 7.826 (1.2); 7.823 (1.3); 7.817 (1.4); 7.814 (1.5); 7.795 (0.5); 7.385 (0.7); 7.381 (0.7); 7.368 (1.2); 7.364 (1.2); 7.351 (0.6); 7.347 (0.6); 5.757 (0.5); 5.283 (4.7); 3.814 (8.0); 3.325 (18.9); 2.526 (0.4); 2.521 (0.6); 2.513 (9.2); 2.508 (19.5); 2.504 (26.2); 2.499 (18.5); 2.495 (8.5); 2.140 (16.0); 1.235 (0.4); 0.008 (0.7); 0.000 (20.7); −0.009 (0.6)

### Example I-1-078

¹H-NMR (400.0 MHz, d₆-DMSO): δ=9.802 (3.4); 9.785 (3.4); 8.313 (2.0); 7.935 (0.8); 7.922 (6.4); 7.920 (6.7); 7.915 (4.0); 7.907 (3.2); 7.904 (2.9); 7.885 (0.7); 7.882 (0.7); 7.871 (8.9); 7.572 (0.7); 7.438 (3.2); 7.434 (3.5); 7.419 (3.5); 7.415 (3.7); 7.405 (1.9); 7.399 (1.6); 7.390 (2.3); 7.383 (2.4); 7.374 (1.4); 7.367 (1.6); 7.228 (1.5); 7.224 (1.4); 7.207 (2.5); 7.188 (2.1); 7.184 (1.9); 6.989 (2.9); 6.986 (3.4); 6.968 (2.6); 6.965 (2.7); 6.950 (2.0); 6.946 (1.8); 6.930 (3.1); 6.927 (2.9); 6.912 (1.6); 6.908 (1.5); 6.518 (0.4); 5.754 (0.5); 5.440 (10.7); 5.355 (16.0); 4.581 (0.6); 4.565 (0.6); 3.317 (488.4); 2.675 (3.6); 2.670 (5.0); 2.666 (3.7); 2.661 (1.8); 2.615 (0.4); 2.524 (13.6); 20.519 (21.6); 2.510 (297.5); 2.506 (625.0); 2.501 (836.6); 2.497 (598.2); 2.492 (283.4); 2.337 (1.6); 2.332 (3.5); 2.328 (4.9); 2.323 (3.4); 1.236 (2.4); 1.045 (0.9); 1.030 (0.8); 0.854 (0.3); 0.146 (4.7); 0.137 (0.4); 0.008 (40.6); 0.000 (1162.2); −0.009 (44.3); −0.036 (0.5); −0.150 (4.6)

### Example I-1-079

¹H-NMR (400.0 MHz, d₆-DMSO): δ=10.040 (2.7); 10.022 (2.8); 8.508 (3.5); 8.502 (3.6); 8.290 (3.7); 7.850 (2.1); 7.844 (2.1); 7.830 (2.4); 7.823 (2.4); 7.691 (2.1); 7.687 (2.1); 7.674 (2.1); 7.669 (20.1); 7.512 (4.4); 7.492 (3.9); 5.339 (9.1); 4.593 (15.1); 3.961 (0.7); 3.585 (2.3); 3.568 (7.6); 3.550 (7.7); 3.533 (2.4); 3.320 (75.3); 3.169 (1.5); 2.676 (0.6); 2.671 (0.8); 2.667 (0.6); 2.524 (1.8); 2.520 (2.9); 2.511 (47.7); 2.507 (102.5); 2.502 (139.6); 2.498 (100.8); 2.493 (47.8); 2.473 (0.7); 2.333 (0.6); 2.329 (0.9); 2.324 (0.6); 1.278 (0.3); 1.176 (7.8); 1.159 (16.0); 1.141 (7.6); 1.130 (0.3); 1.113 (0.5); 0.146 (0.8); 0.008 (6.2); 0.000 (188.9); −0.009 (6.5); −0.150 (0.8)

### Example I-1-080

¹H-NMR (400.0 MHz, d₆-DMSO): δ=7.938 (1.5); 7.917 (2.5); 7.898 (2.1); 7.864 (5.9); 7.789 (2.4); 7.768 (1.7); 7.306 (2.0); 7.287 (1.9); 5.415 (8.3); 4.915 (16.0); 4.273 (1.1); 4.040 (0.7); 4.022 (0.8); 3.332 (9.5); 2.543 (14.2); 2.527 (0.6); 2.513 (11.2); 2.509 (23.6); 2.505 (32.1); 2.500 (23.1); 2.496 (11.0); 1.991 (3.3); 1.195 (0.9); 1.177 (1.7); 1.159 (0.8); 0.008 (0.8); 0.000 (23.5); −0.008 (0.9)

### Example I-1-081

¹H-NMR (400.0 MHz, d₆-DMSO): δ=9.873 (1.6); 9.857 (1.6); 7.711 (1.6); 7.692 (6.2); 7.350 (1.7); 7.333 (2.0); 7.331 (2.0); 7.314 (1.5); 5.538 (5.9); 5.536 (5.9); 4.830 (16.0); 4.762 (0.8); 3.569 (2.5); 3.321 (29.9); 2.672 (0.4); 2.668 (0.4); 2.641 (11.1); 2.526 (0.9); 2.521 (1.3); 2.512 (15.7); 2.508 (33.2); 2.503 (44.8); 2.498 (31.9); 2.494 (14.9); 2.369 (0.5); 1.356 (0.4); 0.008 (1.4); 0.000 (38.5); −0.009 (1.3)

### Example I-1-082

¹H-NMR (400.0 MHz, d₆-DMSO): δ=9.919 (2.5); 9.902 (2.5); 7.681 (5.8); 7.648 (2.3); 7.629 (2.5); 7.311 (2.3); 7.301 (0.4); 7.293 (3.0); 7.284 (0.4); 7.275 (2.1); 7.166 (0.5); 5.518 (8.8); 5.403 (0.8); 4.561 (15.9); 3.958 (2.6); 3.372 (49.4); 3.363 (42.8); 3.359 (46.4); 3.354 (44.3); 3.345 (39.2); 3.343 (36.8); 3.339 (37.7); 2.674 (0.4); 2.666 (1.6); 2.636 (16.0); 2.528 (0.6); 2.523 (0.9); 2.523 (0.9); 2.514 (17.1); 2.510 (36.9); 2.505 (50.1); 2.501 (36.1); 2.496 (17.0); 1.990 (0.4); 0.008 (0.6); 0.000 (20.9); −0.008 (0.7)

### Example I-1-083

¹H-NMR (400.0 MHz, d₆-DMSO): δ=9.925 (2.9); 9.908 (3.0); 7.682 (6.0); 7.645 (2.4); 7.626 (2.7); 7.309 (1.9); 7.292 (3.1); 7.274 (1.7); 7.140 (0.6); 5.755 (0.7); 5.519 (9.5); 5.399 (1.0); 4.599 (13.1); 4.355 (0.6); 4.338 (0.6); 4.338 (0.6); 3.591 (1.9); 3.573 (5.9); 3.556 (6.0); 3.538 (2.0); 3.323 (29.6); 2.664 (1.8); 2.636 (16.0); 2.507 (49.4); 2.504 (53.9); 2.331 (0.3); 2.039 (0.3); 1.327 (0.6); 1.309 (1.2); 1.292 (0.6); 1.181 (6.1); 1.163 (12.1); 1.146 (5.9); 0.002 (18.0); 0.000 (23.7)

### Example I-1-084

¹H-NMR (400.0 MHz, d₆-DMSO): δ=9.776 (3.5); 9.760 (3.4); 8.112 (4.7); 8.104 (4.7); 7.935 (0.6); 7.922 (6.2); 7.920 (6.9); 7.914 (3.8); 7.908 (3.3); 7.905 (3.1); 7.886 (0.5); 7.883 (0.5); 7.864 (8.3); 7.450 (2.0); 7.442 (1.8); 7.428 (4.3); 7.420 (4.4); 7.404 (1.6); 7.391 (7.5); 7.380 (2.1); 7.370 (3.4); 7.366 (1.7); 5.754 (1.3); 5.433 (10.6); 5.361 (0.3); 5.341 (16.0); 3.322 (109.3); 2.676 (0.5); 2.672 (0.7); 2.667 (0.6); 2.525 (1.6); 2.512 (43.3); 2.507 (93.4); 2.503 (128.0); 2.498 (92.9); 2.494 (44.6); 2.334 (0.5); 2.330 (0.7); 2.325 (0.6); 1.046 (0.7); 1.031 (0.7); 0.008 (2.2); 0.000 (71.5); −0.009 (2.5)

### Example I-1-085

¹H-NMR (400.0 MHz, d₆-DMSO): δ=9.473 (1.6); 9.456 (1.7); 7.842 (4.7); 7.826 (2.2); 7.786 (1.1); 7.783 (1.1); 7.768 (1.3); 7.765 (1.6); 7.746 (0.7); 7.743 (0.7); 7.358 (0.9); 7.355 (0.9); 7.338 (1.6); 7.323 (0.8); 7.320 (0.8); 5.377 (6.7); 3.741 (16.0); 3.326 (19.9); 2.515 (8.0); 2.511 (16.4); 2.506 (21.8); 2.502 (15.8); 2.498 (7.7); 1.048 (1.0); 1.033 (1.0); 0.000 (0.4)

### Example I-1-086

¹H-NMR (400.0 MHz, d₆-DMSO): δ=9.803 (1.5); 9.786 (1.5); 7.916 (2.7); 7.913 (3.1); 7.904 (2.7); 7.859 (3.8); 7.406 (0.6); 7.397 (0.7); 7.394 (0.7); 7.390 (0.7); 7.385 (0.7); 7.381 (0.7); 7.377 (0.7); 7.368 (0.6); 7.148 (2.8); 7.143 (3.0); 5.754 (0.7); 5.552 (3.0); 5.547 (3.1); 5.421 (4.9); 5.260 (7.6); 3.623 (16.0); 3.322 (21.0); 2.525 (0.5); 2.512 (14.0); 2.508 (30.2); 2.503 (41.6); 2.498 (30.5); 2.494 (14.9); 0.008 (0.7); 0.000 (23.4); −0.008 (0.9)

### Example I-1-087

¹H-NMR (400.0 MHz, d₆-DMSO): δ=9.885 (3.1); 9.868 (3.1); 7.892 (0.6); 7.879 (5.6); 7.877 (6.0); 7.871 (3.4); 7.865 (2.9); 7.862 (2.7); 7.845 (7.5); 7.395 (1.3); 7.387 (1.3); 7.379 (2.0); 7.372 (1.8); 7.364 (1.3); 7.356 (1.3); 5.754 (0.6); 5.392 (9.8); 5.256 (0.3); 4.608 (16.0); 3.667 (3.9); 3.656 (5.4); 3.651 (3.7); 3.643 (5.4); 3.506 (5.3); 3.497 (3.5); 3.493 (5.3); 3.482 (3.9); 3.318 (54.0); 3.289 (0.4); 3.270 (36.0); 3.251 (1.4); 3.244 (0.4); 2.676 (0.4); 2.672 (0.6); 2.667 (0.5); 2.525 (1.3); 2.511 (36.8); 2.507 (78.2); 2.503 (105.6); 2.498 (76.9); 2.494 (37.3); 2.334 (0.5); 2.329 (0.6); 2.325 (0.5); 1.233 (0.5); 0.146 (0.6); 0.008 (4.6); −0.001 (139.2); −0.009 (5.4); −0.150 (0.6)

### Example I-1-088

¹H-NMR (400.0 MHz, d₆-DMSO): δ=9.751 (3.7); 9.734 (3.8); 7.941 (1.2); 7.919 (4.9); 7.915 (5.2); 7.897 (3.4); 7.895 (3.4); 7.880 (9.4); 7.847 (0.4); 7.634 (2.8); 7.630 (3.0); 7.617 (3.0); 7.613 (2.9); 7.469 (1.6); 7.464 (1.6); 7.452 (1.9); 7.447 (2.9); 7.441 (1.8); 7.429 (2.0); 7.425 (1.7); 7.416 (0.4); 7.410 (0.5); 7.400 (2.0); 7.396 (2.1); 7.383 (3.6); 7.379 (3.7); 7.367 (1.9); 7.362 (1.9); 6.394 (3.9); 6.372 (3.7); 6.315 (0.4); 6.292 (0.4); 6.243 (1.9); 6.226 (3.7); 6.210 (1.9); 5.451 (12.1); 5.401 (0.6); 5.267 (0.5); 5.235 (16.0); 4.514 (0.4); 3.382 (170.8); 3.374 (135.6); 3.365 (143.8); 3.359 (121.7); 3.350 (180.1); 2.963 (1.0); 2.679 (0.5); 2.674 (0.7); 2.670 (0.5); 2.514 (44.4); 2.510 (84.8); 2.505 (109.4); 2.501 (81.4); 2.497 (41.9); 2.336 (0.5); 2.332 (0.7); 2.328 (0.6); 0.000 (8.4)

### Example I-1-089

¹H-NMR (601.6 MHz, d₆-DMSO): δ=9.858 (3.2); 9.847 (3.2); 8.530 (0.6); 7.915 (1.5); 7.901 (3.9); 7.892 (2.7); 7.890 (2.7); 7.880 (2.6); 7.878 (2.8); 7.875 (1.1); 7.866 (1.2); 7.864 (1.2); 7.855 (7.7); 7.411 (1.7); 7.408 (1.7); 7.399 (2.8); 7.397 (2.8); 7.388 (1.6); 7.385 (1.6); 5.407 (12.1); 4.506 (16.0); 3.405 (0.3); 3.330 (11.9); 3.255 (0.3); 3.170 (1.3); 3.094 (0.5); 3.086 (0.6); 3.082 (1.5); 3.074 (1.5); 3.070 (1.5); 3.062 (1.5); 3.058 (0.6); 3.050 (0.5); 2.616 (0.4); 2.538 (9.1); 2.528 (20.1); 2.519 (10.5); 2.510 (25.8); 2.507 (55.9); 2.504 (78.1); 2.501 (57.7); 2.498 (28.2); 2.440 (0.5); 2.388 (0.6); 2.385 (0.4); 1.233 (0.7); 1.207 (3.2); 1.195 (6.7); 1.183 (3.2); 0.005 (0.7); 0.000 (26.8); −0.006 (1.3)

### Example I-1-090

¹H-NMR (400.0 MHz, d₆-DMSO): δ=9.980 (2.9); 9.978 (2.8); 9.964 (3.1); 9.961 (2.8); 8.419 (3.3); 8.414 (3.3); 7.981 (2.8); 7.979 (2.8); 7.961 (3.1); 7.959 (2.9); 7.778 (1.7); 7.771 (1.8); 7.757 (2.1); 7.751 (2.0); 7.497 (3.5); 7.477 (3.0); 7.417 (2.1); 7.400 (2.7); 7.397 (2.5); 7.380 (2.0); 5.546 (9.2); 4.844 (16.0); 3.322 (64.8); 2.671 (0.3); 2.506 (46.4); 2.502 (57.7); 2.498 (44.3); 2.329 (0.3); 1.989 (0.4); 0.000 (6.2)

### Example I-1-091

¹H-NMR (400.0 MHz, d₆-DMSO): δ=9.702 (3.4); 8.506 (3.0); 8.500 (3.0); 7.818 (1.7); 7.811 (1.7); 7.797 (2.0); 7.791 (2.0); 7.767 (1.3); 7.763 (1.3); 7.744 (2.3); 7.741 (2.3); 7.688 (3.7); 7.666 (2.1); 7.499 (3.6); 7.479 (3.2); 5.238 (8.5); 4.798 (16.0); 3.662 (0.3); 3.571 (0.6); 3.333 (8.7); 2.517 (5.1); 2.513 (10.6); 2.508 (14.2); 2.504 (10.3); 2.500 (4.9); 2.373 (14.4); 0.000 (6.1)

### Example I-1-092

¹H-NMR (400.0 MHz, d₆-DMSO): δ=9.820 (1.8); 9.803 (2.2); 9.785 (0.5); 7.713 (5.0); 7.684 (2.2); 7.665 (2.3); 7.601 (3.0); 7.596 (3.0); 7.317 (1.7); 7.300 (2.5); 7.282 (2.4); 6.048 (0.9); 6.032 (3.1); 6.027 (3.1); 5.569 (6.6); 5.483 (2.5); 5.470 (0.6); 5.441 (9.4); 4.039 (0.4); 4.021 (0.4); 3.323 (70.2); 2.699 (0.8); 2.671 (0.7); 2.653 (13.8); 2.524 (1.1); 2.507 (56.1); 2.502 (73.5); 2.498 (52.9); 2.498 (52.9); 2.334 (0.3); 2.329 (0.4); 2.325 (0.3); 2.248 (0.8); 2.217 (3.8); 2.140 (16.0); 1.989 (1.9); 1.299 (0.4); 1.259 (0.7); 1.235 (1.3); 1.193 (0.5); 1.175 (1.0); 1.158 (0.5); 0.000 (1.1)

### Example I-1-093

¹H-NMR (400.0 MHz, d₆-DMSO): δ=9.789 (2.5); 9.772 (2.5); 7.831 (0.4); 7.822 (5.8); 7.816 (5.9); 7.711 (6.9); 7.698 (2.5); 7.679 (2.7); 7.327 (2.4); 7.309 (3.1); 7.292 (2.2); 6.343 (6.4); 6.337 (6.4); 5.567 (8.5); 5.535 (0.5); 5.527 (0.6); 5.507 (14.3); 4.925 (0.6); 3.494 (6.0); 3.390 (0.4); 2.871 (3.4); 2.693 (0.5); 2.676 (0.6); 2.671 (0.8); 2.667 (0.8); 2.651 (16.0); 2.547 (0.4); 2.532 (0.4); 2.52 (0.4); 2.524 (1.3); 2.511 (33.5); 2.507 (70.6); 2.502 (95.2); 2.498 (69.0); 2.493 (33.1); 2.334 (0.4); 2.329 (0.6); 2.324 (0.4); 2.255 (0.5); 2.075 (1.3); 1.989 (0.6); 1.910 (0.7); 1.299 (0.5); 1.259 (0.9); 1.234 (1.3); 1.180 (0.4); 1.175 (0.4); 0.000 (1.8)

### Example I-1-094

¹H-NMR (400.0 MHz, d₆-DMSO): δ=9.813 (1.2); 9.797 (1.2); 7.712 (3.0); 7.687 (1.2); 7.668 (1.2); 7.491 (0.3); 7.483 (3.1); 7.481 (3.2); 7.320 (1.1); 7.302 (1.5); 7.284 (1.0); 7.198 (3.4); 7.197 (3.4); 5.567 (3.9); 5.427 (0.5); 5.427 (0.5); 5.406 (4.056 (0.5); 4.038 (1.5); 4.021 (1.5); 4.003 (0.5); 3.771 (1.1); 3.676 (16.0); 3.320 (41.6); 2.694 (0.6); 2.671 (0.4); 2.652 (7.1); 2.524 (0.7); 2.511 (14.9); 2.506 (30.7); 2.502 (40.7); 2.497 (29.3); 2.493 (14.0); 1.989 (6.8); 1.259 (0.4); 1.234 (0.7); 1.193 (1.8); 1.175 (3.5); 1.157 (1.7); 0.000 (0.8)

### Example I-1-095

¹H-NMR (400.0 MHz, d₆-DMSO): δ=9.779 (2.2); 9.763 (2.2); 7.972 (7.0); 7.970 (7.4); 7.711 (6.5); 7.697 (2.1); 7.678 (2.3); 7.540 (7.1); 7.538 (7.3); 7.328 (2.2); 7.311 (2.6); 7.310 (2.7); 7.293 (2.0); 5.567 (7.2); 5.516 (13.1); 4.056 (1.2); 4.039 (3.6); 4.021 (3.6); 4.003 (1.2); 3.324 (125.9); 2.676 (0.5); 2.671 (0.7); 2.667 (0.7); 2.651 (14.0); 2.525 (1.3); 2.520 (2.1); 2.511 (30.3); 2.507 (65.1); 2.502 (87.9); 2.498 (62.9); 2.493 (29.4); 2.334 (0.4); 2.329 (0.5); 2.325 (0.4); 2.075 (1.4); 1.989 (16.0); 1.259 (0.6); 1.234 (0.9); 1.193 (4.5); 1.175 (9.0); 1.157 (4.4); 0.000 (1.6)

### Example I-1-096

¹H-NMR (400.0 MHz, d₆-DMSO): δ=9.769 (2.5); 9.753 (2.6); 8.522 (8.1); 8.464 (0.9); 7.964 (8.6); 7.716 (6.4); 7.706 (2.8); 7.686 (2.6); 7.329 (2.2); 7.311 (3.0); 7.293 (2.0); 5.646 (13.0); 5.573 (8.5); 5.496 (0.7); 4.039 (0.8); 4.02139 (0.8); 4.0213.321 (72.6); 2.676 (0.5); 2.671 (0.7); 2.653 (16.0); 2.525 (1.2); 2.511 (28.5); 2.507 (59.1); 2.502 (78.3); 2.498 (55.9); 2.494 (26.6); 2.334 (0.3); 2.329 (0.5); 2.325 (0.3); 1.989 (3.3); 1.336 (0.5); 1.299 (1.8); 1.259 (3.0); 1.235 (3.7); 1.193 (1.1); 1.175 (2.1); 1.158 (1.0); 0.853 (0.6); 0.835 (0.3); 0.000 (1.5)

### Example I-1-097

¹H-NMR (400.0 MHz, d₆-DMSO): δ=9.927 (2.9); 9.911 (2.9); 7.681 (5.8); 7.644 (2.2); 7.625 (2.4); 7.307 (1.9); 7.288 (3.0); 7.278 (0.7); 7.271 (1.7); 7.261 (0.4); 7.141 (1.1); 5.518 (8.7); 5.414 (0.4); 5.399 (1.5); 4.602 (15.0); 4.269 (0.9); 4.252 (1.9); 4.236 (0.9); 3.489 (4.5); 3.472 (9.2); 3.456 (4.6); 3.348 (216.8); 3.345 (191.8); 3.341 (131.6); 2.665 (3.3); 2.635 (16.0); 2.509 (53.7); 2.505 (72.5); 2.501 (53.7); 2.463 (0.9); 2.332 (0.4); 1.990 (1.2); 1.735 (0.5); 1.718 (1.0); 1.700 (1.0); 1.683 (0.5); 1.609 (0.6); 1.590 (2.4); 1.573 (4.6); 1.555 (4.9); 1.537 (2.5); 1.520 (0.6); 1.232 (0.5); 1.194 (0.4); 1.177 (0.7); 1.159 (0.4); 1.147 (0.4); 1.132 (0.4); 0.931 (1.7); 0.919 (8.0); 0.913 (4.0); 0.900 (15.4); 0.882 (7.1); 0.008 (0.4); 0.000 (12.2); −0.009 (0.5)

### Example I-1-098

¹H-NMR (400.0 MHz, d₆-DMSO): δ=8.520 (3.4); 8.514 (3.5); 7.882 (1.7); 7.861 (3.0); 7.842 (2.2); 7.828 (1.9); 7.822 (1.9); 7.807 (2.1); 7.801 (2.2); 7.612 (2.7); 7.591 (2.3); 7.504 (3.9); 7.483 (3.4); 7.290 (2.6); 7.271 (2.5); 5.263 (10.2); 4.926 (16.0); 3.569 (1.0); 3.325 (24.2); 2.555 (17.5); 2.509 (21.5); 2.505 (28.9); 2.501 (22.3); 2.233 (0.4); 0.000 (0.5)

### Example I-1-099

¹H-NMR (400.0 MHz, d₆-DMSO): δ=9.902 (1.9); 9.896 (2.1); 9.891 (2.2); 9.885 (1.9); 8.506 (3.5); 8.500 (3.5); 8.019 (0.9); 8.013 (0.9); 7.995 (1.8); 7.990 (1.5); 7.975 (1.3); 7.969 (1.2); 7.860 (2.1); 7.848 (2.2); 7.837 (3.1); 7.824 (1.7); 7.818 (2.3); 7.811 (2.2); 7.512 (4.0); 7.491 (3.5); 5.757 (0.5); 5.272 (10.2); 4.808 (16.0); 4.343 (0.4); 3.362 (0.9); 3.172 (2.1); 2.806 (0.4); 2.609 (0.4); 2.506 (16.6); 0.000 (4.6)

### Example I-1-100

¹H-NMR (400.0 MHz, d₆-DMSO): δ=9.740 (3.3); 9.724 (3.3); 8.430 (3.7); 8.366 (0.4); 7.887 (1.6); 7.866 (1.9); 7.859 (2.0); 7.837 (1.8); 7.818 (2.0); 7.812 (2.1); 7.798 (2.5); 7.792 (2.6); 7.513 (3.9); 7.493 (3.5); 7.449 (0.5); 7.429 (0.4); 7.407 (1.2); 7.394 (1.6); 7.391 (1.8); 7.386 (1.7); 7.378 (1.7); 7.373 (1.6); 7.370 (1.6); 7.357 (1.1); 5.757 (1.1); 5.334 (0.4); 5.302 (8.7); 4.860 (0.4); 4.833 (16.60 (0.4); 4.833 (16.0); 4.562 (0.6); 4.547 (0.6); 3.326 (17.4); 3.179 (0.4); 3.166 (0.4); 2.894 (0.9); 2.734 (0.8); 2.509 (23.8); 2.505 (29.4); 0.000 (5.7)

### Example I-1-101

¹H-NMR (400.0 MHz, d₆-DMSO): δ=9.483 (1.6); 9.467 (1.7); 8.498 (2.1); 8.492 (2.2); 7.800 (1.2); 7.794 (1.3); 7.780 (1.4); 7.773 (1.4); 7.733 (0.6); 7.730 (0.6); 7.712 (1.5); 7.709 (1.3); 7.694 (1.4); 7.691 (1.4); 7.666 (2.1); 7.646 (0.9); 7.489 (2.5); 7.468 (2.2); 7.343 (0.9); 7.339 (0.9); 7.325 (1.5); 7.322 (1.6); 7.308 (0.8); 7.305 (0.8); 5.230 (6.5); 3.739 (16.0); 3.329 (9.6); 2.515 (6.8); 2.510 (14.2); 2.506 (19.2); 2.501 (14.2); 2.497 (7.1); 0.000 (0.3)

### Example I-1-102

¹H-NMR (400.0 MHz, d₆-DMSO): δ=9.929 (3.3); 9.911 (3.5); 8.543 (4.3); 8.538 (4.3); 8.347 (4.7); 7.896 (2.3); 7.889 (2.3); 7.875 (2.6); 7.869 (2.6); 7.742 (5.2); 7.737 (5.3); 7.707 (2.6); 7.703 (2.7); 7.685 (2.6); 7.537 (5.0); 7.537 (5.0); 7.517 (4.4); 7.438 (5.4); 7.434 (5.6); 6.282 (3.3); 6.277 (5.9); 6.272 (3.4); 5.546 (16.0); 5.393 (11.1); 4.341 (0.6); 4.330 (0.6); 3.318 (468.6); 2.670 (2.3); 2.505 (291.1); 2.501 (387.6); 2.497 (291.6); 2.328 (2.2); 1.351 (0.7); 1.249 (0.4); 1.234 (1.3); 1.044 (3.3); 1.029 (3.3); −0.001 (2.6)

### Example I-1-103

¹H-NMR (400.0 MHz, d₆-DMSO): δ=8.542 (3.8); 8.536 (3.9); 7.858 (4.1); 7.852 (2.4); 7.838 (5.8); 7.831 (2.8); 7.818 (2.5); 7.714 (4.6); 7.709 (4.7); 7.609 (3.0); 7.588 (2.5); 7.521 (4.5); 7.500 (4.0); 7.414 (4.8); 7.410 (4.9); 7.262 (2.9); 7.243 (2.7); 6.253 (3.2); 6.248 (5.4); 6.243 (3.2); 5.600 (16.0); 5.294 (10.7); 4.342 (0.5); 4.332 (0.5); 3.321 (78.6); 2.676 (0.4); 2.671 (0.6); 2.667 (0.4); 2.524 (21.5); 2.511 (35.5); 2.507 (70.9); 2.502 (93.7); 2.498 (68.4); 2.334 (0.4); 2.329 (0.5); 2.325 (0.4); 1.046 (4.3); 1.030 (4.2); 0.008 (2.1); 0.000 (51.5); −0.008 (2.3)

### Example I-1-104

¹H-NMR (400.0 MHz, d₆-DMSO): δ=9.662 (4.0); 9.660 (3.6); 8.529 (3.4); 8.524 (3.4); 7.843 (2.2); 7.837 (2.1); 7.822 (2.4); 7.816 (2.4); 7.743 (1.3); 7.739 (1.4); 7.733 (4.2); 7.732 (4.3); 7.728 (4.4); 7.726 (4.4); 7.721 (3.0); 7.717 (3.0); 7.688 (4.4); 7.666 (1.9); 7.516 (4.0); 7.496 (3.6); 7.425 (4.1); 7.424 (4.3); 7.420 (4.5); 7.419 (4.2); 6.269 (3.4); 6.264 (5.3); 6.259 (3.4); 5.520 (14.8); 5.268 (9.0); 3.729 (0.5); 3.334 (51.6); 2.672 (0.3); 2.525 (0.9); 2.520 (1.4); 2.512 (18.9); 2.507 (40.2); 2.503 (56.5); 2.498 (42.1); 2.494 (19.8); 2.335 (16.0); 0.008 (1.6); 0.000 (48.1); −0.009 (1.5)

### Example I-1-105

¹H-NMR (400.0 MHz, d₆-DMSO): δ=9.673 (4.2); 9.656 (4.4); 8.452 (4.5); 7.859 (2.1); 7.838 (4.6); 7.823 (2.9); 7.817 (2.9); 7.809 (2.4); 7.772 (0.5); 7.745 (5.4); 7.742 (5.4); 7.528 (4.8); 7.507 (4.3); 7.436 (5.9); 7.434 (6.4); 7.375 (1.3); 7.360 (2.0); 7.346 (1.9); 7.324 (1.2); 6.276 (5.5); 5.572 (1.0); 5.556 (16.0); 5.333 (10.5); 4.818 (0.6); 3.318 (108.5); 2.670 (0.8); 2.501 (134.8); 2.328 (0.8); 1.234 (0.7); 0.000 (57.6); −0.002 (48.8)

### Example I-1-106

¹H-NMR (400.0 MHz, d₆-DMSO): δ=9.848 (1.2); 9.842 (1.3); 9.837 (1.4); 9.831 (1.2); 8.531 (2.1); 8.525 (2.1); 7.991 (0.6); 7.985 (0.5); 7.967 (1.1); 7.961 (0.8); 7.947 (0.8); 7.941 (0.7); 7.866 (1.3); 7.859 (2.4); 7.846 (2.4); 7.839 (1.6); 7.823 (0.9); 7.739 (2.6); 7.734 (2.6); 7.528 (2.5); 7.508 (2.2); 7.433 (2.7); 7.432 (2.6); 7.429 (2.8); 6.277 (1.8); 6.272 (2.9); 6.267 (1.7); 5.535 (8.3); 5.303 (5.7); 4.341 (2.2); 4.331 (2.3); 3.794 (0.6); 3.783 (0.7); 3.778 (0.8); 3.768 (0.8); 3.763 (0.6); 3.752 (0.6); 3.320 (30.1); 3.176 (10.4); 3.163 (1.4); 2.506 (30.6); 2.502 (38.8); 2.498 (27.5); 1.045 (16.0); 1.030 (15.6); 0.008 (1.1); 0.000 (24.3); −0.009 (0.9)

### Example I-1-107

¹H-NMR (400.0 MHz, d₆-DMSO): δ=9.485 (1.7); 9.469 (1.7); 9.112 (4.3); 8.822 (9.3); 7.752 (0.3); 7.738 (3.7); 7.731 (2.1); 7.724 (1.8); 7.350 (0.8); 7.342 (0.7); 7.334 (1.0); 7.327 (1.1); 7.319 (0.7); 7.311 (0.7); 5.760 (0.6); 5.259 (6.7); 3.738 (16.0); 3.329 (7.2); 2.512 (11.0); 2.507 (14.4); 2.503 (10.3); 1.231 (0.4); 1.048 (1.1); 1.033 (1.1)

### Example I-1-108

¹H-NMR (400.0 MHz, d₆-DMSO): δ=9.889 (3.7); 9.883 (3.8); 8.095 (1.6); 8.090 (1.6); 8.072 (2.6); 8.067 (2.6); 7.994 (3.9); 7.970 (2.5); 7.831 (5.8); 5.424 (8.5); 4.791 (16.0); 4.057 (0.7); 4.039 (2.2); 4.021 (2.2); 4.003 (0.8); 3.321 (45.1); 2.525 (0.6); 2.512 (14.5); 2.507 (30.2); 2.503 (40.6); 2.498 (29.2); 2.494 (14.0); 1.990 (9.4); 1.194 (2.4); 1.176 (4.8); 1.158 (2.4); 0.007 (0.5); 0.000 (16.4); −0.001 (15.3); −0.009 (0.6)

### Example I-1-109

¹H-NMR (400.0 MHz, d₆-DMSO): δ=9.909 (2.9); 9.892 (3.0); 8.600 (4.2); 8.596 (4.4); 8.567 (4.1); 8.561 (4.3); 7.933 (2.4); 7.928 (4.1); 7.923 (2.5); 7.842 (1.0); 7.840 (1.1); 7.821 (2.6); 7.802 (1.9); 7.799 (1.9); 7.743 (3.5); 7.721 (2.1); 7.384 (1.4); 7.382 (1.5); 7.365 (2.8); 7.349 (1.4); 7.347 (1.4); 5.256 (10.5); 4.548 (16.0); 3.369 (30.0); 3.320 (29.7); 2.509 (27.7); 2.505 (38.9); 2.500 (29.8); 0.008 (1.5); 0.000 (45.2); −0.008 (2.0)

### Example I-1-110

¹H-NMR (400.0 MHz, d₆-DMSO): δ=9.840 (1.5); 9.838 (1.6); 9.833 (1.8); 9.830 (1.6); 8.425 (2.5); 8.419 (2.7); 8.289 (0.9); 8.284 (0.9); 8.267 (1.0); 8.261 (1.7); 8.255 (1.0); 8.238 (0.9); 8.232 (0.9); 7.817 (1.5); 7.810 (1.5); 7.796 (1.7); 7.790 (1.7); 7.522 (3.5); 7.501 (3.0); 5.755 (0.9); 5.290 (6.4); 4.834 (16.0); 3.320 (28.6); 2.524 (0.5); 2.510 (11.6); 2.506 (25.5); 2.502 (36.7); 2.497 (28.2); 2.493 (14.1); 0.008 (0.8); 0.000 (26.5); −0.008 (1.0)

### Example I-1-111

¹H-NMR (400.0 MHz, d₆-DMSO): δ=9.652 (4.5); 9.636 (4.6); 7.953 (3.9); 7944 (3.9); 7.869 (3.8); 7.847 (5.2); 7.839 (11.8); 7.716 (2.3); 7.713 (2.6); 7.697 (2.8); 7.694 (4.2); 7.675 (2.0); 7.673 (2.2); 7.344 (2.2); 7.342 (2.5); 7.325 (4.4); 7.309 (2.0); 7.307 (2.2); 5.757 (1.5); 5.431 (16.0); 3.322 (93.1); 2.797 (0.4); 2.788 (1.2); 2.779 (1.8); 2.770 (2.7); 2.760 (2.7); 2.752 (1.8); 2.743 (1.3); 2.733 (0.6); 2.677 (0.4); 2.673 (0.6); 2.669 (0.4); 2.526 (1.4); 2.513 (30.1); 2.508 (63.7); 2.504 (90.2); 2.500 (70.0); 2.335 (0.4); 2.331 (0.5); 2.326 (0.4); 1.232 (0.9); 0.783 (0.4); 0.750 (1.6); 0.737 (4.6); 0.732 (6.1); 0.720 (5.9); 0.715 (4.9); 0.703 (1.9); 0.506 (1.9); 0.494 (5.6); 0.489 (5.4); 0.485 (5.2); 0.479 (5.2); 0.467 (1.5); 0.008 (1.2); 0.000 (33.5); −0.008 (1.2)

### Example I-1-112

¹H-NMR (400.0 MHz, d₆-DMSO): δ=9.955 (1.3); 9.949 (1.3); 9.943 (1.4); 9.937 (1.3); 8.497 (2.1); 8.491 (2.2); 7.954 (2.2); 7.942 (0.7); 7.936 (0.7); 7.922 (0.8); 7.918 (1.1); 7.916 (0.9); 7.912 (1.0); 7.898 (1.0); 7.892 (0.9); 7.820 (1.4); 7.814 (1.4); 7.808 (1.4); 7.800 (1.8); 7.794 (2.1); 7.784 (1.0); 7.772 (0.9); 7.505 (2.6); 7.484 (2.3); 5.246 (5.9); 4.580 (10.2); 3.583 (1.5); 3.565 (5.0); 3.548 (5.1); 3.530 (1.6); 3.321 (32.5); 2.892 (16.0); 2.732 (13.6); 2.675 (0.4); 2.596 (0.6); 2.595 (0.6); 2.584 (0.6); 2.583 (0.6); 2.526 (0.4); 2.521 (0.6); 2.512 (10.2); 2.508 (22.3); 2.503 (31.8); 2.499 (23.6); 2.494 (11.2); 1.174 (4.9); 1.156 (10.1); 1.139 (4.8); 1.046 (0.5); 1.031 (0.5); 0.008 (0.5); 0.000 (18.1); −0.009 (0.6)

### Example I-1-113

¹H-NMR (400.0 MHz, d₆-DMSO): δ=9.761 (3.9); 9.759 (3.6); 8.495 (3.4); 8.489 (3.4); 7.797 (2.1); 7.791 (2.0); 7.776 (2.4); 7.770 (2.4); 7.697 (1.2); 7.694 (1.3); 7.675 (2.8); 7.671 (2.9); 7.639 (4.4); 7.616 (2.0); 7.493 (4.1); 7.473 (3.6); 5.212 (9.1); 4.570 (16.0); 3.580 (2.3); 3.563 (7.7); 3.545 (7.8); 3.528 (2.4); 3.322 (38.2); 2.893 (0.6); 2.733 (0.5); 2.527 (0.6); 2.522 (1.0); 2.513 (14.1); 2.509 (30.4); 2.504 (43.1); 2.499 (32.2); 2.495 (15.4); 2.361 (15.8); 1.171 (7.6); 1.154 (15.6); 1.136 (7.3); 1.047 (0.5); 1.031 (00.5); 0.008 (0.6); 0.000 (19.6); −0.009 (0.6)

### Example I-1-114

¹H-NMR (400.0 MHz, d₆-DMSO): δ=8.510 (3.2); 8.505 (3.2); 7.823 (1.8); 7.810 (2.2); 7.803 (4.6); 7.789 (2.4); 7.783 (4.6); 7.576 (2.5); 7.555 (2.1); 7.499 (3.8); 7.479 (3.4); 7.252 (2.4); 7.234 (2.3); 5.758 (1.1); 5.248 (9.0); 4.666 (16.0); 3.560 (2.2); 3.542 (7.5); 3525 (7.7); 3.507 (2.3); 3.322 (29.7); 2.545 (15.6); 2.527 (0.7); 2522 (0.8); 2.513 (11.7); 2.509 (25.1); 2.504 (35.3); 2.500 (26.1); 2.495 (12.3); 1.165 (7.4); 1.147 (15.2); 1.130 (7.2); 1.047 (1.1); 1.032 (1.1); 0.008 (0.7); 0.000 (20.9); −0.009 (0.7)

### Example I-1-115

¹H-NMR (400.0 MHz, d₆-DMSO): δ=9.584 (3.9); 9.568 (3.9); 8.375 (3.8); 8.369 (3.9); 7.722 (2.1); 7.716 (2.0); 7.702 (2.4); 7.695 (2.3); 7.483 (4.8); 7.462 (4.0); 7.416 (2.9); 7.395 (3.9); 7.280 (2.7); 7.263 (2.8); 7.259 (2.4); 7.242 (2.1); 5.756 (1.5); 5.381 (10.3); 4.604 (0.6); 4.589 (16.0); 4.184 (1.7); 4.166 (5.4); 4.149 (5.5); 4.132 (1.7); 3.578 (2.2); 3.560 (7.1); 3.543 (7.2); 3.525 (2.3); 3.318 (138.0); 2.671 (0.8); 2.506 (99.5); 2.502 (139.3); 2.498 (106.3); 2.453 (0.3); 2.329 (0.8); 1.230 (5.8); 1.212 (11.9); 1.195 (5.5); 1.172 (7.2); 1.155 (14.6); 1.137 (7.0); 1.045 (1.3); 1.030 (1.4); 0.008 (1.1); 0.000 (31.8); −0.008 (1.2)

### Example I-1-116

¹H-NMR (400.0 MHz, d₆-DMSO): δ=10.030 (3.2); 10.028 (3.3); 10.013 (3.4); 10.011 (3.3); 8.406 (3.4); 8.400 (3.5); 7.912 (3.0); 7.910 (3.2); 7.892 (3.4); 7.890 (3.3); 7.753 (1.9); 7.746 (1.9); 7.732 (2.3); 7.726 (2.3); 7.490 (4.3); 7.469 (3.8); 7.378 (2.7); 7.362 (2.9); 7.358 (2.8); 7.342 (2.6); 5.755 (0.5); 5.526 (9.5); 4.609 (16.0); 3.589 (2.3); 3.571 (7.6); 3.554 (7.7); 3.536 (2.4); 3.319 (145.5); 2.671 (0.8); 2.524 (1.6); 2.510 (44.6); 2.506 (96.3); 2.501 (136.3); 2.497 (101.8); 2.493 (49.2); 2.328 (0.8); 1.179 (7.5); 1.161 (15.3); 1.144 (7.3); 1.045 (0.5); 1.030 (0.5); 0.008 (1.1); 0.000 (36.0); −0.008 (1.3)

### Example I-1-117

¹H-NMR (400.0 MHz, d₆-DMSO): δ=9.954 (4.8); 9.949 (4.7); 8.023 (1.9); 8.018 (1.9); 8.000 (3.6); 7.995 (3.8); 7.951 (5.1); 7.927 (2.6); 7.824 (7.7); 5.424 (0.3); 5.401 (10.0); 4.569 (16.0); 3.967 (00.4); 3.483 (4.7); 3.466 (9.9); 3.449 (4.9); 3.322 (16.5); 2.526 (0.7); 2.513 (17.3); 2.508 (37.7); 2.504 (54.1); 2.499 (41.2); 2.495 (20.2); 2.443 (0.4); 2.331 (0.3); 1.990 (0.5); 1.603 (0.6); 1.585 (2.3); 1.567 (4.3); 1.549 (4.7); 1.532 (2.3); 1.514 (0.6); 0.915 (7.8); 0.896 (15.6); 0.878 (7.1); 0.865 (0.5); 0.008 (0.8); 0.000 (27.4); −0.008 (1.0)

### Example I-1-118

¹H-NMR (400.0 MHz, d₆-DMSO): δ=9.942 (4.1); 9.938 (4.2); 9.938 (4.2); 9.937 (4.0); 8.027 (2.1); 8.022 (2.0); 8.004 (3.9); 7.999 (4.0); 7.956 (4.7); 7.954 (4.7); 7.932 (2.5); 7.931 (2.4); 7.825 (7.7); 5.403 (9.3); 4.528 (16.0); 4.058 (0.9); 4.040 (2.7); 4.022 (2.7); 4.005 (0.9); 3.368 (35.5); 3.324 (30.7); 2.528 (0.5); 2.523 (0.7); 2.514 (10.9); 2.510 (23.8); 2.505 (33.7); 2.500 (25.2); 2.496 (11.9); 1.991 (12.0); 1.231 (0.3); 1.195 (3.2); 1.177 (6.4); 1.160 (3.1); 0.008 (0.6); 0.000 (21.3); −0.009 (0.7)

### Example I-1-119

¹H-NMR (400.0 MHz, d₆-DMSO): δ=9.952 (4.9); 9.947 (5.1); 8.022 (1.8); 8.017 (2.0); 7.998 (3.4); 7.993 (3.9); 7.972 (0.4); 7.951 (5.5); 7.927 (2.8); 7.837 (0.6); 7.823 (7.8); 5.754 (6.2); 5.400 (11.7); 4.564 (15.8); 4.056 (0.8); 4.039 (2.4); 4.021 (2.4); 4.003 (0.8); 3.584 (2.1); 3.566 (6.9); 3.549 (7.0); 3.531 (2.3); 3.319 (59.0); 2.672 (0.6); 2.503 (94.2); 2.499 (80.6); 2.444 (0.6); 2.329 (0.6); 1.989 (10.0); 1.397 (0.6); 1.299 (0.4); 1.259 (0.4); 1.249 (0.3); 1.232 (1.3); 1.194 (2.7); 1.175 (11.3); 1.158 (16.0); 1.140 (6.7); 0.008 (1.5); 0.000 (46.2)

### Example I-1-120

¹H-NMR (400.0 MHz, d₆-DMSO): δ=9.785 (2.9); 9.768 (3.0); 8.251 (2.8); 8.246 (2.9); 7.867 (4.8); 7.518 (1.7); 7.512 (1.7); 7.500 (1.7); 7.495 (1.7); 5.382 (6.4); 4.771 (12.3); 4.056 (1.2); 4.039 (3.8); 4.021 (3.8); 4.003 (1.3); 3.319 (30.7); 2.525 (0.8); 2.511 (14.9); 2.507 (31.6); 2.503 (44.8); 2.498 (33.6); 2.494 (16.3); 1.989 (16.0); 1.194 (4.1); 1.176 (8.1); 1.158 (4.0); 0.000 (5.7)

### Example I-1-121

¹H-NMR (400.0 MHz, d₆-DMSO): δ=7.852 (5.2); 7.836 (2.7); 7.799 (2.0); 7.779 (2.6); 7.758 (1.2); 7.500 (2.3); 7.481 (2.0); 5.391 (7.5); 4.057 (0.9); 4.039 (2.7); 4.021 (2.7); 4.003 (0.9); 3.718 (16.0); 3.321 (66.2); 2.672 (0.4); 2.506 (50.6); 2.503 (68.4); 2.499 (54.0); 2.329 (0.4); 1.989 (11.2); 1.909 (0.7); 1.235 (0.6); 1.193 (2.8); 1.176 (5.6); 1.158 (2.8); −0.001 (5.4)

### Example I-1-122

¹H-NMR (400.0 MHz, d₆-DMSO): δ=7.849 (4.3); 7.782 (0.8); 7.760 (1.9); 7.742 (1.9); 7.715 (2.0); 7.694 (0.9); 7.220 (1.5); 7.202 (1.4); 5.372 (6.3); 3.705 (16.0); 3.325 (15.9); 3.180 (0.5); 3.167 (00.5); 2.546 (10.4); 2.529 (0.4); 2.515 (6.2); 2.510 (13.5); 2.506 (19.2); 2.502 (15.3); 0.000 (2.2)

### Example I-1-123

¹H-NMR (400.0 MHz, d₆-DMSO): δ=9.668 (3.1); 9.649 (3.2); 7.864 (5.1); 7.864 (5.1); 7.468 (2.8); 7.461 (3.0); 7.070 (1.7); 7.063 (1.7); 7.051 (1.7); 7.044 (1.7); 5.373 (7.0); 4.725 (12.6); 3.998 (16.0); 3.323 (40.2); 2.509 (24.1); 2.504 (33.2); 2.500 (26.0); 1.990 (0.4); 0.000 (1.4)

### Example I-1-124

¹H-NMR (400.0 MHz, d₆-DMSO): δ=9.715 (3.0); 9.697 (3.1); 7.857 (5.1); 7.429 (2.7); 7.423 (2.7); 7.038 (1.8); 7.031 (1.7); 7.019 (1.7); 7.012 (1.7); 5.349 (6.4); 4.471 (10.9); 4.058 (0.9); 4.040 (2.7); 4.022 (2.7); 4.004 (1.0); 3.983 (16.0); 3.348 (22.2); 3.321 (36.0); 2.527 (0.6); 2.513 (13.0); 2.509 (26.6); 2.504 (36.4); 2.500 (26.9); 2.496 (12.8); 1.990 (11.6); 1.195 (3.0); 1.177 (6.1); 1.177 (6.1); 1.159 (3.0); 0.000 (0.5)

### Example I-1-125

¹H-NMR (400.0 MHz, d₆-DMSO): δ=9.696 (3.3); 7.859 (1.2); 7.845 (6.2); 7.838 (4.3); 7.823 (2.9); 7.820 (2.8); 7.801 (0.8); 7.797 (0.8); 5.393 (8.1); 4.789 (16.0); 3.328 (35.1); 2.532 (0.5); 2.518 (11.4); 2.514 (23.4); 2.510 (32.0); 2.505 (23.7); 2.501 (11.4); 2.384 (13.5); 1.996 (0.4)

### Example I-1-126

¹H-NMR (400.0 MHz, d₆-DMSO): δ=9.699 (3.2); 9.682 (3.3); 7.869 (6.3); 7.786 (3.8); 7.279 (2.1); 7.264 (2.0); 7.262 (2.0); 5.357 (9.1); 4.769 (16.0); 3.328 (30.2); 2.514 (22.2); 2.510 (31.1); 2.505 (31.1); 1.996 (0.6); 1.182 (0.3)

### Example I-1-127

¹H-NMR (400.0 MHz, d₆-DMSO): δ=9.559 (7.0); 9.544 (7.0); 9.543 (7.1); 8.408 (6.7); 8.402 (6.8); 8.314 (0.6); 8.071 (5.1); 8.061 (5.1); 7.767 (3.9); 7.761 (3.8); 7.746 (4.5); 7.740 (4.4); 7.629 (3.0); 7.608 (3.6); 7.602 (3.3); 7.581 (3.4); 7.494 (8.5); 7.473 (7.2); 7.309 (2.4); 7.296 (3.0); 7.293 (3.0); 7.288 (2.7); 7.279 (2.9); 7.275 (2.7); 7.272 (2.5); 7.258 (2.0); 5.754 (6.7); 5.315 (16.0); 4.340 (1.1); 4.329 (1.1); 3.793 (0.3); 3.783 (0.4); 3.778 (0.5); 3.767 (0.4); 3.763 (0.4); 3.752 (0.3); 3.317 (213.9); 2.814 (0.6); 2.805 (1.7); 2.796 (2.5); 2.787 (3.8); 2.78 (3.); 2778 (3.9); 2.769 (2.6); 2.760 (1.8); 2.750 (0.7); 2.675 (1.4); 2.671 (2.0); 2.666 (1.4); 2.524 (5.1); 2.510 (111.2); 2.506 (228.6); 2.502 (315.8); 2.497 (238.9); 2.493 (118.2); 2.333 (1.3); 2.329 (1.9); 2.324 (1.5); 1.234 (0.4); 1.045 (8.3); 1.030 (8.2); 0.757 (2.1); 0.744 (6.3); 0.739 (8.4); 0.727 (8.2); 0.722 (6.7); 0.710 (2.7); 0.510 (2.6); 0.498 (7.4); 0.493 (7.6); 0.488 (7.2); 0.484 (7.2); 0.471 (2.1); 0.000 (13.5)

### Example I-1-128

¹H-NMR (400.0 MHz, d₆-DMSO): δ=9.683 (3.9); 9.666 (4.0); 7.868 (3.5); 7.850 (12.3); 7.828 (2.2); 7.816 (0.8); 7.699 (2.1); 7.697 (2.3); 7.681 (2.6); 7.678 (3.9); 7.675 (2.2); 7.659 (2.0); 7.656 (2.0); 7.329 (2.2); 7.326 (2.4); 7.309 (3.8); 7.294 (1.9); 7.291 (2.0); 5.756 (1.4); 5.452 (14.2); 3.334 (7.0); 3.173 (2.2); 2.829 (16.0); 2.817 (15.9); 2.674 (0.4); 2.537 (0.4); 2.528 (1.0); 2.514 (20.0); 2.510 (42.2); 2.505 (59.3); 2.501 (44.2); 2.496 (21.2); 2.332 (0.3); 1.357 (0.4); 1.232 (2.5); 0.853 (0.4); 0.000 (0.7)

### Example I-1-129

¹H-NMR (400.0 MHz, d₆-DMSO): δ=9.675 (3.5); 9.658 (3.6); 8.002 (1.4); 7.988 (2.8); 7.973 (1.4); 7.866 (3.1); 7.851 (9.5); 7.845 (4.3); 7.701 (1.9); 7.699 (2.0); 7.683 (2.3); 7.680 (3.3); 7.661 (1.7); 7.658 (1.7); 7.326 (1.8); 7.324 (1.9); 7.307 (3.4); 7.291 (1.7); 7.289 (1.7); 5.450 (12.4); 3.324 (59.7); 3.286 (2.1); 3.269 (4.8); 3.253 (4.8); 3.237 (2.2); 2.674 (0.4); 2.528 (1.0); 2.514 (20.8); 2.510 (42.3); 2.505 (58.2); 2.501 (44.3); 2.497 (22.3); 2.332 (0.3); 1.561 (0.5); 1.542 (2.4); 1.524 (4.7); 1.506 (4.8); 1.488 (2.6); 1.470 (0.6); 1.231 (0.4); 0.930 (8.0); 0.912 (16.0); 0.893 (7.0); 0.146 (0.3); 0.008 (2.6); 0.000 (71.1); −0.008 (3.0); −0.150 (0.3)

### Example I-1-130

¹H-NMR (400.0 MHz, d₆-DMSO): δ=9.596 (2.6); 9.579 (2.7); 8.375 (3.2); 7.806 (2.6); 7.786 (2.7); 7.706 (1.7); 7.690 (2.0); 7.476 (3.0); 7.456 (2.4); 7.337 (1.6); 7.319 (2.4); 7.300 (1.5); 5.525 (8.3); 3.761 (16.0); 3.320 (149.8); 2.671 (1.1); 2.598 (0.4); 2.502 (163.1); 2.328 (1.0); 0.146 (0.6); 0.000 (111.3); −0.150 (0.6)

### Example I-1-131

¹H-NMR (400.0 MHz, d₆-DMSO): δ=9.675 (2.1); 9.658 (2.2); 7.862 (1.9); 7.847 (5.6); 7.841 (2.8); 7.832 (1.8); 7.812 (1.6); 7.697 (1.2); 7.679 (2.0); 7.660 (1.0); 7.657 (1.0); 7.321 (1.2); 7.304 (2.1); 7.286 (1.0); 5.437 (7.6); 4.092 (0.7); 4.076 (1.0); 4.058 (1.1); 4.041 (0.7); 3.322 (66.0); 2.673 (0.3); 2.508 (39.9); 2.504 (54.4); 2.500 (42.6); 1.232 (0.3); 1.181 (16.0); 1.165 (16.0); 1.047 (1.3); 1.032 (1.2); 0.007 (2.4); 0.000 (57.3); −0.008 (2.8)

### Example I-1-132

¹H-NMR (400.0 MHz, d₆-DMSO): δ=9.621 (1.7); 9.603 (1.7); 8.497 (2.1); 8.491 (2.2); 8.218 (2.3); 7.829 (1.3); 7.822 (1.3); 7.808 (1.5); 7.802 (1.5); 7.645 (1.2); 7.641 (1.3); 7.627 (1.2); 7.623 (1.3); 7.498 (2.6); 7.477 (2.3); 5.331 (5.8); 3.770 (16.0); 3.322 (20.7); 2.527 (0.3); 2.513 (8.4); 2.509 (18.0); 2.504 (25.4); 2.500 (19.1); 2.496 (9.3); 1.047 (2.0); 1.032 (1.9)

### Example I-1-133

¹H-NMR (400.0 MHz, d₆-DMSO): δ=9.513 (4.3); 8.488 (3.4); 8.483 (3.6); 8.029 (2.5); 8.020 (2.6); 7.772 (1.8); 7.766 (1.8); 7.751 (2.0); 7.745 (2.1); 7.635 (2.6); 7.612 (3.7); 7.520 (2.7); 7.498 (1.9); 7.482 (3.7); 7.462 (3.2); 5.249 (9.4); 5.249 (9.4); 3.319 (55.8); 2.776 (0.7); 2.767 (1.1); 2.758 (1.5); 2.749 (1.5); 2.741 (1.1); 2.731 (0.7); 2.672 (0.5); 2.503 (75.1); 2.342 (16.0); 0.738 (0.9); 0.721 (3.7); 0.708 (3.5); 0.692 (1.0); 0.489 (1.1); 0.478 (3.5); 0.472 (3.7); 0.463 (3.4); 0.451 (0.9); 0.000 (2.2)

### Example I-1-134

¹H-NMR (400.0 MHz, d₆-DMSO): δ=9.682 (3.8); 9.665 (3.9); 7.937 (1.5); 7.923 (3.0); 7.909 (1.6); 7.865 (3.4); 7.849 (10.0); 7.699 (2.0); 7.697 (2.0); 7.678 (3.5); 7.659 (1.8); 7.322 (2.0); 7.306 (3.7); 7.289 (1.8); 5.755 (4.0); 5.445 (13.9); 3.353 (1.2); 3.335 (4.3); 3.321 (60.4); 3.302 (4.3); 3.284 (1.2); 2.673 (0.5); 2.508 (59.9); 2.504 (82.6); 2.500 (65.7); 20.331 (0.5); 1.233 (0.3); 1.143 (7.7); 1.125 (16.0); 1.107 (7.5); 0.000 (0.7)

### Example I-1-135

¹H-NMR (400.0 MHz, d₆-DMSO): δ=9.639 (4.6); 9.623 (4.7); 8.127 (3.5); 8.107 (3.6); 7.867 (4.0); 7.848 (14.6); 7.707 (2.5); 7.704 (2.5); 7.689 (3.1); 7.686 (4.3); 7.667 (2.2); 7.664 (2.1); 7.324 (2.4); 7.321 (2.5); 7.305 (4.5); 7.289 (2.2); 7.286 (2.2); 5.444 (16.0); 4.457 (1.1); 4.436 (2.2); 4.415 (2.2); 4.395 (1.2); 4.342 (0.5); 4.331 (0.5); 3.320 (144.5); 2.677 (0.6); 2.672 (0.9)(0.6); 2.672 (09); 2668 (0.6); 2.526 (2.6); 2.512 (51.4); 2.508 (103.8); 2.503 (141.4); 2.499 (105.6); 2.494 (51.3); 2.335 (0.6); 2.330 (0.8); 2.326 (0.6); 2.303 (0.8); 2.295 (1.2); 2.284 (1.9); 2.277 (2.9); 2.266 (2.5); 2.255 (3.0); 2.248 (2.2); 2.237 (1.4); 2.230 (1.1); 1.976 (0.6); 1.970 (0.5); 1.954 (2.1); 1.928 (3.3); 1.922 (2.8); 1.906 (2.4); 1.899 (2.4); 1.883 (0.7); 1.877 (0.8); 1.713 (1.6); 1.704 (1.7); 1.697 (2.2); 1.689 (2.2); 1.682 (2.5); 1.671 (3.0); 1.661 (1.4); 1.652 (1.4); 1.645 (1.4); 1.626 (0.7); 1.233 (0.5); 1.047 (3.8); 1.031 (3.7); 0.146 (0.8); 0.008 (6.7); 0.000 (174.2); −0.008 (6.6); −0.150 (0.8)

### Example I-1-136

¹H-NMR (400.0 MHz, d₆-DMSO): δ=9.567 (2.2); 9.551 (2.1); 8.622 (3.7); 8.615 (2.8); 8.104 (2.0); 8.098 (1.6); 8.083 (2.2); 8.076 (1.7); 7.815 (3.1); 7.794 (2.7); 7.726 (1.1); 7.707 (2.1); 7.687 (1.3); 7.430 (1.4); 7.413 (2.6); 7.400 (3.1); 7.379 (2.1); 5.753 (0.8); 3.756 (16.0); 3.319 (33.9); 3.169 (1.5); 2.670 (0.6); 2.501 (96.8); 2.328 (0.6); 0.006 (1.1); 0.000 (2.8)

### Example I-1-137

¹H-NMR (400.0 MHz, d₆-DMSO): δ=9.595 (4.7); 9.579 (4.9); 8.333 (1.8); 8.316 (4.0); 8.300 (1.9); 7.908 (3.9); 7.886 (5.3); 7.858 (12.9); 7.775 (2.7); 7.772 (2.8); 7.756 (3.3); 7.753 (4.6); 7.750 (2.5); 7.735 (2.3); 7.731 (2.3); 7.370 (2.5); 7.367 (2.6); 7.351 (4.5); 7.335 (2.4); 7.332 (2.3); 5.756 (0.8); 5.460 (16.0); 4.342 (0.8); 4.332 (0.8); 4.211 (1.0); 4.187 (3.3); 4.170 (3.4); 4.163 (3.6); 4.146 (3.3); 4.138 (1.4); 4.121 (1.1); 3.321 (63.7); 2.678 (0.4); 2.674 (0.6); 2.669 (0.4); 2.527 (1.6); 2.514 (32.9); 2.509 (67.7); 2.505 (92.6); 2.500 (69.1); 2.496 (33.4); 2.336 (0.4); 2.332 (0.6); 2.327 (0.4); 1.048 (5.5); 1.032 (5.4); 0.146 (0.5); 0.008 (3.7); 0.000 (105.0); −0.008 (3.8); −0.150 (0.4)

### Example I-1-138

¹H-NMR (400.0 MHz, d₆-DMSO): δ=9.666 (4.7); 9.650 (4.9); 7.972 (3.8); 7.953 (3.8); 7.862 (4.2); 7.846 (12.9); 7.840 (5.6); 7.701 (2.6); 7.698 (2.6); 7.682 (3.3); 7.679 (4.5); 7.660 (2.3); 7.657 (2.2); 7.326 (2.5); 7.324 (2.5); 7.308 (4.5); 7.291 (2.3); 7.289 (2.2); 5.440 (16.0); 4.344 (0.6); 4.334 (0.7); 4.264 (0.4); 4.248 (1.5); 4.230 (2.8); 4.213 (2.8); 4.196 (1.5); 4.179 (0.4); 3.324 (44.6); 2.675 (0.4); 2.528 (1.3); 2.515 (24.2); 2.511 (47.6); 2.506 (63.4); 2.502 (47.1); 2.497 (22.9); 2.333 (0.4); 1.965 (0.8); 1.947 (2.0); 1.934 (2.9); 1.918 (3.1); 1.903 (2.3); 1.887 (1.2); 1.724 (0.5); 1.705 (1.7); 1.697 (2.2); 1.685 (3.2); 1.667 (2.6); 1.642 (1.3); 1.634 (1.0); 1.628 (1.4); 1.619 (0.8); 1.602 (2.5); 1.595 (2.2); 1.585 (3.2); 1.580 (2.1); 1.573 (2.4); 1.566 (1.7); 1.557 (1.1); 1.542 (0.5); 1.535 (0.4); 1.464 (1.2); 1.448 (2.5); 1.432 (3.0); 1.418 (2.8); 1.402 (1.9); 1.383 (0.7); 1.048 (4.6); 1.033 (4.6); 0.008 (2.2); 0.000 (50.3); −0.008 (1.9)

### Example I-1-139

¹H-NMR (400.0 MHz, d₆-DMSO): δ=9.665 (4.7); 9.648 (4.8); 8.013 (2.0); 7.998 (4.1); 7.983 (2.0); 7.864 (4.0); 7.843 (16.0); 7.704 (2.5); 7.702 (2.5); 7.683 (4.2); 7.664 (2.2); 7.662 (2.1); 7.358 (6.8); 7.337 (14.5); 7.325 (2.9); 7.323 (2.8); 7.300 (13.6); 7.290 (3.3); 7.287 (3.4); 7.279 (6.4); 7.253 (0.8); 7.232 (0.5); 5.755 (4.7); 5.438 (15.6); 3.560 (2.1); 3.543 (5.3); 3.527 (5.4); 3.510 (2.3); 3.322 (59.5); 3.307 (0.7); 2.838 (4.2); 2.820 (7.8); 2.803 (3.8); 2.736 (0.4); 2.718 (0.6); 2.700 (0.3); 2.674 (0.6); 2.509 (74.1); 2.504 (97.3); 2.500 (75.2); 2.331 (0.6); 2.327 (0.4); 1.047 (0.9); 1.032 (0.9); 0.146 (0.4); −0.001 (87.4); −0.150 (0.4)

### Example I-1-140

¹H-NMR (400.0 MHz, d₆-DMSO): δ=9.808 (5.6); 9.794 (5.4); 9.792 (5.6); 8.392 (5.7); 8.386 (5.7); 8.313 (0.5); 8.142 (4.3); 8.133 (4.2); 7.733 (5.4); 7.713 (8.4); 7.706 (3.3); 7.691 (3.7); 7.685 (3.7); 7.477 (7.0); 7.456 (5.9); 7.324 (4.3); 7.307 (4.7); 7.304 (4.7); 7.287 (3.9); 5.754 (6.5); 5.564 (16.0); 3.318 (255.2); 2.819 (0.4); 2.809 (1.3); 2.800 (2.0); 2.791 (3.0); 2.782 (3.0); 2.773 (2.0); 2.764 (1.4); 2.754 (0.5); 2.675 (1.1); 2.671 (1.5); 2.667 (1.2); 2.524 (4.6); 2.506 (184.5); 2.502 (252.8); 2.497 (193.5); 2.333 (1.1); 2.329 (1.5); 2.324 (1.1); 1.045 (1.4); 1.030 (1.4); 0.757 (1.7); 0.744 (5.1); 0.740 (6.7); 0.727 (6.6); 0.722 (5.3); 0.710 (2.1); 0.509 (2.1); 0.497 (6.1); 0.492 (6.0); 0.487 (5.7); 0.470 (1.7); 0.146 (1.2); 0.008 (9.4); 0.000 (245.3); −0.008 (10.4); −0.150 (1.1)

### Example I-1-141

¹H-NMR (400.0 MHz, d₆-DMSO): δ=9.804 (4.9); 9.786 (5.1); 8.504 (6.2); 8.499 (6.2); 8.279 (6.4); 8.058 (4.6); 8.048 (4.5); 7.828 (3.7); 7.822 (3.6); 7.807 (4.2); 7.801 (4.2); 7.639 (3.7); 7.635 (3.7); 7.622 (3.6); 7.618 (3.6); 7.500 (7.0); 7.479 (6.3); 5.755 (6.4); 5.382 (16.0); 4.341 (0.4); 4.331 (0.4); 3.320 (89.8); 2.825 (0.5); 2.816 (1.4); 2.807 (2.0); 2.798 (3.1); 2.788 (3.1); 2.780 (2.0); 2.770 (1.4); 2.761 (0.5); 2.677 (0.5); 2.673 (0.6); 2.668 (0.5); 2.526 (1.8); 2.513 (38.0); 2.508 (76.8); 2.504 (104.1); 2.499 (78.0); 2.495 (38.1); 2.335 (0.5); 2.331 (0.6); 2.326 (0.4); 1.047 (3.3); 1.032 (3.2); 0.761 (1.8); 0.748 (5.3); 0.743 (7.0); 0.731 (6.9); 0.726 (5.5); 0.714 (2.2); 0.517 (2.2); 0.506 (6.2); 0.500 (6.2); 0.496 (5.8); 0.491 (5.8); 0.479 (1.8); 0.146 (0.6); 0.008 (4.9); 0.000 (130.1); −0.008 (5.2); −0.150 (0.6)

### Example I-1-142

¹H-NMR (400.0 MHz, d₆-DMSO): δ=9.589 (1.2); 9.573 (1.3); 8.345 (1.1); 8.325 (1.1); 7.874 (1.0); 7.853 (4.4); 7.716 (0.6); 7.713 (0.7); 7.694 (1.1); 7.676 (0.6); 7.673 (0.6); 7.392 (16.0); 7.319 (0.6); 7.316 (0.7); 7.300 (1.2); 7.284 (0.6); 7.281 (0.6); 5.452 (3.9); 5.143 (0.6); 5.125 (0.8); 5.106 (0.6); 4.332 (0.4); 3.774 (0.4); 3.321 (27.9); 2.526 (0.7); 2.512 (15.3); 2.508 (31.7); 2.503 (43.7); 2.499 (33.3); 2.494 (16.6); 1.474 (3.7); 1.457 (3.6); 1.047 (11.5); 1.031 (11.4); 0.008 (1.7); 0.000 (48.9); −0.008 (1.9)

### Example I-1-143

¹H-NMR (400.0 MHz, d₆-DMSO): δ=9.679 (4.6); 9.662 (4.6); 8.059 (1.9); 8.045 (3.7); 8.031 (1.9); 7.870 (4.0); 7.856 (10.5); 7.849 (5.6); 7.704 (2.6); 7.685 (3.9); 7.683 (3.9); 7.664 (2.3); 7.326 (2.6); 7.308 (4.5); 7.291 (2.3); 5.755 (0.4); 5.452 (16.0); 3.325 (85.8); 3.199 (4.9); 3.183 (8.5); 3.167 (4.9); 2.674 (0.5); 2.505 (86.5); 2.332 (0.5); 1.357 (0.6); 1.234 (0.4); 1.186 (0.5); 1.035 (0.8); 1.023 (1.4); 1.016 (1.3); 1.004 (2.0); 0.991 (1.4); 0.987 (1.5); 0.974 (0.8); 0.478 (1.5); 0.466 (5.7); 0.447 (5.7); 0.434 (1.7); 0.247 (1.8); 0.236 (7.1); 0.224 (6.8); 0.211 (1.5); 0.147 (0.4); 0.000 (71.6); −0.149 (0.4)

### Example I-1-144

¹H-NMR (400.0 MHz, d₆-DMSO): δ=9.764 (11.4); 9.747 (11.6); 8.651 (15.5); 8.645 (16.0); 8.313 (0.6); 8.132 (8.8); 8.126 (8.9); 8.111 (10.0); 8.104 (10.1); 7.974 (10.0); 7.965 (10.2); 7.816 (15.9); 7.795 (13.9); 7.668 (5.2); 1.649 (10.4); 7.649 (10.4); 7.630 (6.3); 7.628 (7.2); 7.454 (12.6); 7.432 (10.6); 7.423 (6.7); 7.421 (6.8); 7.404 (11.3); 7.388 (5.1); 7.385 (5.6); 5.753 (7.7); 4.340 (0.5); 4.330 (0.5); 3.319 (302.7); 2.818 (1.0); 2.808 (2.9); 2.799 (4.7); 2.790 (6.6); 2.781 (6.7); 2.772 (4.7); 2.763 (3.1); 2.753 (1.1); 2.671 (2.1); 2.585 (0.3); 2.506 (262.8); 2.502 (352.0); 2.498 (293.9); 2.329 (2.0); 1.508 (0.6); 1.234 (0.6); 1.045 (3.4); 1.030 (3.3); 0.754 (3.7); 0.737 (15.8); 0.724 (15.1); 0.720 (13.5); 0.708 (4.6); 0.685 (0.5); 0.668 (0.4); 0.523 (0.5); 0.515 (0.5); 0.485 (4.6); 0.473 (15.0); 0.468 (15.5); 0.464 (15.2); 0.459 (14.4); 0.446 (3.9); 0.146 (1.4); 0.000 (291.8); −0.150 (1.4)

### Example I-1-145

¹H-NMR (400.0 MHz, d₆-DMSO): δ=10.190 (7.9); 9.675 (4.8); 9.658 (5.0); 7.947 (4.0); 7.925 (5.3); 7.880 (12.8); 7.803 (2.6); 7.800 (2.8); 7.784 (3.2); 7.781 (4.6); 7.762 (2.3); 7.759 (2.3); 7.646 (70.8); 7.627 (8.6); 7.408 (2.5); 7.405 (2.6); 7.389 (4.7); 7.373 (2.3); 7.370 (2.4); 7.352 (5.5); 7.333 (8.3); 7.312 (5.6); 7.054 (2.8); 7.036 (4.9); 7.017 (2.3); 5.756 (0.5); 5.511 (16.0); 3.322 (91.2); 2.678 (0.5); 2.673 (0.7); 2.669 (0.5); 2.526 (1.9); 2.513 (38.8); 2.509 (78.8); 2.504 (107.5); 2.499 (80.5); 2.495 (39.1); 2.335 (0.5); 2.331 (0.6); 2.326 (0.5); 0.146 (0.6); 0.008 (4.4); 0.000 (117.7); −0.008 (4.3); −0.150 (0.5)

### Example I-1-146

¹H-NMR (400.0 MHz, d₆-DMSO): δ=10.280 (8.7); 9.617 (5.0); 9.600 (5.1); 8.697 (7.2); 8.690 (7.2); 8.313 (0.9); 8.205 (4.2); 8.198 (4.0); 8.184 (4.5); 8.176 (4.4); 7.965 (4.1); 7.943 (5.5); 7.875 (12.7); 7.840 (2.7); 7.837 (2.8); 7.818 (4.5); 7.799 (2.3); 7.797 (2.2); 7.481 (7.2); 7.460 (7.0); 7.427 (2.6); 7.424 (2.6); 7.408 (4.9); 7.392 (2.3); 7.389 (2.3); 5.753 (8.5); 5.510 (16.0); 3.320 (615.3); 2.676 (1.9); 2.671 (2.6); 2.667 (1.9); 2.524 (7.8); 2.507 (323.3); 2.502 (431.5); 2.498 (324.5); 2.494 (161.3); 2.334 (1.9); 2.329 (2.5); 2.324 (1.8); 1.232 (0.7); 1.046 (1.3); 1.030 (1.2); 0.146 (2.0); 0.008 (16.6); 0.000 (426.1); −0.008 (17.3); −0.150 (2.0)

### Example I-1-147

¹H-NMR (400.0 MHz, d₆-DMSO): δ=9.667 (2.0); 9.651 (2.0); 8.075 (0.8); 8.060 (1.6); 8.045 (0.8); 7.869 (1.7); 7.854 (5.1); 7.847 (2.3); 7.704 (1.0); 7.701 (1.0); 7.682 (1.8); 7.664 (0.9); 7.661 (0.9); 7.328 (1.0); 7.325 (1.0); 7.309 (1.9); 7.293 (1.0); 7.290 (0.9); 5.457 (6.8); 3.327 (19.9); 3.161 (2.3); 3.145 (4.1); 3.129 (2.4); 2.511 (20.3); 2.506 (27.2); 2.502 (20.2); 1.802 (0.5); 1.785 (0.9); 1.768 (1.2); 1.752 (1.0); 1.735 (0.5); 0.922 (16.0); 0.905 (15.6); 0.008 (1.1); 0.000 (29.9); −0.008 (1.2)

### Example I-1-148

¹H-NMR (400.0 MHz, d₆-DMSO): δ=9.678 (3.1); 9.662 (3.2); 8.374 (1.3); 8.359 (2.7); 8.344 (1.3); 7.879 (2.6); 7.857 (3.6); 7.849 (7.7); 7.720 (1.7); 7.701 (2.8); 7.681 (1.4); 7.342 (16.0); 7.331 (15.1); 7.322 (4.2); 7.305 (1.7); 7.279 (0.4); 7.269 (1.2); 7.258 (1.9); 7.248 (2.0); 7.237 (1.2); 7.227 (0.5); 5.448 (10.7); 4.530 (6.3); 4.334 (0.3); 3.325 (66.7); 2.673 (0.4); 2.504 (59.1); 2.332 (0.4); 1.048 (2.3); 1.033 (2.3); 0.000 (57.1)

### Example I-1-149

¹H-NMR (400.0 MHz, d₆-DMSO): δ=9.660 (3.8); 9.643 (3.9); 8.406 (1.5); 8.390 (3.2); 8.375 (1.6); 7.881 (3.1); 7.859 (4.2); 7.850 (10.0); 7.724 (2.0); 7.721 (2.1); 7.706 (2.5); 7.703 (3.5); 7.700 (2.1); 7.684 (1.8); 7.681 (1.8); 7.399 (3.5); 7.394 (1.8); 7.384 (2.7); 7.378 (16.0); 7.364 (13.3); 7.348 (1.5); 7.342 (3.3); 7.337 (2.5); 7.334 (2.3); 7.317 (3.6); 7.302 (1.9); 7.299 (2.1); 7.275 (0.4); 5.499 (12.4); 4.515 (6.8); 4.500 (6.8); 4.343 (0.5); 4.332 (0.5); 4.294 (0.5); 4.279 (0.5); 3.323 (62.4); 2.678 (0.4); 2.673 (0.5); 2.669 (0.4); 2.527 (1.3); 2.513 (30.1); 2.509 (63.7); 2.504 (89.4); 2.500 (67.3); 2.495 (32.6); 2.335 (0.4); 2.331 (0.5); 2.326 (0.4); 1.047 (5.1); 1.032 (5.0); 0.146 (0.5); 0.008 (4.0); 0.000 (116.5); −0.008 (4.2); −0.150 (0.5)

### Example I-1-150

¹H-NMR (400.0 MHz, d₆-DMSO): δ=10.244 (8.6); 9.639 (5.0); 9.623 (5.2); 7.951 (4.1); 7.929 (5.5); 7.874 (13.2); 7.814 (2.8); 7.811 (2.8); 7.795 (3.4); 7.792 (4.6); 7.773 (2.4); 7.770 (2.3); 7.696 (1.2); 7.688 (11.4); 7.683 (3.7); 7.671 (4.0); 7.666 (13.3); 7.658 (1.3); 7.414 (2.7); 7.411 (2.7); 7.395 (5.1); 7.382 (14.0); 7.377 (6.2); 7.365 (3.8); 7.360 (11.7); 7.352 (1.1); 5.755 (1.3); 5.507 (16.0); 3.322 (87.7); 2.678 (0.6); 2.673 (0.8); 2.668 (0.6); 2.526 (2.3); 2.513 (47.4); 2.508 (94.8); 2.504 (127.6); 2.499 (94.5); 2.495 (45.6); 2.335 (0.6); 2.331 (0.7); 2.326 (0.5); 0.146 (0.7); 0.008 (5.9); 0.000 (149.5); −0.008 (5.3); −0.150 (0.7)

### Example I-1-151

¹H-NMR (400.0 MHz, d₆-DMSO): δ=9.695 (0.8); 9.678 (0.8); 7.943 (1.2); 7.853 (0.8); 7.845 (2.1); 7.832 (0.9); 7.688 (0.5); 7.685 (0.4); 7.670 (0.6); 7.667 (0.8); 7.648 (0.4); 7.645 (0.4); 7.302 (0.4); 7.299 (0.4); 7.283 (0.4); 7.283 (0.7); 7.26 (04 (0.4); 5.756 (0.6); 5.435 (2.7); 3.321 (15.1); 2.527 (0.4); 2.513 (6.8); 2.509 (13.5); 2.504 (17.9); 2.500 (13.2); 2.495 (6.3); 1.387 (16.0); 1.233 (0.3); 0.008 (0.9); 0.000 (21.8); −0.009 (0.8)

### Example I-1-152

¹H-NMR (400.0 MHz, d₆-DMSO): δ=9.666 (3.0); 9.649 (3.0); 9.114 (7.2); 8.826 (16.0); 8.005 (2.4); 7.996 (2.4); 7.799 (2.4); 7.777 (3.3); 7.678 (1.6); 7.675 (1.6); 7.660 (2.1); 7.657 (2.7); 7.638 (1.4); 7.635 (1.3); 7.334 (1.5); 7.332 (1.5); 7.315 (2.8); 7.299 (1.4); 7.296 (1.4); 5.756 (1.1); 5.306 (10.5); 3.323 (13.0); 2.788 (0.8); 2.779 (1.2); 2.770 (1.8); 2.761 (1.8); 2.752 (1.2); 2.743 (0.8); 2.526 (0.8); 2.508 (38.6); 2.504 (52.0); 2.499 (38.7); 2.495 (18.9); 1.047 (3.4); 1.032 (3.3); 0.741 (1.1); 0.728 (3.2); 0.724 (4.0); 0.711 (4.1); 0.706 (3.2); 0.694 (1.3); 0.496 (1.3); 0.485 (3.7); 0.480 (3.6); 0.475 (3.4); 0.470 (3.4); 0.458 (1.0); 0.008 (1.3); 0.000 (34.3); −0.008 (1.3)

### Example I-1-153

¹H-NMR (400.0 MHz, d₆-DMSO): δ=10.692 (8.8); 9.629 (4.9); 9.613 (5.1); 8.341 (7.1); 8.334 (7.2); 8.314 (0.4); 8.275 (6.6); 8.252 (7.6); 7.972 (4.0); 7.950 (5.4); 7.914 (4.3); 7.908 (4.1); 7.892 (3.9); 7.885 (3.9); 7.875 (12.4); 7.848 (2.7); 7.827 (4.3); 7.808 (2.2); 7.805 (2.1); 7.434 (2.6); 7.432 (2.6); 7.415 (4.9); 7.399 (2.3); 5.755 (4.4); 5.508 (16.0); 3.318 (84.1); 2.676 (0.9); 2.672 (1.2); 2.668 (0.9); 2.525 (3.7); 2.507 (149.4); 2.503 (201.3); 2.499 (151.6); 2.334 (0.9); 2.330 (1.2); 2.325 (0.9); 0.146 (0.6); 0.008 (5.0); 0.000 (124.9); −0.008 (4.8); −0.150 (0.6)

### Example I-1-154

¹H-NMR (400.0 MHz, d₆-DMSO): δ=9.664 (4.5); 9.647 (4.6); 8.506 (5.6); 8.500 (5.7); 8.022 (3.9); 8.013 (3.9); 7.797 (3.2); 7.791 (3.2); 7.777 (3.7); 7.770 (3.6); 7.724 (3.0); 7.702 (5.4); 7.662 (2.8); 7.660 (2.9); 7.644 (3.3); 7.641 (4.0); 7.622 (1.8); 7.619 (1.7); 7.489 (6.5); 7.468 (5.7); 7.329 (2.3); 7.326 (2.4); 7.309 (4.1); 7.294 (2.1); 7.291 (2.0); 5.755 (4.6); 5.278 (16.0); 4.342 (0.9); 4.331 (4.331 (0.9); 3.780 (0.3); 3.321 (44.1); 2.800 (0.4); 2.791 (1.2); 2.782 (1.9); 2.773 (2.8); 2.764 (2.8); 2.755 (1.9); 2.746 (1.3); 2.736 (0.5); 2.677 (0.4); 2.673 (0.5); 2.669 (0.4); 2.526 (1.5); 2.509 (61.3); 2.504 (83.4); 2.500 (63.3); 2.335 (0.4); 2.331 (0.5); 2.327 (0.4); 1.233 (0.5); 1.047 (5.9); 1.032 (5.8); 0.745 (1.6); 0.732 (4.8); 0.727 (6.3); 0.715 (6.2); 0.710 (5.0); 0.698 (2.0); 0.543 (0.3); 0.531 (0.4); 0.526 (0.4); 0.496 (2.0); 0.486 (5.8); 0.480 (5.7); 0.475 (5.3); 0.470 (5.4); 0.458 (1.6); 0.008 (2.2); 0.000 (55.7); −0.008 (2.3)

### Example I-1-155

¹H-NMR (400.0 MHz, d₆-DMSO): δ=9.501 (1.3); 9.495 (1.4); 9.489 (1.5); 9.483 (1.3); 8.482 (2.4); 8.477 (2.4); 7.827 (0.6); 7.821 (0.6); 7.803 (1.4); 7.797 (2.4); 7.791 (1.5); 7.783 (1.3); 7.777 (2.5); 7.770 (1.6); 7.743 (1.5); 7.731 (1.6); 7.719 (0.9); 7.707 (0.8); 7.490 (2.7); 7.469 (2.3); 5.755 (1.6); 5.234 (6.8); 3.754 (16.0); 3.318 (16.8); 2.507 (32.2); 2.503 (42.4); 2.498 (32.8); 1.046 (1.3); 1.031 (1.3); 0.000 (24.6)

### Example I-1-156

¹H-NMR (400.0 MHz, d₆-DMSO): δ=9.441 (1.0); 9.438 (1.1); 9.436 (1.1); 9.432 (1.1); 9.430 (1.1); 9.427 (1.0); 8.389 (1.7); 8.383 (1.7); 8.089 (0.7); 8.084 (0.6); 8.067 (0.8); 8.061 (1.2); 8.055 (0.7); 8.038 (0.7); 8.032 (0.6); 7.772 (1.0); 7.766 (1.0); 7.752 (1.2); 7.745 (1.2); 7.501 (2.3); 7.480 (2.0); 5.754 (3.0); 5.260 (4.2); 3.775 (16.0); 3.317 (23.4); 2.524 (0.7); 2.511 (15.8); 2.506 (32.5); 2.502 (44.5); 2.497 (33.2); 2.493 (16.1); 0.008 (1.0); 0.000 (29.6); −0.008 (1.1)

### Example I-1-157

¹H-NMR (400.0 MHz, d₆-DMSO): δ=8.497 (2.6); 8.492 (2.7); 7.793 (1.4); 7.788 (1.4); 7.773 (1.6); 7.767 (1.6); 7.722 (1.2); 7.702 (2.3); 7.682 (1.6); 7.533 (2.2); 7.512 (1.8); 7.494 (2.9); 7.473 (2.5); 7.203 (2.0); 7.184 (1.9); 5.223 (7.8); 4.342 (0.6); 4.331 (0.6); 3.702 (16.0); 3.320 (27.6); 2.554 (12.8); 2.503 (47.5); 1.046 (4.0); 1.031 (4.0); 0.000 (23.0)

### Example I-1-158

¹H-NMR (400.0 MHz, d₆-DMSO): δ=9.487 (2.1); 9.470 (2.2); 8.592 (3.6); 8.558 (3.1); 8.554 (3.1); 7.902 (3.2); 7.741 (0.6); 7.719 (1.9); 7.701 (4.2); 7.678 (0.8); 7.346 (1.0); 7.342 (1.0); 7.329 (1.9); 7.313 (0.9); 5.245 (8.0); 5.245 (842 (16.0); 3.742 (16.0); 3.326 (23.3); 2.505 (31.2); 1.048 (1.4); 1.032 (1.3); 0.000 (21.7)

### Example I-1-159

¹H-NMR (400.0 MHz, d₆-DMSO): δ=9.908 (2.1); 9.892 (2.1); 8.360 (2.7); 8.354 (2.7); 7.687 (1.4); 7.680 (1.4); 7.665 (2.6); 7.660 (3.3); 7.642 (2.1); 7.502 (3.4); 7.482 (2.8); 7.343 (1.9); 7.325 (2.5); 7.307 (1.7); 5.475 (7.6); 4.844 (16.0); 3.319 (19.4); 2.525 (0.6); 2.512 (13.1); 2.507 (26.8); 2.503 (36.9); 2.498 (27.5); 2.494 (13.2); 2.432 (13.3); 0.008 (0.5); 0.000 (13.2); −0.009 (0.4)

### Example I-1-160

¹H-NMR (400.0 MHz, d₆-DMSO): δ=9.526 (2.1); 9.510 (2.2); 8.315 (3.1); 7.608 (1.4); 7.602 (1.2); 7.587 (2.0); 7.485 (3.7); 7.472 (2.8); 7.464 (2.6); 7.261 (1.5); 7.244 (2.4); 7.226 (1.3); 5.452 (7.4); 4.341 (0.7); 4.332 (0.6); 3.779 (0.4); 3.743 (16.0); 3.320 (37.5); 3.309 (3.3); 2.672 (0.4); 2.506 (56.2); 2.503 (61.2); 2.418 (12.0); 2.329 (0.4); 1.049 (3.5); 1.046 (5.7); 1.034 (3.7); 1.031 (5.8); 0.003 (24.8); 0.000 (40.8)

### Example I-1-161

¹H-NMR (400.0 MHz, d₆-DMSO): δ=9.901 (2.3); 9.885 (2.4); 8.501 (3.0); 8.495 (3.0); 7.891 (1.6); 7.885 (1.6); 7.870 (1.8); 7.864 (1.8); 7.833 (1.0); 7.831 (1.0); 7.812 (2.2); 7.793 (1.4); 7.790 (1.3); 7.595 (2.4); 7.573 (2.0); 7.513 (3.5); 7.492 (3.2); 7.405 (1.3); 7.403 (1.3); 7.386 (2.4); 7.370 (1.2); 6.040 (0.5); 6.022 (1.6); 6.004 (1.7); 5.986 (0.5); 4.795 (16.0); 3.320 (33.5); 2.512 (16.8); 2.508 (33.5); 2.503 (45.3); 2.499 (33.7); 1.924 (7.8); 1.906 (7.7); 0.008 (0.6); 0.000 (12.3); −0.008 (0.5)

### Example I-1-162

¹H-NMR (400.0 MHz, d₆-DMSO): δ=9.863 (2.6); 9.847 (2.6); 8.608 (3.9); 8.604 (3.8); 8.571 (3.7); 8.565 (3.7); 8.314 (0.9); 7.939 (3.7); 7.902 (1.0); 7.883 (2.4); 7.864 (1.6); 7.780 (3.0); 7.758 (2.1); 7.419 (1.4); 7.403 (2.6); 7.386 (1.3); 5.276 (9.5); 4.810 (16.0); 3.568 (0.9); 3.322 (83.1); 2.672 (0.6); 2.507 (69.7); 2.503 (90.2); 2.498 (68.9); 2.329 (0.5); 0.146 (0.4); 0.000 (74.5); −0.149 (0.4)

### Example I-1-163

¹H-NMR (400.0 MHz, d₆-DMSO): δ=9.874 (2.9); 9.857 (3.0); 8.854 (4.0); 8.315 (0.5); 8.024 (1.8); 8.004 (2.4); 7.906 (1.3); 7.887 (6.3); 7.867 (4.7); 7.783 (3.3); 7.762 (2.3); 7.429 (1.6); 7.412 (2.9); 7.395 (1.5); 5.386 (9.4); 4.810 (16.0); 3.570 (1.2); 3.326 (11.7); 2.506 (33.3); 0.000 (23.8)

### Example I-1-164

¹H-NMR (400.0 MHz, d₆-DMSO): δ=10.026 (4.3); 10.010 (4.5); 8.406 (5.2); 7.913 (4.1); 7.893 (4.5); 7.752 (2.7); 7.747 (2.3); 7.731 (3.2); 7.727 (2.7); 7.488 (4.7); 7.467 (4.2); 7.380 (2.4); 7.379 (2.5); 7.362 (3.7); 7.343 (2.4); 5.754 (1.0); 5.528 (12.8); 4.571 (16.0); 3.372 (28.0); 3.371 (28.3); 3.318 (51.9); 2.672 (0.6); 2.502 (100.2); 2.328 (0.6); 0.000 (54.3); −0.002 (54.8); −0.016 (2.7)

### Example I-1-165

¹H-NMR (400.0 MHz, d₆-DMSO): δ=9.950 (2.2); 9.944 (2.4); 9.938 (2.4); 9.932 (2.1); 8.497 (3.8); 8.491 (3.7); 7.945 (1.0); 7.939 (1.0); 7.924 (1.3); 7.921 (1.8); 7.915 (1.6); 7.900 (1.5); 7.894 (1.4); 7.820 (2.2); 7.812 (2.9); 7.799 (4.6); 7.793 (2.8); 7.787 (1.7); 7.774 (1.6); 7.504 (4.4); 7.483 (3.9); 5.247 (10.6); 4.543 (16.0); 3.365 (31.2); 3.319 (92.6); 2.676 (0.5); 2.671 (0.6); 2.667 (0.5); 2.507 (75.4); 2.502 (102.3); 2.498 (76.2); 2.334 (0.5); 2.329 (0.6); 2.325 (0.5); 0.146 (0.4); 0.000 (91.0); −0.008 (3.5); −0.150 (0.4)

### Example I-1-166

¹H-NMR (400.0 MHz, d₆-DMSO): δ=8.510 (3.4); 8.504 (3.4); 7.826 (1.8); 7.804 (4.4); 7.789 (2.6); 7.785 (3.3); 7.577 (2.7); 7.555 (2.2); 7.498 (4.1); 7.477 (3.6); 7.255 (2.5); 7.236 (2.4); 5.754 (8.4); 5.246 (9.8); 4.620 (16.0); 3.806 (0.5); 3.338 (31.1); 3.320 (114.8); 2.676 (0.4); 2.671 (0.6); 2.667 (0.4); 2.551 (16.8); 2.525 (1.4); 2.511 (31.3); 2.507 (65.9); 2.502 (92.3); 2.498 (69.0); 2.498 (69.0); 2.4 (33.1); 2.334 (0.4); 2.329 (0.6); 2.325 (0.4); 0.146 (0.4); 0.008 (2.8); 0.000 (84.2); −0.008 (2.8); −0.150 (0.4)

### Example I-1-167

¹H-NMR (400.0 MHz, d₆-DMSO): δ=9.754 (4.0); 8.496 (3.4); 8.490 (3.3); 7.798 (1.9); 7.792 (1.9); 7.777 (2.2); 7.771 (2.1); 7.700 (1.3); 7.697 (1.3); 7.678 (2.8); 7.674 (2.8); 7.642 (4.2); 7.620 (1.9); 7.492 (3.8); 7.472 (3.4); 5.213 (9.4); 4.532 (14.3); 3.363 (28.3); 3.321 (55.5); 2.673 (0.4); 2.508 (43.4); 2.503 (57.3); 2.499 (43.1); 2.365 (16.0); 2.330 (0.4); 0.000 (48.8); −0.008 (2.0)

### Example I-1-168

¹H-NMR (400.0 MHz, d₆-DMSO): δ=9.860 (2.7); 9.844 (2.7); 8.478 (3.9); 8.473 (3.9); 8.355 (0.4); 8.314 (0.3); 8.148 (4.1); 8.143 (3.9); 8.034 (0.4); 8.030 (0.3); 7.903 (1.1); 7.901 (1.1); 7.882 (2.5); 7.863 (1.7); 7.775 (3.0); 7.754 (2.1); 7.420 (1.5); 7.403 (2.7); 7.387 (1.3); 5.271 (9.9); 4.805 (16.0); 4.506 (0.5); 4.491 (0.5); 3.569 (0.9); 3.321 (35.2); 2.673 (0.3); 2.508 (41.0); 2.504 (52.8); 2.500 (40.4); 1.234 (0.6); 0.000 (49.0)

### Example I-1-169

¹H-NMR (400.0 MHz, d₆-DMSO): δ=9.944 (3.3); 9.928 (3.4); 8.657 (4.5); 8.650 (5.4); 8.313 (6.8); 8.306 (2.8); 8.139 (2.3); 8.133 (2.9); 8.118 (2.8); 8.112 (3.3); 7.887 (1.6); 7.868 (3.2); 7.838 (5.0); 7.817 (3.9); 7.505 (2.1); 7.485 (6.3); 7.463 (4.1); 4.796 (16.0); 3.568 (0.5); 3.319 (54.0); 2.668 (0.8); 2.501 (126.5); 2.500 (126.5); 2.328 (0.8); 0.145 (0.3); 0.000 (67.0); −0.150 (0.4)

### Example I-1-1170

¹H-NMR (400.0 MHz, d₆-DMSO): δ=9.694 (3.3); 9.688 (3.6); 9.682 (3.7); 9.676 (3.1); 8.489 (5.9); 8.484 (5.8); 8.314 (0.5); 7.988 (4.2); 7.979 (4.2); 7.795 (4.6); 7.788 (3.7); 7.773 (7.3); 7.760 (4.1); 7.743 (2.6); 7.737 (2.4); 7.722 (3.0); 7.718 (2.8); 7.699 (1.2); 7.693 (1.1); 7.492 (6.4); 7.471 (5.6); 5.282 (16.0); 4.341 (0.7); 4.330 (0.7); 3.318 (276.0); 2.800 (0.4); 2.791 (1.2); 2.782 (1.8); 2.773 (2.7); 2.764 (2.7); 2.755 (1.9); 2.746 (1.2); 2.736 (0.5); 2.671 (2.1); 2.506 (275.2); 2.502 (345.6); 2.328 (2.0); 1.045 (4.1); 1.030 (4.1); 0.752 (1.5); 0.734 (6.1); 0.722 (6.3); 0.717 (5.0); 0.705 (1.8); 0.505 (1.9); 0.494 (6.1); 0.488 (6.1); 0.479 (5.5); 0.466 (1.5); 0.147 (0.7); 0.000 (146.4); −0.149 (0.7)

### Example I-1-171

¹H-NMR (400.0 MHz, d₆-DMSO): δ=8.504 (4.6); 8.498 (4.5); 8.081 (2.6); 7.792 (2.6); 7.786 (2.6); 7.771 (2.9); 7.765 (2.7); 7.653 (1.4); 7.632 (3.1); 7.613 (2.7); 7.572 (3.8); 7.551 (2.1); 7.495 (4.3); 7.475 (3.8); 7.170 (3.1); 7.152 (2.8); 5.755 (0.9); 5.278 (11.6); 3.318 (55.5); 3.313 (13.9); 2.744 (16.0); 2.723 (2.0); 2.714 (1.8); 2.671 (0.9); 2.502 (124.4); 2.373 (0.4); 2.329 (0.8); 2.300 (0.6); 2.232 (0.6); 1.743 (0.5); 1.356 (1.0); 1.234 (0.6); 0.702 (1.2); 0.689 (4.1); 0.685 (4.7); 0.673 (4.8); 0.668 (4.0); 0.656 (1.5); 0.635 (0.4); 0.617 (0.4); 0.487 (1.7); 0.476 (5.1); 0.470 (5.4); 0.461 (4.5); 0.450 (1.2); 0.000 (46.0); −0.006 (9.6)

### Example I-1-1172

¹H-NMR (400.0 MHz, d₆-DMSO): δ=10.164 (2.5); 10.161 (2.7); 10.147 (2.6); 10.145 (2.7); 8.416 (2.8); 8.410 (2.9); 8.393 (2.5); 8.391 (2.6); 8.374 (2.7); 8.371 (2.6); 7.821 (1.6); 7.814 (1.6); 7.800 (1.9); 7.793 (1.8); 7.558 (2.2); 7.541 (2.6); 7.538 (2.8); 7.532 (3.6); 7.521 (2.2); 7.511 (3.0); 5.493 (8.1); 4.829 (16.0); 3.318 (19.6); 2.506 (21.6); 2.502 (29.7); 2.497 (22.5); 0.008 (0.7); 0.000 (17.9); −0.008 (0.7)

### Example I-1-1173

¹H-NMR (400.0 MHz, d₆-DMSO): δ=9.737 (2.2); 7.765 (0.5); 7.743 (3.0); 7.735 (2.3); 7.732 (2.1); 7.713 (0.4); 7.709 (0.4); 7.382 (3.8); 7.311 (0.3); 5.256 (0.5); 5.231 (5.3); 4.519 (8.6); 3.958 (1.0); 3.946 (16.0); 3.881 (1.2); 3.783 (0.6); 3.362 (18.4); 3.325 (2.1); 3.260 (2.7); 2.543 (0.7); 2.526 (0.5); 2.513 (12.1); 2.508 (25.8); 2.504 (36.1); 2.499 (26.9); 2.495 (13.0); 2.367 (9.1); 2.268 (0.7); 0.000 (10.0); −0.008 (0.4)

### Example I-1-174

¹H-NMR (400.0 MHz, d₆-DMSO): δ=9.738 (3.9); 8.077 (0.3); 7.831 (6.5); 7.812 (2.1); 7.790 (4.3); 7.775 (0.7); 7.753 (2.8); 7.750 (2.9); 7.728 (1.4); 7.540 (0.5); 5.393 (0.8); 5.365 (9.6); 4.522 (14.0); 4.041 (0.3); 3.921 (1.0); 3.364 (26.0); 3.319 (40.7); 3.291 (2.0); 2.673 (0.4); 2.543 (1.2); 2.508 (48.4); 2.504 (67.7); 2.500 (53.3); 2.371 (16.0); 2.331 (0.5); 2.304 (1.2); 2.212 (0.8); 2.076 (0.8); 0.146 (0.3); 0.007 (2.9); 0.000 (68.8); −0.008 (4.2); −0.150 (0.3)

### Example I-1-1175

¹H-NMR (400.0 MHz, d₆-DMSO): δ=9.747 (4.3); 7.831 (6.6); 7.809 (2.1); 7.787 (4.2); 7.749 (2.9); 7.746 (2.9); 7.727 (1.5); 5.364 (9.4); 4.559 (13.7); 3.963 (0.6); 3.583 (1.8); 3.565 (6.0); 3.548 (6.1); 3.530 (2.0); 3.320 (20.9); 2.544 (1.1); 2.505 (46.1); 2.501 (37.1); 2.368 (16.0); 2.331 (0.3); 2.267 (0.4); 1.174 (5.9); 1.156 (12.1); 1.139 (5.9); 1.115 (0.5); 0.000 (44.7)

### Example I-1-1176

¹H-NMR (400.0 MHz, d₆-DMSO): δ=9.736 (2.7); 9.720 (2.7); 8.623 (1.2); 8.608 (2.7); 8.592 (1.3); 8.338 (3.5); 8.332 (3.5); 8.135 (0.4); 7.622 (1.8); 7.615 (1.8); 7.601 (2.5); 7.595 (2.5); 7.487 (4.8); 7.466 (3.6); 7.429 (2.4); 7.410 (2.9); 7.401 (2.5); 7.395 (1.4); 7.386 (1.8); 7.379 (14.8); 7.369 (12.3); 7.363 (1.8); 7.354 (1.0); 7.347 (2.1); 7.295 (0.6); 7.273 (0.4); 7.239 (2.7); 7.221 (3.5); 7.203 (2.2); 5.510 (8.8); 4.522 (5.7); 4.507 (5.7); 4.340 (1.2); 4.330 (1.3); 4.293 (0.5); 4.278 (0.5); 3.779 (0.4); 3.769 (0.4); 3.764 (0.3); 3.320 (93.9); 2.676 (0.3); 2.672 (0.5); 2.667 (0.3); 2.525 (1.0); 2.520 (1.4); 2.512 (25.6); 2.507 (55.9); 2.503 (79.2); 2.498 (57.8); 2.494 (26.8); 2.446 (0.6); 2.430 (16.0); 2.334 (0.3); 2.32934 (0.5); 2.3295 (0.4); 1.046 (8.4); 1.031 (8.3); 0.146 (0.4); 0.008 (3.1); 0.000 (103.4); −0.009 (3.2); −0.150 (0.5)

### Example I-1-177

¹H-NMR (400.0 MHz, d₆-DMSO): δ=9.917 (2.9); 9.901 (3.0); 8.605 (4.2); 8.601 (4.4); 8.573 (4.2); 8.567 (4.3); 7.937 (2.4); 7.932 (4.2); 7.928 (2.5); 7.842 (1.1); 7.823 (2.5); 7.802 (2.0); 7.747 (3.5); 7.725 (2.1); 7.386 (1.6); 7.369 (2.8); 7.351 (1.4); 5.761 (0.4); 5.260 (10.4); 4.591 (16.0); 3.594 (2.1); 3.577 (6.8); 3.559 (6.9); 3.542 (2.2); 3.325 (60.7); 2.679 (0.4); 2.674 (0.4); 2.531 (1.0); 2.514 (52.1); 2.510 (73.8); 2.505 (56.6); 2.336 (0.4); 1.240 (0.4); 1.182 (6.9); 1.165 (13.9); 1.147 (6.7)

### Example I-1-178

¹H-NMR (400.0 MHz, d₆-DMSO): δ=9.943 (4.6); 9.927 (4.6); 8.491 (5.7); 8.485 (6.1); 8.313 (0.6); 8.265 (0.4); 8.260 (0.5); 7.873 (3.2); 7.867 (3.2); 7.852 (3.6); 7.846 (3.5); 7.765 (1.8); 7.762 (2.1); 7.744 (4.3); 7.725 (2.6); 7.722 (3.0); 7.547 (4.6); 7.525 (3.8); 7.506 (7.2); 7.485 (6.4); 7.362 (2.7); 7.345 (4.8); 7.329 (2.2); 7.327 (2.6); 6.798 (0.6); 6.776 (0.5); 6.018 (0.9); 6.000 (3.2); 5.982 (3.2); 5.964 (1.0); 4.565 (0.8); 4.538 (1.4); 4.524 (13.9); 4.520 (14.5); 4.478 (0.7); 3.828 (3.7); 3.361 (5.6); 3.356 (51.8); 3.316 (212.7); 2.676 (1.0); 2.671 (1.4); 2.667 (1.0); 2.524 (2.8); 2.506 (160.9); 2.502 (230.4); 2.497 (176.5); 2.493 (87.2); 2.333 (1.0); 2.329 (1.3); 2.324 (1.0); 1.913 (15.0); 1.895 (16.0); 1.877 (1.4); 1.235 (0.4); 1.030 (0.3); 0.146 (1.2); 0.008 (8.2); 0.000 (266.6); −0.008 (10.4); −0.150 (1.2)

### Example I-1-1179

¹H-NMR (400.0 MHz, d₆-DMSO): δ=9.905 (2.7); 9.888 (2.9); 8.468 (4.1); 8.462 (4.2); 8.137 (4.4); 8.132 (4.3); 7.841 (1.1); 7.838 (1.1); 7.819 (2.7); 7.816 (1.9); 7.800 (1.9); 7.797 (1.8); 7.738 (3.2); 7.716 (2.0); 7.385 (1.4); 7.382 (1.4); 7.366 (2.6); 7.350 (1.5); 7.347 (1.3); 5.250 (10.1); 5.154 (0.4); 4.541 (16.0); 3.910 (1.3); 3.366 (33.3); 3.321 (57.4); 2.527 (0.7); 2.522 (1.0); 2.513 (18.0); 2.509 (38.4); 2.504 (53.8); 2.500 (39.4); 2.495 (18.5); 0.008 (2.1); 0.000 (67.5); −0.008 (2.2)

### Example I-1-180

¹H-NMR (400.0 MHz, d₆-DMSO): δ=9.918 (2.8); 9.902 (2.9); 8.845 (3.4); 8.008 (1.5); 8.004 (1.5); 7.988 (2.0); 7.983 (2.0); 7.881 (4.1); 7.861 (3.0); 7.843 (1.1); 7.840 (1.1); 7.821 (2.6); 7.819 (1.9); 7.803 (1.8); 7.800 (1.8); 7.741 (3.3); 7.720 (2.1); 7.392 (1.4); 7.390 (1.5); 7.373 (2.7); 7.357 (1.3); 7.355 (1.3); 5.363 (8.8); 4.547 (16.0); 3.368 (31.2); 3.320 (28.1); 2.527 (0.5); 2.514 (12.8); 2.509 (27.1); 2.505 (38.0); 2.501 (28.3); 2.496 (13.6); 0.008 (1.5); 0.000 (47.5); −0.008 (1.7)

### Example I-1-181

¹H-NMR (400.0 MHz, d₆-DMSO): δ=9.732 (2.7); 9.716 (2.7); 8.322 (3.4); 8.316 (3.4); 8.316 (3.4); 8.190 (2.5); 8.180 (2.5); 7.598 (1.6); 7.592 (1.6); 7.578 (2.2); 7.571 (2.3); 7.482 (4.6); 7.461 (3.2); 7.420 (2.2); 7.401 (2.7); 7.244 (2.3); 7.226 (3.3); 7.209 (1.9); 5.753 (1.0); 5.490 (9.2); 3.315 (114.0); 2.800 (0.8); 2.790 (1.2); 2.782 (1.7); 2.772 (1.8); 2.763 (1.2); 2.754 (0.8); 2.675 (0.7); 2.671 (1.0); 2.666 (0.8); 2.523 (2.2); 2.506 (119.9); 2.501 (169.0); 2.497 (128.0); 2.440 (0.6); 2.425 (16.0); 2.333 (0.7); 2.328 (1.0); 2.324 (0.7); 1.045 (0.3); 1.030 (0.3); 0.745 (1.0); 0.733 (3.0); 0.728 (3.9); 0.715 (3.9); 0.711 (3.0); 0.699 (1.2); 0.494 (1.2); 0.483 (3.7); 0.478 (3.4); 0.473 (3.2); 0.468 (3.3); 0.456 (1.0); 0.146 (0.9); 0.008 (5.7); 0.000 (186.2); −0.008 (6.9); −0.150 (0.9)

### Example I-1-182

¹H-NMR (400.0 MHz, d₆-DMSO): δ=9.960 (2.6); 9.944 (2.7); 8.345 (3.4); 8.339 (3.5); 7.657 (1.8); 7.650 (1.7); 7.636 (2.3); 7.629 (2.2); 7.593 (2.3); 7.574 (2.6); 7.495 (4.4); 7.474 (3.5); 7.301 (2.3); 7.283 (3.2); 7.266 (2.0); 5.457 (9.0); 4.609 (15.4); 3.589 (2.1); 3.571 (6.9); 3.554 (7.0); 3.536 (2.2); 3.319 (55.0); 2.672 (0.3); 2.525 (0.8); 2.507 (40.6); 2.503 (56.3); 2.498 (42.0); 2.422 (16.0); 2.329 (0.4); 1.178 (6.9); 1.160 (14.1); 1.143 (6.8); 1.074 (0.5); 1.057 (0.9); 1.039 (0.5); 0.008 (2.2); 0.000 (64.5); −0.008 (2.5)

### Example I-1-183

¹H-NMR (400.0 MHz, d₆-DMSO): δ=9.685 (2.3); 9.668 (2.3); 8.319 (2.9); 8.160 (2.2); 8.140 (2.3); 7.610 (1.2); 7.605 (1.3); 7.584 (1.8); 7.487 (2.9); 7.466 (2.1); 7.114 (1.5); 7.094 (2.2); 7.077 (1.5); 5.752 (9.4); 5.634 (6.6); 3.854 (0.5); 3.843 (0.5); 3.823 (0.6); 3.776 (1.0); 3.742 (16.0); 3.691 (1.4); 3.684 (1.4); 3.667 (1.0); 3.638 (1.3); 3.617 (1.4); 3.555 (0.6); 3.505 (0.9); 3.496 (1.0); 3.483 (0.9); 3.447 (0.9); 3.431 (0.6); 3.418 (0.6); 3.391 (0.5); 3.381 (0.6); 3.316 (241.8); 2.670 (2.5); 2.501 (428.9); 2.497 (383.0); 2.402 (0.3); 2.327 (2.6); 1.235 (0.8); 1.176 (0.5); 1.158 (0.5); 1.138 (0.4); 1.121 (0.6); 1.045 (0.4); 1.030 (0.4); 0.000 (2.9)

### Example I-1-184

¹H-NMR (400.0 MHz, d₆-DMSO): δ=9.597 (6.2); 9.580 (6.5); 9.580 (6.33 (0.3); 8.313 (0.3); 8.179 (6.1); 8.174 (6.1); 7.873 (3.8); 7.863 (3.9); 7.852 (10.9); 7.438 (3.7); 7.433 (3.7); 7.420 (3.6); 7.415 (3.7); 5.400 (14.1); 4.338 (0.7); 4.329 (0.7); 3.785 (0.3); 3.775 (0.5); 3.757 (0.4); 3.317 (132.8); 2.792 (0.4); 2.782 (1.1); 2.773 (1.7); 2.764 (2.4); 2.755 (2.5); 2.746 (1.7); 2.737 (1.1); 2.727 (0.4); 2.676 (0.7); 2.671 (1.0); 2.667 (0.7); 2.525 (2.1); 2.507 (121.6); 2.502 (171.9); 2.498 (130.4); 2.334 (0.7); 2.329 (1.0); 2.325 (0.8); 1.046 (16.0); 1.031 (15.8); 0.749 (1.4); 0.736 (4.3); 0.731 (5.6); 0.719 (5.5); 0.714 (4.4); 0.702 (1.7); 0.505 (1.7); 0.494 (5.2); 0.488 (5.0); 0.484 (4.7); 0.478 (4.7); 0.466 (1.4); 0.146 (0.8); 0.008 (5.9); 0.000 (183.1); −0.008 (6.6); −0.150 (0.9)

### Example I-1-185

¹H-NMR (400.0 MHz, d₆-DMSO): δ=9.988 (2.6); 9.972 (2.9); 8.644 (3.9); 8.637 (3.9); 8.352 (0.4); 8.346 (0.4); 8.126 (2.2); 8.120 (2.2); 8.105 (2.6); 8.098 (2.5); 7.826 (4.9); 7.805 (4.7); 7.790 (1.5); 7.787 (1.5); 7.469 (1.6); 7.453 (6.4); 7.433 (3.4); 7.417 (0.3); 7.074 (0.4); 7.052 (0.4); 4.532 (16.0); 3.943 (2.7); 3.369 (29.1); 3.315 (105.3); 2.674 (0.6); 2.670 (0.9); 2.523 (1.9); 2.509 (49.6); 2.505 (105.7); 2.501 (149.8); 2.497 (114.7); 2.332 (0.6); 2.328 (0.9); 2.324 (0.7); 0.146 (0.8); 0.008 (5.7); 0.000 (173.0); −0.008 (6.8); −0.150 (0.8)

### Example I-1-186

¹H-NMR (400.0 MHz, d₆-DMSO): δ=9.948 (2.8); 9.932 (2.9); 8.492 (3.7); 8.486 (3.7); 7.874 (1.9); 7.868 (1.9); 7.853 (2.2); 7.846 (2.1); 7.763 (1.2); 7.760 (1.3); 7.744 (1.6); 7.741 (2.7); 7.738 (1.8); 7.722 (1.7); 7.719 (1.7); 7.545 (2.8); 7.523 (2.4); 7.506 (4.5); 7.485 (4.0); 7.364 (1.6); 7.361 (1.6); 7.345 (2.9); 7.329 (1.5); 7.326 (1.5); 6.021 (0.6); 6.003 (2.0); 5.985 (2.0); 5.967 (0.6); 5.755 (5.0); 4.605 (0.5); 4.564 (9.4); 4.561 (9.4); 4.520 (0.5); 3.575 (2.3); 3.558 (7.9); 3.540 (8.0); 3.523 (2.4); 3.320 (46.3); 2.526 (0.6); 2.522 (0.8); 2.513 (15.5); 2.508 (33.7); 2.504 (47.6); 2.499 (35.0); 2.495 (16.4); 1.914 (9.4); 1.896 (9.4); 1.172 (7.8); 1.155 (16.0); 1.137 (7.6); 1.047 (2.1); 1.031 (2.0); 0.008 (1.9); 0.000 (64.9); −0.009 (2.1)

### Example I-1-187

¹H-NMR (400.0 MHz, d₆-DMSO): δ=10.517 (5.3); 9.710 (2.9); 8.379 (3.6); 8.373 (3.7); 8.313 (0.5); 8.283 (0.5); 8.278 (0.5); 7.693 (0.7); 7.685 (7.2); 7.668 (2.6); 7.663 (9.5); 7.655 (3.3); 7.623 (0.7); 7.600 (0.8); 7.520 (2.5); 7.501 (7.4); 7.481 (3.7); 7.378 (8.3); 7.361 (2.9); 7.356 (7.4); 7.320 (2.5); 7.301 (3.5); 7.284 (2.1); 5.753 (0.5); 5.560 (8.8); 3.317 (156.7); 2.675 (0.8); 2.671 (1.2); 2.666 (0.9); 2.524 (2.6); 2.510 (62.3); 2.506 (136.6); 2.501 (197.0); 2.497 (155.9); 2.457 (16.0); 2.333 (0.8); 2.328 (1.1); 2.324 (0.9); 2.095 (0.5); 1.241 (0.4); 1.233 (0.5); 0.146 (1.0); 0.008 (7.6); 0.000 (233.7); −0.008 (10.3); −0.150 (1.0)

### Example I-1-188

¹H-NMR (400.0 MHz, d₆-DMSO): δ=9.920 (2.7); 9.904 (2.7); 8.847 (3.1); 8.843 (3.1); 8.005 (1.4); 8.001 (1.4); 7.985 (1.8); 7.980 (1.9); 7.881 (3.8); 7.860 (2.8); 7.839 (1.0); 7.836 (1.1); 7.817 (2.5); 7.814 (1.8); 7.798 (1.8); 7.795 (1.8); 7.737 (3.1); 7.715 (1.9); 7.390 (1.4); 7.387 (1.4); 7.372 (2.5); 7.355 (1.4); 7.352 (1.3); 5.755 (2.7); 5.361 (7.9); 4.584 (16.0); 3.586 (2.2); 3.568 (7.6); 3.551 (7.7); 3.533 (2.3); 3.320 (62.3); 2.673 (0.4); 2.526 (0.7); 2.521 (1.0); 2.513 (19.8); 2.508 (43.2); 2.504 (61.3); 2.499 (45.0); 2.495 (21.0); 2.331 (0.4); 1.176 (7.3); 1.158 (15.3); 1.141 (7.2)

### Example I-1-189

¹H-NMR (400.0 MHz, d₆-DMSO): δ=9.550 (2.0); 9.535 (2.0); 9.533 (2.0); 8.317 (1.9); 8.312 (2.0); 7.766 (0.8); 7.764 (0.9); 7.745 (1.0); 7.743 (1.0); 7.685 (1.1); 7.678 (1.1); 7.664 (1.3); 7.657 (1.3); 7.482 (2.5); 7.461 (2.1); 7.391 (1.6); 7.374 (1.7); 7.370 (1.6); 7.353 (1.5); 5.311 (5.6); 3.775 (16.0); 3.320 (13.3); 2.513 (5.7); 2.509 (12.5); 2.504 (17.9); 2.500 (13.5); 2.496 (6.5); 1.990 (0.6); 1.177 (0.3)

### Example I-1-190

¹H-NMR (400.0 MHz, d₆-DMSO): δ=8.013 (2.0); 8.005 (2.0); 7.849 (9.5); 7.733 (1.4); 7.715 (4.0); 7.711 (3.7); 7.701 (4.1); 7.683 (3.9); 7.661 (1.6); 7.185 (2.6); 7.168 (2.4); 5.755 (0.5); 5.428 (12.7); 3.319 (90.2); 2.730 (16.0); 2.710 (1.6); 2.702 (1.1); 2.693 (0.7); 2.681 (0.4); 2.677 (0.5); 2.672 (0.7); 2.667 (0.5); 2.525 (1.2); 2.520 (1.8); 2.512 (32.4); 2.507 (70.4); 2.503 (99.6); 2.498 (73.0); 2.494 (34.1); 2.334 (0.4); 2.330 (0.6); 2.325 (0.4); 1.046 (1.3); 1.031 (1.3); 0.707 (1.1); 0.695 (3.4); 0.690 (4.4); 0.677 (4.3); 0.672 (3.5); 0.660 (1.4); 0.496 (1.7); 0.486 (4.7); 0.480 (4.3); 0.475 (4.0); 0.470 (4.2); 0.458 (1.3); 0.146 (0.5); 0.008 (3.4); 0.000 (116.6); −0.008 (3.5); −0.149 (0.5)

### Example I-1-191

¹H-NMR (400.0 MHz, d₆-DMSO): δ=9.748 (2.0); 9.731 (2.0); 8.334 (2.5); 8.330 (2.7); 8.304 (0.9); 8.290 (1.8); 8.275 (0.9); 7.616 (1.1); 7.610 (1.2); 7.595 (1.6); 7.589 (1.7); 7.493 (3.0); 7.472 (2.2); 7.407 (1.7); 7.388 (2.1); 7.226 (1.5); 7.208 (2.4); 7.191 (1.3); 5.754 (0.5); 5.517 (6.7); 3.318 (23.8); 3.164 (2.4); 3.148 (4.3); 3.133 (2.5); 2.507 (26.4); 2.503 (37.7); 2.429 (11.6); 1.800 (0.5); 1.783 (1.0); 1.766 (1.2); 1.750 (1.0); 1.733 (0.5); 0.920 (16.0); 0.904 (15.6); 0.825 (0.5); 0.808 (0.5); 0.008 (1.3); 0.000 (37.6); −0.002 (23.1)

### Example I-1-192

¹H-NMR (400.0 MHz, d₆-DMSO): δ=9.907 (2.8); 9.891 (2.9); 8.466 (4.2); 8.461 (4.4); 8.136 (4.4); 8.130 (4.4); 7.836 (1.0); 7.833 (1.1); 7.815 (2.6); 7.812 (2.0); 7.796 (1.9); 7.793 (1.9); 7.734 (3.3); 7.713 (2.1); 7.382 (1.4); 7.379 (1.5); 7.363 (2.7); 7.347 (1.4); 7.344 (1.4); 5.754 (1.0); 5.248 (10.1); 4.577 (16.0); 3.585 (2.2); 3.568 (7.2); 3.550 (7.4); 3.533 (2.3); 3.317 (69.8); 2.677 (0.4); 2.672 (0.5); 2.667 (0.4); 2.525 (1.1); 2.512 (28.1); 2.507 (59.6); 2.503 (83.7); 2.498 (62.4); 2.494 (30.1); 2.334 (0.4); 2.330 (0.5); 2.325 (0.4); 1.313 (0.5); 1.175 (7.3); 1.157 (14.8); 1.140 (7.1); 0.146 (0.4); 0.008 (3.3); 0.000 (96.0); −0.008 (3.6); −0.150 (0.4)

### Example I-1-193

¹H-NMR (400.0 MHz, d₆-DMSO): δ=9.956 (2.5); 9.940 (2.6); 8.345 (3.3); 8.338 (3.4); 8.313 (0.4); 7.657 (1.7); 7.650 (1.7); 7.636 (2.2); 7.629 (2.2); 7.596 (2.3); 7.577 (2.5); 7.494 (4.5); 7.473 (3.5); 7.303 (2.3); 7.285 (3.2); 1.261 (2.1); 7.267 (2.1); 5.753 (0.7); 5.456 (9.1); 4.570 (15.7); 3.369 (32.1); 3.316 (178.3); 2.675 (0.7); 2.671 (1.0); 2.666 (0.7); 2.524 (1.9); 2.510 (52.7); 2.506 (115.6); 2.501 (166.4); 2.497 (126.6); 2.493 (61.9); 2.421 (16.0); 2.333 (0.7); 2.328 (1.0); 2.324 (0.7); 1.235 (0.4); 0.146 (1.0); 0.008 (6.4); 0.000 (216.0); −0.008 (8.1); −0.150 (1.0)

### Example I-1-194

¹H-NMR (400.0 MHz, d₆-DMSO): δ=9.525 (1.9); 9.509 (1.9); 8.336 (2.2); 8.331 (2.5); 7.683 (1.8); 7.664 (2.1); 7.652 (1.3); 7.646 (1.3); 7.631 (1.4); 7.625 (1.6); 7.465 (2.5); 7.444 (2.0); 7.345 (1.3); 7.326 (1.8); 7.309 (1.2); 5.599 (6.1); 4.336 (0.5); 3.857 (0.5); 3.830 (0.5); 3.805 (0.9); 3.789 (1.3); 3.773 (1.7); 3.745 (14.6); 3.720 (1.0); 3.712 (1.0); 3.701 (1.1); 3.694 (1.1); 3.685 (1.0); 3.669 (1.0); 3.651 (1.0); 3.625 (0.8); 3.573 (0.6); 3.552 (0.5); 3.535 (0.6); 3.505 (0.7); 3.490 (0.8); 3.472 (0.7); 3.440 (0.6); 3.369 (0.4); 3.322 (10.9); 3.170 (1.0); 3.095 (0.3); 3.089 (0.4); 3.077 (0.3); 2.541 (0.4); 2.503 (45.0); 2.499 (38.7); 2.443 (14.8); 1.234 (0.9); 1.201 (0.8); 1.183 (1.5); 1.165 0.8); 1.123 (0.5); 1.105 (0.4); 1.046 (15.8); 1.031 (16.0); 0.000 (0.4)

### Example I-1-195

¹H-NMR (400.0 MHz, d₆-DMSO): δ=9.668 (4.5); 9.651 (4.6); 8.589 (6.6); 8.584 (6.9); 8.563 (6.5); 8.557 (6.7); 8.014 (3.8); 8.005 (3.9); 7.912 (3.7); 7.906 (6.4); 7.901 (3.8); 7.747 (3.3); 7.725 (5.3); 7.669 (2.6); 7.667 (2.7); 7.648 (4.1); 7.629 (1.8); 7.626 (1.9); 7.332 (2.2); 7.329 (2.4); 7.312 (4.3); 7.297 (2.0); 7.294 (2.1); 5.292 (16.0); 3.317 (111.2); 2.802 (0.4); 2.792 (1.2); 2.783 (1.9); 2.774 (2.7); 2.765 (2.8); 2.756 (1.9); 2.747 (1.3); 2.737 (0.4); 2.676 (0.6); 2.672 (0.8); 2.667 (0.6); 2.525 (1.7); 2.511 (46.6); 2.507 (99.3); 2.503 (140.2); 2.498 (106.6); 2.334 (0.6); 2.329 (0.8); 2.325 (0.6); 1.743 (0.7); 1.234 (0.3); 1.046 (1.2); 1.031 (1.2); 0.743 (1.6); 0.731 (4.7); 0.726 (6.2); 0.713 (6.1); 0.708 (4.9); 0.696 (2.0); 0.503 (2.0); 0.492 (5.7); 0.486 (5.5); 0.482 (5.2); 0.477 (5.3); 0.465 (1.6); 0.146 (0.7); 0.008 (4.7); 0.000 (153.9); −0.008 (6.6); −0.150 (0.7)

### Example I-1-196

¹H-NMR (400.0 MHz, d₆-DMSO): δ=9.750 (6.1); 9.734 (6.2); 8.328 (5.8); 8.322 (6.1); 8.096 (4.4); 8.087 (4.6); 7.682 (4.7); 7.675 (4.0); 7.667 (3.3); 7.661 (5.7); 7.654 (4.2); 7.481 (7.4); 7.460 (6.1); 7.377 (4.2); 7.360 (4.3); 7.356 (4.4); 7.339 (3.7); 5.349 (16.0); 4.340 (0.9); 4.330 (0.9); 3.318 (55.5); 2.829 (0.4); 2.820 (1.2); 2.811 (2.0); 2.802 (2.8); 2.793 (2.9); 2.784 (2.0); 2.775 (1.3); 2.765 (0.5); 2.672 (0.5); 2.668 (0.4); 2.525 (1.0); 2.508 (57.5); 2.503 (82.6); 2.499 (65.2); 2.334 (0.4); 2.330 (0.5); 2.326 (0.4); 1.046 (5.7); 1.031 (5.6); 0.766 (1.6); 0.753 (5.0); 0.748 (6.7); 0.735 (6.4); 0.731 (5.4); 0.718 (2.1); 0.519 (2.0); 0.508 (6.1); 0.503 (6.0); 0.498 (5.8); 0.493 (5.7); 0.481 (1.7); 0.008 (2.0); 0.000 (62.2); −0.008 (2.8)

### Example I-1-197

¹H-NMR (400.0 MHz, d₆-DMSO): δ=9.645 (4.0); 9.639 (4.1); 9.636 (4.1); 8.401 (6.4); 8.396 (6.5); 8.314 (0.4); 8.222 (0.4); 8.039 (4.9); 8.030 (4.9); 8.000 (2.1); 7.995 (2.2); 7.977 (2.6); 7.971 (3.9); 7.965 (2.4); 7.948 (2.2); 7.942 (2.0); 7.773 (3.6); 7.767 (3.5); 7.753 (4.2); 7.746 (4.1); 7.503 (7.8); 7.483 (6.9); 5.754 (16.0); 5.347 (0.4); 5.302 (15.5); 4.340 (0.8); 4.330 (0.8); 3.793 (0.3); 3.778 (0.4); 3.318 (147.8); 2.816 (0.5); 2.806 (1.4); 2.797 (2.2); 2.788 (3.3); 2.779 (3.3); 2.771 (2.4); 2.761 (1.6); 2.752 (0.6); 2.671 (1.2); 2.524 (2.2); 2.506 (142.6); 2.502 (192.5); 2.498 (147.0); 2.333 (1.0); 2.329 (1.3); 1.235 (0.5); 1.045 (5.7); 1.030 (5.5); 0.765 (2.0); 0.752 (6.1); 0.747 (8.1); 0.735 (7.9); 0.730 (6.8); 0.718 (2.5); 0.696 (0.4); 0.521 (2.4); 0.510 (7.3); 0.504 (7.6); 0.500 (7.2); 0.495 (7.2); 0.482 (2.2); 0.146 (0.6); 0.008 (5.4); 0.000 (139.5); −0.008 (7.3); −0.149 (0.6)

### Example I-1-198

¹H-NMR (400.0 MHz, d₆-DMSO): δ=9.515 (1.8); 9.499 (1.9); 8.465 (2.4); 8.459 (2.5); 7.843 (1.3); 7.837 (1.3); 7.822 (1.5); 7.817 (1.5); 7.659 (0.8); 7.656 (0.8); 7.637 (1.8); 7.619 (1.2); 7.616 (1.1); 7.495 (2.7); 7.474 (4.0); 7.451 (1.7); 7.326 (1.0); 7.323 (1.0); 7.307 (1.9); 7.291 (1.0); 7.288 (0.9); 6.024 (0.4); 6.007 (1.3); 5.989 (1.4); 5.971 (0.5); 5.754 (0.9); 3.726 (16.0); 3.333 (50.5); 2.509 (25.0); 2.504 (32.6); 2.500 (25.3); 1.917 (0.4); 1.899 (6.3); 1.881 (6.2); 1.047 (1.1); 1.032 (1.1); 0.000 (29.6); −0.006 (3.6)

### Example I-1-199

¹H-NMR (400.0 MHz, d₆-DMSO): δ=9.484 (2.0); 9.467 (2.1); 8.459 (3.1); 8.456 (3.0); 8.113 (3.3); 8.109 (3.0); 7.741 (0.5); 7.719 (1.7); 7.699 (3.3); 7.676 (0.8); 7.347 (0.9); 7.330 (1.8); 7.326 (1.6); 7.313 (0.9); 7.310 (0.8); 5.239 (7.9); 3.740 (16.0); 3.322 (25.2); 3.320 (24.0); 2.505 (37.4); 1.047 (1.0); 1.032 (1.0); 0.000 (33.2)

### Example I-1-200

¹H-NMR (400.0 MHz, d₆-DMSO): δ=9.677 (5.4); 9.661 (5.7); 8.835 (6.3); 8.313 (0.9); 8.014 (4.6); 8.005 (4.7); 7.969 (2.6); 7.965 (2.7); 7.945 (4.1); 7.869 (7.9); 7.849 (5.2); 7.737 (3.8); 7.715 (6.4); 7.667 (3.3); 7.665 (3.5); 7.649 (3.8); 7.646 (5.1); 7.627 (2.4); 7.625 (2.3); 7.339 (2.8); 7.336 (2.8); 7.319 (4.9); 7.304 (2.5); 7.301 (2.6); 5.398 (16.0); 3.568 (2.1); 3.351 (0.5); 3.347 (0.4); 3.316 (216.3); 3.098 (0.4); 2.802 (0.6); 2.792 (1.5); 2.783 (2.3); 2.774 (3.4); 2.765 (3.2); 2.756 (2.3); 2.747 (1.7); 2.737 (0.6); 2.675 (1.7); 2.671 (2.3); 2.667 (1.5); 2.581 (0.4); 2.524 (5.3); 2.520 (7.6); 2.511 (121.3); 2.506 (259.2); 2.502 (364.1); 2.497 (270.6); 2.493 (128.5); 2.333 (1.4); 2.329 (2.2); 2.324 (1.5); 1.233 (0.5); 1.197 (0.4); 1.177 (0.8); 1.160 (0.5); 1.045 (0.8); 1.030 (0.8); 0.744 (2.0); 0.732 (5.8); 0.727 (7.3); 0.714 (7.4); 0.709 (5.7); 0.697 (2.4); 0.523 (0.3); 0.494 (2.4); 0.483 (6.5); 0.477 (6.4); 0.473 (5.9); 0.468 (6.1); 0.456 (1.9); 0.146 (1.2); 0.008 (8.8); 0.000 (302.8); −0.009 (10.5); −0.149 (1.3)

### Example I-1-201

¹H-NMR (400.0 MHz, d₆-DMSO): δ=9.664 (4.6); 9.647 (4.7); 8.458 (6.9); 8.453 (7.1); 8.120 (7.3); 8.115 (7.2); 8.000 (3.8); 7.991 (3.9); 7.742 (3.2); 7.721 (5.3); 7.668 (2.7); 7.665 (2.9); 7.650 (3.2); 7.647 (4.2); 7.644 (2.0); 7.628 (2.0); 7.625 (1.9); 7.332 (2.3); 7.329 (2.5); 7.312 (4.1); 7.297 (2.1); 7.294 (2.1); 5.755 (3.9); 5.286 (16.0); 3.319 (67.2); 2.799 (0.4); 2.789 (1.2); 2.780 (1.9); 2.772 (2.8); 2.762 (2.9); 2.753 (1.9); 2.744 (1.3); 2.735 (0.5); 2.677 (0.5); 2.672 (0.7); 2.668 (0.5); 2.526 (1.5); 2.521 (2.1); 2.512 (36.4); 2.508 (78.1); 2.503 (110.1); 2.499 (81.4); 2.494 (38.3); 2.335 (0.5); 2.330 (0.6); 2.326 (0.5); 1.232 (0.4); 1.047 (1.9); 1.031 (1.9); 0.743 (1.7); 0.731 (4.7); 0.726 (6.2); 0.714 (6.2); 0.708 (4.8); 0.697 (2.0); 0.499 (2.1); 0.488 (5.5); 0.482 (5.3); 0.478 (4.9); 0.473 (5.1); 0.460 (1.6); 0.146 (0.4); 0.008 (2.7); 0.000 (9.7); −0.008 (2.9); −0.150 (0.4)

### Example I-1-202

¹H-NMR (400.0 MHz, d₆-DMSO): δ=9.706 (5.5); 9.689 (5.7); 8.486 (6.6); 8.48 (6.6); 8.480 (7.1); 8.314 (0.3); 8.062 (4.8); 8.053 (5.0); 7.866 (3.5); 7.861 (3.6); 7.845 (3.9); 7.839 (3.9); 7.613 (0.5); 7.590 (8.2); 7.579 (11.6); 7.498 (7.4); 7.477 (6.7); 7.315 (2.1); 7.306 (2.2); 7.302 (2.7); 7.299 (2.9); 7.294 (2.7); 7.290 (2.7); 7.286 (2.6); 7.277 (1.9); 6.027 (1.1); 6.009 (3.7); 5.991 (3.8); 5.974 (1.1); 5.754 (13.5); 4.339 (0.7); 4.329 (0.7); 3.318 (90.2); 2.791 (0.4); 2.781 (1.4); 2.772 (2.1); 2.763 (3.0); 2.754 (3.1); 2.745 (2.2); 2.736 (1.4); 2.726 (0.5); 2.672 (0.9); 2.507 (108.0); 2.503 (150.9); 2.499 (124.6); 2.330 (1.0); 1.927 (16.0); 1.909 (16.0); 1.234 (0.3); 1.046 (4.6); 1.031 (4.6); 0.731 (1.6); 0.715 (7.3); 0.702 (6.8); 0.697 (6.3); 0.685 (2.0); 0.487 (2.2); 0.476 (6.8); 0.470 (6.9); 0.466 (6.8); 0.461 (6.6); 0.449 (1.8); 0.145 (0.4); 0.000 (90.8); −0.149 (0.4)

### Example I-1-203

¹H-NMR (400.0 MHz, d₆-DMSO): δ=9.701 (2.6); 9.685 (2.6); 8.121 (2.4); 8.112 (2.5); 7.669 (6.6); 7.472 (2.2); 7.453 (2.6); 7.252 (2.4); 7.234 (3.3); 7.216 (2.0); 5.754 (0.5); 5.554 (9.0); 3.318 (58.5); 2.796 (0.8); 2.787 (1.2); 2.778 (1.8); 2.768 (1.8); 2.760 (1.2); 2.751 (0.8); 2.676 (0.4); 2.672 (0.6); 2.667 (0.4); 2.633 (16.0); 2.525 (1.3); 2.511 (30.5); 2.507 (63.4); 2.502 (87.8); 2.498 (65.2); 2.494 (31.3); 2.334 (0.4); 2.329 (0.5); 2.325 (0.4); 1.046 (0.5); 1.031 (0.5); 0.752 (1.0); 0.739 (3.0); 0.734 (3.9); 0.722 (3.9); 0.717 (3.1); 0.705 (1.3); 0.507 (1.3); 0.496 (3.6); 0.490 (3.5); 0.486 (3.2); 0.481 (3.3); 0.468 (1.0); 0.008 (2.1); 0.000 (63.2); −0.009 (2.2)

### Example I-1-204

¹H-NMR (400.0 MHz, d₆-DMSO): δ=9.741 (2.1); 9.724 (2.1); 8.349 (2.2); 8.343 (2.3); 8.168 (1.6); 8.158 (1.7); 7.650 (1.1); 7.644 (1.1); 7.630 (1.4); 7.623 (1.4); 7.612 (1.9); 7.592 (2.1); 7.465 (2.8); 7.444 (2.3); 7.331 (1.4); 7.312 (1.8); 7.295 (1.3); 5.636 (5.8); 3.724 (0.5); 3.316 (54.7); 2.798 (0.5); 2.789 (0.7); 2.780 (1.1); 2.771 (1.1); 2.762 (0.8); 2.753 (0.5); 2.671 (0.5); 2.666 (0.4); 2.505 (62.4); 2.501 (88.0); 2.497 (69.4); 2.454 (16.0); 2.328 (0.5); 1.234 (0.3); 1.045 (0.6); 1.030 (0.6); 0.746 (0.6); 0.733 (1.9); 0.728 (2.5); 0.716 (2.4); 0.711 (2.0); 0.699 (0.8); 0.494 (0.8); 0.483 (2.4); 0.477 (2.3); 0.473 (2.2); 0.468 (2.2); 0.455 (0.7); 0.007 (1.8); 0.000 (50.8); −0.008 (2.3)

### Example I-1-205

¹H-NMR (400.0 MHz, d₆-DMSO): δ=9.782 (4.0); 9.765 (4.2); 9.573 (0.4); 9.556 (0.5); 8.421 (5.0); 7.817 (1.9); 7.796 (4.9); 7.775 (3.6); 7.770 (4.4); 7.746 (0.5); 7.504 (4.5); 7.484 (4.2); 7.457 (0.6); 7.435 (0.4); 7.415 (0.6); 7.366 (1.2); 7.352 (2.1); 7.336 (2.0); 7.332 (2.0); 7.316 (1.1); 7.292 (0.3); 7.275 (0.4); 5.753 (1.5); 5.368 (0.4); 5.351 (1.4); 5.281 (10.9); 4.567 (16.0); 4.551 (2.5); 3.906 (2.9); 3.371 (28.8); 3.360 (4.8); 3.320 (87.7); 2.671 (0.7); 2.502 (119.7); 2.454 (0.5); 2.328 (0.7); 1.234 (1.3); 0.146 (0.4); 0.000 (69.8); −0.150 (0.4)

### Example I-1-206

¹H-NMR (400.0 MHz, d₆-DMSO): δ=9.490 (1.5); 9.474 (1.5); 7.676 (3.8); 7.544 (1.3); 7.525 (1.5); 7.268 (1.4); 7.250 (2.0); 7.232 (1.2); 5.505 (5.6); 3.744 (16.0); 3.320 (29.1); 2.638 (9.6); 2.526 (0.4); 2.512 (9.4); 2.508 (20.5); 2.504 (29.3); 2.499 (22.0); 2.495 (10.6); 1.047 (0.8); 1.031 (0.8); 0.008 (1.1); 0.000 (35.4); −0.008 (1.2)

### Example I-1-207

¹H-NMR (400.0 MHz, d₆-DMSO): δ=9.857 (2.6); 9.840 (2.7); 8.354 (3.0); 8.309 (2.3); 8.003 (0.9); 7.997 (1.0); 7.982 (1.8); 7.976 (1.8); 7.962 (1.1); 7.956 (1.0); 7.904 (1.1); 7.885 (2.4); 7.867 (1.6); 7.864 (1.7); 7.790 (3.0); 7.769 (2.0); 7.418 (1.4); 7.401 (2.6); 7.385 (1.3); 7.382 (1.4); 7.184 (1.9); 7.177 (1.9); 7.163 (1.8); 7.156 (1.8); 5.263 (8.9); 4.811 (16.0); 3.683 (0.5); 3.572 (2.2); 3.383 (102.9); 2.516 (14.9); 2.512 (20.3); 2.507 (16.1); 0.000 (2.5)

### Example I-1-208

¹H-NMR (400.0 MHz, d₆-DMSO): δ=10.215 (3.1); 9.716 (1.6); 9.700 (1.7); 7.706 (3.8); 7.571 (0.4); 7.563 (3.6); 7.540 (4.5); 7.517 (1.7); 7.302 (1.3); 7.285 (2.0); 7.267 (1.1); 6.934 (0.4); 6.925 (3.8); 6.903 (3.7); 5.756 (5.6); 5.626 (5.0); 3.741 (16.0); 3.720 (0.8); 3.322 (19.1); 2.659 (9.1); 2.508 (28.1); 2.503 (37.6); 2.499 (29.1); 0.000 (1.3)

### Example I-1-209

¹H-NMR (400.0 MHz, d₆-DMSO): δ=9.841 (2.1); 9.825 (2.1); 8.310 (1.0); 7.939 (0.7); 7.937 (0.7); 7.918 (1.8); 7.899 (1.5); 7.897 (1.4); 7.868 (2.5); 7.847 (1.3); 7.787 (4.6); 7.421 (1.0); 7.418 (1.0); 7.403 (1.9); 7.387 (1.0); 7.383 (0.9); 5.389 (7.4); 4.809 (0.4); 4.795 (12.6); 3.570 (1.7); 3.355 (126.7); 2.653 (0.3); 2.567 (16.0); 2.553 (0.6); 2.511 (21.0); 2.507 (28.0); 2.502 (21.8); 0.000 (5.1)

### Example I-1-210

¹H-NMR (400.0 MHz, d₆-DMSO): δ=9.634 (0.6); 9.617 (0.6); 8.520 (0.6); 8.500 (0.6); 7.681 (1.3); 7.473 (0.5); 7.454 (0.7); 7.394 (7.6); 7.373 (0.5); 7.340 (0.4); 7.227 (0.5); 7.210 (0.7); 7.192 (0.4); 5.754 (16.0); 5.579 (1.7); 5.128 (0.4); 3.750 (1.4); 3.377 (1.3); 3.318 (10.6); 2.632 (3.2); 2.507 (18.7); 2.503 (25.3); 2.498 (19.5); 1.475 (1.8); 1.458 (1.8); 1.347 (0.5); 1.330 (0.5); 0.008 (0.9); 0.000 (19.2)

### Example I-1-211

¹H-NMR (400.0 MHz, d₆-DMSO): δ=10.375 (4.7); 9.717 (2.6); 9.700 (2.6); 8.313 (0.6); 8.266 (0.6); 8.261 (0.6); 7.709 (6.7); 7.647 (4.7); 7.628 (5.3); 7.587 (0.7); 7.560 (2.4); 7.541 (2.7); 7.352 (3.3); 7.332 (5.4); 7.320 (3.0); 7.313 (4.6); 7.303 (3.5); 7.292 (0.9); 7.285 (2.2); 7.085 (0.4); 7.066 (0.5); 7.054 (1.7); 7.036 (3.0); 7.017 (1.3); 5.754 (0.5); 5.632 (8.4); 3.316 (81.4); 2.663 (16.0); 2.524 (3.9); 2.510 (83.3); 2.506 (171.9); 2.501 (236.9); 2.497 (178.9); 2.493 (88.2); 2.333 (1.0); 2.328 (1.4); 2.324 (1.0); 1.474 (0.5); 0.008 (0.6); 0.000 (16.8); −0.009 (0.6)

### Example I-1-212

¹H-NMR (400.0 MHz, d₆-DMSO): δ=9.666 (5.5); 9.584 (2.7); 9.567 (2.8); 7.722 (6.9); 7.571 (2.4); 7.552 (2.8); 7.384 (0.4); 7.368 (1.0); 7.362 (0.8); 7.347 (1.9); 7.326 (1.4); 7.309 (2.9); 7.291 (3.3); 7.273 (2.2); 7.220 (0.7); 7.211 (3.9); 7.203 (0.7); 7.191 (6.0); 7.179 (0.7); 7.171 (2.5); 7.162 (0.5); 5.756 (13.6); 5.628 (8.6); 3.321 (19.1); 3.178 (0.5); 3.165 (0.5); 2.664 (16.0); 2.526 (1.1); 2.513 (21.3); 2.508 (42.7); 2.504 (57.9); 2.499 (43.6); 2.495 (21.7); 2.330 (0.3); 0.008 (0.7); 0.000 (16.8); −0.008 (0.7)

### Example I-1-213

¹H-NMR (400.0 MHz, d₆-DMSO): δ=9.744 (0.8); 9.727 (0.8); 8.115 (1.3); 7.666 (1.8); 7.444 (0.7); 7.427 (0.8); 7.209 (0.6); 7.191 (1.0); 7.173 (0.5); 5.560 (2.6); 3.319 (24.1); 2.628 (4.5); 2.505 (35.7); 2.502 (47.1); 2.498 (37.4); 1.389 (16.0); 1.355 (1.4); 1.330 (0.5); 1.308 (0.6); 1.297 (1.0); 1.272 (0.4); 1.255 (2.1); 1.244 (0.5); 1.228 (1.5); 1.223 (1.1); 1.187 (1.5); 1.169 (0.7)

### Example I-1-214

¹H-NMR (400.0 MHz, d₆-DMSO): δ=9.524 (1.7); 9.508 (1.7); 8.111 (2.1); 7.770 (0.6); 7.764 (0.6); 7.750 (1.1); 7.744 (1.2); 7.730 (0.6); 7.723 (0.6); 7.490 (1.5); 7.471 (1.8); 7.258 (1.4); 7.240 (2.2); 7.223 (1.2); 7.163 (1.3); 7.157 (1.4); 7.142 (1.2); 7.135 (1.3); 5.451 (5.6); 3.744 (16.0); 3.321 (14.1); 2.508 (21.0); 2.503 (29.4); 2.499 (23.7); 2.433 (10.8); 1.233 (0.5); 0.000 (0.4)

### Example I-1-215

¹H-NMR (400.0 MHz, d₆-DMSO): δ=9.486 (1.8); 9.469 (1.8); 8.591 (3.3); 8.390 (3.3); 7.710 (0.8); 7.689 (1.8); 7.670 (1.3); 7.577 (2.2); 7.555 (1.5); 7.335 (1.0); 7.317 (1.8); 7.300 (0.9); 5.334 (6.9); 3.721 (16.0); 3.320 (12.5); 2.506 (27.8); 2.502 (36.4); 2.498 (27.8); 2.450 (12.8); 1.045 (2.1); 1.030 (2.1)

### Example I-1-216

¹H-NMR (400.0 MHz, d₆-DMSO): δ=10.584 (5.4); 9.68 (2.7); 9.687 (2.7); 9.670 (2.8); 8.280 (3.8); 7.713 (8.1); 7.698 (2.2); 7.585 (2.7); 7.582 (2.6); 7.566 (3.2); 7.561 (3.5); 7.540 (1.5); 7.385 (2.4); 7.366 (2.0); 7.330 (2.5); 7.312 (3.4); 7.295 (2.2); 5.756 (0.8); 5.631 (8.5); 3.322 (33.4); 2.662 (16.0); 2.526 (1.4); 2.513 (24.9); 2.508 (50.4); 2.504 (68.7); 2.499 (51.8); 2.495 (25.7); 2.331 (0.4); 0.000 (2.9)

### Example I-1-217

¹H-NMR (400.0 MHz, d₆-DMSO): δ=9.663 (2.5); 9.647 (2.6); 8.605 (4.0); 8.602 (4.1); 8.390 (4.0); 7.979 (2.1); 7.970 (2.1); 7.623 (3.8); 7.614 (5.4); 7.612 (5.2); 7.601 (0.4); 7.324 (1.1); 7.315 (1.1); 7.311 (1.2); 7.307 (1.3); 7.302 (1.2); 7.298 (1.2); 7.295 (1.1); 7.286 (0.9); 5.385 (8.3); 3.322 (27.1); 2.778 (0.7); 2.769 (1.0); 2.760 (1.5); 2.751 (1.5); 2.742 (1.0); 2.733 (0.7); 2.508 (30.2); 2.503 (40.7); 2.499 (30.8); 2.450 (16.0); 1.046 (0.4); 1.031 (0.4); 0.731 (0.9); 0.718 (2.6); 0.713 (3.3); 0.701 (3.3); 0.696 (2.6); 0.684 (1.0); 0.473 (1.1); 0.462 (3.1); 0.457 (3.0); 0.452 (2.8); 0.447 (2.8); 0.435 (0.9); 0.000 (0.7)

### Example I-1-218

¹H-NMR (400.0 MHz, d₆-DMSO): δ=10.231 (8.3); 9.689 (5.2); 9.672 (5.5); 8.665 (8.1); 8.662 (8.5); 8.413 (8.0); 8.411 (8.0); 7.740 (0.6); 7.738 (0.6); 7.718 (4.4); 7.715 (5.2); 7.709 (5.9); 7.704 (10.9); 7.702 (10.9); 7.689 (0.9); 7.689 (0.9); 7.623 (8.1); 7.604 (9.0); 7.392 (2.3); 7.385 (2.2); 7.378 (2.8); 7.376 (3.0); 7.370 (2.7); 7.368 (2.6); 7.362 (2.2); 7.354 (2.2); 7.335 (5.7); 7.316 (8.6); 7.295 (5.8); 7.037 (2.9); 7.019 (5.1); 7.000 (2.3); 5.465 (16.0); 4.343 (0.5); 4.333 (0.5); 3.322 (74.7); 2.676 (0.5); 2.672 (0.7); 2.667 (0.5); 2.525 (2.4); 2.512 (42.1); 2.507 (85.0); 2.503 (116.0); 2.498 (87.3); 2.494 (43.2); 2.462 (32.4); 2.334 (0.4); 2.329 (0.7); 2.325 (0.5); 1.232 (0.3); 1.046 (4.0); 1.031 (3.9); 0.000 (2.2)

### Example I-1-219

¹H-NMR (400.0 MHz, d₆-DMSO): δ=10.441 (5.2); 9.721 (2.8); 9.705 (2.8); 8.380 (3.6); 8.374 (3.6); 7.681 (2.2); 7.674 (5.0); 7.661 (5.7); 7.651 (4.9); 7.644 (1.6); 7.638 (3.7); 7.630 (0.4); 7.504 (6.1); 7.488 (3.2); 7.484 (4.6); 7.310 (2.5); 7.293 (3.4); 7.275 (2.1); 7.192 (0.4); 7.183 (3.7); 7.178 (1.3); 7.161 (6.6); 7.145 (1.2); 7.139 (3.4); 7.130 (0.3); 5.755 (1.3); 5.562 (8.7); 3.321 (41.0); 2.677 (0.3); 2.672 (0.4); 2.668 (0.3); 2.512 (26.1); 2.507 (52.7); 2.503 (72.2); 2.498 (54.7); 2.494 (27.4); 2.457 (16.0); 2.330 (0.4); 2.325 (0.3); 1.046 (0.7); 1.031 (0.7); 0.000 (1.4)

### Example I-1-220

¹H-NMR (400.0 MHz, d₆-DMSO): δ=9.927 (1.9); 9.911 (2.0); 7.632 (1.7); 7.613 (1.9); 7.551 (4.4); 7.302 (1.6); 7.284 (2.3); 7.266 (1.4); 5.518 (6.5); 4.560 (10.8); 3.932 (0.4); 3.370 (20.4); 3.350 (0.8); 3.322 (2.6); 3.292 (0.7); 2.623 (11.7); 2.575 (16.0); 2.508 (24.5); 2.504 (32.3); 2.499 (24.7); 2.456 (0.5); 0.000 (12.5)

### Example I-1-221

¹H-NMR (400.0 MHz, d₆-DMSO): δ=9.885 (1.9); 9.869 (1.9); 7.874 (0.5); 7.871 (0.5); 7.852 (1.5); 7.849 (1.4); 7.835 (1.7); 7.832 (1.8); 7.823 (2.2); 7.804 (0.7); 7.776 (4.5); 7.381 (0.9); 7.377 (0.9); 7.364 (1.7); 7.360 (1.6); 7.348 (0.9); 7.343 (0.8); 5.366 (6.4); 4.531 (10.9); 3.364 (22.2); 3.321 (16.1); 2.563 (16.0); 2.526 (0.8); 2.513 (15.0); 2.508 (30.5); 2.504 (41.7); 2.499 (31.1); 2.495 (15.2); 0.008 (0.7); 0.000 (17.7); −0.008 (0.6)

### Example I-1-222

¹H-NMR (400.0 MHz, d₆-DMSO): δ=9.732 (2.6); 9.716 (2.6); 8.203 (2.5); 8.193 (2.5); 8.123 (2.9); 8.119 (3.0); 7.763 (0.9); 7.757 (0.9); 7.742 (1.7); 7.736 (1.7); 7.722 (1.0); 7.716 (0.9); 7.419 (2.2); 7.400 (2.7); 7.242 (2.4); 7.224 (3.3); 7.206 (2.0); 7.162 (2.0); 7.155 (2.0); 7.141 (1.9); 7.134 (1.9); 5.490 (7.9); 3.318 (45.7); 2.801 (0.8); 2.792 (1.2); 2.783 (1.8); 2.774 (1.8); 2.765 (1.2); 2.756 (0.9); 2.676 (0.6); 2.671 (0.7); 2.667 (0.5); 2.524 (2.4); 2.511 (43.3); 2.507 (87.6); 2.502 (120.2); 2.498 (91.2); 2.493 (45.8); 2.441 (16.0); 2.333 (0.5); 2.329 (0.7); 2.325 (0.5); 0.747 (1.1); 0.734 (3.1); 0.730 (4.0); 0.717 (4.0); 0.712 (3.1); 0.700 (1.3); 0.497 (1.3); 0.486 (3.7); 0.480 (3.6); 0.476 (3.4); 0.471 (3.4); 0.459 (1.1); 0.008 (1.8); 0.000 (43.8); −0.008 (1.8)

### Example I-1-223

¹H-NMR (400.0 MHz, d₆-DMSO): δ=9.704 (1.8); 9.687 (1.8); 8.167 (1.7); 8.158 (1.7); 7.539 (4.3); 7.453 (1.5); 7.434 (1.8); 7.242 (1.6); 7.224 (2.2); 7.206 (1.4); 5.553 (6.0); 3.319 (19.1); 2.796 (0.6); 2.786 (0.9); 2.778 (1.3); 2.768 (1.3); 2.760 (0.9); 2.750 (0.6); 2.671 (0.4); 2.667 (0.3); 2.621 (11.1); 2.564 (16.0); 2.525 (1.4); 2.511 (23.0); 2.507 (46.1); 2.502 (62.8); 2.498 (47.3); 2.494 (23.6); 2.329 (0.4); 0.749 (0.7); 0.736 (2.1); 0.732 (2.8); 0.719 (2.7); 0.714 (2.2); 0.702 (0.9); 0.503 (0.9); 0.492 (2.5); 0.486 (2.4); 0.482 (2.3); 0.477 (2.3); 0.4 (23); 0464 (0.7); 0.008 (1.0); 0.000 (22.9); −0.008 (0.9)

### Example I-1-224

¹H-NMR (400.0 MHz, d₆-DMSO): δ=9.908 (2.4); 9.892 (2.4); 8.631 (3.8); 8.627 (4.1); 8.400 (3.9); 8.398 (3.9); 7.819 (1.0); 7.816 (1.2); 7.800 (1.3); 7.797 (2.4); 7.794 (1.7); 7.779 (1.5); 7.776 (1.5); 7.638 (2.6); 7.617 (2.1); 7.377 (1.3); 7.374 (1.4); 7.358 (2.3); 7.342 (1.2); 7.339 (1.2); 5.755 (0.6); 5.352 (8.3); 4.506 (13.9); 3.931 (1.4); 3.350 (28.3); 3.324 (1.8); 3.292 (2.5); 2.526 (0.9); 2.512 (13.9); 2.508 (28.4); 2.503 (39.1); 2.499 (30.0); 2.495 (15.2); 2.456 (16.0); 1.234 (0.3); 0.008 (0.7); 0.000 (17.0); −0.009 (0.7)

### Example I-1-225

¹H-NMR (400.0 MHz, d₆-DMSO): δ=9.957 (2.3); 9.940 (2.3); 8.148 (2.6); 8.143 (2.6); 8.018 (0.5); 8.013 (0.5); 7.816 (0.8); 7.810 (0.8); 7.795 (1.5); 7.789 (1.5); 7.775 (0.9); 7.769 (0.9); 7.596 (2.1); 7.586 (0.6); 7.577 (2.4); 7.495 (0.4); 7.489 (0.3); 7.301 (2.2); 7.283 (2.8); 7.271 (0.6); 7.265 (2.0); 7.254 (0.4); 7.175 (1.8); 7.169 (1.8); 7.154 (1.7); 7.147 (1.7); 6.804 (0.6); 6.782 (0.6); 5.456 (7.1); 5.372 (1.3); 4.576 (16.0); 3.816 (4.9); 3.371 (37.7); 3.320 (21.7); 3.177 (1.1); 3.163 (1.1); 2.672 (0.4); 2.525 (1.3); 2.512 (23.7); 2.507 (48.3); 2.503 (66.1); 2.498 (49.6); 2.494 (24.2); 2.472 (2.6); 2.437 (14.6); 2.330 (0.4); 1.046 (0.5); 1.031 (0.5); 0.008 (1.0); 0.000 (26.0); −0.009 (0.9)

### Example I-1-226

¹H-NMR (400.0 MHz, d₆-DMSO): δ=9.901 (2.4); 9.884 (2.6); 9.873 (0.7); 8.350 (2.5); 8.346 (2.6); 8.293 (0.7); 8.287 (0.7); 7.986 (0.9); 7.980 (0.9); 7.965 (1.6); 7.959 (1.6); 7.945 (0.9); 7.939 (0.9); 7.839 (0.8); 7.837 (1.0); 7.828 (0.4); 7.818 (2.2); 7.815 (1.9); 7.799 (1.6); 7.796 (1.8); 7.792 (0.6); 7.788 (0.5); 7.749 (2.8); 7.737 (0.8); 7.727 (1.7); 7.715 (0.5); 7.703 (0.5); 7.697 (0.5); 7.681 (0.5); 7.675 (0.5); 7.378 (1.2); 7.375 (1.3); 7.360 (2.4); 7.358 (2.4); 7.343 (1.6); 7.340 (1.4); 7.176 (1.7); 7.169 (1.7); 7.154 (1.7); 7.147 (1.6); 6.795 (0.8); 6.773 (0.8); 5.237 (8.1); 5.142 (2.2); 4.547 (16.0); 3.812 (6.7); 3.365 (34.6); 3.319 (29.6); 2.672 (0.5); 2.668 (0.3); 2.512 (26.6); 2.508 (54.3); 2.503 (75.6); 2.499 (58.5); 2.495 (29.8); 2.335 (0.3); 2.330 (0.5); 2.326 (0.3); 0.008 (0.9); 0.000 (24.7); −0.008 (1.0)

### Example I-1-227

¹H-NMR (400.0 MHz, d₆-DMSO): δ=10.174 (8.4); 9.652 (4.9); 9.636 (5.2); 8.315 (0.4); 7.947 (4.1); 7.925 (5.4); 7.876 (13.1); 7.803 (2.6); 7.800 (2.8); 7.785 (3.2); 7.782 (4.6); 7.763 (2.3); 7.760 (2.3); 7.687 (0.5); 7.678 (5.5); 7.673 (2.1); 7.666 (6.0); 7.660 (3.5); 7.655 (6.4); 7.648 (2.3); 7.643 (6.0); 7.634 (0.6); 7.408 (2.5); 7.405 (2.7); 7.389 (4.8); 7.373 (2.4); 7.370 (2.4); 7.199 (0.6); 7.190 (6.0); 7.185 (2.0); 7.168 (10.7); 7.151 (1.9); 7.146 (5.6); 7.137 (0.5); 5.756 (9.9); 5.507 (16.0); 3.322 (70.1); 2.677 (0.7); 2.672 (1.0); 2.668 (0.7); 2.526 (2.3); 2.521 (3.4); 2.512 (54.7); 2.508 (118.1); 2.503 (167.0); 2.499 (124.0); 2.494 (58.9); 2.335 (0.7); 2.330 (1.0); 2.326 (0.7); 1.990 (0.6); 0.000 (7.7)

### Example I-1-228

¹H-NMR (400.0 MHz, d₆-DMSO): δ=9.735 (3.6); 9.718 (3.7); 8.503 (4.3); 8.496 (4.4); 8.050 (1.3); 8.036 (2.8); 8.021 (1.4); 7.882 (2.3); 7.875 (2.3); 7.861 (2.6); 7.854 (2.5); 7.580 (6.6); 7.570 (7.0); 7.506 (5.1); 7.485 (4.6); 7.299 (1.5); 7.288 (2.6); 7.282 (1.7); 7.277 (1.6); 7.271 (2.5); 7.260 (1.3); 6.049 (0.7); 6.031 (2.4); 6.013 (2.4); 5.995 (0.7); 5.757 (4.9); 4.346 (0.5); 4.336 (0.5); 3.570 (3.8); 3.339 (1.0); 3.326 (16.3); 3.305 (4.1); 3.289 (3.2); 3.271 (1.0); 2.528 (0.6); 2.514 (13.6); 2.510 (28.5); 2.506 (39.7); 2.501 (30.0); 1.945 (10.7); 1.927 (10.8); 1.901 (0.4); 1.132 (7.5); 1.114 (16.0); 1.096 (7.4); 1.048 (3.8); 1.033 (3.8); 0.000 (2.2)

### Example I-1-229

¹H-NMR (400.0 MHz, d₆-DMSO): δ=10.312 (9.1); 9.729 (5.4); 9.712 (5.7); 8.539 (6.8); 8.532 (6.9); 8.315 (0.4); 7.929 (3.6); 7.923 (3.6); 7.909 (4.0); 7.902 (4.0); 7.700 (1.0); 7.681 (4.0); 7.679 (4.3); 7.663 (10.9); 7.635 (8.9); 7.616 (9.8); 7.524 (7.9); 7.503 (7.2); 7.382 (2.4); 7.376 (2.2); 7.366 (4.1); 7.360 (3.8); 7.350 (2.3); 7.343 (2.6); 7.336 (6.1); 7.317 (9.4); 7.297 (6.0); 7.040 (3.1); 7.022 (5.4); 7.003 (2.4); 6.102 (1.0); 6.084 (3.6); 6.067 (3.6); 6.049 (1.0); 5.755 (2.0); 4.828 (0.4); 3.373 (0.3); 3.320 (66.2); 2.676 (0.9); 2.671 (1.2); 2.667 (0.9); 2.525 (2.9); 2.511 (69.4); 2.507 (145.0); 2.502 (201.0); 2.498 (149.9); 2.333 (0.9); 2.329 (1.1); 2.325 (0.8); 1.983 (16.0); 1.965 (15.9); 1.046 (0.7); 1.030 (0.6); 0.008 (0.4); 0.000 (9.6); −0.009 (0.4)

### Example I-1-230

¹H-NMR (400.0 MHz, d₆-DMSO): δ=10.366 (8.9); 9.643 (5.0); 9.627 (5.2); 7.984 (5.3); 7.962 (4.1); 7.940 (5.5); 7.879 (13.2); 7.831 (2.7); 7.828 (2.8); 7.812 (3.4); 7.809 (4.6); 7.790 (2.3); 7.787 (2.3); 7.471 (2.3); 7.450 (6.6); 7.431 (6.3); 7.417 (8.3); 7.414 (6.7); 7.397 (6.7); 7.382 (2.5); 7.379 (2.4); 7.020 (3.0); 7.000 (2.7); 6.998 (2.4); 5.756 (1.5); 5.511 (16.0); 3.323 (75.1); 2.678 (0.6); 2.673 (0.8); 2.669 (0.6); 2.527 (2.0); 2.522 (3.0); 2.513 (46.3); 2.509 (98.8); 2.504 (138.5); 2.500 (102.5); 2.495 (48.7); 2.336 (0.6); 2.331 (0.8); 2.327 (0.6); 1.990 (0.8); 1.177 (0.4); 0.000 (6.8)

### Example I-1-231

¹H-NMR (400.0 MHz, d₆-DMSO): δ=9.548 (4.9); 9.532 (5.1); 9.488 (8.9); 8.315 (0.4); 7.949 (4.0); 7.927 (5.5); 7.883 (13.1); 7.814 (2.6); 7.811 (2.8); 7.796 (3.2); 7.793 (4.6); 7.774 (2.3); 7.771 (2.3); 7.397 (2.5); 7.394 (2.7); 7.378 (4.9); 7.368 (1.8); 7.362 (3.6); 7.352 (1.2); 7.347 (3.1); 7.342 (1.4); 7.326 (2.3); 7.310 (1.0); 7.222 (0.6); 7.217 (1.0); 7.209 (6.4); 7.201 (1.0); 7.197 (1.1); 7.189 (9.8); 7.168 (4.1); 7.159 (0.8); 5.756 (2.2); 5.507 (16.0); 4.039 (0.4); 4.022 (0.4); 3.322 (95.0); 2.677 (0.7); 2.673 (0.9); 2.668 (0.7); 2.526 (2.1); 2.521 (3.1); 2.512 (50.3); 2.508 (109.0); 2.503 (155.1); 2.499 (116.3); 2.495 (56.1); 2.335 (0.6); 2.330 (0.9); 2.326 (0.7); 1.990 (1.6); 1.194 (0.4); 1.177 (0.9); 1.159 (0.4); 0.000 (7.3)

### Example I-1-232

¹H-NMR (400.0 MHz, d₆-DMSO): δ=10.347 (4.8); 10.341 (5.0); 9.619 (4.9); 9.603 (5.0); 8.462 (1.5); 8.447 (1.7); 8.439 (3.2); 8.424 (3.2); 8.417 (1.8); 8.401 (1.6); 7.954 (4.0); 7.932 (5.4); 7.877 (12.1); 7.822 (2.8); 7.804 (4.5); 7.784 (2.1); 7.782 (2.3); 7.419 (2.7); 7.402 (5.2); 7.392 (2.1); 7.386 (2.6); 7.384 (2.7); 7.377 (2.0); 7.370 (3.3); 7.363 (2.0); 7.348 (1.7); 7.341 (1.7); 7.107 (1.3); 7.084 (2.5); 7.064 (1.3); 5.756 (0.4); 5.512 (16.0); 3.323 (48.5); 2.678 (0.5); 2.674 (0.7); 2.526 (1.7); 2.509 (87.6); 2.504 (124.7); 2.500 (99.4); 2.331 (0.7); 2.327 (0.6); 1.990 (0.4); 0.008 (0.4); 0.000 (10.4)

### Example I-1-233

¹H-NMR (400.0 MHz, d₆-DMSO): δ=10.401 (8.5); 9.655 (4.9); 9.639 (5.0); 8.315 (0.5); 8.285 (6.3); 7.967 (4.0); 7.945 (5.3); 7.881 (12.1); 7.832 (2.7); 7.813 (4.3); 7.793 (2.2); 7.791 (2.2); 7.722 (2.7); 7.701 (3.5); 7.581 (2.5); 7.561 (4.7); 7.541 (2.4); 7.417 (2.6); 7.400 (4.9); 7.385 (6.0); 7.366 (3.2); 5.756 (1.0); 5.514 (16.0); 3.321 (75.9); 2.672 (1.3); 2.668 (0.9); 2.525 (3.1); 2.507 (152.8); 2.503 (210.8); 2.499 (160.4); 2.330 (1.2); 2.326 (0.9); 0.000 (18.4)

### Example I-1-234

¹H-NMR (400.0 MHz, d₆-DMSO): δ=10.595 (8.6); 9.669 (4.8); 9.652 (5.0); 8.584 (4.0); 8.581 (4.5); 8.563 (4.4); 8.560 (4.6); 7.943 (3.9); 7.921 (5.4); 7.878 (12.3); 7.828 (2.6); 7.826 (2.8); 7.807 (4.5); 7.788 (2.1); 7.785 (2.2); 7.520 (4.1); 7.517 (4.7); 7.500 (4.7); 7.497 (5.0); 7.424 (2.4); 7.421 (2.6); 7.405 (4.7); 7.389 (2.3); 7.386 (2.3); 7.353 (2.0); 7.349 (2.1); 7.331 (4.0); 7.313 (2.3); 7.310 (2.3); 7.076 (2.5); 7.072 (2.7); 7.056 (3.9); 7.053 (4.1); 7.037 (2.1); 7.034 (2.2); 5.756 (2.4); 5.525 (16.0); 3.322 (53.1); 2.677 (0.6); 2.673 (0.8); 2.669 (0.6); 2.526 (2.0); 2.512 (45.8); 2.508 (96.9); 2.504 (136.3); 2.499 (104.4); 2.335 (0.6); 2.330 (0.8); 2.326 (0.6); 1.990 (0.4); 0.008 (0.4); 0.000 (11.0)

### Example I-1-235

¹H-NMR (400.0 MHz, d₆-DMSO): δ=10.149 (4.5); 9.673 (2.6); 9.657 (2.7); 7.947 (2.2); 7.925 (2.8); 7.879 (6.8); 7.798 (1.3); 7.795 (1.4); 7.779 (1.7); 7.777 (2.4); 7.758 (1.2); 7.755 (1.2); 7.457 (5.9); 7.440 (2.0); 7.402 (1.3); 7.400 (1.4); 7.383 (2.5); 7.367 (1.2); 7.365 (1.3); 7.227 (1.3); 7.208 (2.3); 7.186 (1.4); 6.863 (2.1); 6.845 (1.8); 5.507 (8.6); 3.321 (3.7); 2.676 (0.5); 2.672 (0.6); 2.667 (0.5); 2.525 (1.5); 2.512 (35.4); 2.507 (75.4); 2.503 (106.2); 2.498 (79.3); 2.494 (38.1); 2.334 (0.5); 2.330 (0.7); 2.325 (0.6); 2.307 (16.0); 1.989 (0.3); 0.000 (8.6)

### Example I-1-236

¹H-NMR (400.0 MHz, d₆-DMSO): δ=10.032 (2.6); 9.673 (1.5); 9.657 (1.6); 7.935 (1.2); 7.913 (1.6); 7.876 (3.9); 7.781 (0.8); 7.778 (0.8); 7.759 (1.4); 7.741 (0.7); 7.738 (0.7); 7.561 (3.3); 7.556 (1.1); 7.544 (1.1); 7.539 (3.7); 7.530 (0.4); 7.392 (0.7); 7.389 (0.8); 7.373 (1.4); 7.357 (0.7); 7.354 (0.7); 6.925 (3.5); 6.920 (1.1); 6.908 (1.0); 6.902 (3.4); 5.756 (0.8); 5.504 (4.7); 3.741 (16.0); 3.321 (21.2); 2.672 (0.4); 2.525 (0.8); 2.520 (1.3); 2.512 (19.4); 2.507 (41.6); 2.503 (58.6); 2.498 (43.5); 2.494 (20.7); 2.330 (0.3); 0.000 (5.0)

### Example I-1-237

¹H-NMR (400.0 MHz, d₆-DMSO): δ=10.155 (2.7); 9.728 (1.6); 9.712 (1.7); 8.534 (1.9); 8.528 (2.0); 7.923 (1.0); 7.917 (1.0); 7.902 (1.1); 7.896 (1.1); 7.658 (2.0); 7.648 (3.1); 7.560 (0.3); 7.552 (3.4); 7.547 (1.2); 7.535 (1.2); 7.529 (3.7); 7.521 (2.6); 7.500 (2.1); 7.366 (0.7); 7.358 (0.6); 7.353 (0.7); 7.349 (0.8); 7.344 (0.7); 7.341 (0.7); 7.337 (0.7); 7.328 (0.6); 6.919 (0.4); 6.911 (3.6); 6.905 (1.2); 6.893 (1.1); 6.888 (3.4); 6.879 (0.3); 6.078 (1.0); 6.060 (1.0); 3.734 (16.0); 3.323 (34.0); 2.525 (0.9); 2.512 (18.3); 2.507 (37.5); 2.503 (51.7); 2.498 (38.5); 2.494 (18.7); 1.989 (1.5); 1.979 (4.5); 1.961 (4.4); 1.176 (0.6); 0.008 (2.5); 0.000 (63.6); −0.008 (2.3)

### Example I-1-238

¹H-NMR (400.0 MHz, d₆-DMSO): δ=10.528 (8.8); 9.708 (5.0); 9.691 (5.3); 8.543 (6.2); 8.537 (6.4); 8.316 (0.3); 8.282 (6.3); 7.938 (3.4); 7.932 (3.3); 7.918 (3.7); 7.911 (3.7); 7.738 (0.9); 7.715 (4.1); 7.700 (12.0); 7.682 (4.6); 7.562 (2.5); 7.542 (4.8); 7.528 (7.7); 7.507 (6.7); 7.396 (2.2); 7.390 (2.0); 7.379 (4.0); 7.373 (6.6); 7.364 (3.1); 7.357 (3.0); 7.352 (3.3); 6.102 (0.9); 6.085 (3.3); 6.067 (3.4); 6.049 (0.9); 5.756 (1.2); 4.057 (0.4); 4.039 (1.1); 4.021 (1.1); 4.004 (0.4); 3.327 (62.1); 2.677 (0.6); 2.673 (0.8); 2.668 (0.6); 2.526 (2.1); 2.508 (100.6); 2.504 (139.9); 2.499 (106.0); 2.335 (0.6); 2.330 (0.8); 2.326 (0.6); 1.988 (16.0); 1.970 (14.4); 1.194 (1.2); 1.176 (2.4); 1.159 (1.2); 0.146 (0.5); 0.008 (3.8); 0.000 (107.4); −0.008 (4.2); −0.150 (0.5)

### Example I-1-239

¹H-NMR (400.0 MHz, d₆-DMSO): δ=10.131 (4.1); 9.695 (2.5); 9.679 (2.6); 8.334 (2.4); 8.315 (2.6); 7.939 (2.1); 7.918 (2.8); 7.879 (6.4); 7.802 (1.3); 7.799 (1.4); 7.781 (2.3); 7.762 (1.2); 7.759 (1.2); 7.411 (1.3); 7.409 (1.4); 7.392 (2.5); 7.376 (1.2); 7.374 (1.2); 7.241 (1.9); 7.222 (2.1); 7.202 (1.0); 7.182 (1.9); 7.163 (1.1); 6.976 (1.4); 6.957 (2.3); 6.939 (1.0); 5.532 (8.5); 3.324 (70.1); 2.672 (0.5); 2.668 (0.4); 2.508 (61.9); 2.503 (85.9); 2.499 (65.2); 2.341 (16.0); 0.008 (2.1); 0.000 (57.2); −0.008 (2.4)

### Example I-1-240

¹H-NMR (400.0 MHz, d₆-DMSO): δ=9.338 (2.3); 9.336 (2.3); 9.322 (2.4); 8.315 (0.3); 8.059 (1.8); 8.050 (1.8); 7.690 (5.4); 7.320 (1.6); 7.300 (2.8); 7.253 (2.1); 7.236 (2.1); 7.232 (1.4); 7.216 (1.2); 5.513 (7.3); 4.047 (16.0); 3.319 (41.8); 2.777 (0.6); 2.768 (0.8); 2.759 (1.2); 2.750 (1.2); 2.741 (0.9); 2.732 (0.6); 2.671 (0.8); 2.507 (101.9); 2.502 (137.6); 2.498 (103.4); 2.333 (0.6); 2.329 (0.8); 2.325 (0.6); 1.045 (0.6); 1.030 (0.6); 0.742 (0.7); 0.729 (2.2); 0.724 (2.8); 0.712 (2.8); 0.707 (2.2); 0.695 (0.9); 0.494 (0.9); 0.483 (2.6); 0.477 (2.5); 0.468 (2.4); 0.456 (0.7); 0.146 (0.4); 0.008 (3.7); 0.000 (94.7); −0.008 (4.0); −0.150 (0.4)

### Example I-1-241

¹H-NMR (400.0 MHz, d₆-DMSO): δ=9.640 (5.2); 9.624 (5.4); 8.515 (6.6); 8.509 (6.8); 8.445 (2.2); 8.429 (4.6); 8.412 (2.3); 8.315 (0.5); 7.901 (3.5); 7.894 (3.6); 7.880 (4.0); 7.873 (3.9); 7.670 (1.3); 7.651 (3.8); 7.648 (3.9); 7.630 (6.6); 7.624 (6.4); 7.605 (1.7); 7.510 (7.7); 7.489 (7.0); 7.342 (2.4); 7.338 (2.4); 7.325 (4.5); 7.321 (4.4); 7.308 (2.2); 7.304 (2.2); 6.055 (1.1); 6.037 (3.6); 6.019 (3.6); 6.001 (1.1); 5.755 (6.8); 4.334 (0.3); 4.169 (2.6); 4.162 (2.2); 4.153 (2.3); 4.145 (3.8); 4.138 (2.4); 4.128 (2.2); 4.121 (2.6); 4.104 (0.8); 4.097 (0.8); 3.321 (169.7); 2.676 (1.2); 2.672 (1.6); 2.667 (1.2); 2.524 (4.5); 2.507 (192.0); 2.502 (265.0); 2.498 (203.0); 2.333 (1.1); 2.329 (1.5); 1.950 (16.0); 1.932 (15.9); 1.045 (2.0); 1.030 (2.0); 0.146 (0.8); 0.008 (6.8); 0.000 (174.2); −0.008 (7.8); −0.150 (0.8)

### Example I-1-242

¹H-NMR (400.0 MHz, d₆-DMSO): δ=9.605 (2.5); 9.588 (2.6); 8.641 (3.9); 8.638 (4.1); 8.397 (4.0); 8.355 (1.0); 8.338 (2.1); 8.321 (1.0); 7.706 (0.6); 7.703 (0.6); 7.684 (1.8); 7.681 (1.8); 7.667 (2.2); 7.664 (2.4); 7.656 (3.0); 7.636 (0.9); 7.352 (1.1); 7.348 (1.2); 7.336 (2.2); 7.331 (2.1); 7.319 (1.1); 7.314 (1.0); 5.755 (0.5); 5.416 (8.1); 4.193 (0.5); 4.168 (1.7); 4.152 (1.8); 4.144 (1.9); 4.127 (1.7); 4.120 (0.8); 4.103 (0.6); 3.320 (11.3); 2.676 (0.4); 2.671 (0.5); 2.667 (0.4); 2.525 (1.2); 2.511 (27.1); 2.507 (57.6); 2.502 (80.8); 2.498 (60.7); 2.494 (29.5); 2.454 (16.0); 2.333 (0.3); 2.329 (0.5); 2.325 (0.3); 0.008 (2.0); 0.000 (61.0); −0.008 (2.2)

### Example I-1-243

¹H-NMR (400.0 MHz, d₆-DMSO): δ=9.656 (2.3); 9.639 (2.3); 8.523 (2.7); 8.518 (2.8); 8.049 (1.9); 8.040 (2.0); 7.690 (1.2); 7.669 (2.8); 7.646 (1.5); 7.644 (1.6); 7.629 (1.7); 7.626 (2.0); 7.608 (1.9); 7.604 (2.4); 7.589 (1.7); 7.584 (1.8); 7.315 (1.1); 7.312 (1.1); 7.298 (1.9); 7.295 (2.0); 7.281 (1.0); 7.277 (1.0); 7.206 (2.7); 7.186 (2.5); 5.223 (7.6); 3.323 (16.0); 2.792 (0.6); 2.783 (0.9); 2.774 (1.4); 2.765 (1.4); 2.756 (1.0); 2.747 (0.6); 2.526 (0.7); 2.512 (17.1); 2.508 (35.8); 2.504 (49.9); 2.499 (37.9); 2.414 (16.0); 0.745 (0.8); 0.732 (2.4); 0.727 (3.1); 0.715 (3.1); 0.710 (2.5); 0.698 (1.0); 0.500 (1.0); 0.489 (2.9); 0.484 (2.8); 0.479 (2.6); 0.474 (2.7); 0.462 (0.8); 0.008 (1.4); 0.000 (42.0); −0.008 (1.7)

### Example I-1-244

¹H-NMR (400.0 MHz, d₆-DMSO): δ=9.691 (2.6); 9.674 (2.7); 8.615 (3.8); 8.612 (4.0); 8.395 (3.9); 7.957 (0.9); 7.943 (1.8); 7.928 (0.9); 7.610 (4.8); 7.600 (5.1); 7.305 (1.1); 7.294 (1.8); 7.288 (1.2); 7.283 (1.1); 7.278 (1.8); 7.267 (1.0); 5.756 (0.3); 5.399 (8.3); 3.336 (0.9); 3.322 (12.2); 3.304 (3.0); 3.301 (3.1); 3.286 (2.7); 3.268 (0.8); 2.672 (0.4); 2.525 (0.9); 2.511 (20.1); 2.507 (41.7); 2.503 (57.9); 2.498 (43.6); 2.453 (16.0); 2.329 (0.3); 1.121 (5.5); 1.103 (11.8); 1.085 (5.4); 1.046 (2.4); 1.030 (2.3); 0.008 (1.7); 0.000 (48.4); −0.008 (1.8)

### Example I-1-245

¹H-NMR (400.0 MHz, d₆-DMSO): δ=9.691 (2.5); 9.675 (2.6); 8.610 (3.8); 8.607 (4.0); 8.392 (3.9); 7.864 (0.5); 7.853 (1.4); 7.841 (1.4); 7.829 (0.5); 7.605 (4.2); 7.593 (5.4); 7.583 (0.3); 7.308 (1.1); 7.298 (1.1); 7.295 (1.2); 7.291 (1.2); 7.286 (1.1); 7.279 (1.2); 7.269 (1.0); 5.756 (2.3); 5.403 (8.5); 3.321 (24.9); 2.810 (10.0); 2.798 (10.0); 2.671 (0.4); 2.511 (20.9); 2.507 (43.1); 2.502 (60.3); 2.498 (46.0); 2.494 (22.9); 2.450 (16.0); 2.329 (0.4); 1.045 (0.4); 1.030 (0.4); 0.008 (2.0); 0.000 (54.1); −0.008 (2.2)

### Example I-1-246

¹H-NMR (400.0 MHz, d₆-DMSO): δ=9.732 (4.1); 9.716 (4.3); 8.497 (4.7); 8.490 (4.9); 7.961 (0.8); 7.950 (2.3); 7.938 (2.3); 7.926 (0.8); 7.873 (2.5); 7.867 (2.5); 7.853 (2.8); 7.846 (2.8); 7.575 (6.9); 7.566 (7.8); 7.563 (7.7); 7.503 (5.6); 7.482 (5.1); 7.301 (1.8); 7.290 (2.1); 7.285 (2.0); 7.280 (1.8); 7.274 (2.4); 7.263 (1.6); 6.052 (0.7); 6.034 (2.6); 6.016 (2.6); 5.998 (0.8); 5.756 (8.8); 4.345 (1.3); 4.335 (1.3); 4.039 (0.5); 4.021 (0.5); 3.794 (0.4); 3.784 (0.4); 3.779 (0.6); 3.768 (0.5); 3.764 (0.4); 3.753 (0.4); 3.323 (34.9); 2.812 (15.8); 2.800 (16.0); 2.677 (0.4); 2.673 (0.5); 2.668 (0.5); 2.526 (1.1); 2.521 (1.7); 2.512 (25.4); 2.508 (54.6); 2.503 (77.2); 2.499 (58.1); 2.495 (28.0); 2.330 (0.4); 2.326 (0.3); 1.990 (2.2); 1.943 (12.3); 1.925 (12.3); 1.233 (0.4); 1.194 (0.6); 1.176 (1.2); 1.158 (0.6); 1.046 (11.6); 1.031 (11.5); 0.008 (0.8); 0.000 (26.3); −0.008 (0.9)

### Example I-1-247

¹H-NMR (400.0 MHz, d₆-DMSO): δ=9.684 (2.2); 9.667 (2.3); 8.616 (3.7); 8.398 (3.7); 7.865 (1.7); 7.846 (1.7); 7.615 (4.5); 7.605 (3.9); 7.304 (0.9); 7.294 (1.1); 7.287 (1.1); 7.282 (1.0); 7.278 (1.1); 7.266 (0.8); 5.756 (0.5); 5.391 (7.4); 4.084 (0.7); 4.067 (1.1); 4.049 (1.0); 4.033 (0.7); 3.322 (6.9); 2.507 (31.9); 2.503 (42.3); 2.499 (32.4); 2.455 (13.9); 1.157 (16.0); 1.141 (16.0); 0.000 (2.1)

### Example I-1-248

¹H-NMR (400.0 MHz, d₆-DMSO): δ=9.725 (1.8); 9.709 (1.8); 8.004 (1.5); 7.985 (1.6); 7.677 (4.6); 7.458 (1.6); 7.439 (1.9); 7.231 (1.6); 7.213 (2.3); 7.196 (1.4); 5.563 (6.3); 4.344 (0.5); 4.334 (0.5); 4.093 (0.6); 4.076 (1.0); 4.058 (1.0); 4.042 (0.7); 3.322 (12.8); 2.634 (11.1); 2.526 (0.5); 2.512 (12.9); 2.508 (28.0); 2.504 (40.0); 2.499 (30.8); 1.185 (15.9); 1.169 (16.0); 1.046 (4.3); 1.031 (4.3); 1.011 (0.7); 0.994 (0.6); 0.008 (0.4); 0.000 (12.3); −0.008 (0.5)

### Example I-1-249

¹H-NMR (400.0 MHz, d₆-DMSO): δ=9.727 (3.8); 9.711 (4.0); 8.501 (4.6); 8.494 (4.8); 8.109 (1.5); 8.095 (3.0); 8.080 (1.5); 7.877 (2.4); 7.871 (2.4); 7.856 (2.7); 7.850 (2.7); 7.598 (0.3); 7.572 (5.4); 7.563 (7.8); 7.550 (0.6); 7.506 (5.2); 7.485 (4.7); 7.296 (1.5); 7.288 (1.6); 7.283 (1.8); 7.280 (1.9); 7.275 (1.8); 7.271 (1.7); 7.267 (1.7); 7.258 (1.4); 6.050 (0.7); 6.033 (2.5); 6.015 (2.6); 5.997 (0.8); 3.569 (1.9); 3.321 (44.7); 3.272 (1.5); 3.269 (1.5); 3.254 (4.1); 3.236 (4.2); 3.222 (1.6); 2.677 (0.5); 2.672 (0.7); 2.668 (0.5); 2.507 (83.4); 2.503 (115.0); 2.499 (88.9); 2.330 (0.7); 2.325 (0.5); 1.942 (11.2); 1.924 (11.2); 1.548 (0.5); 1.530 (2.5); 1.511 (5.1); 1.493 (5.2); 1.476 (2.8); 1.458 (0.6); 1.046 (0.3); 1.031 (0.3); 0.921 (8.0); 0.903 (16.0); 0.884 (7.1); 0.820 (0.3); 0.008 (1.5); 0.000 (40.1)

### Example I-1-250

¹H-NMR (400.0 MHz, d₆-DMSO): δ=9.730 (5.6); 9.714 (5.8); 8.507 (7.0); 8.502 (7.5); 8.171 (2.4); 8.157 (4.8); 8.143 (2.6); 7.887 (3.6); 7.881 (3.8); 7.866 (4.0); 7.860 (4.2); 7.583 (10.9); 7.573 (11.7); 7.509 (7.3); 7.488 (6.6); 7.309 (0.3); 7.297 (2.1); 7.287 (3.6); 7.281 (3.0); 7.276 (2.9); 7.270 (3.7); 7.260 (2.0); 6.051 (1.2); 6.033 (3.9); 6.015 (3.9); 5.997 (1.3); 5.756 (4.9); 4.346 (1.4); 4.335 (1.5); 3.795 (0.5); 3.780 (0.6); 3.769 (0.6); 3.753 (0.4); 3.324 (50.9); 3.198 (0.5); 3.180 (3.6); 3.176 (4.0); 3.162 (7.3); 3.149 (4.1); 3.145 (4.1); 3.126 (0.6); 3.110 (0.3); 2.673 (0.7); 2.504 (117.7); 2.330 (0.8); 1.948 (15.9); 1.930 (16.0); 1.445 (0.4); 1.429 (0.4); 1.233 (0.4); 1.046 (10.0); 1.031 (10.1); 1.005 (1.7); 0.999 (1.7); 0.987 (2.6); 0.975 (1.8); 0.969 (1.9); 0.957 (1.0); 0.469 (1.9); 0.455 (7.3); 0.444 (3.9); 0.435 (7.0); 0.425 (2.5); 0.408 (0.4); 0.233 (2.3); 0.221 (8.8); 0.208 (8.5); 0.197 (2.2); 0.000 (4.7)

### Example I-1-251

¹H-NMR (400.0 MHz, d₆-DMSO): δ=9.811 (2.6); 9.795 (2.7); 7.952 (0.6); 7.747 (4.4); 7.741 (4.4); 7.715 (6.8); 7.687 (2.5); 7.668 (2.8); 7.431 (4.5); 7.428 (4.5); 7.318 (2.4); 7.300 (3.2); 7.282 (2.2); 6.276 (3.1); 6.271 (5.2); 6.266 (3.2); 5.574 (8.8); 5.551 (15.2); 3.321 (50.2); 2.891 (3.5); 2.732 (3.1); 2.671 (1.0); 2.655 (16.0); 2.525 (1.5); 2.511 (33.2); 2.507 (70.5); 2.502 (99.3); 2.498 (75.9); 2.494 (38.0); 2.334 (0.4); 2.329 (0.6); 2.325 (0.4); 0.146 (0.4); 0.008 (2.8); 0.000 (84.6); −0.008 (3.4); −0.150 (0.4)

### Example I-1-252

¹H-NMR (400.0 MHz, d₆-DMSO): δ=9.645 (2.7); 9.629 (2.8); 8.655 (5.5); 8.344 (3.5); 8.339 (3.6); 7.631 (1.7); 7.625 (1.7); 7.610 (2.3); 7.604 (2.3); 7.495 (2.5); 7.487 (4.9); 7.476 (2.9); 7.467 (3.6); 7.292 (2.3); 7.274 (3.3); 7.256 (2.0); 5.494 (8.9); 4.039 (0.8); 4.021 (0.8); 3.323 (40.8); 2.672 (0.4); 2.507 (51.8); 2.503 (70.5); 2.499 (53.5); 2.430 (16.0); 2.330 0.4); 1.990 (3.3); 1.563 (1.7); 1.549 (4.5); 1.542 (4.7); 1.529 (2.0); 1.305 (2.2); 1.291 (4.5); 1.285 (4.7); 1.270 (1.7); 1.260 (0.3); 1.234 (0.4); 1.194 (0.9); 1.176 (1.7); 1.158 (1.0); 0.008 (1.0); 0.000 (24.0)

### Example I-1-253

¹H-NMR (400.0 MHz, d₆-DMSO): δ=9.745 (2.7); 9.729 (2.8); 8.338 (3.6); 8.333 (3.7); 8.214 (2.0); 8.194 (2.1); 7.614 (1.8); 7.607 (1.8); 7.593 (2.5); 7.587 (2.5); 7.492 (4.6); 7.471 (3.3); 7.405 (2.3); 7.386 (2.8); 7.226 (2.4); 7.208 (3.4); 7.208 (3.4); 7.190 (2.1); 5.505 (8.7); 4.039 (0.4); 4.021 (0.4); 3.852 (0.3); 3.829 (0.9); 3.818 (0.8); 3.808 (0.9); 3.785 (0.3); 3.323 (19.2); 2.526 (0.8); 2.513 (16.1); 2.508 (33.7); 2.504 (47.0); 2.499 (35.8); 2.426 (16.0); 1.990 (1.8); 1.860 (1.6); 1.847 (1.4); 1.837 (1.6); 1.829 (1.6); 1.689 (1.4); 1.678 (1.3); 1.666 (1.6); 1.656 (1.7); 1.563 (0.7); 1.554 (0.7); 1.532 (0.8); 1.406 (0.5); 1.397 (0.5); 1.379 (1.5); 1.352 (1.7); 1.320 (1.1); 1.295 (1.0); 1.288 (1.2); 1.265 (2.3); 1.240 (2.1); 1.217 (1.0); 1.194 (0.7); 1.176 (1.0); 1.159 (0.5); 0.008 (0.7); 0.000 (20.6); −0.008 (0.8)

### Example I-1-254

¹H-NMR (400.0 MHz, d₆-DMSO): δ=9.703 (3.7); 9.686 (3.8); 8.007 (3.2); 7.998 (3.3); 7.804 (8.7); 7.803 (8.5); 7.755 (2.4); 7.733 (3.9); 7.675 (2.1); 7.672 (2.2); 7.656 (2.6); 7.653 (3.3); 7.634 (1.6); 7.632 (1.5); 7.340 (1.9); 7.338 (2.0); 7.321 (3.6); 7.305 (1.8); 7.303 (1.7); 6.222 (0.8); 6.204 (2.6); 6.186 (2.6); 6.169 (0.8); 5.756 (16.0); 4.335 (0.3); 3.324 (2.0); 2.793 (0.4); 2.783 (1.0); 2.774 (1.6); 2.765 (2.3); 2.756 (2.3); 2.747 (1.6); 2.738 (1.1); 2.728 (0.4); 2.674 (0.4); 2.527 (1.2); 2.509 (49.0); 2.505 (67.5); 2.500 (50.8); 2.332 (0.4); 1.930 (12.3); 1.912 (12.3); 1.047 (2.8); 1.032 (2.8); 0.741 (1.4); 0.729 (3.7); 0.724 (5.0); 0.712 (4.9); 0.706 (4.0); 0.695 (1.6); 0.505 (1.7); 0.494 (4.7); 0.488 (4.5); 0.484 (4.2); 0.479 (4.2); 0.467 (1.3); 0.008 (0.7); 0.000 (21.1); −0.008 (0.7)

### Example I-1-255

¹H-NMR (400.0 MHz, d₆-DMSO): δ=9.745 (2.7); 9.728 (2.8); 8.333 (3.9); 8.328 (3.7); 8.284 (1.3); 8.269 (2.6); 8.255 (1.3); 7.615 (1.8); 7.608 (1.7); 7.594 (2.6); 7.588 (2.4); 7.493 (4.5); 7.472 (3.2); 7.405 (2.4); 7.386 (2.9); 7.224 (2.3); 7.207 (3.3); 7.189 (2.0); 5.513 (9.0); 3.323 (27.8); 3.177 (3.0); 3.161 (5.4); 3.145 (3.1); 2.673 (0.3); 2.508 (46.9); 2.504 (60.0); 2.499 (45.3); 2.427 (16.0); 2.331 (0.4); 1.990 (1.3); 1.732 (2.2); 1.702 (4.5); 1.671 (2.4); 1.634 (1.3); 1.611 (1.0); 1.485 (0.3); 1.468 (0.7); 1.459 (0.8); 1.451 (1.0); 1.442 (0.8); 1.433 (0.7); 1.422 (0.6); 1.414 (0.4); 1.259 (0.4); 1.250 (0.5); 1.219 (1.4); 1.194 (2.1); 1.189 (2.2); 1.176 (1.6); 1.162 (2.7); 1.140 (1.2); 1.110 (0.7); 0.995 (1.0); 0.964 (2.2); 0.936 (1.7); 0.912 (0.6); 0.902 (0.4); 0.000 (12.5)

### Example I-1-256

¹H-NMR (400.0 MHz, d₆-DMSO): δ=9.749 (2.8); 9.733 (2.8); 8.333 (3.7); 8.328 (3.8); 8.288 (1.3); 8.273 (2.5); 8.259 (1.3); 7.613 (1.7); 7.607 (1.7); 7.593 (2.3); 7.587 (2.3); 7.494 (4.2); 7.473 (3.0); 7.406 (2.4); 7.387 (2.8); 7.225 (2.1); 7.208 (3.3); 7.190 (1.8); 5.513 (9.1); 3.322 (18.8); 3.262 (3.1); 3.246 (5.0); 3.230 (3.2); 2.673 (0.4); 2.503 (67.2); 2.428 (16.0); 2.330 (0.4); 2.094 (0.5); 2.076 (1.3); 2.057 (1.8); 2.039 (1.4); 2.020 (0.6); 1.990 (1.2); 1.744 (0.6); 1.727 (1.3); 1.715 (1.8); 1.697 (2.0); 1.685 (1.5); 1.667 (0.9); 1.636 (0.4); 1.597 (2.2); 1.580 (1.9); 1.559 (1.0); 1.548 (1.3); 1.525 (1.9); 1.516 (1.7); 1.508 (2.1); 1.498 (1.5); 1.490 (1.3); 1.280 (0.7); 1.264 (1.7); 1.246 (2.0); 1.233 (1.9); 1.217 (1.4); 1.194 (0.6); 1.176 (0.6); 0.000 (13.7)

### Example I-1-257

¹H-NMR (400.0 MHz, d₆-DMSO): δ=9.528 (0.8); 9.512 (0.9); 8.403 (1.4); 8.398 (1.4); 7.825 (0.5); 7.806 (0.6); 7.551 (0.9); 7.532 (1.0); 7.471 (1.7); 7.451 (1.5); 7.268 (0.9); 7.250 (1.4); 7.232 (0.8); 4.343 (1.3); 4.335 (1.3); 3.792 (0.6); 3.785 (0.6); 3.776 (0.8); 3.770 (0.8); 3.762 (0.6); 3.755 (0.6); 3.665 (6.7); 3.323 (4.5); 2.670 (0.4); 2.629 (0.5); 2.503 (29.1); 1.947 (3.0); 1.930 (2.9); 1.234 (0.4); 1.046 (16.0); 1.030 (16.0); 0.000 (10.7)

### Example I-1-258

¹H-NMR (400.0 MHz, d₆-DMSO): δ=9.786 (2.5); 9.769 (2.6); 8.432 (4.2); 8.426 (4.3); 8.146 (1.6); 7.848 (1.4); 7.829 (1.5); 7.485 (6.2); 7.464 (8.2); 7.445 (3.3); 7.231 (3.2); 7.214 (4.7); 7.196 (2.8); 6.200 (1.0); 6.189 (1.0); 5.755 (2.4); 4.341 (0.7); 4.331 (0.7); 3.319 (19.5); 3.274 (0.4); 3.257 (0.9); 3.241 (1.8); 3.224 (2.6); 3.208 (2.2); 3.191 (1.3); 3.182 (1.5); 3.166 (1.8); 3.149 (1.2); 3.134 (0.7); 2.671 (1.5); 2.667 (1.5); 2.662 (1.4); 2.641 (1.6); 2.525 (1.4); 2.511 (30.6); 2.507 (62.2); 2.503 (82.3); 2.498 (60.7); 2.334 (0.4); 2.329 (0.6); 2.325 (0.4); 1.986 (9.7); 1.969 (9.6); 1.509 (0.6); 1.491 (2.6); 1.472 (5.3); 1.454 (5.5); 1.436 (2.9); 1.418 (0.7); 1.046 (4.9); 1.031 (4.8); 0.884 (8.1); 0.866 (16.0); 0.847 (7.2); 0.008 (0.6); 0.000 (18.1); −0.008 (0.7)

### Example I-1-259

¹H-NMR (400.0 MHz, d₆-DMSO): δ=8.416 (0.3); 7.479 (0.7); 7.458 (0.6); 7.234 (0.4); 3.804 (0.4); 3.788 (1.1); 3.773 (1.5); 3.758 (1.1); 3.742 (0.5); 2.524 (0.3); 2.511 (7.3); 2.507 (14.4); 2.502 (18.4); 2.498 (12.6); 2.493 (5.7); 1.966 (0.8); 1.949 (0.8); 1.045 (16.0); 1.039 (0.4); 1.030 (15.8); 1.024 (0.3); 0.675 (0.4); 0.662 (0.4); 0.436 (0.4); 0.431 (0.4); 0.426 (0.4); 0.421 (0.3); 0.000 (4.1)

### Example I-1-262

¹H-NMR (400.0 MHz, d₆-DMSO): δ=9.508 (1.7); 9.492 (1.8); 7.797 (4.2); 7.737 (0.5); 7.718 (1.4); 7.700 (1.6); 7.698 (1.6); 7.688 (2.0); 7.668 (0.6); 7.354 (0.9); 7.350 (0.8); 7.337 (1.6); 7.333 (1.5); 7.320 (0.8); 7.316 (0.8); 6.191 (0.4); 6.174 (1.3); 6.156 (1.3); 6.138 (0.4); 5.757 (1.8); 3.732 (16.0); 3.352 (4.5); 2.509 (24.9); 2.504 (34.0); 2.500 (25.4); 1.902 (6.2); 1.884 (6.1); 1.047 (0.5); 1.031 (0.5); 0.000 (3.8)

### Example I-1-263

¹H-NMR (400.0 MHz, d₆-DMSO): δ=9.626 (2.6); 9.610 (2.6); 8.535 (5.2); 8.355 (3.4); 8.349 (3.5); 7.642 (1.8); 7.636 (1.8); 7.621 (2.3); 7.615 (2.4); 7.494 (4.8); 7.487 (2.5); 7.473 (4.3); 7.469 (3.0); 7.280 (2.5); 7.262 (3.3); 7.244 (2.2); 5.512 (8.4); 4.056 (0.6); 4.038 (1.8); 4.021 (1.8); 4.003 (0.6); 3.318 (17.4); 2.676 (0.4); 2.671 (0.5); 2.666 (0.4); 2.524 (1.2); 2.520 (1.9); 2.511 (32.5); 2.506 (67.9); 2.502 (90.7); 2.497 (65.3); 2.493 (31.7); 2.434 (16.0); 2.333 (0.5); 2.329 (0.7); 2.324 (0.6); 2.299 (1.3); 2.269 (1.5); 1.989 (7.9); 1.881 (1.0); 1.857 (1.7); 1.832 (1.0); 1.683 (1.0); 1.656 (1.5); 1.643 (1.5); 1.562 (2.1); 1.548 (1.9); 1.538 (2.0); 1.506 (0.6); 1.405 (0.4); 1.397 (0.5); 1.380 (0.7); 1.370 (0.7); 1.356 (0.7); 1.345 (0.6); 1.299 (0.4); 1.259 (0.5); 1.235 (0.5); 1.193 (2.1); 1.175 (4.1); 1.157 (2.1); 0.000 (9.2); −0.009 (0.3)

### Example I-1-264

¹H-NMR (400.0 MHz, d₆-DMSO): δ=10.835 (4.9); 9.738 (2.5); 922 (26); 9.722 (2.6); 8.384 (3.4); 8.379 (3.5); 8.287 (2.1); 8.285 (2.2); 8.283 (2.0); 8.275 (2.3); 8.273 (2.1); 8.271 (2.0); 8.255 (3.3); 8.234 (3.6); 7.801 (1.2); 7.796 (1.2); 7.779 (2.0); 7.762 (1.2); 7.757 (1.2); 7.695 (1.9); 7.689 (1.8); 7.674 (2.3); 7.668 (2.3); 7.536 (2.3); 7.517 (2.8); 7.507 (4.4); 7.486 (3.4); 7.329 (2.4); 7.312 (3.2); 7.294 (2.0); 7.067 (1.7); 7.065 (1.8); 7.055 (1.7); 7.052 (2.0); 7.049 (1.9); 7.046 (1.8); 7.037 (1.7); 7.034 (1.7); 5.757 (1.2); 5.564 (8.6); 3.320 (32.7); 2.676 (0.5); 2.671 (0.6); 2.667 (0.5); 2.525 (1.6); 2.511 (36.9); 2.507 (75.6); 2.502 (99.4); 2.498 (71.6); 2.493 (34.7); 2.458 (16.0); 2.334 (0.4); 2.329 (0.6); 2.324 (0.4); 1.352 (0.4); 1.226 (0.5); 0.146 (0.3); 0.008 (2.9); 0.000 (81.1); −0.009 (3.0); −0.150 (0.3)

### Example I-1-265

¹H-NMR (400.0 MHz, d₆-DMSO): δ=11.039 (5.2); 9.728 (2.7); 9.712 (2.7); 8.642 (9.1); 8.630 (9.3); 8.387 (3.4); 8.382 (3.7); 7.699 (1.7); 7.692 (1.9); 7.678 (2.1); 7.672 (2.3); 7.554 (2.3); 7.535 (2.8); 7.507 (4.1); 7.486 (3.3); 7.355 (2.1); 7.337 (3.2); 7.319 (1.8); 7.144 (2.6); 7.132 (4.8); 7.120 (2.5); 5.757 (5.5); 5.560 (8.8); 3.319 (44.3); 2.671 (0.8); 2.506 (101.9); 2.502 (137.2); 2.498 (110.7); 2.459 (16.0); 2.442 (0.9); 2.328 (0.8); 1.351 (0.4); 1.230 (0.5); 0.146 (0.4); 0.008 (3.4); 0.000 (76.3); −0.150 (0.4)

### Example I-1-267

¹H-NMR (400.0 MHz, d₆-DMSO): δ=10.025 (2.4); 10.009 (2.5); 7.697 (6.3); 7.619 (2.2); 7.600 (2.5); 7.275 (2.2); 7.257 (3.1); 7.239 (2.0); 5.533 (9.0); 3.321 (12.7); 2.915 (3.8); 2.897 (5.6); 2.878 (4.1); 2.677 (0.4); 2.673 (0.6); 2.665 (0.8); 2.643 (16.0); 2.526 (1.0); 2.512 (28.1); 2.508 (55.4); 2.504 (70.9); 2.499 (50.4); 2.330 (0.4); 1.677 (0.5); 1.658 (2.1); 1.640 (3.9); 1.621 (3.9); 1.603 (2.2); 1.584 (0.5); 0.956 (6.8); 0.938 (13.7); 0.919 (6.1); 0.845 (0.4); 0.841 (0.5); 0.822 (0.4); 0.803 (0.5); 0.008 (1.0); 0.000 (25.8); −0.008 (1.1)

### Example I-1-269

¹H-NMR (400.0 MHz, d₆-DMSO): δ=9.561 (5.0); 9.545 (5.3); 9.522 (9.1); 8.679 (8.1); 8.676 (8.5); 8.423 (8.1); 8.422 (8.2); 7.750 (1.1); 7.747 (1.2); 7.728 (3.7); 7.725 (4.0); 7.708 (9.5); 7.705 (6.7); 7.685 (1.5); 7.384 (2.4); 7.379 (2.4); 7.374 (1.2); 7.367 (4.5); 7.362 (4.5); 7.351 (3.4); 7.345 (2.6); 7.337 (3.2); 7.319 (1.6); 7.316 (2.3); 7.300 (1.1); 7.211 (1.0); 7.202 (6.6); 7.182 (10.0); 7.161 (4.2); 7.152 (0.9); 5.758 (0.4); 5.466 (16.0); 4.345 (0.6); 4.335 (0.1); 3.569 (0.9); 3.323 (19.5); 2.676 (0.4); 2.672 (0.6); 2.667 (0.5); 2.525 (1.5); 2.512 (38.3); 2.508 (79.5); 2.503 (105.4); 2.499 (75.7); 2.494 (37.0); 2.467 (32.9); 2.334 (0.4); 2.330 (0.6); 2.325 (0.5); 1.046 (4.3); 1.031 (4.2); 0.000 (1.5)

### Example I-1-270

¹H-NMR (400.0 MHz, d₆-DMSO): δ=9.584 (4.6); 9.568 (4.7); 8.437 (8.4); 8.437 (8.4); 7.903 (3.7); 7.881 (5.2); 7.841 (11.9); 7.788 (2.6); 7.785 (2.7); 7.769 (3.1); 7.766 (4.3); 7.747 (2.1); 7.745 (2.1); 7.387 (2.4); 7.384 (2.5); 7.368 (4.5); 7.352 (2.2); 7.349 (2.2); 5.758 (10.5); 5.438 (16.0); 3.569 (0.6); 3.328 (14.5); 2.677 (0.6); 2.672 (0.9); 2.668 (0.6); 2.526 (2.2); 2.512 (53.6); 2.508 (110.6); 2.503 (146.5); 2.499 (105.7); 2.495 (51.8); 2.335 (0.6); 2.330 (0.9); 2.326 (0.6); 1.562 (2.8); 1.547 (7.3); 1.540 (7.7); 1.527 (3.5); 1.487 (0.3); 1.349 (0.3); 1.309 (3.6); 1.296 (7.2); 1.289 (7.7); 1.275 (2.8); 1.235 (0.7); 1.046 (0.4); 1.031 (0.4); 0.008 (0.4); 0.000 (12.1); −0.008 (0.5)

### Example I-1-271

¹H-NMR (400.0 MHz, d₆-DMSO): δ=9.630 (0.8); 9.613 (0.8); 8.538 (1.4); 8.498 (1.0); 8.492 (1.0); 7.879 (0.5); 7.873 (0.5); 7.858 (0.6); 7.852 (0.6); 7.666 (0.6); 7.648 (0.7); 7.633 (1.0); 7.613 (0.4); 7.503 (1.1); 7.482 (1.0); 7.358 (0.4); 7.341 (0.7); 7.327 (0.3); 7.324 (0.4); 6.013 (0.6); 5.995 (0.6); 3.834 (0.4); 3.777 (0.6); 3.568 (16.0); 2.507 (19.9); 2.503 (26.8); 2.499 (21.9); 1.930 (2.3); 1.912 (2.3); 1.543 (0.5); 1.529 (1.2); 1.523 (1.4); 1.510 (0.6); 1.290 (0.6); 1.277 (1.2); 1.270 (1.3); 1.256 (0.5)

### Example I-1-272

¹H-NMR (400.0 MHz, d₆-DMSO): δ=9.602 (10.7); 9.581 (5.4); 8.551 (6.6); 8.544 (6.7); 7.938 (3.4); 7.931 (3.5); 7.917 (3.9); 7.910 (3.8); 7.711 (1.3); 7.708 (1.4); 7.689 (3.8); 7.686 (3.8); 7.672 (4.7); 7.669 (5.1); 7.661 (6.1); 7.641 (1.8); 7.528 (7.7); 7.507 (7.0); 7.370 (2.6); 7.366 (2.5); 7.353 (5.9); 7.349 (5.0); 7.337 (5.4); 7.332 (3.7); 7.316 (2.5); 7.300 (10.1); 7.216 (0.6); 7.211 (1.0); 7.202 (6.7); 7.182 (10.2); 7.169 (1.1); 7.161 (4.3); 7.152 (0.9); 6.100 (1.1); 6.082 (3.5); 6.064 (3.6); 6.046 (1.0); 5.757 (2.2); 3.321 (58.5); 2.676 (1.0); 2.671 (1.4); 2.667 (1.0); 2.525 (3.5); 2.511 (88.1); 2.507 (181.7); 2.502 (239.7); 2.498 (172.1); 2.493 (83.1); 2.333 (1.1); 2.329 (1.4); 1.982 (16.0); 1.965 (15.9); 1.232 (0.3); 1.045 (0.6); 1.030 (0.7); 0.000 (5.4)

### Example I-1-273

¹H-NMR (400.0 MHz, d₆-DMSO): δ=9.124 (4.6); 9.107 (4.7); 7.857 (12.4); 7.806 (3.8); 7.784 (4.8); 7.630 (2.5); 7.627 (2.6); 7.612 (3.1); 7.609 (4.4); 7.606 (2.5); 7.590 (2.3); 7.587 (2.3); 7.227 (2.4); 7.224 (2.5); 7.208 (4.2); 7.207 (4.2); 7.192 (2.3); 7.188 (2.2); 5.758 (7.3); 5.400 (16.0); 3.705 (0.8); 3.491 (0.8); 3.323 (44.3); 2.677 (0.5); 2.673 (0.7); 2.668 (0.5); 2.526 (1.7); 2.521 (2.6); 2.513 (41.3); 2.508 (86.0); 2.504 (114.1); 2.499 (80.5); 2.495 (37.8); 2.335 (0.5); 2.330 (0.7); 2.326 (0.5); 1.853 (4.7); 1.837 (12.4); 1.820 (4.6); 1.234 (1.6); 0.008 (0.7); 0.000 (24.0); −0.009 (0.8)

### Example I-1-274

¹H-NMR (400.0 MHz, d₆-DMSO): δ=10.390 (4.4); 9.625 (2.6); 9.609 (2.6); 8.243 (2.5); 8.231 (2.6); 7.806 (1.3); 7.784 (1.9); 7.760 (1.4); 7.721 (5.9); 7.591 (2.3); 7.572 (2.6); 7.334 (1.9); 7.316 (3.1); 7.299 (2.1); 7.293 (1.9); 7.282 (2.0); 7.272 (1.6); 7.262 (0.9); 5.757 (13.6); 5.634 (8.7); 3.320 (15.4); 3.175 (0.6); 3.162 (0.6); 2.706 (0.4); 2.667 (16.0); 2.502 (136.7); 2.329 (0.8); 0.146 (0.4); 0.000 (81.6); −0.150 (0.4)

### Example I-1-275

¹H-NMR (400.0 MHz, d₆-DMSO): δ=9.665 (4.5); 9.636 (4.6); 9.620 (4.5); 8.494 (7.6); 8.488 (7.7); 7.919 (2.6); 7.901 (2.8); 7.581 (4.8); 7.562 (5.4); 7.511 (10.8); 7.490 (9.9); 7.350 (1.0); 7.334 (2.2); 7.329 (2.0); 7.313 (4.9); 7.308 (7.4); 7.291 (10.4); 7.273 (5.2); 7.184 (1.5); 7.176 (8.3); 7.155 (12.9); 7.135 (5.7); 7.126 (1.1); 6.251 (1.8); 6.236 (1.8); 4.344 (1.7); 4.334 (1.7); 3.794 (0.4); 3.783 (0.5); 3.778 (0.6); 3.763 (0.5); 3.752 (0.4); 3.568 (1.7); 3.321 (36.2); 2.676 (3.5); 2.672 (3.7); 2.525 (3.0); 2.507 (147.0); 2.502 (191.5); 2.498 (138.3); 2.333 (0.9); 2.329 (1.2); 2.325 (0.9); 2.029 (16.0); 2.012 (15.9); 1.046 (11.4); 1.030 (11.3); 0.146 (0.7); 0.008 (5.6); 0.000 (150.1); −0.008 (6.1); −0.150 (0.7)

### Example I-1-276

¹H-NMR (400.0 MHz, d₆-DMSO): δ=9.815 (5.6); 9.576 (2.6); 9.560 (2.6); 7.729 (6.5); 7.572 (2.3); 7.554 (2.6); 7.440 (1.3); 7.437 (1.3); 7.433 (1.1); 7.424 (2.6); 7.418 (2.2); 7.377 (0.5); 7.361 (0.9); 7.356 (2.2)); 7.347 (2.5); 7.34); 7.323 (3.1); 7.319 (2.4); 7.309 (2.5); 7.304 (0.8); 7.291 (3.3); 7.273 (2.1); 7.217 (0.3); 5.758 (2.9); 5.631 (8.4); 3.418 (0.5); 2.664 (16.0); 2.525 (1.5); 2.511 (38.5); 2.507 (78.5); 2.503 (103.9); 2.498 (74.2); 2.494 (35.6); 2.334 (0.4); 2.329 (0.6); 2.325 (0.4); 1.046 (3.0); 1.030 (2.9); 0.008 (2.0); 0.000 (56.8); −0.008 (2.0)

### Example I-1-277

¹H-NMR (400.0 MHz, d₆-DMSO): δ=9.875 (5.3); 9.571 (2.6); 9.555 (2.6); 7.735 (6.2); 7.583 (7.9); 7.563 (10.2); 7.550 (2.6); 7.369 (2.6); 7.349 (3.6); 7.329 (1.9); 7.304 (2.1); 7.287 (3.1); 7.269 (1.8); 5.757 (3.3); 5.631 (8.2); 3.320 (14.3); 2.666 (16.0); 2.506 (128.3); 2.502 (166.8); 2.498 (122.1); 2.329 (1.0); 2.325 (0.7); 1.045 (1.0); 1.030 (1.0); 0.146 (0.3); 0.008 (2.6); 0.000 (72.7); −0.150 (0.3)

### Example I-1-278

¹H-NMR (400.0 MHz, d₆-DMSO): δ=9.698 (3.5); 9.681 (3.5); 8.680 (5.0); 8.677 (7.0); 8.657 (6.0); 8.654 (5.4); 7.932 (1.3); 7.918 (2.5); 7.903 (1.3); 7.639 (0.4); 7.628 (6.1); 7.626 (7.1); 7.616 (5.3); 7.613 (3.7); 7.594 (0.4); 7.591 (0.4); 7.318 (1.4); 7.309 (1.5); 7.306 (1.6); 7.302 (1.7); 7.297 (1.6); 7.293 (1.7); 7.289 (1.4); 7.280 (1.3); 5.757 (3.3); 5.471 (13.1); 3.335 (1.1); 3.320 (16.8); 3.302 (4.1); 3.299 (4.3); 3.284 (3.7); 3.266 (1.1); 2.676 (0.4); 2.671 (0.6); 2.667 (0.4); 2.525 (1.2); 2.511 (32.2); 2.507 (68.6); 2.502 (93.0); 2.498 (68.3); 2.493 (33.9); 2.333 (0.4); 2.329 (0.6); 2.324 (0.4); 1.234 (0.4); 1.118 (7.5); 1.100 (16.0); 1.082 (7.4); 0.008 (0.5); 0.000 (18.3); −0.008 (0.8)

### Example I-1-279

¹H-NMR (400.0 MHz, d₆-DMSO): δ=9.691 (3.5); 9.675 (3.5); 8.681 (5.4); 8.678 (7.1); 8.659 (6.3); 7.999 (1.3); 7.984 (2.6); 7.969 (1.3); 7.638 (0.4); 7.626 (6.9); 7.616 (6.0); 7.319 (1.5); 7.310 (1.6); 7.307 (1.6); 7.302 (1.7); 7.297 (1.6); 7.293 (1.6); 7.290 (1.4); 7.281 (1.3); 5.757 (2.0); 5.473 (13.0); 3.321 (20.7); 3.268 (2.1); 3.251 (4.7); 3.236 (4.7); 3.219 (2.1); 2.676 (0.4); 2.671 (0.5); 2.667 (0.4); 2.525 (1.3); 2.511 (32.4); 2.507 (67.3); 2.502 (89.8); 2.498 (64.7); 2.494 (31.5); 2.333 (0.4); 2.329 (0.5); 2.325 (0.4); 1.536 (0.5); 1.517 (2.4); 1.499 (4.7); 1.482 (4.8); 1.464 (2.6); 1.446 (0.6); 0.910 (7.9); 0.891 (16.0); 0.873 (7.0); 0.008 (0.5); 0.000 (17.4); −0.008 (0.7)

### Example I-1-280

¹H-NMR (400.0 MHz, d₆-DMSO): δ=9.699 (3.9); 9.682 (4.0); 8.677 (6.3); 8.673 (7.9); 8.653 (7.2); 7.840 (0.8); 7.829 (2.3); 7.817 (2.3); 7.805 (0.8); 7.622 (7.8); 7.612 (7.8); 7.322 (1.7); 7.311 (2.5); 7.305 (1.9); 7.300 (1.8); 7.295 (2.2); 7.283 (1.5); 5.476 (15.8); 4.344 (0.6); 4.334 (0.6); 3.321 (28.2); 2.809 (16.0); 2.797 (15.9); 2.676 (0.5); 2.671 (0.7); 2.667 (0.5); 2.525 (1.7); 2.511 (42.4); 2.507 (85.4); 2.502 (111.7); 2.498 (80.3); 2.334 (0.5); 2.329 (0.7); 2.325 (0.5); 1.045 (4.4); 1.030 (4.4); 0.008 (0.6); 0.000 (16.4); −0.008 (0.7)

### Example I-1-281

¹H-NMR (400.0 MHz, d₆-DMSO): δ=9.510 (1.6); 9.493 (1.6); 8.682 (3.1); 8.679 (3.5); 8.634 (3.2); 8.632 (3.0); 7.511 (1.4); 7.492 (1.6); 7.265 (1.4); 7.247 (2.1); 7.229 (1.3); 5.601 (7.0); 3.715 (16.0); 3.320 (7.4); 2.506 (34.2); 2.502 (44.8); 2.498 (32.3); 2.469 (10.4); 0.008 (1.1); 0.000 (31.0); −0.008 (1.3)

### Example I-1-282

¹H-NMR (400.0 MHz, d₆-DMSO): δ=9.703 (8.8); 9.540 (4.7); 9.523 (4.8); 7.954 (3.9); 7.932 (5.1); 7.893 (11.4); 7.813 (2.5); 7.811 (2.6); 7.792 (4.2); 7.773 (2.2); 7.582 (12.9); 7.561 (15.8); 7.392 (2.6); 7.375 (5.1); 7.370 (5.3); 7.359 (2.9); 7.350 (6.1); 7.329 (3.1); 5.513 (15.5); 4.345 (2.3); 4.335 (2.3); 3.794 (0.6); 3.783 (0.7); 3.779 (0.9); 3.768 (0.8); 3.763 (0.7); 3.753 (0.6); 3.322 (49.4); 2.672 (1.1); 2.507 (149.3); 2.503 (188.8); 2.499 (139.2); 2.334 (1.0); 2.330 (1.2); 2.326 (0.9); 1.046 (16.0); 1.031 (15.9); 0.000 (15.2)

### Example I-1-283

¹H-NMR (400.0 MHz, d₆-DMSO): δ=9.640 (9.0); 9.544 (4.7); 9.527 (5.0); 7.953 (3.9); 7.931 (5.3); 7.888 (11.6); 7.815 (2.7); 7.796 (4.3); 7.775 (2.2); 7.439 (2.4); 7.434 (1.9); 7.424 (4.2); 7.418 (3.8); 7.397 (2.7); 7.379 (5.3); 7.362 (3.9); 7.357 (4.0); 7.346 (4.2); 7.340 (7.7); 7.323 (5.8); 7.318 (3.7); 7.302 (1.0); 7.298 (0.6); 5.513 (16.0); 4.347 (1.5); 4.336 (1.6); 3.795 (0.5); 3.785 (0.5); 3.780 (0.6); 3.770 (0.6); 3.765 (0.5); 3.754 (0.4); 3.325 (29.2); 2.674 (0.7); 2.509 (88.6); 2.505 (115.2); 2.501 (87.0); 2.341 (0.6); 2.331 (0.7); 1.047 (13.2); 1.032 (13.1); 0.000 (9.7)

### Example I-1-284

¹H-NMR (400.0 MHz, d₆-DMSO): δ=10.217 (7.3); 9.589 (4.7); 9.573 (4.9); 9.472 (0.4); 9.455 (0.4); 8.242 (4.7); 8.230 (4.8); 7.964 (3.9); 7.943 (5.3); 7.885 (12.1); 7.840 (1.5); 7.835 (2.8); 7.832 (20.8); 7.814 (4.7); 7.810 (4.1); 7.806 (2.7); 7.795 (2.6); 7.792 (2.9); 7.789 (3.0); 7.784 (3.6); 7.779 (2.6); 7.762 (2.7); 7.759 (2.5); 7.422 (2.5); 7.420 (2.6); 7.404 (4.7); 7.387 (2.3); 7.385 (2.3); 7.337 (0.4); 7.303 (2.3); 7.294 (3.0); 7.283 (4.1); 7.273 (2.6); 7.262 (1.9); 5.758 (4.1); 5.517 (16.0); 5.374 (1.5); 3.738 (3.7); 3.325 (32.1); 2.677 (0.8); 2.672 (1.1); 2.668 (0.8); 2.512 (73.3); 2.508 (145.5); 2.503 (190.9); 2.499 (139.8); 2.335 (0.8); 2.330 (1.1); 2.326 (0.8); 1.232 (0.4); 1.046 (1.3); 1.031 (1.2); 0.008 (0.8); 0.000 (18.0)

### Example I-1-285

¹H-NMR (400.0 MHz, d₆-DMSO): δ=10.700 (8.5); 9.621 (4.8); 9.604 (5.0); 8.516 (5.0); 8.495 (5.4); 7.955 (3.7); 7.933 (5.5); 7.873 (11.8); 7.853 (2.9); 7.851 (2.9); 7.832 (4.4); 7.813 (2.4); 7.811 (20.6); 7.805 (4.2); 7.802 (4.4); 7.786 (4.3); 7.783 (4.4); 7.696 (2.1); 7.693 (2.1); 7.675 (4.0); 7.656 (2.2); 7.653 (2.1); 7.436 (2.6); 7.419 (4.8); 7.403 (2.4); 7.401 (2.3); 7.218 (2.9); 7.199 (5.2); 7.180 (2.6); 5.758 (1.1); 5.531 (16.0); 3.324 (36.4); 2.673 (0.7); 2.509 (95.4); 2.504 (123.1); 2.500 (91.3); 2.331 (0.7); 1.047 (0.5); 1.031 (0.5); 0.000 (2.4)

### Example I-1-286

¹H-NMR (400.0 MHz, d₆-DMSO): δ=10.430 (4.5); 10.424 (4.6); 9.646 (4.6); 9.630 (4.8); 8.487 (1.9); 8.466 (3.8); 8.449 (2.0); 7.954 (3.8); 7.932 (5.2); 7.881 (11.0); 7.824 (2.7); 7.805 (4.3); 7.784 (2.2); 7.421 (2.6); 7.404 (4.7); 7.386 (2.3); 7.318 (2.0); 7.298 (2.6); 7.292 (2.3); 7.269 (2.3); 7.197 (1.9); 7.178 (4.1); 7.158 (2.5); 7.070 (1.4); 7.055 (2.4); 7.038 (2.2); 7.022 (0.9); 5.759 (0.7); 5.516 (16.0); 3.325 (23.1); 2.674 (0.6); 2.505 (101.0); 2.331 (0.6); 1.047 (0.8); 1.032 (0.8); 0.000 (1.8)

### Example I-1-287

¹H-NMR (400.0 MHz, d₆-DMSO): δ=9.499 (1.1); 9.482 (1.1); 7.755 (2.5); 7.574 (1.7); 7.555 (1.9); 7.273 (1.3); 7.255 (2.0); 7.237 (1.1); 6.324 (0.6); 6.308 (0.6); 5.757 (2.2); 3.723 (0.6); 3.709 (16.0); 3.327 (5.0); 2.758 (2.1); 2.672 (0.4); 2.668 (0.4); 2.507 (47.1); 2.503 (60.4); 2.499 (42.8428); 2.329 (0.4); 1.942 (4.7); 1.925 (4.6); 1.234 (0.5); 1.046 (1.0); 1.030 (1.0); 0.146 (0.3); 0.008 (2.5); 0.000 (67.9); −0.008 (2.7); −0.150 (0.3)

### Example I-1-288

¹H-NMR (400.0 MHz, d₆-DMSO): δ=9.595 (5.1); 9.578 (5.3); 9.538 (9.2); 7.849 (12.3); 7.825 (3.0); 7.803 (5.8); 7.769 (3.4); 7.751 (4.0); 7.748 (4.1); 7.729 (2.0); 7.392 (2.8); 7.374 (5.0); 7.366 (2.2); 7.358 (3.5); 7.345 (3.2); 7.324 (2.3); 7.309 (1.3); 7.207 (6.2); 7.187 (9.8); 7.167 (4.3); 7.157 (1.1); 6.292 (1.0); 6.275 (3.6); 6.257 (3.7); 6.240 (1.1); 5.757 (0.8); 3.321 (40.0); 2.672 (1.2); 2.507 (166.8); 2.503 (203.1); 2.498 (144.9); 2.329 (1.2); 1.987 (16.0); 1.969 (15.9); 1.046 (2.1); 1.031 (2.1); 0.146 (0.4); 0.000 (96.6); −0.150 (0.5)

### Example I-1-289

¹H-NMR (400.0 MHz, d₆-DMSO): δ=9.628 (3.3); 9.612 (3.3); 8.489 (6.1); 8.489 (6.1); 7.807 (8.0); 7.792 (1.9); 7.770 (3.8); 7.745 (2.3); 7.725 (2.7); 7.705 (1.2); 7.380 (1.7); 7.364 (3.1); 7.348 (1.5); 7.068 (2.1); 6.225 (0.7); 6.208 (2.4); 6.191 (2.4); 6.173 (0.7); 4.344 (2.4); 4.333 (2.5); 4.022 (0.4); 4.014 (0.4); 4.006 (0.4); 3.998 (0.4); 3.829 (0.8); 3.821 (0.8); 3.793 (0.8); 3.782 (0.7); 3.778); 3.767 (0.9); 3.763 (0.7); 3.752 (0.6); 3.320 (37.0); 3.209 (0.8); 3.186 (6.2); 2.671 (0.9); 2.506 (126.5); 2.502 (161.7); 2.498 (121.6); 2.329 (0.9); 2.301 (0.5); 1.932 (10.4); 1.915 (10.4); 1.549 (1.9); 1.536 (4.8); 1.530 (5.2); 1.518 (2.6); 1.418 (1.5); 1.411 (1.6); 1.305 (3.5); 1.292 (7.1); 1.273 (20.0); 1.259 (0.4); 1.234 (0.9); 1.221 (0.3); 1.202 (0.4); 1.192 (0.5); 1.184 (0.5); 1.177 (0.4); 1.167 (0.4); 1.102 (0.3); 1.093 (0.3); 1.088 (0.4); 1.079 (0.5); 1.063 (0.4); 1.045 (16.0); 1.030 (15.8); 1.008 (0.4); 0.984 (1.2); 0.978 (1.2); 0.970 (1.2); 0.957 (0.5); 0.946 (0.6); 0.937 (0.4); 0.928 (0.4); 0.919 (0.4); 0.146 (0.4); 0.000 (75.6); −0.150 (0.4)

### Example I-1-290

¹H-NMR (400.0 MHz, d₆-DMSO): δ=9.737 (4.0); 9.721 (3.9); 8.316 (0.4); 8.126 (4.4); 8.118 (4.3); 7.754 (10.3); 7.503 (5.8); 7.484 (6.7); 7.257 (4.6); 7.239 (7.2); 7.221 (3.9); 6.375 (2.9); 6.358 (2.8); 5.757 (3.0); 3.728 (0.3); 3.320 (67.2); 2.758 (10.6); 2.750 (11.9); 2.741 (8.8); 2.732 (5.6); 2.722 (3.5); 2.713 (1.7); 2.671 (2.5); 2.667 (2.0); 2.630 (0.4); 2.506 (282.8); 2.502 (370.6); 2.498 (273.0); 2.329 (2.1); 1.969 (16.0); 1.952 (15.9); 1.625 (0.4); 1.423 (0.9); 1.406 (0.8); 1.235 (0.5); 1.176 (0.4); 1.163 (0.6); 1.156 (0.7); 1.006 (0.6); 0.997 (0.5); 0.725 (2.2); 0.714 (7.2); 0.709 (8.9); 0.697 (8.7); 0.692 (7.6); 0.681 (3.0); 0.656 (0.4); 0.538 (0.4); 0.528 (0.4); 0.512 (0.5); 0.496 (3.4); 0.486 (9.6); 0.479 (9.3); 0.470 (7.9); 0.460 (2.8); 0.146 (0.5); 0.000 (88.4); −0.150 (0.5)

### Example I-1-291

¹H-NMR (400.0 MHz, d₆-DMSO): δ=9.669 (7.5); 9.633 (0.6); 9.611 (4.2); 9.596 (4.1); 8.317 (0.4); 7.806 (10.7); 7.602 (6.4); 7.583 (7.2); 7.392 (0.4); 7.372 (1.3); 7.357 (2.7); 7.352 (2.4); 7.335 (5.3); 7.313 (7.5); 7.295 (8.1); 7.277 (4.6); 7.206 (2.0); 7.198 (10.4); 7.178 (16.0); 7.157 (7.2); 7.148 (1.6); 7.128 (0.3); 7.022 (0.4); 6.999 (0.4); 6.467 (0.4); 6.432 (2.8); 6.417 (2.9); 5.757 (3.0); 5.155 (0.4); 4.498 (0.5); 4.484 (0.5); 3.569 (0.9); 3.321 (66.8); 2.920 (0.5); 2.878 (2.7); 2.792 (8.2); 2.712 (0.6); 2.676 (1.6); 2.672 (2.1); 2.667 (1.6); 2.619 (0.5); 2.511 (121.5); 2.507 (241.5); 2.503 (315.8); 2.498 (224.8); 2.494 (107.5); 2.335 (1.9); 2.330 (2.0); 2.067 (0.9); 2.035 (15.5); 2.018 (15.3); 1.626 (0.4); 1.232 (0.5); 1.175 (0.4); 1.161 (0.4); 1.156 (0.3); 1.124 (2.1); 1.112 (6.3); 1.105 (6.6); 1.094 (2.6); 1.054 (0.4); 0.922 (0.3); 0.882 (2.7); 0.871 (6.8); 0.864 (6.6); 0.852 (2.2); 0.146 (0.4); 0.008 (2.8); 0.000 (72.4); −0.009 (2.7); −0.150 (0.3)

### Example I-1-292

¹H-NMR (400.0 MHz, d₆-DMSO): δ=9.933 (1.6); 9.916 (1.6); 7.685 (3.9); 7.642 (1.4); 7.623 (1.6); 7.305 (1.3); 7.287 (1.9); 7.269 (1.1); 5.520 (5.6); 4.607 (8.7); 3.716 (0.4); 3.701 (1.1); 3.685 (1.5); 3.670 (1.1); 3.655 (0.5); 3.324 (23.6); 2.636 (9.9); 2.508 (31.6); 2.504 (41.5); 2.499 (31.9); 1.149 (16.0); 1.134 (15.9); 0.000 (22.5)

### Example I-1-293

¹H-NMR (400.0 MHz, d₆-DMSO): δ=9.944 (1.8); 9.928 (1.9); 7.686 (4.3); 7.641 (1.6); 7.623 (1.8); 7.302 (1.4); 7.284 (2.1); 7.266 (1.2); 5.520 (6.2); 4.630 (9.3); 3.325 (10.7); 3.307 (1.5); 3.293 (1.9); 3.278 (1.2); 3.264 (0.4); 2.638 (11.0); 2.508 (30.6); 2.504 (37.2); 2.500 (27.4); 1.536 (1.1); 1.517 (3.9); 1.503 (4.9); 1.499 (4.7); 1.484 (4.3); 1.466 (1.4); 1.047 (0.5); 1.031 (0.5); 0.893 (8.1); 0.875 (16.0); 0.856 (7.2); 0.000 (20.0)

### Example I-1-294

¹H-NMR (400.0 MHz, d₆-DMSO): δ=9.935 (2.6); 9.919 (2.7); 7.686 (6.5); 7.641 (2.4); 7.622 (2.7); 7.301 (2.1); 7.284 (3.1); 7.266 (1.9); 5.757 (1.1); 5.518 (8.9); 4.639 (13.9); 3.390 (0.8); 3.379 (1.0); 3.368 (1.4); 3.359 (1.2); 3.346 (0.8); 3.320 (18.5); 2.676 (0.6); 2.672 (0.7); 2.635 (16.0); 2.507 (85.9); 2.503 (107.3); 2.498 (78.6); 2.330 (0.6); 1.915 (1.7); 1.890 (2.4); 1.689 (2.1); 1.675 (2.2); 1.489 (1.2); 1.476 (1.0); 1.281 (1.5); 1.257 (3.1); 1.233 (3.8); 1.204 (3.5); 1.182 (2.1); 0.000 (31.7)

### Example I-1-295

¹H-NMR (400.0 MHz, d₆-DMSO): δ=10.424 (7.6); 9.612 (4.4); 9.595 (4.5); 8.545 (6.0); 8.540 (5.8); 7.856 (3.1); 7.851 (3.1); 7.835 (3.8); 7.829 (4.3); 7.802 (6.1); 7.790 (4.0); 7.771 (4.3); 7.755 (15.6); 7.752 (16.0); 7.514 (5.6); 7.493 (5.0); 7.413 (2.2); 7.395 (4.0); 7.379 (2.0); 7.217 (7.0); 5.347 (14.1); 3.321 (50.7); 2.671 (1.2); 2.502 (194.2); 2.329 (1.1); 2.076 (1.6); 0.146 (0.6); 0.000 (122.8); −0.150 (0.6)

### Example I-1-296

¹H-NMR (400.0 MHz, d₆-DMSO): δ=9.939 (0.5); 9.923 (0.5); 7.687 (1.3); 7.638 (0.5); 7.619 (0.5); 7.306 (0.4); 7.289 (0.6); 7.271 (0.4); 5.528 (1.8); 5.503 (0.6); 5.494 (0.6); 5.490 (0.7); 5.483 (0.8); 4.572 (3.0); 3.899 (0.4); 3.329 (3.7); 2.939 (0.8); 2.936 (0.8); 2.933 (0.5); 2.926 (1.1); 2.637 (3.1); 2.508 (13.6); 2.503 (17.6); 2.499 (12.8); 1.645 (1.9); 1.633 (1.4); 1.188 (16.0); 1.135 (1.8); 0.008 (1.2); 0.000 (20.5); −0.008 (1.0)

### Example I-1-297

¹H-NMR (400.0 MHz, d₆-DMSO): δ=9.881 (2.6); 9.864 (2.7); 7.679 (6.4); 7.652 (2.7); 7.330 (1.9); 7.312 (3.0); 7.294 (1.8); 5.522 (9.1); 4.809 (11.8); 4.261 (1.7); 4.237 (5.2); 4.213 (5.3); 4.190 (1.9); 3.707 (0.3); 3.321 (56.1); 2.672 (0.9); 2.636 (16.0); 2.502 (142.2); 2.450 (0.3); 2.329 (0.8); 1.045 (0.5); 1.030 (0.5); 0.000 (63.4)

### Example I-1-298

¹H-NMR (400.0 MHz, d₆-DMSO): δ=10.450 (8.6); 9.654 (4.9); 9.638 (5.1); 8.551 (6.2); 8.546 (6.4); 7.986 (5.8); 7.862 (3.4); 7.856 (3.4); 7.841 (3.9); 7.835 (3.9); 7.818 (2.6); 7.797 (6.1); 7.776 (3.6); 7.758 (4.1); 7.737 (1.7); 7.514 (7.0); 7.493 (6.3); 7.463 (2.1); 7.443 (6.1); 7.423 (5.5); 7.405 (7.0); 7.385 (6.5); 7.367 (2.2); 7.012 (3.2); 6.993 (2.9); 5.354 (16.0); 3.326 (92.6); 2.673 (0.8); 2.508 (112.1); 2.504 (147.1); 2.330 (0.8); 2.076 (1.7); 0.146 (0.7); 0.008 (6.5); 0.000 (134.7); −0.150 (0.6)

### Example I-1-299

¹H-NMR (400.0 MHz, d₆-DMSO): δ=10.275 (3.1); 9.685 (1.8); 9.668 (1.9); 8.550 (2.3); 8.544 (2.3); 7.857 (1.3); 7.851 (1.2); 7.836 (1.4); 7.830 (1.4); 7.801 (1.1); 7.780 (2.2); 7.751 (1.2); 7.732 (1.5); 7.712 (0.7); 7.511 (2.5); 7.490 (2.3); 7.388 (3.4); 7.371 (1.7); 7.355 (0.8); 7.241 (1.1); 7.220 (2.5); 7.200 (1.6); 7.101 (1.5); 7.079 (1.1); 6.621 (1.2); 6.616 (1.2); 6.602 (1.2); 6.596 (1.2); 5.350 (5.9); 3.760 (16.0); 3.323 (33.9); 2.671 (0.5); 2.507 (71.2); 2.502 (92.0); 2.498 (68.8); 2.329 (0.5); 0.146 (0.4); 0.008 (4.1); 0.000 (86.9); −0.008 (4.2); −0.150 (0.4)

### Example I-1-300

¹H-NMR (400.0 MHz, d₆-DMSO): δ=10.231 (4.3); 9.685 (2.6); 9.668 (2.7); 8.553 (3.2); 8.547 (3.3); 7.860 (1.8); 7.854 (1.8); 7.839 (2.0); 7.833 (2.1); 7.799 (1.6); 7.778 (3.1); 7.743 (1.7); 7.725 (2.2); 7.705 (1.0); 7.511 (3.7); 7.490 (3.3); 7.458 (3.6); 7.429 (2.0); 7.386 (1.3); 7.370 (2.4); 7.354 (1.2); 7.220 (1.7); 7.200 (3.2); 7.181 (1.6); 6.856 (2.2); 6.838 (1.9); 5.756 (2.7); 5.350 (8.6); 3.325 (72.9); 2.944 (0.4); 2.784 (0.4); 2.672 (0.7); 2.506 (90.0); 2.502 (118.3); 2.498 (91.2); 2.329 (0.8); 2.325 (0.6); 2.301 (16.0); 2.086 (1.8); 2.075 (1.0); 1.957 (0.3); 1.232 (0.7); 0.146 (0.5); 0.007 (4.6); 0.000 (101.6); −0.008 (6.7); −0.150 (0.5)

### Example I-1-301

¹H-NMR (400.0 MHz, d₆-DMSO): δ=10.601 (8.9); 10.202 (3.4); 9.629 (4.9); 9.613 (5.1); 8.359 (4.2); 8.355 (4.7); 8.347 (4.6); 8.343 (4.6); 7.996 (4.4); 7.992 (4.6); 7.976 (4.9); 7.972 (4.9); 7.957 (4.2); 7.935 (5.5); 7.899 (0.4); 7.883 (12.0); 7.836 (2.8); 7.817 (4.4); 7.798 (2.2); 7.796 (2.2); 7.432 (2.7); 7.416 (4.9); 7.399 (2.4); 7.205 (4.4); 7.193 (4.3); 7.185 (4.2); 7.174 (4.1); 5.757 (2.7); 5.520 (16.0); 5.223 (0.4); 3.324 (42.5); 2.672 (1.2); 2.668 (0.9); 2.507 (148.4); 2.503 (197.2); 2.499 (152.1); 2.330 (1.2); 1.241 (1.8); 1.232 (1.5); 1.226 (2.0); 1.046 (0.6); 1.030 (0.6); 0.146 (1.0); 0.008 (8.5); 0.000 (194.8); −0.150 (0.9)

### Example I-1-302

¹H-NMR (400.0 MHz, d₆-DMSO): δ=9.930 (2.6); 9.914 (2.7); 7.687 (6.5); 7.643 (2.3); 7.624 (2.6); 7.306 (2.2); 7.289 (3.2); 7.271 (2.0); 5.519 (8.9); 4.573 (14.9); 4.037 (1.5); 4.033 (1.5); 4.024 (1.4); 4.011 (0.5); 3.323 (26.7); 2.672 (0.5); 2.636 (16.0); 2.508 (50.1); 2.503 (65.2); 2.499 (49.3); 2.330 (0.4); 1.698 (1.0); 1.680 (2.3); 1.666 (5.4); 1.654 (7.4); 1.638 (3.4); 1.617 (1.1); 1.598 (0.4); 1.491 (2.2); 1.483 (2.3); 1.475 (1.9); 1.460 (0.7); 1.231 (0.6); 0.008 (2.8); 0.000 (59.7)

### Example I-1-303

¹H-NMR (400.0 MHz, d₆-DMSO): δ=10.639 (8.8); 10.203 (4.1); 9.667 (5.0); 9.650 (5.1); 8.387 (4.5); 8.383 (4.6); 8.375 (4.8); 8.371 (4.3); 7.940 (4.4); 7.930 (5.2); 7.926 (5.8); 7.919 (7.1); 7.911 (6.0); 7.907 (5.0); 7.876 (11.4); 7.827 (3.0); 7.806 (4.4); 7.787 (2.4); 7.428 (3.1); 7.411 (5.2); 7.394 (2.6); 7.150 (3.9); 7.138 (4.2); 7.131 (3.9); 7.119 (3.7); 6.527 (0.4); 6.514 (0.4); 6.103 (0.5); 5.503 (16.0); 5.212 (0.4); 4.680 (12.2); 4.338 (0.4); 3.325 (50.1); 2.672 (1.6); 2.585 (0.5); 2.503 (253.0); 2.329 (1.5); 1.543 (0.5); 1.463 (5.2); 1.233 (2.2); 1.045 (5.2); 1.030 (5.2); 0.000 (22.6)

### Example I-1-304

¹H-NMR (400.0 MHz, d₆-DMSO): δ=9.647 (4.3); 9.579 (2.5); 9.563 (2.5) 7.943 (2.1); 7.921 (2.7); 7.890 (6.2); 7.791 (1.4); 7.772 (2.3); 7.753 (1.1); 7.750 (1.1); 7.393 (2.0); 7.374 (2.6); 7.365 (2.6); 7.346 (1.2); 7.286 (1.4); 7.268 (2.6); 7.233 (2.6); 7.213 (3.2); 7.194 (1.2); 5.757 (5.8); 5.515 (8.2); 3.322 (93.2); 2.671 (1.1); 2.668 (0.9); 2.507 (143.7); 2.502 (189.2); 2.498 (145.4); 2.334 (0.9); 2.329 (1.1); 2.280 (16.0); 1.045 (1.2); 1.030 (1.2); 0.146 (0.8); 0.008 (8.1); 0.000 (178.0); −0.150 (0.8)

### Example I-1-305

¹H-NMR (400.0 MHz, d₆-DMSO): δ=9.780 (9.7); 9.519 (4.9); 9.503 (5.0); 8.318 (8.1); 8.305 (8.4); 7.961 (4.1); 7.939 (5.4); 7.893 (12.6); 7.825 (2.8); 7.807 (4.4); 7.788 (2.4); 7.785 (2.3); 7.730 (9.0); 7.716 (8.7); 7.396 (2.6); 7.379 (4.8); 7.363 (2.3); 7.361 (2.3); 5.757 (12.3); 5.511 (16.0); 3.776 (0.4); 3.324 (139.3); 2.892 (2.4); 2.732 (2.1); 2.672 (1.4); 2.667 (1.1); 2.507 (178.8); 2.503 (236.3); 2.498 (175.8); 2.334 (1.0); 2.330 (1.4); 2.325 (1.0); 2.087 (0.5); 1.234 (1.0); 1.046 (3.2); 1.030 (3.1); 0.146 (1.0); 0.008 (8.9); 0.000 (228.9); −0.008 (10.4); −0.150 (1.1)

### Example I-1-306

¹H-NMR (400.0 MHz, d₆-DMSO): δ=10.563 (3.9); 10.559 (4.0); 9.607 (4.7); 9.591 (4.8); 8.345 (1.6); 8.337 (1.7); 8.330 (1.8); 8.321 (1.9); 8.318 (2.2); 8.310 (1.7); 8.302 (1.8); 8.294 (1.6); 7.965 (3.9); 7.944 (5.4); 7.879 (12.0); 7.845 (2.8); 7.827 (4.3); 7.807 (2.0); 7.805 (2.2); 7.432 (2.6); 7.415 (4.7); 7.399 (2.3); 7.397 (2.4); 7.383 (1.5); 7.370 (1.6); 7.360 (1.9); 7.356 (2.0); 7.347 (2.0); 7.343 (2.0); 7.333 (1.7); 7.320 (1.6); 6.895 (0.9); 6.886 (1.7); 6.876 (1.6); 6.865 (2.5); 6.857 (1.5); 6.844 (1.5); 6.835 (0.8); 5.759 (1.6); 5.513 (16.0); 3.327 (44.9); 2.679 (0.4); 2.675 (0.5); 2.510 (60.5); 2.506 (81.0); 2.501 (62.1); 2.337 (0.3); 2.332 (0.5); 2.329 (0.4); 1.048 (0.8); 1.032 (0.8); 0.146 (0.4); 0.008 (3.8); 0.000 (85.7); −0.008 (4.6); −0.150 (0.4)

### Example I-1-307

¹H-NMR (400.0 MHz, d₆-DMSO): δ=9.994 (8.8); 9.542 (4.8); 9.526 (4.9); 8.033 (0.4); 8.022 (0.4); 7.970 (4.0); 7.948 (5.5); 7.891 (11.6); 7.845 (2.8); 7.825 (4.3); 7.805 (2.2); 7.737 (4.4); 7.718 (6.5); 7.697 (3.9); 7.675 (2.5); 7.515 (0.6); 7.575 (0.6); 7.488 (2.0); 7.475 (2.3); 7.467 (3.1); 7.455 (3.1); 7.447 (1.7); 7.434 (1.5); 7.420 (2.8); 7.403 (5.0); 7.386 (2.5); 7.204 (0.3); 6.520 (0.4); 6.500 (0.6); 6.475 (0.5); 5.757 (1.0); 5.516 (16.0); 4.583 (0.6); 4.568 (0.6); 3.322 (79.9); 2.672 (1.3); 2.503 (214.8); 2.499 (168.4); 2.329 (1.3); 1.233 (0.6); 1.046 (1.1); 1.031 (1.1); 0.146 (0.9); 0.000 (183.0); −0.149 (0.9)

### Example I-1-308

¹H-NMR (400.0 MHz, d₆-DMSO): δ=10.651 (8.3); 9.616 (4.7); 9.600 (4.9); 8.908 (3.6); 8.905 (4.3); 8.888 (3.9); 8.884 (4.4); 8.080 (3.8); 8.077 (4.5); 8.069 (4.4); 8.066 (4.6); 7.958 (3.7); 7.937 (5.4); 7.880 (11.1); 7.851 (2.9); 7.832 (4.2); 7.811 (2.2); 7.450 (3.5); 7.438 (5.6); 7.430 (4.2); 7.418 (8.0); 7.401 (2.4); 5.531 (16.0); 3.326 (39.5); 2.673 (0.8); 2.504 (125.2); 2.501 (105.0); 2.331 (0.7); 1.047 (0.8); 1.031 (0.8); 0.000 (10.0)

### Example I-1-309

¹H-NMR (400.0 MHz, d₆-DMSO): δ=9.741 (4.3); 9.552 (2.4); 9.535 (2.5); 8.190 (3.2); 8.178 (3.4); 7.953 (2.1); 7.931 (2.7); 7.891 (5.6); 7.812 (1.4); 7.791 (2.2); 7.772 (1.2); 7.579 (0.5); 7.509 (0.4); 7.498 (0.4); 7.390 (4.2); 7.376 (4.4); 7.357 (1.3); 5.758 (1.5); 5.517 (8.0); 5.237 (0.6); 4.588 (0.5); 4.573 (0.4); 3.327 (9.2); 2.672 (0.6); 2.503 (87.2); 2.499 (68.8); 2.317 (16.0); 2.229 (1.4); 2.206 (0.4); 2.152 (1.8); 2.075 (0.4); 1.233 (1.1); 0.000 (7.7)

### Example I-1-310

¹H-NMR (400.0 MHz, d₆-DMSO): δ=10.544 (9.1); 9.573 (4.8); 9.556 (5.0); 8.679 (4.0); 8.675 (4.7); 8.667 (4.4); 8.663 (4.6); 8.350 (3.9); 8.345 (4.3); 8.330 (4.2); 8.326 (4.3); 7.985 (3.9); 7.963 (5.5); 7.888 (12.0); 7.871 (3.1); 7.852 (4.4); 7.830 (2.3); 7.449 (2.7); 7.433 (4.8); 7.415 (2.6); 7.405 (4.3); 7.393 (4.1); 7.386 (4.2); 7.374 (4.0); 5.757 (0.4); 5.521 (16.0); 4.347 (0.4); 4.336 (0.4); 3.325 (87.0); 2.673 (0.7); 2.508 (90.5); 2.504 (121.2); 2.500 (95.0); 2.330 (0.7); 1.046 (2.6); 1.031 (2.6); 0.145 (0.5); 0.007 (4.2); −0.001 (101.3); −0.150 (0.5)

### Example I-1-311

¹H-NMR (400.0 MHz, d₆-DMSO): δ=10.455 (7.5); 9.632 (4.6); 9.616 (4.7); 8.388 (4.3); 8.367 (4.5); 8.317 (0.3); 8.045 (0.6); 8.035 (0.6); 7.940 (3.9); 7.918 (5.5); 7.870 (11.5); 7.829 (2.8); 7.810 (4.4); 7.788 (2.1); 7.721 (4.0); 7.702 (4.3); 7.677 (2.0); 7.657 (3.8); 7.638 (2.1); 7.575 (1.8); 7.418 (2.7); 7.402 (4.9); 7.385 (2.5); 7.283 (2.4); 7.264 (4.2); 7.245 (2.1); 7.207 (0.4); 6.576 (0.5); 6.560 (0.6); 6.547 (0.5); 6.522 (0.8); 6.501 (0.8); 5.757 (2.1); 5.519 (16.0); 4.583 (1.5); 4.568 (1.6); 3.323 (82.2); 2.672 (1.4); 2.507 (184.9); 2.503 (240.7); 2.499 (192.5); 2.330 (1.4); 1.352 (1.1); 1.337 (0.4); 1.259 (0.4); 1.231 (2.1); 0.146 (0.9); 0.000 (172.0); −0.150 (0.9)

### Example I-1-312

¹H-NMR (400.0 MHz, d₆-DMSO): δ=9.602 (4.5); 9.585 (4.7); 8.528 (5.9); 8.523 (6.4); 8.397 (1.9); 8.380 (4.0); 8.364 (2.2); 7.827 (3.0); 7.821 (3.3); 7.806 (3.5); 7.800 (3.7); 7.758 (2.4); 7.737 (6.1); 7.719 (3.6); 7.701 (4.0); 7.679 (1.6); 7.502 (6.0); 7.481 (5.4); 7.353 (2.3); 7.336 (4.3); 7.319 (2.3); 5.304 (16.0); 5.226 (0.4); 4.208 (1.0); 4.184 (3.4); 4.167 (3.7); 4.160 (4.1); 4.143 (3.4); 4.118 (1.2); 3.737 (0.7); 3.323 (122.9); 2.672 (1.5); 2.502 (256.5); 2.330 (1.5); 2.301 (0.7); 1.235 (0.5); 0.146 (0.9); 0.000 (175.6); −0.150 (0.9)

### Example I-1-313

¹H-NMR (400.0 MHz, d₆-DMSO): δ=9.560 (11.5); 9.541 (5.2); 8.560 (6.0); 8.554 (6.2); 7.868 (3.4); 7.861 (3.4); 7.847 (3.9); 7.841 (3.9); 7.800 (2.5); 7.779 (6.0); 7.760 (3.4); 7.758 (3.5); 7.743 (3.7); 7.740 (4.0); 7.721 (1.6); 7.718 (1.7); 7.520 (6.8); 7.500 (6.1); 7.383 (2.5); 7.380 (3.1); 7.363 (5.8); 7.345 (3.6); 7.342 (3.6); 7.321 (2.3); 7.305 (1.0); 7.215 (1.1); 7.207 (6.0); 7.187 (9.3); 7.166 (4.0); 7.158 (0.9); 5.350 (16.0); 3.323 (122.6); 2.676 (0.9); 2.672 (1.3); 2.668 (0.9); 2.507 (166.4); 2.503 (217.9); 2.498 (163.9); 2.334 (1.0); 2.329 (1.2); 2.325 (1.0); 0.146 (0.9); 0.008 (7.9); 0.000 (200.1); −0.008 (10.0); −0.150 (0.9)

### Example I-1-314

¹H-NMR (400.0 MHz, d₆-DMSO): δ=9.768 (8.8); 9.551 (4.6); 9.534 (4.7); 8.568 (5.9); 8.562 (6.0); 7.881 (3.3); 7.875 (3.3); 7.860 (3.8); 7.854 (3.8); 7.812 (2.8); 7.791 (5.6); 7.758 (3.2); 7.740 (4.0); 7.721 (1.7); 7.718 (1.7); 7.578 (13.0); 7.558 (16.0); 7.533 (6.6); 7.512 (5.8); 7.380 (2.4); 7.377 (2.6); 7.365 (6.4); 7.345 (7.8); 7.325 (3.2); 7.237 (1.2); 7.217 (1.3); 6.602 (0.4); 6.581 (0.7); 6.561 (0.4); 5.757 (7.2); 5.468 (0.5); 5.355 (14.9); 3.326 (161.3); 2.672 (1.0); 2.668 (0.8); 2.508 (137.0); 2.503 (179.5); 2.499 (135.2); 2.334 (0.7); 2.330 (1.0); 0.146 (0.5); 0.008 (5.0); 0.000 (103.3); −0.150 (0.5)

### Example I-1-315

¹H-NMR (400.0 MHz, d₆-DMSO): δ=10.688 (8.7); 9.644 (5.0); 9.627 (5.1); 8.555 (6.3); 8.549 (6.4); 8.353 (4.4); 8.350 (4.9); 8.342 (4.9); 8.338 (4.9); 8.317 (0.5); 7.985 (4.4); 7.982 (4.5); 7.965 (4.8); 7.962 (4.6); 7.861 (3.3); 7.855 (3.5); 7.840 (3.9); 7.834 (3.9); 7.804 (1.7); 7.782 (9.1); 7.763 (4.0); 7.741 (1.3); 7.518 (6.8); 7.497 (6.1); 7.422 (2.1); 7.417 (2.2); 7.405 (3.9); 7.400 (4.0); 7.388 (2.0); 7.384 (2.0); 7.194 (4.3); 7.182 (4.3); 7.174 (4.2); 7.162 (4.0); 5.362 (16.0); 3.818 (0.5); 3.737 (0.7); 3.325 (50.1); 2.671 (1.4); 2.667 (1.1); 2.542 (1.8); 2.507 (196.1); 2.502 (254.5); 2.498 (193.3); 2.329 (1.5); 0.146 (0.8); 0.000 (178.4); −0.150 (0.9)

### Example I-1-316

¹H-NMR (400.0 MHz, d₆-DMSO): δ=9.706 (8.9); 9.553 (4.9); 9.537 (5.0); 8.562 (6.1); 8.556 (6.4); 8.138 (0.3); 7.872 (3.2); 7.866 (3.3); 7.852 (3.7); 7.846 (3.7); 7.804 (2.6); 7.783 (6.1); 7.761 (3.5); 7.742 (4.1); 7.721 (1.7); 7.525 (6.5); 7.504 (5.8); 7.434 (2.5); 7.419 (3.9); 7.412 (3.9); 7.397 (0.5); 7.381 (2.5); 7.364 (4.7); 7.350 (5.1); 7.341 (4.4); 7.334 (7.9); 7.317 (5.5); 7.297 (0.8); 5.353 (16.0); 3.53 (16.0); 3.326 (174.8); 2.672 (1.6); 2.502 (272.4); 2.329 (1.6); 2.075 (1.5); 0.146 (0.8); −0.001 (152.5); −0.150 (0.8)

### Example I-1-317

¹H-NMR (400.0 MHz, d₆-DMSO): δ=10.467 (4.7); 9.580 (4.6); 9.563 (4.9); 8.559 (6.2); 8.553 (6.4); 8.321 (0.6); 8.238 (4.5); 8.225 (4.7); 7.873 (5.4); 7.867 (4.3); 7.852 (6.5); 7.846 (6.6); 7.828 (3.0); 7.811 (6.0); 7.801 (4.0); 7.781 (4.0); 7.761 (1.5); 7.519 (7.0); 7.498 (6.4); 7.427 (2.2); 7.423 (2.4); 7.410 (3.9); 7.406 (4.3); 7.393 (2.1); 7.389 (2.1); 7.319 (2.1); 7.309 (2.8); 7.298 (3.8); 7.289 (2.5); 7.277 (2.0); 5.364 (16.0); 4.063 (14.1); 3.821 (0.6); 3.736 (0.4); 2.676 (1.7); 2.671 (2.4); 2.667 (1.8); 2.542 (1.7); 2.507 (310.3); 2.502 (412.3); 2.498 (309.6); 2.334 (1.7); 2.329 (2.4); 2.325 (1.8); 0.146 (1.3); 0.007 (11.3); 0.000 (269.9); −0.008 (13.3); −0.150 (1.3)

### Example I-1-318

¹H-NMR (400.0 MHz, d₆-DMSO): δ=11.334 (7.4); 9.551 (4.7); 9.534 (4.7); 7.993 (3.8); 7.972 (5.3); 7.877 (13.5); 7.860 (4.4); 7.840 (2.1); 7.465 (7.8); 7.456 (8.5); 7.448 (2.8); 7.429 (4.6); 7.413 (2.3); 7.203 (6.2); 7.194 (5.9); 5.757 (1.5); 5.521 (16.0); 3.323 (100.7); 2.672 (1.1); 2.507 (148.4); 2.503 (190.0); 2.330 (1.1); 1.233 (0.4); 1.046 (1.5); 1.030 (1.5); 0.146 (0.5); 0.008 (6.7); 0.000 (117.8); −0.150 (0.5)

### Example I-1-319

¹H-NMR (400.0 MHz, d₆-DMSO): δ=10.189 (3.3); 9.546 (1.7); 9.529 (1.8); 7.976 (1.4); 7.955 (1.9); 7.941 (1.5); 7.938 (1.8); 7.920 (1.6); 7.917 (1.9); 7.897 (4.4); 7.888 (1.7); 7.886 (1.7); 7.869 (1.8); 7.866 (1.7); 7.852 (1.0); 7.833 (1.6); 7.811 (0.8); 7.464 (1.5); 7.444 (2.6); 7.428 (1.1); 7.425 (1.6); 7.412 (1.7); 7.393 (0.9); 5.523 (5.6); 4.347 (1.2); 4.337 (1.2); 3.795 (0.4); 3.785 (0.5); 3.779 (0.6); 3.769 (0.6); 3.764 (0.5); 3.754 (0.4); 3.326 (29.9); 2.509 (33.1); 2.504 (44.0); 2.500 (33.2); 1.047 (16.0); 1.032 (15.8); 0.008 (1.4); 0.000 (36.3); −0.009 (1.9)

### Example I-1-320

¹H-NMR (400.0 MHz, d₆-DMSO): δ=10.836 (3.8); 9.456 (2.0); 9.440 (2.1); 8.686 (1.9); 8.682 (2.1); 8.674 (2.0); 8.670 (2.1); 8.432 (1.8); 8.428 (1.9); 8.412 (1.9); 8.408 (1.9); 7.981 (1.6); 7.959 (2.2); 7.892 (5.3); 7.869 (1.1); 7.866 (1.2); 7.847 (1.8); 7.829 (0.9); 7.826 (0.9); 7.432 (2.0); 7.420 (2.0); 7.411 (2.8); 7.399 (2.8); 7.380 (1.0); 7.377 (1.0); 5.757 (16.0); 5.518 (6.5); 3.325 (88.3); 2.677 (0.4); 2.672 (0.5); 2.668 (0.4); 2.568 (5.7); 2.525 (1.6); 2.512 (31.3); 2.508 (63.9); 2.503 (85.5); 2.499 (63.9); 2.495 (32.5); 2.335 (0.3); 2.330 (0.5); 2.325 (0.4); 1.046 (0.4); 1.031 (0.4); 0.008 (2.6); 0.000 (68.6); −0.008 (3.3); −0.150 (0.3)

### Example I-1-321

¹H-NMR (400.0 MHz, d₆-DMSO): δ=9.616 (1.2); 9.600 (1.2); 9.496 (1.9); 7.932 (1.0); 7.910 (1.3); 7.892 (2.9); 7.767 (0.7); 7.748 (1.1); 7.726 (0.6); 7.369 (0.7); 7.352 (1.2); 7.333 (0.6); 7.129 (0.4); 7.105 (4.5); 7.100 (3.4); 7.090 (0.9); 5.520 (3.9); 4.102 (0.5); 4.088 (0.6); 3.327 (25.4); 3.178 (2.7); 3.165 (2.7); 2.509 (27.1); 2.504 (34.7); 2.500 (26.5); 2.228 (16.0); 0.000 (20.5)

### Example I-1-322

¹H-NMR (400.0 MHz, d₆-DMSO): δ=10.086 (4.3); 9.666 (2.7); 9.649 (2.7); 8.288 (1.6); 8.274 (1.8); 8.266 (1.8); 8.252 (1.6); 7.941 (2.2); 7.919 (2.9); 7.878 (5.8); 7.801 (1.5); 7.781 (2.4); 7.761 (1.3); 7.409 (1.5); 7.391 (2.7); 7.374 (1.4); 7.134 (1.6); 7.128 (1.8); 7.111 (1.7); 7.104 (1.8); 7.043 (1.0); 7.037 (0.9); 7.021 (1.8); 7.015 (1.6); 7.000 (1.0); 6.993 (0.8); 5.529 (8.7); 3.329 (15.0); 2.506 (47.6); 2.341 (16.0); 1.992 (0.6); 1.177 (0.3); 0.000 (20.7)

### Example I-1-323

¹H-NMR (400.0 MHz, d₆-DMSO): δ=9.586 (2.0); 9.570 (2.0); 9.277 (3.6); 7.931 (1.6); 7.909 (2.2); 7.883 (5.0); 7.783 (1.1); 7.764 (1.8); 7.745 (0.9); 7.742 (0.9); 7.375 (1.1); 7.358 (2.0); 7.342 (1.0); 7.340 (0.9); 7.281 (0.6); 7.264 (0.8); 7.260 (1.4); 7.244 (1.4); 7.239 (0.9); 7.223 (0.7); 6.948 (2.0); 6.927 (1.8); 6.903 (1.1); 6.880 (1.8); 6.858 (0.9); 5.500 (6.5); 3.827 (16.0); 3.326 (40.4); 2.672 (0.3); 2.508 (42.5); 2.504 (54.2); 2.499 (39.9); 0.008 (1.7); 0.000 (34.5); −0.008 (1.6)

### Example I-1-324

¹H-NMR (400.0 MHz, d₆-DMSO): δ=11.408 (7.4); 9.563 (4.9); 9.5446 (5.0); 8.550 (6.1); 8.545 (6.3); 7.875 (3.3); 7.869 (3.3); 7.854 (3.7); 7.848 (3.8); 7.833 (1.1); 7.818 (10.1); 7.804 (4.9); 7.782 (0.9); 7.511 (6.2); 7.490 (5.6); 7.455 (7.4); 7.446 (8.0); 7.435 (2.3); 7.428 (2.2); 7.419 (3.2); 7.413 (3.3); 7.403 (1.9); 7.397 (1.9); 7.196 (6.1); 7.187 (5.9); 5.361 (16.0); 3.326 (207.9); 2.671 (1.8); 2.506 (245.7); 2.502 (303.8); 2.329 (1.7); 0.145 (0.7); −0.001 (138.4); −0.150 (0.6)

### Example I-1-325

¹H-NMR (400.0 MHz, d₆-DMSO): δ=10.412 (2.8); 9.592 (1.7); 9.575 (1.7); 8.548 (2.1); 8.542 (2.1); 7.857 (1.2); 7.851 (1.2); 7.836 (1.4); 7.830 (1.4); 7.816 (0.8); 7.796 (2.1); 7.780 (1.2); 7.778 (1.2); 7.762 (1.2); 7.760 (1.4); 7.741 (0.5); 7.738 (0.6); 7.518 (2.4); 7.497 (2.1); 7.407 (0.8); 7.404 (0.8); 7.390 (1.4); 7.387 (1.5); 7.373 (0.8); 7.370 (0.7); 7.327 (2.8); 7.322 (2.9); 6.374 (3.0); 6.370 (3.0); 5.366 (5.5); 3.734 (16.0); 3.325 (40.4); 2.507 (39.6); 2.503 (52.0); 2.498 (38.4); 0.008 (2.2); 0.000 (51.8); −0.008 (2.4)

### Example I-1-326

¹H-NMR (400.0 MHz, d₆-DMSO): δ=10.512 (3.2); 9.735 (1.9); 9.718 (2.0); 8.543 (2.3); 8.537 (2.3); 8.474 (1.6); 8.470 (1.7); 8.455 (1.7); 8.451 (1.7); 7.835 (1.3); 7.829 (1.3); 7.814 (1.5); 7.808 (1.5); 7.751 (0.6); 7.732 (2.3); 7.727 (3.0); 7.709 (1.5); 7.707 (1.5); 7.688 (0.5); 7.685 (0.5); 7.510 (2.7); 7.489 (2.5); 7.380 (0.9); 7.376 (0.9); 7.363 (1.6); 7.359 (1.7); 7.347 (0.8); 7.342 (0.8); 7.308 (0.3); 7.059 (1.0); 7.055 (1.2); 7.038 (2.0); 7.035 (2.1); 7.005 (0.9); 7.001 (1.0); 6.986 (1.5); 6.982 (1.6); 6.966 (0.9); 6.962 (0.9); 6.937 (1.1); 6.933 (1.1); 6.917 (1.4); 6.914 (1.5); 6.899 (0.6); 6.895 (0.6); 5.757 (0.5); 5.358 (6.0); 3.877 (16.0); 3.684 (0.3); 3.324 (112.7); 2.676 (0.5); 2.671 (0.7); 2.667 (0.5); 2.524 (2.5); 2.511 (45.9); 2.507 (92.5); 2.502 (123.4); 2.498 (91.7); 2.493 (46.2); 2.333 (0.5); 2.329 (0.7); 2.324 (0.6); 0.146 (0.4); 0.008 (3.5); 0.000 (86.5); −0.008 (3.9); −0.150 (0.4)

### Example I-1-327

¹H-NMR (400.0 MHz, d₆-DMSO): δ=11.270 (3.9); 9.546 (3.2); 9.530 (3.2); 8.544 (4.0); 8.539 (4.1); 8.134 (0.4); 7.868 (2.3); 7.861 (2.2); 7.847 (2.6); 7.841 (2.5); 7.825 (0.8); 7.809 (6.4); 7.795 (2.9); 7.773 (0.6); 7.507 (4.4); 7.486 (3.9); 7.427 (1.3); 7.421 (1.4); 7.411 (2.2); 7.405 (2.3); 7.396 (1.2); 7.389 (1.3); 7.120 (4.8); 7.117 (4.9); 5.353 (10.2); 3.388 (81.2); 2.676 (0.7); 2.672 (1.0); 2.542 (1.2); 2.507 (133.0); 2.503 (172.1); 2.499 (128.4); 2.356 (15.8); 2.355 (16.0); 2.339 (0.8); 2.334 (0.9); 2.330 (1.1); 0.146 (0.4); 0.008 (4.9); 0.000 (97.4); −0.008 (4.9); −0.150 (0.5)

### Example I-1-328

¹H-NMR (400.0 MHz, d₆-DMSO): δ=10.202 (3.9); 9.710 (2.5); 9.694 (2.5); 8.550 (3.1); 8.544 (3.1); 8.353 (2.2); 8.333 (2.3); 8.137 (8.6); 7.851 (1.8); 7.844 (1.7); 7.830 (2.0); 7.824 (2.0); 7.785 (1.1); 7.764 (2.9); 7.747 (1.7); 7.745 (1.8); 7.730 (1.8); 7.727 (2.0); 7.708 (0.8); 7.705 (0.8); 7.515 (3.5); 7.495 (3.1); 7.398 (1.2); 7.394 (1.2); 7.381 (2.1); 7.377 (2.2); 7.364 (1.1); 7.360 (1.1); 7.233 (1.8); 7.214 (2.1); 7.201 (1.0); 7.181 (1.9); 7.162 (1.1); 6.969 (1.4); 6.953 (2.2); 6.950 (2.3); 6.932 (1.0); 5.375 (8.0); 3.324 (18.1); 2.676 (0.6); 2.671 (0.9); 2.667 (0.6); 2.541 (0.7); 2.511 (57.0); 2.507 (113.6); 2.502 (150.3); 2.498 (111.0); 2.494 (55.9); 2.325 (16.0); 0.146 (0.6); 0.008 (5.6); 0.000 (143.5); −0.008 (6.5); −0.150 (0.7)

### Example I-1-329

¹H-NMR (400.0 MHz, d₆-DMSO): δ=12.740 (0.5); 10.455 (3.6); 9.623 (2.1); 9.607 (2.3); 8.544 (2.9); 8.539 (3.0); 8.133 (2.0); 8.017 (2.8); 8.003 (2.9); 7.855 (1.6); 7.849 (1.6); 7.834 (1.8); 7.828 (2.0); 7.798 (3.8); 7.779 (2.0); 7.757 (0.7); 7.510 (3.1); 7.489 (2.8); 7.414 (1.1); 7.397 (1.9); 7.384 (1.0); 7.234 (3.6); 7.231 (3.7); 7.067 (1.9); 7.063 (1.8); 7.053 (1.9); 7.048 (1.8); 5.346 (7.1); 5.226 (0.4); 3.831 (16.0); 3.736 (0.7); 3.325 (67.4); 2.672 (1.3); 2.502 (231.8); 2.329 (1.3); 1.199 (0.5); 1.180 (1.0); 1.162 (0.5); 0.146 (0.9); 0.000 (173.4); −0.150 (0.8)

### Example I-1-330

¹H-NMR (400.0 MHz, d₆-DMSO): δ=10.323 (3.6); 9.579 (2.1); 9.562 (2.2); 7.962 (1.8); 7.940 (2.4); 7.876 (4.8); 7.831 (1.2); 7.812 (1.9); 7.791 (1.0); 7.418 (1.2); 7.401 (2.1); 7.383 (1.1); 7.331 (3.4); 7.327 (3.6); 6.367 (3.5); 6.363 (3.7); 5.524 (7.1); 3.744 (16.0); 3.327 (26.3); 2.505 (43.1); 0.000 (22.0)

### Example I-1-331

¹H-NMR (400.0 MHz, d₆-DMSO): δ=9.862 (9.2); 9.502 (4.5); 9.486 (4.7); 8.833 (14.0); 7.969 (3.7); 7.948 (5.1); 7.893 (11.5); 7.844 (2.5); 7.841 (2.5); 7.822 (4.1); 7.803 (2.1); 7.801 (2.0); 7.405 (2.4); 7.402 (2.4); 7.386 (4.5); 7.370 (2.2); 7.367 (2.2); 5.756 (16.0); 5.511 (14.7); 4.039 (0.7); 4.021 (0.7); 3.323 (93.4); 2.677 (0.7); 2.672 (1.0); 2.668 (0.7); 2.508 (128.0); 2.503 (166.2); 2.499 (124.6); 2.330 (1.0); 2.326 (0.7); 1.990 (3.0); 1.299 (0.4); 1.259 (0.6); 1.234 (1.0); 1.194 (0.9); 1.176 (1.7); 1.158 (0.8); 0.146 (0.9); 0.008 (9.0); 0.000 (191.5); −0.008 (10.0); −0.150 (0.9)

### Example I-1-332

¹H-NMR (400.0 MHz, d₆-DMSO): δ=9.562 (3.8); 9.515 (2.0); 9.49515 (2.0); 9.498 (2.0); 7.961 (1.6); 7.939 (2.2); 7.883 (4.9); 7.838 (1.2); 7.819 (1.8); 7.798 (0.9); 7.567 (6.5); 7.406 (1.1); 7.390 (2.0); 7.371 (1.0); 5.757 (5.1); 5.508 (6.4); 4.137 (0.5); 3.785 (0.5); 3.718 (16.0); 3.695 (1.0); 3.690 (0.6); 3.630 (0.3); 3.590 (0.4); 3.574 (0.4); 3.551 (0.3); 3.485 (0.4); 3.325 (25.7); 2.673 (0.3); 2.508 (39.7); 2.503 (53.1); 2.499 (41.3); 1.990 (0.4); 1.233 (0.5); 1.046 (5.1); 1.031 (5.1); 0.008 (2.7); 0.000 (61.3)

### Example I-1-334

¹H-NMR (400.0 MHz, d₆-DMSO): δ=10.622 (8.8); 9.693 (13.9); 9.643 (4.9); 9.627 (4.9); 8.315 (1.0); 8.230 (8.1); 8.216 (8.5); 7.963 (3.7); 7.941 (5.4); 7.877 (11.8); 7.859 (3.0); 7.838 (4.3); 7.817 (2.1); 7.634 (8.1); 7.621 (7.8); 7.441 (2.5); 7.424 (4.7); 7.408 (2.3); 5.528 (16.0); 4.342 (0.5); 4.331 (0.6); 3.318 (416.8); 2.693 (0.4); 2.675 (3.5); 2.671 (4.7); 2.667 (3.6); 2.644 (0.4); 2.506 (566.5); 2.502 (768.0); 2.497 (585.7); 2.333 (3.4); 2.328 (4.5); 2.324 (3.3); 1.236 (0.7); 1.045 (3.3); 1.030 (3.2); 0.146 (3.3); 0.008 (29.4); 0.000 (684.4); −0.149 (3.2)

### Example I-1-335

¹H-NMR (400.0 MHz, d₆-DMSO): δ=10.527 (9.5); 9.635 (5.0); 9.619 (5.1); 8.394 (4.5); 8.390 (5.0); 8.382 (4.8); 8.378 (5.0); 8.136 (4.8); 8.132 (5.0); 8.116 (5.2); 8.112 (5.1); 7.953 (4.0); 7.931 (5.6); 7.882 (12.5); 7.836 (2); 833 (0.7); 7.833 (2.9); 7.814 (4.6); 7.795 (2.3); 7.793 (2.3); 7.432 (2.5); 7.429 (2.7); 7.413 (4.8); 7.397 (2.4); 7.394 (2.4); 7.126 (4.7); 7.115 (4.6); 7.107 (4.6); 7.095 (4.5); 5.757 (0.4); 5.518 (16.0); 4.344 (0.4); 4.334 (0.4); 3.323 (45.4); 2.678 (0.4); 2.673 (0.6); 2.669 (0.4); 2.526 (1.7); 2.509 (71.9); 2.504 99.9); 2.500 (77.0); 2.336 (0.4); 2.331 (0.6); 2.327 (0.5); 1.243 (0.4); 1.228 (0.8); 1.047 (4.1); 1.032 (4.0); 0.146 (0.5); 0.008 (4.3); 0.000 (105.5); −0.008 (5.0); −0.150 (0.5)

### Example I-1-336

¹H-NMR (400.0 MHz, d₆-DMSO): δ=10.506 (8.9); 10.201 (1.4); 9.588 (4.8); 9.571 (4.9); 8.696 (3.7); 8.685 (3.7); 8.316 (0.5); 8.218 (3.5); 8.214 (3.6); 8.198 (3.7); 8.195 (3.8); 7.956 (4.0); 7.934 (5.5); 7.879 (12.5); 7.843 (2.7); 7.840 (2.9); 7.822 (4.5); 7.803 (2.2); 7.800 (2.2); 7.428 (2.7); 7.412 (6.2); 7.404 (3.3); 7.395 (5.0); 7.384 (2.7); 5.756 (4.0); 5.516 (16.0); 3.324 (316.1); 2.676 (1.4); 2.672 (1.8); 2.507 (240.3); 2.503 (317.9); 2.498 (240.0); 2.334 (1.3); 2.329 (1.8); 2.325 (1.4); 1.352 (0.3); 1.233 (1.3); 1.046 (3.0); 1.030 (2.9); 0.146 (1.6); 0.008 (15.5); 0.000 (353.4); −0.008 (17.3); −0.150 (1.7)

### Example I-1-337

¹H-NMR (400.0 MHz, d₆-DMSO): δ=10.199 (1.1); 10.118 (2.7); 9.624 (2.2); 9.608 (2.3); 8.238 (1.3); 7.956 (2.3); 7.934 (3.0); 7.887 (6.7); 7.814 (1.6); 7.794 (2.5); 7.774 (1.5); 7.716 (2.1); 7.697 (2.1); 7.409 (1.4); 7.392 (2.6); 7.375 (1.3); 7.179 (1.0); 7.165 (1.3); 7.150 (0.9); 5.756 (0.3); 5.521 (9.1); 5.459 (0.4); 3.321 (60.3); 2.672 (1.0); 2.507 (123.5); 2.503 (163.9); 2.498 (125.3); 2.329 (1.4); 2.313 (16.0); 2.126 (0.3); 2.020 (0.7); 1.928 (0.5); 1.234 (0.9); 1.046 (0.6); 1.031 (0.6); 0.146 (0.7); 0.008 (6.8); 0.000 (142.6); −0.149 (0.7)

### Example I-1-338

¹H-NMR (400.0 MHz, d₆-DMSO): δ=11.672 (8.0); 9.478 (4.6); 9.462 (4.7); 8.440 (16.0); 8.027 (3.4); 8.005 (5.4); 7.945 (2.7); 7.943 (2.8); 7.924 (4.1); 7.905 (1.9); 7.903 (2.0); 7.876 (11.3); 7.484 (2.3); 7.482 (2.4); 7.465 (4.5); 7.449 (2.2); 7.447 (2.2); 5.528 (15.6); 3.319 (53.0); 2.676 (0.7); 2.672 (1.0); 2.667 (0.7); 2.525 (3.1); 2.507 (118.7); 2.503 (163.4); 2.498 (125.1); 2.333 (0.7); 2.329 (1.0); 2.325 (0.7); 1.989 (0.8); 1.176 (0.4); 0.146 (0.7); 0.008 (5.2); 0.000 (132.2); −0.008 (5.9); −0.150 (0.6)

### Example I-4-001

¹H-NMR (400.0 MHz, d₆-DMSO): δ=9.614 (1.3); 9.610 (1.3); 9.598 (1.3); 9.593 (1.3); 8.549 (1.7); 8.545 (1.7); 8.537 (1.8); 8.533 (1.7); 8.445 (1.8); 8.439 (1.8); 7.830 (1.2); 7.824 (1.2); 7.810 (1.3); 7.803 (1.3); 7.485 (2.2); 7.465 (2.0); 7.448 (1.6); 7.436 (1.7); 7.432 (1.7); 7.420 (1.6); 5.177 (5.7); 3.773 (0.3); 3.749 (16.0); 3.735 (1.7); 3.710 (0.7); 3.584 (0.5); 3.570 (0.6); 3.559 (0.7); 3.537 (0.5); 3.522 (0.6); 3.336 (3.4); 2.525 (0.5); 2.512 (13.5); 2.507 (27.8); 2.503 (38.3); 2.498 (28.7); 2.494 (14.0); 1.046 (2.2); 1.030 (2.1)

### Example I-5-001

¹H-NMR (400.0 MHz, d₆-DMSO): δ=8.528 (2.9); 8.522 (3.0); 8.496 (4.6); 8.486 (4.7); 7.895 (1.7); 7.888 (1.7); 7.874 (2.0); 7.868 (2.0); 7.563 (3.3); 7.542 (3.0); 7.473 (4.6); 7.463 (4.6); 5.176 (9.4); 4.837 (0.4); 4.655 (16.0); 4.655 (16.0); 4.612 (0.5); 3.323 (17.5); 3.170 (0.3); 2.512 (13.9); 2.508 (28.5); 2.503 (39.0); 2.499 (30.2); 2.494 (15.6); 0.000 (2.8)

### Example I-5-002

¹H-NMR (400.0 MHz, d₆-DMSO): δ=8.499 (4.4); 8.489 (4.4); 7.875 (6.1); 7.513 (4.5); 7.503 (4.3); 5.370 (10.1); 4.645 (16.0); 3.464 (0.4); 3.368 (1.6); 3.171 (1.8); 2.508 (33.2); 2.504 (45.6); 2.499 (35.3); 1.490 (0.6); 1.353 (1.4); 1.300 (0.6); 1.259 (0.8); 1.234 (2.5); 0.000 (0.7)

a) The determination of the [M+H]⁺ by LC-MS in the acidic range was effected at pH 2.7, with acetonitrile (containing 0.1% formic acid) and water as eluents; linear gradient from 10% acetonitrile to 95% acetonitrile, instrument: Agilent 1100 LC system, Agilent MSD system, HTS PAL.

The log P values reported in the tables and preparation examples above were determined in accordance with EEC directive 79/831 Annex V.A8 by HPLC (High Performance Liquid Chromatography) using a reversed-phase column (C18). Temperature 43° C. The calibration is effected with unbranched alkan-2-ones (having 3 to 16 carbon atoms), for which the log P values are known.

b) The determination of the ¹H NMR data was effected with a Bruker Avance 400 or Bruker Avance III 600 equipped with a sample flow head (capacity 60 μl), with tetramethylsilane as reference (0.0) and the solvents CD₃CN, CDCl₃ or D₆-DMSO.

The NMR data of selected examples are listed either in conventional form (6 values, multiplet splitting, number of hydrogen atoms) or as NMR peak lists.

### NMR Peak List Method

The ¹H NMR data of selected examples are stated in the form of ¹H NMR peak lists. For each signal peak, first the δ value in ppm and then the signal intensity in round brackets are listed. The δ value—signal intensity number pairs for different signal peaks are listed with separation from one another by semicolons.

The peak list for one example therefore has the form:

δ₁(intensity,); δ₂(intensity₂); . . . ; δᵢ(intensityᵢ); . . . ; δₙ(intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

Calibration of the chemical shift of ¹H NMR spectra is accomplished using tetramethylsilane and/or the chemical shift of the solvent, particularly in the case of spectra which are measured in DMSO. Therefore, the tetramethylsilane peak may but need not occur in NMR peak lists.

The lists of the ¹H NMR peaks are similar to the conventional ¹H-NMR printouts and thus usually contain all peaks listed in a conventional NMR interpretation.

In addition, like conventional ¹H NMR printouts, they may show solvent signals, signals of stereoisomers of the target compounds which are likewise provided by the invention, and/or peaks of impurities.

In the reporting of compound signals within the delta range of solvents and/or water, our lists of ¹H NMR peaks show the standard solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the peak picking in question in conventional ¹H NMR interpretation.

Further details of ¹H NMR peak lists can be found in the Research Disclosure Database Number 564025.

### Application Examples

The examples which follow demonstrate the insecticidal and acaricidal action of the compounds according to the invention. In these examples, the inventive compounds cited relate to the compounds listed in Tables 1 to 3 with the corresponding reference numerals (No.):

Ctenocephalides felis—In Vitro Contact Tests with Adult Cat Fleas

For the coating of the test tubes, 9 mg of active ingredient are first dissolved in 1 ml of acetone p.a. and then diluted to the desired concentration with acetone p.a. 250 μl of the solution are distributed homogeneously on the inner walls and the base of a 25 ml test tube by turning and rocking on an orbital shaker (rocking rotation at 30 rpm for 2 h). With 900 ppm of active ingredient solution and internal surface area 44.7 cm², given homogeneous distribution, an area-based dose of 5 μg/cm² is achieved.

After the solvent has evaporated off, the tubes are populated with 5-10 adult cat fleas (Ctenocephalides felis), sealed with a perforated plastic lid and incubated in a horizontal position at room temperature and ambient humidity. After 48 h, efficacy is determined. To this end, the test tubes are stood upright and the fleas are knocked to the base of the tube. Fleas which remain motionless at the base or move in an uncoordinated manner are considered to be dead or moribund.

A substance shows good efficacy against Ctenocephalides felis if at least 80% efficacy was achieved in this test at an application rate of 5 μg/cm². 100% efficacy means that all the fleas were dead or moribund. 0% efficacy means that no fleas were harmed.

In this test, for example, the following compounds from the preparation examples showed an efficacy of 100% at an application rate of 5 μg/cm²: I-1-003, I-1-032, I-1-082, I-1-111, I-1-143, I-1-239, I-1-240, I-1-243

In this test, for example, the following compounds from the preparation examples showed an efficacy of 90% at an application rate of 5 μg/cm²: I-1-033 In this test, for example, the following compounds from the preparation examples showed an efficacy of 80% at an application rate of 5 μg/cm²: I-1-147

Boophilus microplus—Injection Test
Solvent: dimethyl sulphoxide

To produce a suitable active ingredient formulation, 10 mg of active ingredient are mixed with 0.5 ml of solvent and the concentrate is diluted with solvent to the desired concentration.

1 μl of the active ingredient solution is injected into the abdomen of 5 engorged adult female cattle ticks (Boophilus microplus). The animals are transferred into dishes and kept in a climate-controlled room.

Efficacy is assessed after 7 days by laying of fertile eggs. Eggs which are not visibly fertile are stored in a climate-controlled cabinet until the larvae hatch after about 42 days. An efficacy of 100% means that none of the ticks has laid any fertile eggs; 0% means that all the eggs are fertile.

In this test, for example, the following compounds from the preparation examples showed an efficacy of 100% at an application rate of 20 μg/animal: I-1-008, I-1-009, I-1-012, I-1-016, I-1-029, I-1-054, I-1-165

In this test, for example, the following compounds from the preparation examples showed an efficacy of 80% at an application rate of 20 μg/animal: I-1-003, I-1-020, I-1-033, I-1-034, I-1-049, I-1-152, I-1-202, I-1-217, I-1-254

Ctenocephalides felis—Oral Test
Solvent: dimethyl sulphoxide

For the purpose of production of a suitable active ingredient formulation, 10 mg of active ingredient are mixed with 0.5 ml of dimethyl sulphoxide. Dilution with citrated cattle blood gives the desired concentration.

About 20 unfed adult cat fleas (Ctenocephalides felis) are placed into a chamber which is closed at the top and bottom with gauze. A metal cylinder whose bottom end is closed with parafilm is placed onto the chamber. The cylinder contains the blood/active ingredient formulation, which can be imbibed by the fleas through the parafilm membrane.

After 2 days, the kill in % is determined. 100% means that all of the fleas have been killed; 0% means that none of the fleas have been killed.

In this test, for example, the following compounds from the preparation examples showed an efficacy of 95% at an application rate of 100 ppm: I-1-003

Lucilia cuprina Test
Solvent: dimethyl sulphoxide

To produce a suitable active ingredient formulation, 10 mg of active ingredient are mixed with 0.5 ml of dimethyl sulphoxide, and the concentrate is diluted with water to the desired concentration.

About 20 L1 larvae of the Australian sheep blowfly (Lucilia cuprina) are transferred into a test vessel containing minced horsemeat and the active ingredient formulation of the desired concentration.

After 2 days, the kill in % is determined. 100% means that all the larvae have been killed; 0% means that no larvae have been killed.

In this test, for example, the following compounds of the preparation examples showed an efficacy of 100% at an application rate of 100 ppm: I-1-003, I-1-009, I-1-034, I-1-049, I-1-087, I-1-111, I-1-145, I-1-202, I-1-254

In this test, for example, the following compounds of the preparation examples showed an efficacy of 95% at an application rate of 100 ppm: I-1-137, I-1-154 In this test, for example, the following compounds of the preparation examples showed an efficacy of 90% at an application rate of 100 ppm: I-1-008, I-1-027, I-1-032, I-1-033, I-1-089

Musca domestica Test
Solvent: dimethyl sulphoxide

To produce a suitable active ingredient formulation, 10 mg of active ingredient are mixed with 0.5 ml of dimethyl sulphoxide, and the concentrate is diluted with water to the desired concentration.

Vessels containing a sponge treated with sugar solution and the active ingredient formulation of the desired concentration are populated with 10 adult houseflies (Musca domestica).

After 2 days, the kill in % is determined. 100% means that all of the flies have been killed; 0% means that none of the flies have been killed.

In this test, for example, the following compounds of the preparation examples showed an efficacy of 100% at an application rate of 100 ppm: I-1-008, I-1-032, I-1-033, I-1-111

In this test, for example, the following compounds of the preparation examples showed an efficacy of 80% at an application rate of 100 ppm: I-1-003, I-1-034

Diabrotica balteata—Spray Test
Solvent: 78 parts by weight of acetone
1.5 parts by weight of dimethylformamide
   Emulsifier: alkylaryl polyglycol ether

To produce a suitable active ingredient formulation, 1 part by weight of active ingredient is dissolved with the stated parts by weight of solvent and made up with water containing an emulsifier concentration of 1000 ppm until the desired concentration is attained. To produce further test concentrations, the formulation is diluted with emulsifier-containing water.

Pre-swollen wheat grains (Triticum aestivum) are incubated in a multiwell plate filled with agar and a little water for one day (5 seed grains per cavity). The germinated wheat grains are sprayed with an active ingredient formulation of the desired concentration. Subsequently, each cavity is infected with 10-20 beetle larvae of Diabrotica balteata.

After 7 days, the efficacy in % is determined. 100% means that all wheat plants have grown as in the untreated, uninfected control; 0% means that no wheat plant has grown.

In this test, for example, the following compounds from the preparation examples showed an efficacy of 100% at an application rate of 160 μg/well: I-1-004, I-1-008, I-1-020, I-1-027, I-1-028, I-1-029, I-1-032, I-1-033, I-1-034, I-1-036, I-1-082, I-1-083, I-1-087, I-1-097, I-1-111, I-1-128, I-1-129, I-1-137, I-1-143, I-1-145, I-1-147, I-1-148, I-1-203, I-1-216, I-1-227, I-1-230, I-1-231, I-1-232, I-1-235, I-1-246, I-1-254, I-1-267, I-1-276, I-1-277, I-1-282, I-1-283, I-1-288, I-1-290, I-1-292

In this test, for example, the following compounds from the preparation examples showed an efficacy of 80% at an application rate of 160 μg/well: I-1-041, I-1-045, I-1-135, I-1-154, I-1-202, I-1-233, I-1-238, I-1-258, I-1-284, I-1-287, I-1-291

Phaedon cochleariae—Spray Test
Solvent: 78.0 parts by weight of acetone
1.5 parts by weight of dimethylformamide
   Emulsifier: alkylaryl polyglycol ether

To produce a suitable active ingredient formulation, 1 part by weight of active ingredient is dissolved with the stated parts by weight of solvent and made up with water containing an emulsifier concentration of 1000 ppm until the desired concentration is attained. To produce further test concentrations, the formulation is diluted with emulsifier-containing water.

Discs of Chinese cabbage leaves (Brassica pekinensis) are sprayed with an active ingredient formulation of the desired concentration and, after drying, populated with larvae of the mustard beetle (Phaedon cochleariae).

After 7 days, the efficacy in % is determined. 100% means that all the beetle larvae have been killed; 0% means that no beetle larvae have been killed.

In this test, for example, the following compounds from the preparation examples showed an efficacy of 100% at an application rate of 500 g/ha: I-1-082, I-1-083, I-1-093, I-1-094, I-1-097, I-1-154

I-1-198, I-1-202, I-1-203, I-1-212, I-1-216, I-1-217, I-1-240, I-1-242, I-1-243, I-1-244, I-1-246, I-1-254, I-1-256, I-1-267, I-1-271, I-1-274, I-1-276, I-1-297

In this test, for example, the following compounds from the preparation examples showed an efficacy of 83% at an application rate of 500 g/ha: I-1-262, I-1-277

Meloidogyne incognita Test
Solvent: 125.0 parts by weight of acetone

To produce a suitable preparation of active ingredient, 1 part by weight of active ingredient is mixed with the stated amount of solvent and the concentrate is diluted with water to the desired concentration.

Vessels are filled with sand, active ingredient solution, an egg/larvae suspension of the southern root-knot nematode (Meloidogyne incognita) and lettuce seeds. The lettuce seeds germinate and the plants develop. The galls develop on the roots.

After 14 days, the nematicidal efficacy in % is determined by the formation of galls. 100% means that no galls were found; 0% means that the number of galls on the treated plants corresponds to the untreated control.

In this test, for example, the following compounds from the preparation examples showed an efficacy of 90% at an application rate of 20 ppm: I-1-116, I-1-143, I-1-147, I-1-161, I-1-176, I-1-196, I-1-276, I-1-277, I-1-314, I-1-316, I-1-321, I-1-323

Tetranychus urticae—Spray Test, OP-Resistant
Solvent: 78.0 parts by weight of acetone
1.5 parts by weight of dimethylformamide
   Emulsifier: alkylaryl polyglycol ether

To produce a suitable active ingredient formulation, 1 part by weight of active ingredient is dissolved with the stated parts by weight of solvent and made up with water containing an emulsifier concentration of 1000 ppm until the desired concentration is attained. To produce further test concentrations, the formulation is diluted with emulsifier-containing water.

Discs of bean leaves (Phaseolus vulgaris) infested with all stages of the greenhouse red spider mite (Tetranychus urticae) are sprayed with an active ingredient formulation of the desired concentration.

After 6 days, the efficacy in % is determined. 100% means that all the spider mites have been killed; 0% means that no spider mites have been killed.

In this test, for example, the following compounds from the preparation examples showed an efficacy of 90% at an application rate of 500 g/ha: I-1-002, I-1-009, I-1-014, I-1-017, I-1-019, I-1-137

### I-1-139, I-1-143, I-1-158, I-1-160, I-1-166, I-1-178, I-1-271, I-1-299, I-1-304.

Myzus persicae—Spray Test
Solvent: 78 parts by weight of acetone
1.5 parts by weight of dimethylformamide
   Emulsifier: alkylaryl polyglycol ether

To produce a suitable active ingredient formulation, 1 part by weight of active ingredient is dissolved with the stated parts by weight of solvent and made up with water containing an emulsifier concentration of 1000 ppm until the desired concentration is attained. To produce further test concentrations, the formulation is diluted with emulsifier-containing water.

Discs of Chinese cabbage leaves (Brassica pekinensis) infested by all stages of the green peach aphid (Myzus persicae) are sprayed with an active ingredient formulation of the desired concentration.

After 6 days, the efficacy in % is determined. 100% means that all the aphids have been killed; 0% means that no aphids have been killed.

In this test, for example, the following compounds from the preparation examples showed an efficacy of 90% at an application rate of 500 g/ha: I-1-004, I-1-005, I-1-009, I-1-010, I-1-012, I-1-015, I-1-020, I-1-028, I-1-034, I-1-036, I-1-049, I-1-056, I-1-083, I-1-085, I-1-092, I-1-097, I-1-101, I-1-106, I-1- 107, I-1-112, I-1-122, I-1-152, I-1-165, I-1-178, I-1-224, I-1-246, I-1-254, I-1-267, I-1-274, I-1-276, I-1- 279, I-1-282, I-1-284, I-1-292, I-1-296, I-1-297, I-1-303, I-1-305, I-1-307, I-1-308, I-1-310, I-1-313, I-1- 319, I-1-325, I-1-328

In this test, for example, the following compounds from the preparation examples showed an efficacy of 100% at an application rate of 500 g/ha: I-1-003, I-1-008, I-1-016, I-1-022, I-1-023, I-1-026, I-1-027, I-1-029, I-1-043, I-1-054, I-1-061, I-1-068, I-1-070, I-1-072, I-1-076, I-1-080, I-1-081, I-1-082, I- 1-086, I-1-087, I-1-111, I-1-210, I-1-217, I-1-220, I-1-221, I-1-226, I-1-283, I-1-293, I-1-314, I-1-301, I- 1-304, I-1-309, I-1-311, I-1-315, I-1-316

Myzus persicae—Oral Test
Solvent: 100 parts by weight of acetone

To produce an appropriate active ingredient formulation, 1 part by weight of active ingredient is dissolved with the stated parts by weight of solvent and made up with water until the desired concentration is attained.

50 μl of the active ingredient formulation are transferred into microtitre plates and made up to a final volume of 200 μl with 150 μl of IPL41 insect medium (33%+15% sugar). Subsequently, the plates are sealed with parafilm, which a mixed population of green peach aphids (Myzus persicae) within a second microtitre plate is able to puncture and imbibe the solution through it.

After 5 days, the efficacy in % is determined. 100% means that all the aphids have been killed; 0% means that no aphids have been killed.

In this test, for example, the following compounds from the preparation examples showed an efficacy of 100% at an application rate of 20 ppm: I-1-001, I-1-002, I-1-003, I-1-004, I-1-005, I-1-006, I-1-008, I-1-009, I-1-010, I-1-011, I-1-012, I-1-013, I-1-014, I-1-015, I-1-016, I-1-017, I-1-018, I-1-020, I-1- 022, I-1-023, I-1-025, I-1-026, I-1-027, I-1-028, I-1-029, I-1-031, I-1-032, I-1-033, I-1-034, I-1-035, I-1- 036, I-1-037, I-1-038, I-1-039, I-1-040, I-1-041, I-1-042, I-1-043, I-1-044, I-1-045, I-1-046, I-1-047, I-1- 049, I-1-051, I-1-052, I-1-054, I-1-055, I-1-059, I-1-060, I-1-061, I-1-062, I-1-064, I-1-065, I-1-068, I-1- 070, I-1-072, I-1-075, I-1-076, I-1-077, I-1-081, I-1-082, I-1-083, I-1-084, I-1-086, I-1-087, I-1-088, I-1- 089, I-1-091, I-1-092, I-1-093, I-1-096, I-1-097, I-1-098, I-1-101, I-1-103, I-1-107, I-1-108, I-1-109, I-1- 110, I-1-111, I-1-112, I-1-113, I-1-114, I-1-115, I-1-116, I-1-117, I-1-118, I-1-119, I-1-121, I-1-122, I-1- 124, I-1-129, I-1-131, I-1-137, I-1-138, I-1-142, I-1-145, I-1-146, I-1-147, I-1-148, I-1-151, I-1-152, I-1- 154, I-1-155, I-1-157, I-1-158, I-1-159, I-1-160, I-1-161, I-1-165, I-1-166, I-1-167, I-1-168, I-1-169, I-1- 170, I-1-171, I-1-173, I-1-174, I-1-175, I-1-177, I-1-178, I-1-179, I-1-180, I-1-181, I-1-182, I-1-183, I-1- 185, I-1-186, I-1-188, I-1-190, I-1-192, I-1-193, I-1-198, I-1-199, I-1-201, I-1-202, I-1-203, I-1-205, I-1- 206, I-1-207, I-1-208, I-1-209, I-1-210, I-1-212, I-1-213, I-1-214, I-1-215, I-1-216, I-1-217, I-1-218, I-1- 219, I-1-220, I-1-221, I-1-222, I-1-223, I-1-224, I-1-225, I-1-226, I-1-227, I-1-228, I-1-229, I-1-231, I-1- 234, I-1-236, I-1-239, I-1-240, I-1-241, I-1-243, I-1-247, I-1-249, I-1-250, I-1-251, I-1-254, I-1-257, I-1- 269, I-1-271, I-1-272, I-1-274, I-1-275, I-1-276, I-1-277, I-1-278, I-1-279, I-1-282, I-1-283, I-1-284, I-1- 285, I-1-286, I-1-287, I-1-288, I-1-289, I-1-292, I-1-293, I-1-296, I-1-297, I-1-299, I-1-301, I-1-302, I-1- 304, I-1-305, I-1-306, I-1-308, I-1-309, I-1-310, I-1-311, I-1-312, I-1-313, I-1-314, I-1-315, I-1-316, I-1- 317, I-1-319, I-1-321, I-1-323, I-1-325, I-1-326, I-1-328, I-4-001, I-5-001, I-5-002

In this test, for example, the following compounds from the preparation examples showed an efficacy of 90% at an application rate of 20 ppm: I-1-053, I-1-056, I-1-057, I-1-066, I-1-071, I-1-078, I-1-079, I-1-106, I-1-125, I-1-126, I-1-133, I-1-135, I-1-164, I-1-172, I-1-183, I-1-191, I-1-195, I-1-200, I-1- 211, I-1-232, I-1-235, I-1-237, I-1-244, I-1-245, I-1-246, I-1-248, I-1-252, I-1-259, I-1-265, I-1-294

Myzus persicae—Spray Test
Solvent: 14 parts by weight of dimethylformamide
Emulsifier: alkylaryl polyglycol ether

To produce a suitable active ingredient formulation, 1 part by weight of active ingredient is dissolved with the stated parts by weight of solvent and made up with water containing an emulsifier concentration of 1000 ppm until the desired concentration is attained. To produce further test concentrations, the formulation is diluted with emulsifier-containing water. If the addition of ammonium salts or/and penetrants is required, these are each added in a concentration of 1000 ppm to the formulation solution.

Bell pepper plants (Capsicum annuum) severely infested with the green peach aphid (Myzus persicae) are treated by spraying with the active ingredient formulation in the desired concentration.

After 6 days, the kill in % is determined. 100% means that all of the aphids have been killed; 0% means that none of the aphids have been killed.

In this test, for example, the following compounds from the preparation examples showed an efficacy of 100% at an application rate of 100 ppm: I-1-032, I-1-033, I-1-145, I-1-147, I-1-154, I-1-193

In this test, for example, the following compounds from the preparation examples showed an efficacy of 99% at an application rate of 100 ppm: I-1-227, I-1-231

In this test, for example, the following compounds from the preparation examples showed an efficacy of 95% at an application rate of 100 ppm: I-1-046, I-1-045, I-1-062, I-1-113, I-1-137, I-1-179, I-1-252

In this test, for example, the following compounds from the preparation examples showed an efficacy of 90% at an application rate of 100 ppm: I-1-002, I-1-202

In this test, for example, the following compounds from the preparation examples showed an efficacy of 85% at an application rate of 100 ppm: I-1-013, I-1-114

In this test, for example, the following compounds from the preparation examples showed an efficacy of 80% at an application rate of 100 ppm: I-1-044

1. Compound of formula (I) 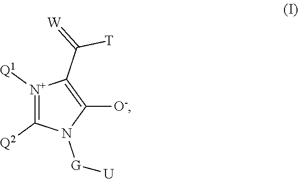
in which
Q¹ and Q² together are one of the A-1 to A-5 radicals which, together with the carbon and nitrogen atom to which they are bonded, form a 5- or 6-membered aromatic ring, 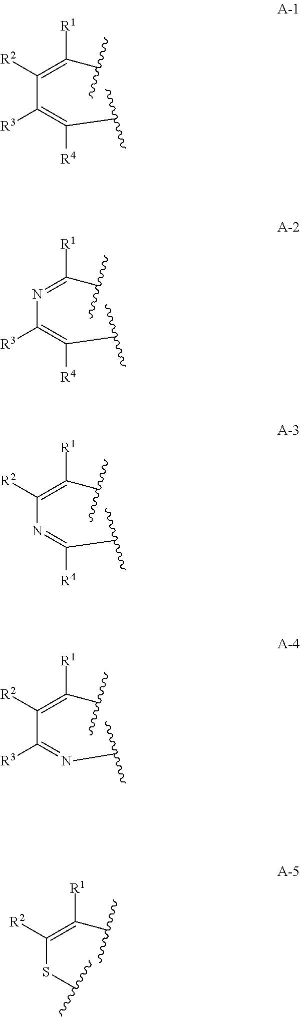
T is R⁵, —OR⁶, —N(R⁷)(R⁸) or —N(R^{7a})—N(R⁷)(R⁸)
and
W is O,
or
T is —N(R⁷)(R⁸) or —N(R^{7a})—N(R⁷)(R⁸)
and
W is S,
G is a bond, is —C(R⁹)(R¹⁰)—, or is C(R⁹)(R¹⁰)C(R^{9a})(R^{10a}),
U is a cycle from the group of U-1 to U-28 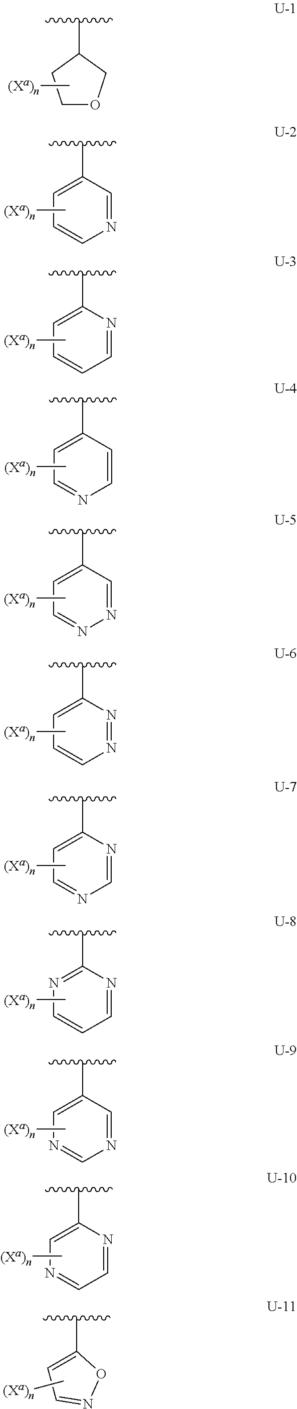 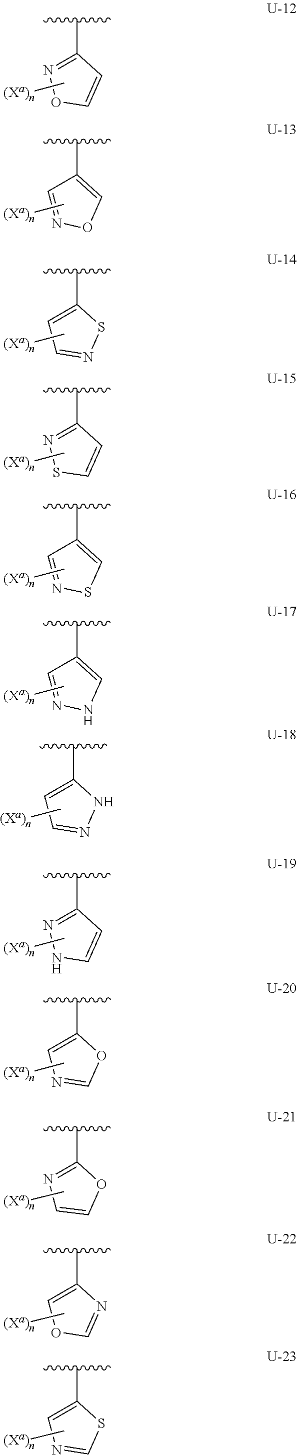 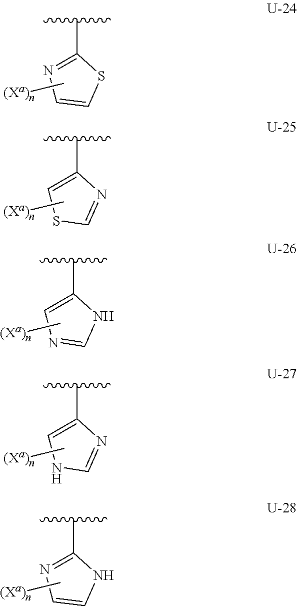
X^{a} is halogen, nitro, OH, cyano, SCN, SF₅, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkoxyimino-C₁-C₆-alkyl, C₁-C₆-alkylsulphinyl, C₁-C₆-alkylsulphonyl, C₁-C₆-haloalkylsulphonyl, C₁-C₆-alkylaminocarbonyl or di-(C₁-C₆)-alkylaminocarbonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy and where the ring nitrogen atoms in U-17, U-18, U-19, U-26, U-27 and U-28 are not substituted by halogen, nitro, OH, cyano, SCN, SF₅, C₁-C₆-alkoxy, C₁-C₆-haloalkoxy, C₁-C₆-alkylthio, C₁-C₆-alkylsulphinyl or C₁-C₄-alkoxy-C₁-C₄-alkyloxy,
n is 0, 1, 2 or 3,
R¹, R², R³, R⁴ are each independently hydrogen, halogen, cyano, nitro, SF₅, SCN, amino, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₂-C₆-alkenyloxy, C₃-C₆-alkynyl, C₃-C₆-alkynyloxy, SH, C₁-C₆-alkylthio, C₁-C₆-alkylsulphinyl, C₁-C₆-alkylsulphonyl, C₁-C₆-haloalkylsulphonyl, C₁-C₆-alkylcarbonyl, C₃-C₆-cycloalkylcarbonyl, C₁-C₆-haloalkylcarbonyl, C₁-C₆-alkoxyimino-C₁-C₆-alkyl, C₁-C₆-alkoxycarbonyl, C₁-C₆-alkylaminocarbonyl, di-(C₁-C₆)-alkylaminocarbonyl, aryl, hetaryl, aryl-C₁-C₆-alkyl or hetaryl-C₁-C₆-alkyl,
where aryl, hetaryl, aryl-C₁-C₆-alkyl and hetaryl-C₁-C₆-alkyl may optionally be mono- or polysubstituted identically or differently by halogen, cyano, nitro, hydroxyl, C₁-C₆-alkyl, C₂-C₆-alkenyl, C₂-C₆-alkynyl, C₃-C₆-cycloalkyl, C₁-C₆-alkoxy, C₁-C₆-haloalkyl, C₁-C₆-haloalkoxy or C₁-C₆-alkylthio,
or
R¹ and R² or
R² and R³ or
R³ and R⁴ in each case together form a 5- or 6-membered aliphatic, aromatic, heteroaromatic or heterocyclic ring which may optionally contain 1 to 2 atoms from the group of O, S and N and which may optionally be mono- or polysubstituted by halogen or by C₁-C₄-alkyl,
R⁵ is hydrogen or
is C₁-C₆-alkyl, C₂-C₆-alkenyl or C₂-C₆-alkynyl, where the aforementioned radicals may optionally independently be mono- to pentasubstituted by halogen or monosubstituted by nitro, cyano, C₃-C₆-cycloalkyl, —O—R⁵¹, —S(O)ₚ—R⁵², —N(R⁵³)(R⁵⁴), —C(═O)N(R⁵³)(R⁵⁴), —O—C(═O)—R⁵⁵, —C(═O)—R⁵⁵, O—SO₂—R⁵⁶, aryl, hetaryl, heterocyclyl or oxoheterocyclyl, where aryl, hetaryl, heterocyclyl or oxoheterocyclyl may in turn each be mono- to trisubstituted by halogen, nitro, cyano, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, C₁-C₆-alkylcarbonylamino, aryl or hetaryl, where aryl and hetaryl may in turn optionally be mono- or polysubstituted identically or differently by halogen, cyano, nitro, hydroxyl, C₁-C₆-alkyl, C₂-C₆-alkenyl, C₂-C₆-alkynyl, C₁-C₆-alkoxy, C₁-C₆-haloalkyl, C₁-C₆-haloalkoxy or C₁-C₆-alkylthio,
or
R⁵ is C₃-C₆-cycloalkyl or C₃-C₆-heterocyclyl, where the aforementioned radicals may optionally independently be mono- to trisubstituted by halogen, or monosubstituted by cyano, C₁-C₆-alkyl, C₁-C₆-haloalkyl, C₁-C₆-alkoxy, C₁-C₆-haloalkoxy, C₃-C₆-cycloalkyl, C₁-C₆-alkylthio, C₁-C₆-alkylsulphinyl, C₁-C₆-alkylsulphonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
p is 0, 1 or 2,
R⁵¹ is hydrogen or
is C₁-C₆-alkyl, C₂-C₆-alkenyl, C₂-C₆-alkynyl, C₃-C₆-cycloalkyl or C₃-C₆-heterocyclyl, where the aforementioned radicals may optionally independently be mono- to trisubstituted by halogen or monosubstituted by nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵¹ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵² is C₁-C₆-alkyl, C₂-C₆-alkenyl, C₂-C₆-alkynyl, C₃-C₆-cycloalkyl or C₃-C₆-heterocyclyl, where the aforementioned radicals may optionally be mono- to trisubstituted and the substituents are independently selected from halogen, nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵² is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵³ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl, C₁-C₆-alkylcarbonyl, aryl, hetaryl, arylcarbonyl or hetarylcarbonyl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
R⁵⁴ is hydrogen or
is C₁-C₆-alkyl, C₂-C₆-alkenyl, C₂-C₆-alkynyl, C₃-C₆-cycloalkyl or C₃-C₆-cycloalkyl-C₁-C₆-alkyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₆-alkoxycarbonyl, C₁-C₆-alkylcarbonyl or C₃-C₆-trialkylsilyl,
or
R⁵⁴ is aryl, hetaryl, aryl-C₁-C₆-alkyl or heteroaryl-C₁-C₆-alkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁵³ and R⁵⁴ may be joined to one another via two to six carbon atoms and form a ring which optionally additionally contains a further atom from the group of O, S or N and may optionally be mono- to tetrasubstituted by C₁-C₂-alkyl, halogen, cyano, amino or C₁-C₂-alkoxy,
R⁵⁵ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁵⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl or aryl-C₁-C₆-alkyl, where aryl, hetaryl and aryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁷ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl or C₁-C₆-alkylcarbonyl,
R^{7a} is hydrogen, C₁-C₆-alkyl, C₂-C₆-alkenyl, C₂-C₆-alkynyl, C₃-C₆-cycloalkyl, C₁-C₄-alkoxy-C₁-C₆-alkyl, C₁-C₆-alkoxycarbonyl or C₁-C₆-alkylcarbonyl,
R⁸ is hydrogen or
is C₁-C₆-alkyl, C₂-C₆-alkenyl or C₂-C₆-alkynyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₁-C₄-alkylsulphimino-C₁-C₄-alkyl, C₁-C₄-alkylsulphimino-C₂-C₅-alkylcarbonyl, C₁-C₄-alkylsulphoximino, C₁-C₄-alkylsulphoximino-C₁-C₄-alkyl, C₁-C₄-alkylsulphoximino-C₂-C₅-alkylcarbonyl, C₁-C₆-alkoxycarbonyl, C₁-C₆-alkylcarbonyl or C₃-C₆-trialkylsilyl,
or
R⁸ is aryl-C₁-C₆-alkyl, hetaryl-C₁-C₆-alkyl, C₃-C₁₂-cycloalkyl, C₃-C₁₂-cycloalkyl-C₁-C₆-alkyl or C₄-C₁₂-bicycloalkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁸ is an optionally singly or multiply, identically or differently substituted 5- or 6-membered aromatic or heteroaromatic ring, a 4- to 6-membered, partly saturated ring or saturated heterocyclic ring, or a saturated or aromatic heterobicyclic ring which may optionally contain 1 to 3 heteroatoms from the group of O, S or N and which may optionally be mono- or polysubstituted, where the substituents are independently selected from halogen, cyano, nitro, hydroxyl, amino, C₁-C₆-alkyl, C₂-C₆-alkynyl, C₁-C₆-haloalkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁷ and R⁸ may be joined to one another via two to six carbon atoms and form a ring which optionally additionally contains a further nitrogen, sulphur or oxygen atom and may optionally be mono- to tetrasubstituted by C₁-C₂-alkyl, halogen, cyano, amino or C₁-C₂-alkoxy,
R⁹ is hydrogen, fluorine, chlorine, C₁-C₄-alkyl, C₃-C₆-cycloalkyl or C₁-C₄-haloalkyl,
R^{9a} is hydrogen, fluorine, chlorine, C₁-C₄-alkyl, C₃-C₆-cycloalkyl or C₁-C₄-haloalkyl,
R¹⁰ is hydrogen, fluorine, chlorine or C₁-C₄-alkyl,
and
R^{10a} is hydrogen, fluorine, chlorine or C₁-C₄-alkyl.

2. Compound according to claim 1, in which
Q¹ and Q² together are one of the A-1, A-4 or A-5 radicals which, together with the carbon and nitrogen atom to which they are bonded, form a 5- or 6-membered aromatic ring,
T is R⁵, —OR⁶, —N(R⁷)(R⁸) or —N(R^{7a})—N(R⁷)(R⁸),
W is O,
G is a bond, is —C(R⁹)(R¹⁰)—, or is C(R⁹)(R¹⁰)C(R^{9a})(R^{10a}),
U is a cycle from the group of U-1, U-2, U-5, U-6, U-9, U-10, U-20 or U-23,
X^{a} is halogen, nitro, cyano, SF₅, C₁-C₄-alkyl, C₁-C₄-haloalkyl, C₁-C₄-alkoxy, C₁-C₄-haloalkoxy, C₁-C₄-alkoxy-C₁-C₄-alkyl, cyano-C₁-C₄-alkyl, C₂-C₄-alkenyl, C₃-C₄-alkynyl, C₁-C₄-alkylthio, C₁-C₄-alkylcarbonyl, C₁-C₄-alkoxycarbonyl, C₁-C₄-alkoxyimino-C₁-C₄-alkyl, C₁-C₄-alkylsulphinyl, C₁-C₄-alkylsulphonyl, C₁-C₄-haloalkylsulphonyl, phenyl, pyridyl, phenyl-C₁-C₄-alkyl or pyridyl-C₁-C₄-alkyl, where phenyl, pyridyl, phenyl-C₁-C₄-alkyl and pyridyl-C₁-C₄-alkyl may each be mono- to trisubstituted by halogen, cyano, nitro, C₁-C₄-alkyl, C₁-C₄-haloalkyl, C₁-C₄-alkoxy or C₁-C₄-haloalkoxy,
n is 0, 1, 2 or 3,
R¹, R², R³, R⁴ are each independently hydrogen, halogen, cyano, C₁-C₄-alkyl, C₃-C₆-cycloalkyl, C₁-C₄-haloalkyl, C₁-C₄-alkoxy, C₁-C₄-haloalkoxy, C₁-C₄-alkoxy-C₁-C₄-alkyl, C₂-C₄-alkenyl, C₂-C₄-alkenyloxy, C₃-C₄-alkynyl, C₃-C₄-alkynyloxy, C₁-C₄-alkylthio, C₁-C₄-alkylsulphinyl, C₁-C₄-alkylsulphonyl, C₁-C₄-alkoxyimino-C₁-C₄-alkyl, C₁-C₄-alkoxycarbonyl, aryl or hetaryl,
where aryl and heteroaryl may optionally be mono- to trisubstituted identically or differently by halogen, cyano, C₁-C₄-alkyl, C₁-C₄-alkoxy, C₁-C₄-haloalkyl, C₁-C₄-haloalkoxy or C₁-C₄-alkylthio,
R⁵ is CH₂Y,
Y is hydrogen, halogen, C₁-C₆-alkyl, C₁-C₆-haloalkyl, —O—R⁵¹, —S(O)ₚ—R⁵², —N(R⁵³)(R⁵⁴), —O—C(═O)—R⁵⁵, O—SO₂—R⁵⁶ or one of the Y-1 to Y-23 radicals: 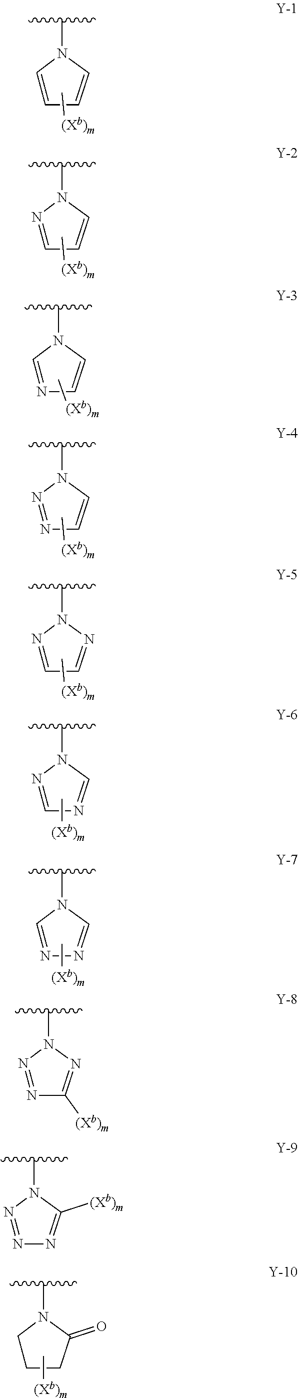 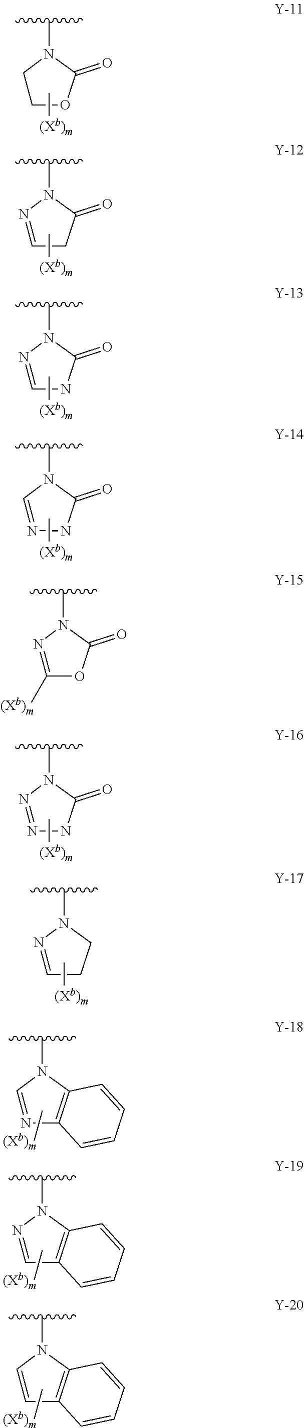 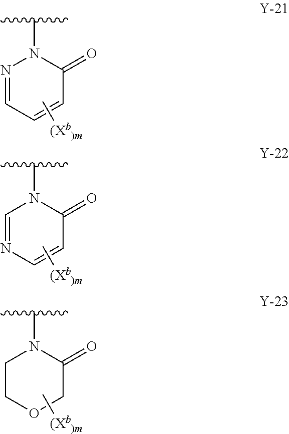
X^{b} is halogen, nitro, cyano, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, C₁-C₆-alkylcarbonylamino, aryl or hetaryl, where aryl and hetaryl may optionally be mono- or polysubstituted identically or differently by halogen, cyano, nitro, hydroxyl, C₁-C₆-alkyl, C₂-C₆-alkenyl, C₂-C₆-alkynyl, C₁-C₆-alkoxy, C₁-C₆-haloalkyl, C₁-C₆-haloalkoxy or C₁-C₆-alkylthio and where the ring nitrogen atoms in Y-13, Y-14 and Y-16 are not substituted by halogen, nitro, cyano, C₁-C₄-alkoxy, C₁-C₄-haloalkoxy or C₁-C₄-alkoxy-C₁-C₄-alkyloxy,
m is 0, 1 or 2,
p is 0, 1 or 2,
R⁵¹ is hydrogen or
is C₁-C₆-alkyl, C₂-C₄-alkenyl, C₂-C₄-alkynyl, C₃-C₆-cycloalkyl or C₃-C₆-heterocyclyl, where the aforementioned radicals may optionally independently be mono- to trisubstituted by halogen or monosubstituted by cyano, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxyimino-C₁-C₄-alkyl, C₁-C₄-alkoxycarbonyl, C₁-C₄-alkylsulphonyl, C₁-C₄-haloalkylsulphonyl, C₁-C₄-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵¹ is aryl or hetaryl, where the aforementioned radicals may optionally be mono- to trisubstituted and the substituents are independently selected from halogen, nitro, cyano, C₁-C₄-alkyl, C₁-C₄-haloalkyl, C₁-C₄-alkoxy, C₁-C₄-haloalkoxy, C₁-C₄-alkylthio or C₁-C₄-alkylsulphonyl,
R⁵² is C₁-C₄-alkyl, C₂-C₄-alkenyl, C₂-C₄-alkynyl, C₃-C₆-cycloalkyl or C₃-C₆-heterocyclyl, where the aforementioned radicals may optionally independently be mono- to trisubstituted by halogen or monosubstituted by cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxyimino-C₁-C₄-alkyl, C₁-C₄-alkoxycarbonyl, C₁-C₄-alkylsulphonyl, C₁-C₄-haloalkylsulphonyl, C₁-C₄-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵² is aryl or hetaryl, where the aforementioned radicals may optionally be mono- to trisubstituted and the substituents are independently selected from halogen, nitro, cyano, C₁-C₄-alkyl, C₁-C₄-haloalkyl, C₁-C₄-alkoxy, C₁-C₄-haloalkoxy, C₁-C₄-alkylthio or C₁-C₄-alkylsulphonyl,
R⁵³ is hydrogen, C₁-C₄-alkyl, C₁-C₄-alkoxycarbonyl, C₁-C₆-alkylcarbonyl, aryl, hetaryl, arylcarbonyl or hetarylcarbonyl, where aryl, hetaryl, arylcarbonyl or hetarylcarbonyl may each be mono- to trisubstituted by halogen, cyano, nitro, C₁-C₄-alkyl, C₁-C₄-haloalkyl, C₁-C₄-alkoxy or C₁-C₄-haloalkoxy,
R⁵⁴ is hydrogen or
is C₁-C₄-alkyl, C₂-C₄-alkenyl, C₂-C₄-alkynyl or C₃-C₄-cycloalkyl, where the aforementioned radicals may optionally independently be mono- to pentasubstituted by halogen or monosubstituted by cyano, C₁-C₄-alkyl, C₃-C₄-cycloalkyl, C₁-C₄-alkoxy, C₁-C₄-haloalkoxy, C₁-C₄-alkylthio, C₁-C₄-alkylsulphinyl, C₁-C₄-alkylsulphonyl or phenyl,
R⁶ is C₁-C₄-alkyl, C₁-C₄-haloalkyl, C₃-C₆-cycloalkyl-C₁-C₄-alkyl, C₁-C₄-alkoxy-C₁-C₄-alkyl, aryl-C₁-C₄-alkyl or hetaryl-C₁-C₄-alkyl, where aryl-C₁-C₄-alkyl and hetaryl-C₁-C₄-alkyl may optionally be mono- to trisubstituted identically or differently by halogen, cyano, nitro, C₁-C₄-alkyl, C₁-C₄-haloalkyl, C₁-C₄-alkoxy or C₁-C₄-haloalkoxy,
R⁷ is hydrogen, C₁-C₄-alkyl, C₂-C₄-alkenyl, C₂-C₄-alkynyl, C₃-C₆-cycloalkyl, C₁-C₄-alkoxy, C₁-C₄-alkoxy-C₁-C₄-alkyl, C₁-C₄-alkoxycarbonyl or C₁-C₄-alkylcarbonyl,
R^{7a} is hydrogen, C₁-C₄-alkyl, C₁-C₄-alkoxycarbonyl or C₁-C₄-alkylcarbonyl,
R⁸ is hydrogen or
is C₁-C₄-alkyl, C₂-C₄-alkenyl or C₂-C₄-alkynyl, where the aforementioned radicals may optionally independently be mono- to trisubstituted by halogen, or monosubstituted by cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkoxycarbonyl, or C₁-C₄-alkylcarbonyl,
or
R⁸ is phenylmethyl, phenylethyl, pyridylmethyl, C₃-C₆-cycloalkyl, C₃-C₆-cycloalkyl-C₁-C₄-alkyl or C₄-C₈-bicycloalkyl, where the aforementioned radicals may optionally be mono- to trisubstituted identically or differently and the substituents are independently selected from halogen, cyano, nitro, hydroxyl, amino, C₁-C₄-alkyl, C₁-C₄-alkoxy, C₁-C₄-haloalkoxy, C₁-C₄-alkylthio, C₁-C₄-alkylsulphinyl, C₁-C₄-alkylsulphonyl, C₂-C₆-alkoxycarbonyl or C₂-C₆-alkylcarbonyl,
or
R⁸ is phenyl, pyridyl, pyrimidinyl, thiazolyl, thiadiazolyl, oxazolyl, pyrazolyl, thienyl or furanyl, where the aforementioned radicals may optionally be mono- to trisubstituted identically or differently and the substituents are independently selected from halogen, cyano, nitro, hydroxyl, amino, C₁-C₄-alkyl, C₂-C₄-alkynyl, C₁-C₄-haloalkyl, C₁-C₄-alkoxy, C₁-C₄-haloalkoxy, C₁-C₄-alkylthio, C₁-C₄-alkylsulphinyl or C₁-C₄-alkylsulphonyl, C₂-C₆-alkoxycarbonyl or C₂-C₆-alkylcarbonyl,
or
R⁷ and R⁸ together are a C₃-C₆-alkyl chain,
R⁹ is hydrogen, fluorine, chlorine or C₁-C₄-alkyl,
R^{9a} is hydrogen, fluorine, chlorine or C₁-C₄-alkyl,
R¹⁰ is hydrogen, fluorine, chlorine or C₁-C₄-alkyl,
and
R^{10a} is hydrogen, fluorine, chlorine or C₁-C₄-alkyl.

3. Compound according to claim 1, in which
Q¹ and Q² together are one of the A-1, A-4 or A-5 radicals which, together with the carbon and nitrogen atom to which they are bonded, form a 5- or 6-membered aromatic ring,
T is R⁵, —OR⁶, —N(R⁷)(R⁸) or —N(R^{7a})—N(R⁷)(R⁸),
W is O,
G is a bond or is —C(R⁹)(R¹⁰)—,
U is a cycle from the group of U-2, U-9, U-10 or U-23,
X^{a} is halogen, cyano, methyl, ethyl, trifluoromethyl, methoxy, ethoxy, trifluoromethoxy, methylthio, methylsulphinyl, methylsulphonyl or methoxycarbonyl,
n is 0, 1 or 2,
R¹, R², R³, R⁴ are each independently a radical from the group of hydrogen, halogen, cyano, C₁-C₄-alkyl, C₁-C₄-haloalkyl, C₁-C₄-alkoxy, C₁-C₄-haloalkoxy, C₁-C₄-alkoxy-C₁-C₄-alkyl, C₂-C₄-alkenyl, C₂-C₄-alkenyloxy, C₃-C₄-alkynyl, C₃-C₄-alkynyloxy, C₁-C₄-alkylthio, C₁-C₄-alkylsulphinyl or C₁-C₄-alkylsulphonyl,
R⁵ is CH₂Y,
Y is hydrogen, Cl, Br, —O—R⁵¹, —S(O)ₚ—R⁵², —N(R⁵³)(R⁵⁴) or is one of the Y-2, Y-3, Y-4, Y-5, Y-6 or Y-7 radicals,
X^{b} is halogen, cyano, methyl, ethyl, trifluoromethyl, difluoromethyl, methylaminocarbonyl, methylcarbonylamino, methoxy, ethoxy, trifluoromethoxy, methylthio, ethylthio, methylsulphinyl, methylsulphonyl or methoxycarbonyl,
m is 0, 1 or 2,
p is 0, 1 or 2,
R⁵¹ is hydrogen or
is C₁-C₆-alkyl, C₂-C₄-alkenyl, C₂-C₄-alkynyl or C₃-C₆-cycloalkyl, where the aforementioned radicals may optionally be mono- to trisubstituted by halogen or monosubstituted by cyano, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxycarbonyl or C₁-C₄-alkylsulphonyl, or may be monosubstituted by phenyl, thienyl, pyridyl, pyrimidinyl or pyrazolyl, which may in turn each independently be mono- to trisubstituted by halogen, cyano, methyl, ethyl, trifluoromethyl, methoxy, ethoxy or trifluoromethoxy,
or
R⁵¹ is phenyl, thienyl, pyridyl, pyrimidinyl or pyrazolyl, where the aforementioned radicals may optionally be mono- to trisubstituted and the substituents are independently selected from halogen, cyano, methyl, ethyl, trifluoromethyl, methoxy, ethoxy or trifluoromethoxy,
R⁵² is C₁-C₄-alkyl or C₃-C₆-cycloalkyl,
where the aforementioned radicals may optionally be mono- to trisubstituted and the substituents are independently selected from halogen, cyano, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylcarbonyl, C₁-C₄-alkoxycarbonyl or C₁-C₄-alkylsulphonyl, and from phenyl, thienyl, pyridyl, pyrimidinyl, pyrazolyl, each of which may independently be mono- to trisubstituted by halogen, cyano, methyl, ethyl, trifluoromethyl, methoxy, ethoxy or trifluoromethoxy,
or
R⁵² is phenyl, thienyl, pyridyl, pyrimidinyl or pyrazolyl, where the aforementioned radicals may optionally be mono- to trisubstituted and the substituents are independently selected from halogen, cyano, methyl, ethyl, trifluoromethyl, methoxy, ethoxy or trifluoromethoxy,
R⁵³ is hydrogen, C₁-C₄-alkyl, C₁-C₄-alkoxycarbonyl or C₁-C₆-alkylcarbonyl,
R⁵⁴ is hydrogen or
is C₁-C₄-alkyl, C₂-C₄-alkenyl, C₂-C₄-alkynyl or C₃-C₄-cycloalkyl, where the aforementioned radicals may optionally be mono- to trisubstituted by halogen, or monosubstituted by cyano, methyl, ethyl, cyclopropyl, methoxy, ethoxy, trifluoromethoxy, methylthio, methylsulphinyl, methylsulphonyl or phenyl,
R⁶ is C₁-C₄-alkyl, C₁-C₄-haloalkyl, C₃-C₆-cycloalkyl-C₁-C₄-alkyl, C₁-C₄-alkoxy-C₁-C₄-alkyl,
R⁷ is hydrogen, C₁-C₄-alkyl, C₁-C₄-alkoxy, C₁-C₄-alkoxy-C₁-C₄-alkyl, C₁-C₄-alkoxycarbonyl or C₁-C₄-alkylcarbonyl,
R^{7a} is hydrogen, C₁-C₄-alkyl, C₁-C₄-alkoxycarbonyl or C₁-C₄-alkylcarbonyl,
R⁸ is hydrogen or
is C₁-C₄-alkyl, C₂-C₄-alkenyl or C₂-C₄-alkynyl, where the aforementioned radicals may optionally independently be mono- to trisubstituted by halogen or monosubstituted by cyano, methoxy, ethoxy, difluoromethoxy, trifluoromethoxy, trifluoroethoxy, methylthio, ethylthio, methylsulphinyl, ethylsulphinyl, methylsulphonyl or ethylsulphonyl,
or
R⁸ is phenylmethyl, phenylethyl, pyridylmethyl, pyridylethyl, C₃-C₆-cycloalkyl or C₃-C₆-cycloalkyl-C₁-C₄-alkyl, where the aforementioned radicals may optionally be mono- to trisubstituted and the substituents are independently selected from halogen, cyano, methyl, ethyl, trifluoromethyl, methoxy, ethoxy, trifluoromethoxy, methylthio, methylsulphinyl, methylsulphonyl or methoxycarbonyl,
or
R⁸ is phenyl, pyridyl, pyrimidinyl, thiazolyl, thiadiazolyl, oxazolyl, pyrazolyl, thienyl or furanyl, where the aforementioned radicals may optionally be mono- to trisubstituted identically or differently and the substituents are independently selected from halogen, cyano, nitro, methyl, ethyl, ethynyl, trifluoromethyl, pentafluoroethyl, methoxy, ethoxy, trifluoromethoxy, methylthio, methylsulphinyl, methylsulphonyl or methoxycarbonyl,
or
R⁷ and R⁸ together are a C₃-C₆-alkyl chain,
R⁹ is hydrogen, fluorine, chlorine, methyl or ethyl,
and
R¹⁰ is hydrogen, fluorine, chlorine, methyl or ethyl.

4. Compound according to claim 1, in which
Q¹ and Q² together are one of the A-1, A-4 or A-5 radicals which, together with the carbon and nitrogen atom to which they are bonded, form a 5- or 6-membered aromatic ring,
T is R⁵, —OR⁶ or —N(R⁷)(R⁸),
W is O,
G is a bond or is —C(R⁹)(R¹⁰)—,
U is U-2, U-9, U-10 or U-23,
X^{a} is fluorine, chlorine, bromine, methyl, ethyl, trifluoromethyl, methoxy, trifluoromethoxy or cyano,
n is 0, 1 or 2,
R¹, R², R³, R⁴ are each independently a radical from the group of hydrogen, fluorine, chlorine, bromine, iodine, cyano, methyl, ethyl, n- or isopropyl, methoxy, ethoxy, n- or isopropoxy, methylthio, ethylthio, difluoromethyl, trifluoromethyl, trifluoromethoxy or difluoromethoxy,
R⁵ is CH₂Y,
Y is hydrogen, chlorine, —O—R⁵¹, —S(O)ₚ—R⁵², —N(R⁵³)(R⁵⁴) or is one of the Y-2, Y-3, Y-4, Y-5, Y-6 or Y-7 radicals,
X^{b} is fluorine, chlorine, bromine, cyano, methyl, ethyl, n- and isopropyl, methoxy, ethoxy, methylthio, ethylthio, difluoromethyl, trifluoromethyl, methylaminocarbonyl or methylcarbonylamino,
m is O or 1,
p is 0, 1 or 2,
R⁵¹ is hydrogen, methyl, ethyl, n- or isopropyl, n-, iso-, sec- or tert-butyl, pent-3-yl, cyclopropyl, cyclobutyl, cyclopentyl, cyclohexyl, cyclopropylmethyl, cyclobutylmethyl, cyclopentylmethyl, cyclohexylmethyl, 2,2,2-trifluoroethyl, 2-methoxyethyl, methoxycarbonylmethyl, phenyl, 2-fluorophenyl, 3-fluorophenyl, 4-fluorophenyl, 2-chlorophenyl, 3-chlorophenyl, 4-chlorophenyl, phenylmethyl, 4-fluorophenylmethyl, 3-fluorophenylmethyl, 2-fluorophenylmethyl, pyrid-3-yl, 6-chloropyrid-3-yl, pyridyl-2-yl or 1-methylpyrazol-5-yl,
R⁵² is methyl, ethyl, n- or isopropyl, phenyl or phenylmethyl,
R⁵³ is hydrogen, methyl, ethyl, methylcarbonyl or ethylcarbonyl,
R⁵⁴ is methyl, ethyl, n- or isopropyl, cyclopropyl or phenylmethyl,
R⁶ is methyl, ethyl or n- or isopropyl,
R⁷ is hydrogen, methyl or ethyl,
R⁸ is methyl, ethyl, n- or isopropyl, n-, iso-, sec- or tert-butyl, trifluoroethyl, cyclopropyl, fluorocyclopropyl, chlorocyclopropyl, cyanocyclopropyl, cyclobutyl, cyclopentyl, cyclohexyl, cyanocyclohexyl, cyclopropylmethyl, cyclopentylmethyl, cyclohexylmethyl, phenyl optionally mono- to disubstituted independently by fluorine, chlorine, methyl, ethyl, methoxy, ethoxy, trifluoromethyl, pentafluoroethyl, trifluoromethoxy, cyano or nitro, phenylmethyl optionally mono- to disubstituted independently by fluorine or chlorine, 1-phenylethyl optionally mono- to disubstituted independently by fluorine or chlorine, 2-phenylethyl optionally mono- to disubstituted independently by fluorine or chlorine, pyrid-3-yl optionally mono- to disubstituted independently by fluorine, chlorine or methyl, pyridyl-2-yl optionally mono- to disubstituted independently by fluorine, chlorine, bromine, methyl, trifluoromethyl, cyano, nitro or ethynyl, pyrid-4-yl optionally mono- to disubstituted independently by fluorine, chlorine or methoxy, 1,3-pyrimidin-2-yl optionally mono- to disubstituted independently by fluorine or chlorine, 1,3-pyrimidin-5-yl optionally mono- to disubstituted independently by fluorine or chlorine, thiazol-2-yl optionally mono- to disubstituted independently by fluorine, chlorine or methyl, 1,2,4-thiadiazol-5-yl, or 1-methylpyrazol-5-yl optionally substituted by chlorine, bromine or methyl,
R⁹ is hydrogen or methyl,
and
R¹⁰ is hydrogen.

5. Compound according to claim 1, comprising a structure according to formula (I-1) 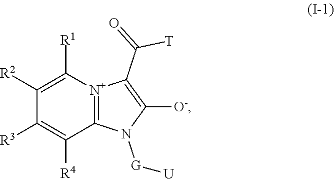
In which Q¹ and Q² together are one of the A-1 to A-5 radicals which, together with the carbon and nitrogen atom to which they are bonded, form a 5- or 6-membered aromatic ring, 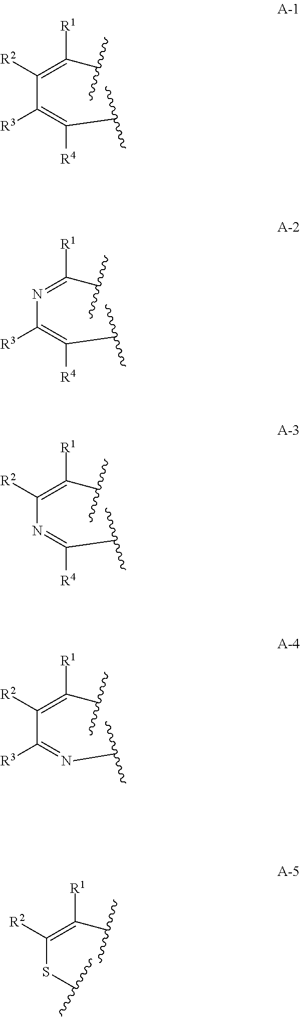
T is R⁵, —OR⁶, —N(R⁷)(R⁸) or —N(R^{7a})—N(R⁷)(R⁸)
and
W is O,
or
T is —N(R⁷)(R⁸) or —N(R^{7a})—N(R⁷)(R⁸)
and
W is S,
G is a bond, is —C(R⁹)(R¹⁰)—, or is C(R⁹)(R¹⁰)C(R^{9a})(R^{10a}),
U is a cycle from the group of U-1 to U-28 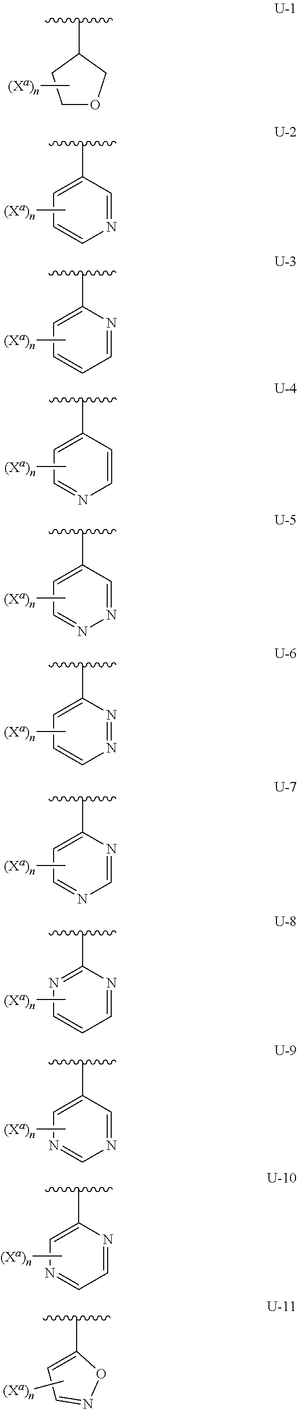 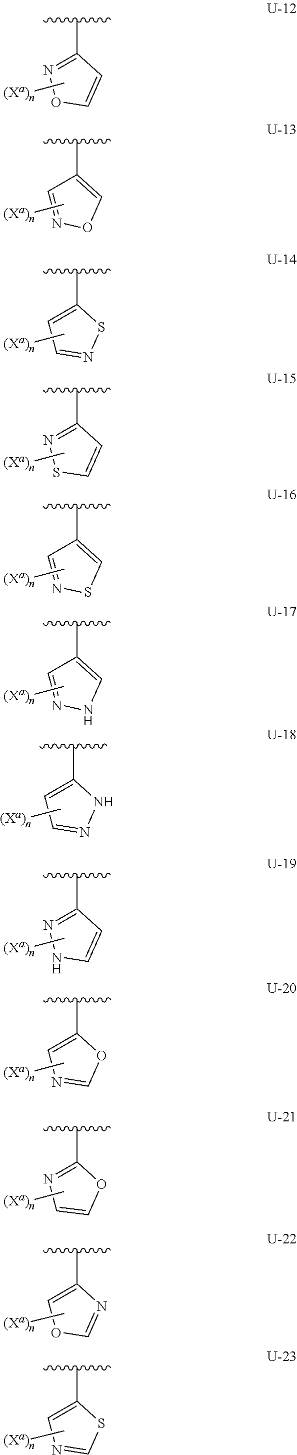 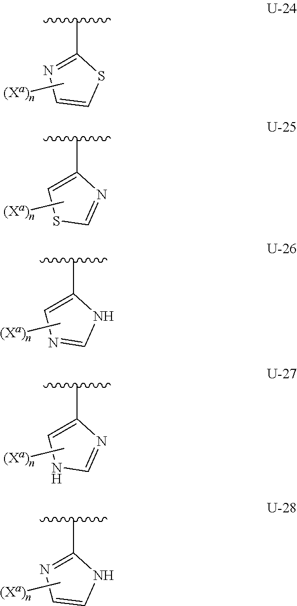
X^{a} is halogen, nitro, OH, cyano, SCN, SF₅, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkoxyimino-C₁-C₆-alkyl, C₁-C₆-alkylsulphinyl, C₁-C₆-alkylsulphonyl, C₁-C₆-haloalkylsulphonyl, C₁-C₆-alkylaminocarbonyl or di-(C₁-C₆)-alkylaminocarbonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy and where the ring nitrogen atoms in U-17, U-18, U-19, U-26, U-27 and U-28 are not substituted by halogen, nitro, OH, cyano, SCN, SF₅, C₁-C₆-alkoxy, C₁-C₆-haloalkoxy, C₁-C₆-alkylthio, C₁-C₆-alkylsulphinyl or C₁-C₄-alkoxy-C₁-C₄-alkyloxy,
n is 0, 1, 2 or 3,
R¹, R², R³, R⁴ are each independently hydrogen, halogen, cyano, nitro, SF₅, SCN, amino, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₂-C₆-alkenyloxy, C₃-C₆-alkynyl, C₃-C₆-alkynyloxy, SH, C₁-C₆-alkylthio, C₁-C₆-alkylsulphinyl, C₁-C₆-alkylsulphonyl, C₁-C₆-haloalkylsulphonyl, C₁-C₆-alkylcarbonyl, C₃-C₆-cycloalkylcarbonyl, C₁-C₆-haloalkylcarbonyl, C₁-C₆-alkoxyimino-C₁-C₆-alkyl, C₁-C₆-alkoxycarbonyl, C₁-C₆-alkylaminocarbonyl, di-(C₁-C₆)-alkylaminocarbonyl, aryl, hetaryl, aryl-C₁-C₆-alkyl or hetaryl-C₁-C₆-alkyl,
where aryl, hetaryl, aryl-C₁-C₆-alkyl and hetaryl-C₁-C₆-alkyl may optionally be mono- or polysubstituted identically or differently by halogen, cyano, nitro, hydroxyl, C₁-C₆-alkyl, C₂-C₆-alkenyl, C₂-C₆-alkynyl, C₃-C₆-cycloalkyl, C₁-C₆-alkoxy, C₁-C₆-haloalkyl, C₁-C₆-haloalkoxy or C₁-C₆-alkylthio,
or
R¹ and R² or
R² and R³ or
R³ and R⁴ in each case together form a 5- or 6-membered aliphatic, aromatic, heteroaromatic or heterocyclic ring which may optionally contain 1 to 2 atoms from the group of O, S and N and which may optionally be mono- or polysubstituted by halogen or by C₁-C₄-alkyl,
R⁵ is hydrogen or
is C₁-C₆-alkyl, C₂-C₆-alkenyl or C₂-C₆-alkynyl, where the aforementioned radicals may optionally independently be mono- to pentasubstituted by halogen or monosubstituted by nitro, cyano, C₃-C₆-cycloalkyl, —O—R⁵¹, —S(O)ₚ—R⁵², —N(R⁵³)(R⁵⁴), —C(═O)N(R⁵³)(R⁵⁴), —O—C(═O)—R⁵⁵, —C(═O)—R⁵⁵, O—SO₂—R⁵⁶, aryl, hetaryl, heterocyclyl or oxoheterocyclyl, where aryl, hetaryl, heterocyclyl or oxoheterocyclyl may in turn each be mono- to trisubstituted by halogen, nitro, cyano, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, C₁-C₆-alkylcarbonylamino, aryl or hetaryl, where aryl and hetaryl may in turn optionally be mono- or polysubstituted identically or differently by halogen, cyano, nitro, hydroxyl, C₁-C₆-alkyl, C₂-C₆-alkenyl, C₂-C₆-alkynyl, C₁-C₆-alkoxy, C₁-C₆-haloalkyl, C₁-C₆-haloalkoxy or C₁-C₆-alkylthio,
or
R⁵ is C₃-C₆-cycloalkyl or C₃-C₆-heterocyclyl, where the aforementioned radicals may optionally independently be mono- to trisubstituted by halogen, or monosubstituted by cyano, C₁-C₆-alkyl, C₁-C₆-haloalkyl, C₁-C₆-alkoxy, C₁-C₆-haloalkoxy, C₃-C₆-cycloalkyl, C₁-C₆-alkylthio, C₁-C₆-alkylsulphinyl, C₁-C₆-alkylsulphonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
p is 0, 1 or 2,
R⁵¹ is hydrogen or
is C₁-C₆-alkyl, C₂-C₆-alkenyl, C₂-C₆-alkynyl, C₃-C₆-cycloalkyl or C₃-C₆-heterocyclyl, where the aforementioned radicals may optionally independently be mono- to trisubstituted by halogen or monosubstituted by nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵¹ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵² is C₁-C₆-alkyl, C₂-C₆-alkenyl, C₂-C₆-alkynyl, C₃-C₆-cycloalkyl or C₃-C₆-heterocyclyl, where the aforementioned radicals may optionally be mono- to trisubstituted and the substituents are independently selected from halogen, nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵² is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵³ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl, C₁-C₆-alkylcarbonyl, aryl, hetaryl, arylcarbonyl or hetarylcarbonyl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
R⁵⁴ is hydrogen or
is C₁-C₆-alkyl, C₂-C₆-alkenyl, C₂-C₆-alkynyl, C₃-C₆-cycloalkyl or C₃-C₆-cycloalkyl-C₁-C₆-alkyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₆-alkoxycarbonyl, C₁-C₆-alkylcarbonyl or C₃-C₆-trialkylsilyl,
or
R⁵⁴ is aryl, hetaryl, aryl-C₁-C₆-alkyl or heteroaryl-C₁-C₆-alkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁵³ and R⁵⁴ may be joined to one another via two to six carbon atoms and form a ring which optionally additionally contains a further atom from the group of O, S or N and may optionally be mono- to tetrasubstituted by C₁-C₂-alkyl, halogen, cyano, amino or C₁-C₂-alkoxy,
R⁵⁵ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁵⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl or aryl-C₁-C₆-alkyl, where aryl, hetaryl and aryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁷ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl or C₁-C₆-alkylcarbonyl,
R^{7a} is hydrogen, C₁-C₆-alkyl, C₂-C₆-alkenyl, C₂-C₆-alkynyl, C₃-C₆-cycloalkyl, C₁-C₄-alkoxy-C₁-C₆-alkyl, C₁-C₆-alkoxycarbonyl or C₁-C₆-alkylcarbonyl,
R⁸ is hydrogen or
is C₁-C₆-alkyl, C₂-C₆-alkenyl or C₂-C₆-alkynyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₁-C₄-alkylsulphimino-C₁-C₄-alkyl, C₁-C₄-alkylsulphimino-C₂-C₅-alkylcarbonyl, C₁-C₄-alkylsulphoximino, C₁-C₄-alkylsulphoximino-C₁-C₄-alkyl, C₁-C₄-alkylsulphoximino-C₂-C₅-alkylcarbonyl, C₁-C₆-alkoxycarbonyl, C₁-C₆-alkylcarbonyl or C₃-C₆-trialkylsilyl,
or
R⁸ is aryl-C₁-C₆-alkyl, hetaryl-C₁-C₆-alkyl, C₃-C₁₂-cycloalkyl, C₃-C₁₂-cycloalkyl-C₁-C₆-alkyl or C₄-C₁₂-bicycloalkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁸ is an optionally singly or multiply, identically or differently substituted 5- or 6-membered aromatic or heteroaromatic ring, a 4- to 6-membered, partly saturated ring or saturated heterocyclic ring, or a saturated or aromatic heterobicyclic ring which may optionally contain 1 to 3 heteroatoms from the group of O, S or N and which may optionally be mono- or polysubstituted, where the substituents are independently selected from halogen, cyano, nitro, hydroxyl, amino, C₁-C₆-alkyl, C₂-C₆-alkynyl, C₁-C₆-haloalkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁷ and R⁸ may be joined to one another via two to six carbon atoms and form a ring which optionally additionally contains a further nitrogen, sulphur or oxygen atom and may optionally be mono- to tetrasubstituted by C₁-C₂-alkyl, halogen, cyano, amino or C₁-C₂-alkoxy,
R⁹ is hydrogen, fluorine, chlorine, C₁-C₄-alkyl, C₃-C₆-cycloalkyl or C₁-C₄-haloalkyl,
R^{9a} is hydrogen, fluorine, chlorine, C₁-C₄-alkyl, C₃-C₆-cycloalkyl or C₁-C₄-haloalkyl,
R¹⁰ is hydrogen, fluorine, chlorine or C₁-C₄-alkyl,
and
R^{10a} is hydrogen, fluorine, chlorine or C₁-C₄-alkyl.

6. Compound according to claim 1, comprising a structure according to formula (I-4) 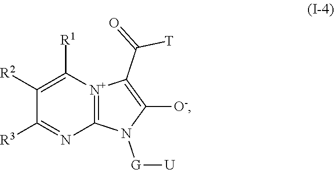
in which
Q¹ and Q² together are one of the A-1 to A-5 radicals which, together with the carbon and nitrogen atom to which they are bonded, form a 5- or 6-membered aromatic ring, 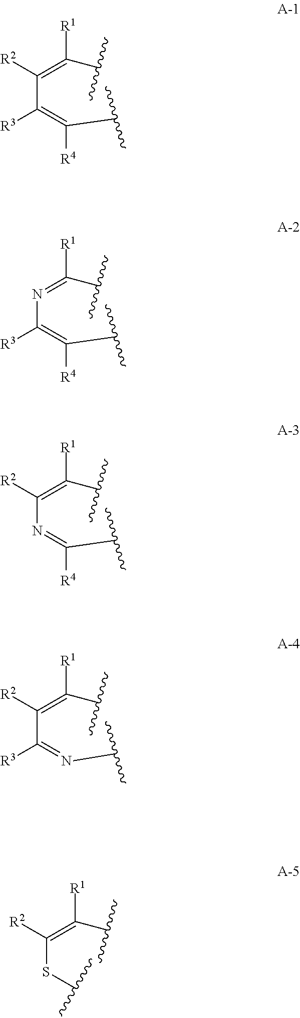
T is R⁵, —OR⁶, —N(R⁷)(R⁸) or —N(R^{7a})—N(R⁷)(R⁸)
and
W is O,
or
T is —N(R⁷)(R⁸) or —N(R^{7a})—N(R⁷)(R⁸)
and
W is S,
G is a bond, is —C(R⁹)(R¹⁰)—, or is C(R⁹)(R¹⁰)C(R^{9a})(R^{10a}),
U is a cycle from the group of U-1 to U-28 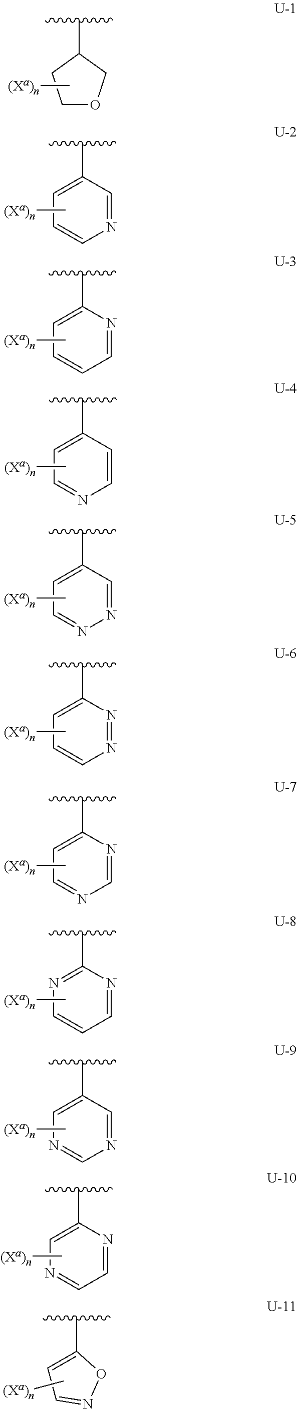 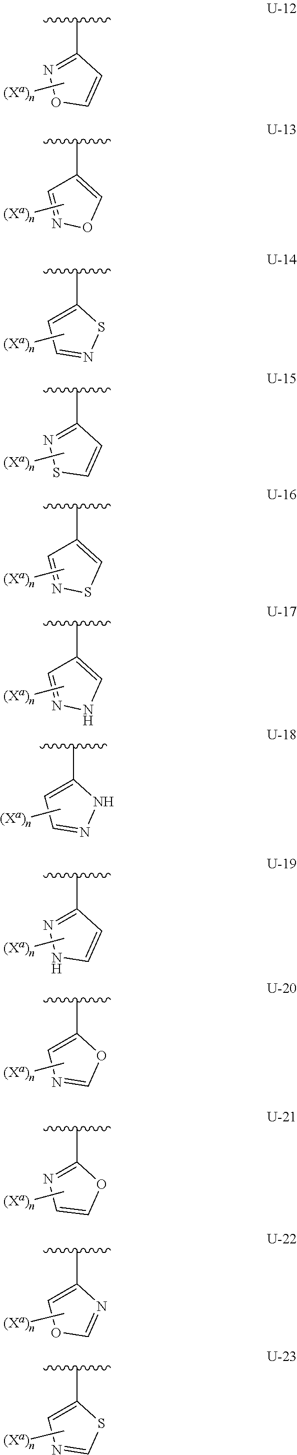 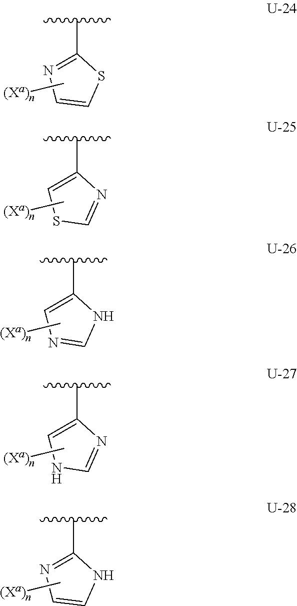
x^{a} is halogen, nitro, OH, cyano, SCN, SF₅, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkoxyimino-C₁-C₆-alkyl, C₁-C₆-alkylsulphinyl, C₁-C₆-alkylsulphonyl, C₁-C₆-haloalkylsulphonyl, C₁-C₆-alkylaminocarbonyl or di-(C₁-C₆)-alkylaminocarbonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy and where the ring nitrogen atoms in U-17, U-18, U-19, U-26, U-27 and U-28 are not substituted by halogen, nitro, OH, cyano, SCN, SF₅, C₁-C₆-alkoxy, C₁-C₆-haloalkoxy, C₁-C₆-alkylthio, C₁-C₆-alkylsulphinyl or C₁-C₄-alkoxy-C₁-C₄-alkyloxy,
n is 0, 1, 2 or 3,
R¹, R², R³, R⁴ are each independently hydrogen, halogen, cyano, nitro, SF₅, SCN, amino, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₂-C₆-alkenyloxy, C₃-C₆-alkynyl, C₃-C₆-alkynyloxy, SH, C₁-C₆-alkylthio, C₁-C₆-alkylsulphinyl, C₁-C₆-alkylsulphonyl, C₁-C₆-haloalkylsulphonyl, C₁-C₆-alkylcarbonyl, C₃-C₆-cycloalkylcarbonyl, C₁-C₆-haloalkylcarbonyl, C₁-C₆-alkoxyimino-C₁-C₆-alkyl, C₁-C₆-alkoxycarbonyl, C₁-C₆-alkylaminocarbonyl, di-(C₁-C₆)-alkylaminocarbonyl, aryl, hetaryl, aryl-C₁-C₆-alkyl or hetaryl-C₁-C₆-alkyl,
where aryl, hetaryl, aryl-C₁-C₆-alkyl and hetaryl-C₁-C₆-alkyl may optionally be mono- or polysubstituted identically or differently by halogen, cyano, nitro, hydroxyl, C₁-C₆-alkyl, C₂-C₆-alkenyl, C₂-C₆-alkynyl, C₃-C₆-cycloalkyl, C₁-C₆-alkoxy, C₁-C₆-haloalkyl, C₁-C₆-haloalkoxy or C₁-C₆-alkylthio,
or
R¹ and R² or
R² and R³ or
R³ and R⁴ in each case together form a 5- or 6-membered aliphatic, aromatic, heteroaromatic or heterocyclic ring which may optionally contain 1 to 2 atoms from the group of O, S and N and which may optionally be mono- or polysubstituted by halogen or by C₁-C₄-alkyl,
R⁵ is hydrogen or
is C₁-C₆-alkyl, C₂-C₆-alkenyl or C₂-C₆-alkynyl, where the aforementioned radicals may optionally independently be mono- to pentasubstituted by halogen or monosubstituted by nitro, cyano, C₃-C₆-cycloalkyl, —O—R⁵¹, —S(O)ₚ—R⁵², —N(R⁵³)(R⁵⁴), —C(═O)N(R⁵³)(R⁵⁴), —O—C(═O)—R⁵⁵, —C(═O)—R⁵⁵, O—SO₂—R⁵⁶, aryl, hetaryl, heterocyclyl or oxoheterocyclyl, where aryl, hetaryl, heterocyclyl or oxoheterocyclyl may in turn each be mono- to trisubstituted by halogen, nitro, cyano, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, C₁-C₆-alkylcarbonylamino, aryl or hetaryl, where aryl and hetaryl may in turn optionally be mono- or polysubstituted identically or differently by halogen, cyano, nitro, hydroxyl, C₁-C₆-alkyl, C₂-C₆-alkenyl, C₂-C₆-alkynyl, C₁-C₆-alkoxy, C₁-C₆-haloalkyl, C₁-C₆-haloalkoxy or C₁-C₆-alkylthio,
or
R⁵ is C₃-C₆-cycloalkyl or C₃-C₆-heterocyclyl, where the aforementioned radicals may optionally independently be mono- to trisubstituted by halogen, or monosubstituted by cyano, C₁-C₆-alkyl, C₁-C₆-haloalkyl, C₁-C₆-alkoxy, C₁-C₆-haloalkoxy, C₃-C₆-cycloalkyl, C₁-C₆-alkylthio, C₁-C₆-alkylsulphinyl, C₁-C₆-alkylsulphonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
p is 0, 1 or 2,
R⁵¹ is hydrogen or
is C₁-C₆-alkyl, C₂-C₆-alkenyl, C₂-C₆-alkynyl, C₃-C₆-cycloalkyl or C₃-C₆-heterocyclyl, where the aforementioned radicals may optionally independently be mono- to trisubstituted by halogen or monosubstituted by nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵¹ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵² is C₁-C₆-alkyl, C₂-C₆-alkenyl, C₂-C₆-alkynyl, C₃-C₆-cycloalkyl or C₃-C₆-heterocyclyl, where the aforementioned radicals may optionally be mono- to trisubstituted and the substituents are independently selected from halogen, nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵² is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵³ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl, C₁-C₆-alkylcarbonyl, aryl, hetaryl, arylcarbonyl or hetarylcarbonyl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
R⁵⁴ is hydrogen or
is C₁-C₆-alkyl, C₂-C₆-alkenyl, C₂-C₆-alkynyl, C₃-C₆-cycloalkyl or C₃-C₆-cycloalkyl-C₁-C₆-alkyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₆-alkoxycarbonyl, C₁-C₆-alkylcarbonyl or C₃-C₆-trialkylsilyl,
or
R⁵⁴ is aryl, hetaryl, aryl-C₁-C₆-alkyl or heteroaryl-C₁-C₆-alkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁵³ and R⁵⁴ may be joined to one another via two to six carbon atoms and form a ring which optionally additionally contains a further atom from the group of O, S or N and may optionally be mono- to tetrasubstituted by C₁-C₂-alkyl, halogen, cyano, amino or C₁-C₂-alkoxy,
R⁵⁵ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁵⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl or aryl-C₁-C₆-alkyl, where aryl, hetaryl and aryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁷ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl or C₁-C₆-alkylcarbonyl,
R^{7a} is hydrogen, C₁-C₆-alkyl, C₂-C₆-alkenyl, C₂-C₆-alkynyl, C₃-C₆-cycloalkyl, C₁-C₄-alkoxy-C₁-C₆-alkyl, C₁-C₆-alkoxycarbonyl or C₁-C₆-alkylcarbonyl,
R⁸ is hydrogen or
is C₁-C₆-alkyl, C₂-C₆-alkenyl or C₂-C₆-alkynyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₁-C₄-alkylsulphimino-C₁-C₄-alkyl, C₁-C₄-alkylsulphimino-C₂-C₅-alkylcarbonyl, C₁-C₄-alkylsulphoximino, C₁-C₄-alkylsulphoximino-C₁-C₄-alkyl, C₁-C₄-alkylsulphoximino-C₂-C₅-alkylcarbonyl, C₁-C₆-alkoxycarbonyl, C₁-C₆-alkylcarbonyl or C₃-C₆-trialkylsilyl,
or
R⁸ is aryl-C₁-C₆-alkyl, hetaryl-C₁-C₆-alkyl, C₃-C₁₂-cycloalkyl, C₃-C₁₂-cycloalkyl-C₁-C₆-alkyl or C₄-C₁₂-bicycloalkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁸ is an optionally singly or multiply, identically or differently substituted 5- or 6-membered aromatic or heteroaromatic ring, a 4- to 6-membered, partly saturated ring or saturated heterocyclic ring, or a saturated or aromatic heterobicyclic ring which may optionally contain 1 to 3 heteroatoms from the group of O, S or N and which may optionally be mono- or polysubstituted, where the substituents are independently selected from halogen, cyano, nitro, hydroxyl, amino, C₁-C₆-alkyl, C₂-C₆-alkynyl, C₁-C₆-haloalkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁷ and R⁸ may be joined to one another via two to six carbon atoms and form a ring which optionally additionally contains a further nitrogen, sulphur or oxygen atom and may optionally be mono- to tetrasubstituted by C₁-C₂-alkyl, halogen, cyano, amino or C₁-C₂-alkoxy,
R⁹ is hydrogen, fluorine, chlorine, C₁-C₄-alkyl, C₃-C₆-cycloalkyl or C₁-C₄-haloalkyl,
R^{9a} is hydrogen, fluorine, chlorine, C₁-C₄-alkyl, C₃-C₆-cycloalkyl or C₁-C₄-haloalkyl,
R¹⁰ is hydrogen, fluorine, chlorine or C₁-C₄-alkyl,
and
R^{10a} is hydrogen, fluorine, chlorine or C₁-C₄-alkyl.

7. Compound according to claim 1, comprising a structure according to formula (I-5) 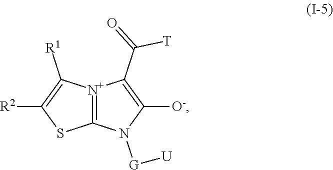
in which
Q¹ and Q² together are one of the A-1 to A-5 radicals which, together with the carbon and nitrogen atom to which they are bonded, form a 5- or 6-membered aromatic ring, 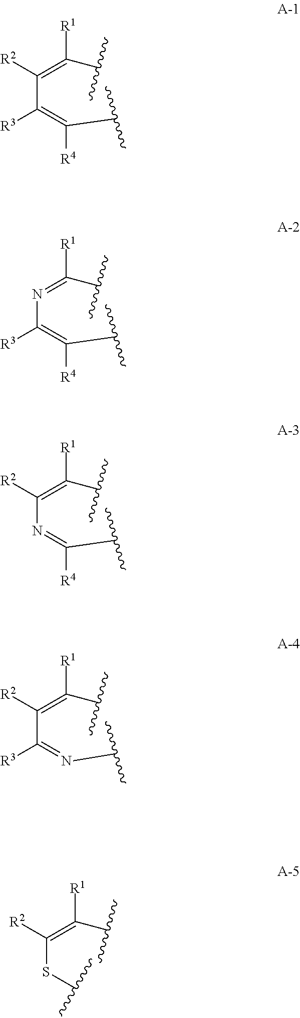
T is R⁵, —OR⁶, —N(R⁷)(R⁸) or —N(R^{7a})—N(R⁷)(R⁸)
and
W is O,
or
T is —N(R⁷)(R⁸) or —N(R^{7a})—N(R⁷)(R⁸)
and
W is S,
G is a bond, is —C(R⁹)(R¹⁰)—, or is C(R⁹)(R¹⁰)C(R^{9a})(R^{10a}),
U is a cycle from the group of U-1 to U-28 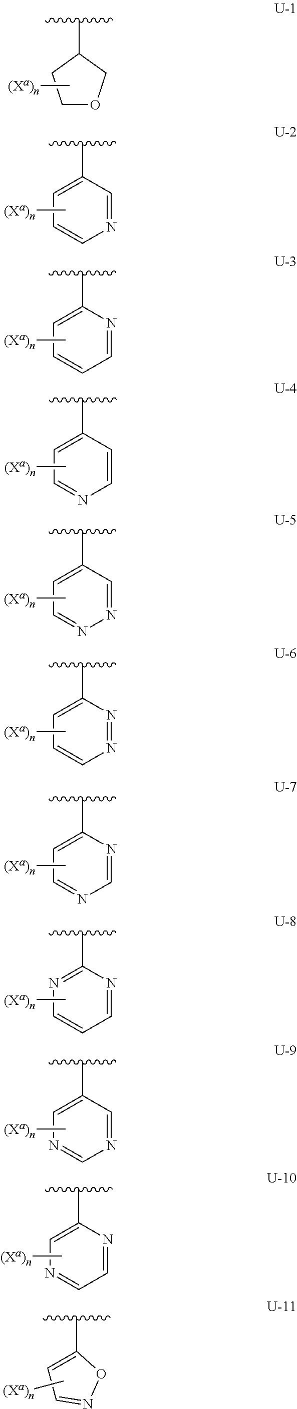 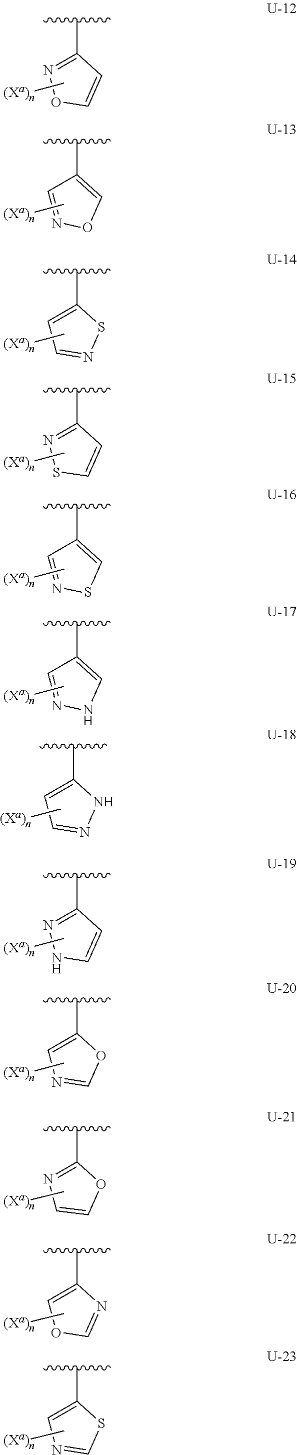 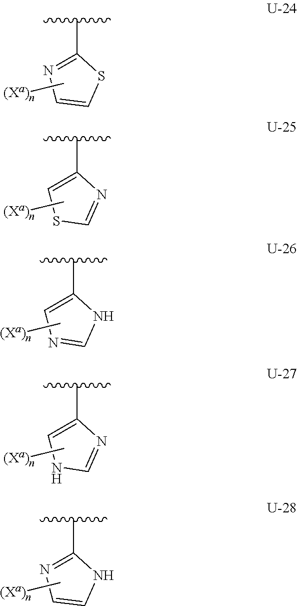
x^{a} is halogen, nitro, OH, cyano, SCN, SF₅, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkoxyimino-C₁-C₆-alkyl, C₁-C₆-alkylsulphinyl, C₁-C₆-alkylsulphonyl, C₁-C₆-haloalkylsulphonyl, C₁-C₆-alkylaminocarbonyl or di-(C₁-C₆)-alkylaminocarbonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy and where the ring nitrogen atoms in U-17, U-18, U-19, U-26, U-27 and U-28 are not substituted by halogen, nitro, OH, cyano, SCN, SF₅, C₁-C₆-alkoxy, C₁-C₆-haloalkoxy, C₁-C₆-alkylthio, C₁-C₆-alkylsulphinyl or C₁-C₄-alkoxy-C₁-C₄-alkyloxy,
n is 0, 1, 2 or 3,
R¹, R², R³, R⁴ are each independently hydrogen, halogen, cyano, nitro, SF₅, SCN, amino, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₂-C₆-alkenyloxy, C₃-C₆-alkynyl, C₃-C₆-alkynyloxy, SH, C₁-C₆-alkylthio, C₁-C₆-alkylsulphinyl, C₁-C₆-alkylsulphonyl, C₁-C₆-haloalkylsulphonyl, C₁-C₆-alkylcarbonyl, C₃-C₆-cycloalkylcarbonyl, C₁-C₆-haloalkylcarbonyl, C₁-C₆-alkoxyimino-C₁-C₆-alkyl, C₁-C₆-alkoxycarbonyl, C₁-C₆-alkylaminocarbonyl, di-(C₁-C₆)-alkylaminocarbonyl, aryl, hetaryl, aryl-C₁-C₆-alkyl or hetaryl-C₁-C₆-alkyl,
where aryl, hetaryl, aryl-C₁-C₆-alkyl and hetaryl-C₁-C₆-alkyl may optionally be mono- or polysubstituted identically or differently by halogen, cyano, nitro, hydroxyl, C₁-C₆-alkyl, C₂-C₆-alkenyl, C₂-C₆-alkynyl, C₃-C₆-cycloalkyl, C₁-C₆-alkoxy, C₁-C₆-haloalkyl, C₁-C₆-haloalkoxy or C₁-C₆-alkylthio,
or
R¹ and R² or
R² and R³ or
R³ and R⁴ in each case together form a 5- or 6-membered aliphatic, aromatic, heteroaromatic or heterocyclic ring which may optionally contain 1 to 2 atoms from the group of O, S and N and which may optionally be mono- or polysubstituted by halogen or by C₁-C₄-alkyl,
R⁵ is hydrogen or
is C₁-C₆-alkyl, C₂-C₆-alkenyl or C₂-C₆-alkynyl, where the aforementioned radicals may optionally independently be mono- to pentasubstituted by halogen or monosubstituted by nitro, cyano, C₃-C₆-cycloalkyl, —O—R⁵¹, —S(O)ₚ—R⁵², —N(R⁵³)(R⁵⁴), —C(═O)N(R⁵³)(R⁵⁴), —O—C(═O)—R⁵⁵, —C(═O)—R⁵⁵, O—SO₂—R⁵⁶, aryl, hetaryl, heterocyclyl or oxoheterocyclyl, where aryl, hetaryl, heterocyclyl or oxoheterocyclyl may in turn each be mono- to trisubstituted by halogen, nitro, cyano, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, C₁-C₆-alkylcarbonylamino, aryl or hetaryl, where aryl and hetaryl may in turn optionally be mono- or polysubstituted identically or differently by halogen, cyano, nitro, hydroxyl, C₁-C₆-alkyl, C₂-C₆-alkenyl, C₂-C₆-alkynyl, C₁-C₆-alkoxy, C₁-C₆-haloalkyl, C₁-C₆-haloalkoxy or C₁-C₆-alkylthio,
or
R⁵ is C₃-C₆-cycloalkyl or C₃-C₆-heterocyclyl, where the aforementioned radicals may optionally independently be mono- to trisubstituted by halogen, or monosubstituted by cyano, C₁-C₆-alkyl, C₁-C₆-haloalkyl, C₁-C₆-alkoxy, C₁-C₆-haloalkoxy, C₃-C₆-cycloalkyl, C₁-C₆-alkylthio, C₁-C₆-alkylsulphinyl, C₁-C₆-alkylsulphonyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
p is 0, 1 or 2,
R⁵¹ is hydrogen or
is C₁-C₆-alkyl, C₂-C₆-alkenyl, C₂-C₆-alkynyl, C₃-C₆-cycloalkyl or C₃-C₆-heterocyclyl, where the aforementioned radicals may optionally independently be mono- to trisubstituted by halogen or monosubstituted by nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵¹ is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵² is C₁-C₆-alkyl, C₂-C₆-alkenyl, C₂-C₆-alkynyl, C₃-C₆-cycloalkyl or C₃-C₆-heterocyclyl, where the aforementioned radicals may optionally be mono- to trisubstituted and the substituents are independently selected from halogen, nitro, cyano, C₃-C₆-cycloalkyl, C₁-C₆-alkoxy, C₁-C₆-haloalkoxy, C₁-C₆-alkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl, aryl or hetaryl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
or
R⁵² is aryl or hetaryl, where the aforementioned radicals may optionally be mono- to trisubstituted and the substituents are independently selected from halogen, nitro, amino, cyano, SF₅, SCN, C₁-C₆-alkylamino, di-(C₁-C₆)-alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di-(C₁-C₆)-alkylaminocarbonyl or tri-(C₁-C₆-alkyl)silyl,
R⁵³ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl, C₁-C₆-alkylcarbonyl, aryl, hetaryl, arylcarbonyl or hetarylcarbonyl, where aryl and hetaryl may each be mono- to trisubstituted by halogen, cyano, nitro, C₁-C₄-alkyl, C₁-C₄-haloalkyl, C₁-C₄-alkoxy or C₁-C₄-haloalkoxy,
R⁵⁴ is hydrogen or
is C₁-C₆-alkyl, C₂-C₆-alkenyl, C₂-C₆-alkynyl, C₃-C₆-cycloalkyl or C₃-C₆-cycloalkyl-C₁-C₆-alkyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₆-alkoxycarbonyl, C₁-C₆-alkylcarbonyl or C₃-C₆-trialkylsilyl,
or
R⁵⁴ is aryl, hetaryl, aryl-C₁-C₆-alkyl or heteroaryl-C₁-C₆-alkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁵³ and R⁵⁴ may be joined to one another via two to six carbon atoms and form a ring which optionally additionally contains a further atom from the group of O, S or N and may optionally be mono- to tetrasubstituted by C₁-C₂-alkyl, halogen, cyano, amino or C₁-C₂-alkoxy,
R⁵⁵ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁵⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, aryl, hetaryl or aryl-C₁-C₆-alkyl, where aryl, hetaryl and aryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁶ is C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy-C₁-C₆-alkyl, C₁-C₆-haloalkoxy-C₁-C₆-alkyl, aryl, hetaryl, aryl-C₁-C₆-alkyl or hetaryl-C₁-C₆-alkyl, where aryl, hetaryl, aryl-C₁-C₆-alkyl and hetaryl-C₁-C₆-alkyl may optionally be mono- or polysubstituted identically or differently by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl or C₁-C₆-alkoxy-C₁-C₆-alkyl,
R⁷ is hydrogen, C₁-C₆-alkyl, C₂-C₆-alkenyl, C₂-C₆-alkynyl, C₃-C₆-cycloalkyl, C₁-C₄-alkoxy, C₁-C₄-alkoxy-C₁-C₆-alkyl, C₁-C₆-alkoxycarbonyl or C₁-C₆-alkylcarbonyl,
R^{7a} is hydrogen, C₁-C₆-alkyl, C₂-C₆-alkenyl, C₂-C₆-alkynyl, C₃-C₆-cycloalkyl, C₁-C₄-alkoxy-C₁-C₆-alkyl, C₁-C₆-alkoxycarbonyl or C₁-C₆-alkylcarbonyl,
R⁸ is hydrogen or
is C₁-C₆-alkyl, C₂-C₆-alkenyl or C₂-C₆-alkynyl, where the aforementioned radicals may optionally independently be mono- to pentasubstituted by halogen or monosubstituted by cyano, nitro, hydroxyl, C₁-C₆-alkyl, C₃-C₆-cycloalkyl, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₁-C₄-alkylsulphimino-C₁-C₄-alkyl, C₁-C₄-alkylsulphimino-C₂-C₅-alkylcarbonyl, C₁-C₄-alkylsulphoximino, C₁-C₄-alkylsulphoximino-C₁-C₄-alkyl, C₁-C₄-alkylsulphoximino-C₂-C₅-alkylcarbonyl, C₁-C₆-alkoxycarbonyl, C₁-C₆-alkylcarbonyl or C₃-C₆-trialkylsilyl,
or
R⁸ is aryl-C₁-C₆-alkyl, hetaryl-C₁-C₆-alkyl, C₃-C₁₂-cycloalkyl, C₃-C₁₂-cycloalkyl-C₁-C₆-alkyl or C₄-C₁₂-bicycloalkyl, where the aforementioned radicals may optionally be substituted and the substituents are independently selected from halogen, cyano, nitro, hydroxyl, amino, C₁-C₆-alkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁸ is an optionally singly or multiply, identically or differently substituted 5- or 6-membered aromatic or heteroaromatic ring, a 4- to 6-membered, partly saturated ring or saturated heterocyclic ring, or a saturated or aromatic heterobicyclic ring which may optionally contain 1 to 3 heteroatoms from the group of O, S or N and which may optionally be mono- or polysubstituted, where the substituents are independently selected from halogen, cyano, nitro, hydroxyl, amino, C₁-C₆-alkyl, C₂-C₆-alkynyl, C₁-C₆-haloalkyl, C₃-C₆-cycloalkyl, C₃-C₆-cycloalkylamino, C₁-C₄-alkoxy, C₁-C₄-haloalkoxy, C₁-C₄-alkylthio, C₁-C₄-alkylsulphinyl, C₁-C₄-alkylsulphonyl, C₁-C₄-alkylsulphimino, C₂-C₆-alkoxycarbonyl or C₂-C₆-alkylcarbonyl,
or
R⁷ and R⁸ may be joined to one another via two to six carbon atoms and form a ring which optionally additionally contains a further nitrogen, sulphur or oxygen atom and may optionally be mono- to tetrasubstituted by C₁-C₂-alkyl, halogen, cyano, amino or C₁-C₂-alkoxy,
R⁹ is hydrogen, fluorine, chlorine, C₁-C₄-alkyl, C₃-C₆-cycloalkyl or C₁-C₄-haloalkyl,
R^{9a} is hydrogen, fluorine, chlorine, C₁-C₄-alkyl, C₃-C₆-cycloalkyl or C₁-C₄-haloalkyl,
R¹⁰ is hydrogen, fluorine, chlorine or C₁-C₄-alkyl,
and
R^{10a} is hydrogen, fluorine, chlorine or C₁-C₄-alkyl.

8. Formulation, optionally an agrochemical formulation,
comprising at least one compound of the formula (I) according to claim 1.

9. Formulation according to claim 8, further comprising at least one extender and/or at least one surface-active substance.

10. Formulation according to claim 8, wherein the compound of the formula (I) is in a mixture with at least one further active ingredient.

11. Method for controlling one or more pests, optionally animal pests, wherein a compound of the formula (I) according to claim 1 is allowed to act on the pests and/or a habitat thereof.

12. Method according to claim 11, wherein the pest is an animal pest and comprises an insect, an arachnid or a nematode, or the pest is an insect, an arachnid or a nematode.

13. A compound of the formula (I) according to claim 1 for controlling animal pests.

14. A compound according to claim 13, wherein the animal pest comprises an insect, an arachnid or a nematode, or the animal pest is an insect, an arachnid or a nematode.

15. A compound according to claim 13 in crop protection.

16. A compound according to claim 13 in the field of animal health.

17. Method for protecting seed or a germinating plant from one or more pests, optionally animal pests, comprising contacting the seed with a compound of the formula (I) according to claim 1.

18. Seed obtained by a method according to claim 17.
